SEQUENCE LISTING (323 sequences)

SEQ ID NO: 1 - DNA, 22 bp, Artificial Sequence
  Forward primer for amplifying biomarker ANUCS501
agcaataatggagtgagtgaac

SEQ ID NO: 2 - DNA, 23 bp, Artificial Sequence
  Reverse primer for amplifying biomarker ANUCS501
agagatcaagaaattgagattcc

SEQ ID NO: 3 - DNA, 22 bp, Artificial Sequence
  Forward primer for amplifying biomarker C11-CANN1
gtggtggtgatgatgataatgg

SEQ ID NO: 4 - DNA, 19 bp, Artificial Sequence
  Reverse primer for amplifying biomarker C11-CANN1
tgaattggttacgatggcg

SEQ ID NO: 5 - DNA, 19 bp, Artificial Sequence
  Forward primer for amplifying biomarker E07-CANN1
caaatgccacaccaccttc

SEQ ID NO: 6 - DNA, 22 bp, Artificial Sequence
  Reverse primer for amplifying biomarker E07-CANN1 and E07-CANN1ana4
gtaggtagccaggtataggtag

SEQ ID NO: 7 - DNA, 19 bp, Artificial Sequence
  Forward primer for amplifying biomarker H06-CANN2
tggtttcagtggtcctctc

SEQ ID NO: 8 - DNA, 19 bp, Artificial Sequence
  Reverse primer for amplifying biomarker H06-CANN2
acgtgagtgatgacacgag

SEQ ID NO: 9 - DNA, 21 bp, Artificial Sequence
  Forward primer for amplifying biomarker B01-CANN1
tggagtcaaatgaaagggaac

SEQ ID NO: 10 - DNA, 22 bp, Artificial Sequence
  Reverse primer for amplifying biomarker B01-CANN1
ccatagcattatcccactcaag

SEQ ID NO: 11 - DNA, 21 bp, Artificial Sequence
  Forward primer for amplifying biomarker D02-CANN1
ggttgggatgttgttgttgtg

SEQ ID NO: 12 - DNA, 21 bp, Artificial Sequence
  Reverse primer for amplifying biomarker D02-CANN1
agaaatccaaggtcctgatgg

SEQ ID NO: 13 - DNA, 20 bp, Artificial Sequence
  Forward primer for amplifying biomarker CAN0055
caccttctggctcttatggt

SEQ ID NO: 14 - DNA, 20 bp, Artificial Sequence
  Reverse primer for amplifying biomarker CAN0055
ctgttactcgaattgggtcc

SEQ ID NO: 15 - DNA, 20 bp, Artificial Sequence
  Forward primer for amplifying biomarker CAN0111
aagagctggaacgaacaaag

SEQ ID NO: 16 - DNA, 20 bp, Artificial Sequence
  Reverse primer for amplifying biomarker CAN0111
ctggtgggatcagaacttgt

SEQ ID NO: 17 - DNA, 20 bp, Artificial Sequence
  Forward primer for amplifying biomarker CAN0509
cagcagaatcagtcgtcatc

SEQ ID NO: 18 - DNA, 20 bp, Artificial Sequence
  Reverse primer for amplifying biomarker CAN0509
taactgttgctgttgcaggt

SEQ ID NO: 19 - DNA, 20 bp, Artificial Sequence
  Forward primer for amplifying biomarker CAN0717
acgcacacaggtgtattctg

SEQ ID NO: 20 - DNA, 20 bp, Artificial Sequence
  Reverse primer for amplifying biomarker CAN0717
gctcatgtttttgagggact

SEQ ID NO: 21 - DNA, 20 bp, Artificial Sequence
  Forward primer for amplifying biomarker CAN2550
gagcccatttcagtttcagt

SEQ ID NO: 22 - DNA, 20 bp, Artificial Sequence
  Reverse primer for amplifying biomarker CAN2550
gattaggcctgaaagtggtg

SEQ ID NO: 23 - DNA, 19 bp, Artificial Sequence
  Forward primer for amplifying biomarker CAN0009
ctccaaacccaacaacaca

SEQ ID NO: 24 - DNA, 20 bp, Artificial Sequence
  Reverse primer for amplifying biomarker CAN0009
tcaagccatgagagttgaga

SEQ ID NO: 25 - DNA, 20 bp, Artificial Sequence
  Forward primer for amplifying biomarker ANUCS301
atatggttgaaatccattgc

SEQ ID NO: 26 - DNA, 20 bp, Artificial Sequence
  Reverse primer for amplifying biomarker ANUCS301
taacaaagtttcgtgagggt

SEQ ID NO: 27 - DNA, 20 bp, Artificial Sequence
  Forward primer for amplifying biomarker ANUCS303
taatcaacaatgacaatggc

SEQ ID NO: 28 - DNA, 20 bp, Artificial Sequence
  Reverse primer for amplifying biomarker ANUCS303
gattaaggtcctcgacgata

SEQ ID NO: 29 - DNA, 20 bp, Artificial Sequence
  Forward primer for amplifying biomarker ANUCS304
tcttcactcacctcctctct

SEQ ID NO: 30 - DNA, 18 bp, Artificial Sequence
  Reverse primer for amplifying biomarker ANUCS304
tctttaagcgggactcgt

SEQ ID NO: 31 - DNA, 21 bp, Artificial Sequence
  Forward primer for amplifying biomarker B02-CANN2
caaccaaatgagaatgcaacc

SEQ ID NO: 32 - DNA, 19 bp, Artificial Sequence
  Reverse primer for amplifying biomarker B02-CANN2
tgttttcttcactgcaccc

SEQ ID NO: 33 - DNA, 19 bp, Artificial Sequence
  Forward primer for amplifying biomarker B05-CANN1
ttgatggtggtgaaacggc

SEQ ID NO: 34 - DNA, 21 bp, Artificial Sequence
  Reverse primer for amplifying biomarker B05-CANN1
ccccaatctcaatctcaaccc

SEQ ID NO: 35 - DNA, 20 bp, Artificial Sequence
  Forward primer for amplifying biomarker CAN0126
gagtaagagaaggcgaacca

SEQ ID NO: 36 - DNA, 20 bp, Artificial Sequence
  Reverse primer for amplifying biomarker CAN0126
cctgtgtaacagaaaacccc

SEQ ID NO: 37 - DNA, 20 bp, Artificial Sequence
  Forward primer for amplifying biomarker CAN1347
tgtttctaaggctcagtccc

SEQ ID NO: 38 - DNA, 20 bp, Artificial Sequence
  Reverse primer for amplifying biomarker CAN1347
ggcaaaggtaaagcaagtgt

SEQ ID NO: 39 - DNA, 20 bp, Artificial Sequence
  Forward primer for amplifying biomarker CAN1482
ttccccaactatccaatcac

SEQ ID NO: 40 - DNA, 20 bp, Artificial Sequence
  Reverse primer for amplifying biomarker CAN1482
atttgagaaggggttaggct

SEQ ID NO: 41 - DNA, 20 bp, Artificial Sequence
  Forward primer for amplifying biomarker CAN2913
aggaacactttgaaagcgag

SEQ ID NO: 42 - DNA, 20 bp, Artificial Sequence
  Reverse primer for amplifying biomarker CAN2913
cggtcatctaccttgagctt

SEQ ID NO: 43 - DNA, 20 bp, Artificial Sequence
  Forward primer for amplifying biomarker NM101
aagcaactccaattccagcc

SEQ ID NO: 44 - DNA, 23 bp, Artificial Sequence
  Reverse primer for amplifying biomarker NM101
taatgatgagacgagtgagaacg

SEQ ID NO: 45 - DNA, 21 bp, Artificial Sequence
  Forward primer for amplifying biomarker ANUCS305
aaagttggtctgagaagcaat

SEQ ID NO: 46 - DNA, 20 bp, Artificial Sequence
  Reverse primer for amplifying biomarker ANUCS305
cctaggaactttcgacaaca

SEQ ID NO: 47 - DNA, 20 bp, Artificial Sequence
  Forward primer for amplifying biomarker CAN0010
tccaaacgttctctctctcc

SEQ ID NO: 48 - DNA, 21 bp, Artificial Sequence
  Reverse primer for amplifying biomarker CAN0010
ctactaacccaatcagaccca

SEQ ID NO: 49 - DNA, 20 bp, Artificial Sequence
  Forward primer for amplifying biomarker CAN0878
gatgaacatgaaacagcagc

SEQ ID NO: 50 - DNA, 20 bp, Artificial Sequence
  Reverse primer for amplifying biomarker CAN0878
atatgagccatgaccgaagt

SEQ ID NO: 51 - DNA, 21 bp, Artificial Sequence
  Forward primer for amplifying biomarker CAN2717
tgatgacagagtgatagcagc

SEQ ID NO: 52 - DNA, 20 bp, Artificial Sequence
  Reverse primer for amplifying biomarker CAN2717
ctaatcaatgcggtaaaccc

SEQ ID NO: 53 - DNA, 20 bp, Artificial Sequence
  Forward primer for amplifying biomarker ANUCS201
ggttcaatggagattctcgt

SEQ ID NO: 54 - DNA, 23 bp, Artificial Sequence
  Reverse primer for amplifying biomarker ANUCS201
ccactaaaccaaaagtactcttc

SEQ ID NO: 55 - DNA, 23 bp, Artificial Sequence
  Forward primer for amplifying biomarker ANUCS203
gctcttcttattaattcctcctt

SEQ ID NO: 56 - DNA, 25 bp, Artificial Sequence
  Reverse primer for amplifying biomarker ANUCS203
gaatatgataagacacaacttcatt

SEQ ID NO: 57 - DNA, 20 bp, Artificial Sequence
  Forward primer for amplifying biomarker ANUCS205
ttgactaaccggcaaagata

SEQ ID NO: 58 - DNA, 21 bp, Artificial Sequence
  Reverse primer for amplifying biomarker ANUCS205
aaattcaaaaccgattctcag

SEQ ID NO: 59 - DNA, 22 bp, Artificial Sequence
  Forward primer for amplifying biomarker C08-CANN2
gcaagaagtagagagacaatcc

SEQ ID NO: 60 - DNA, 22 bp, Artificial Sequence
  Reverse primer for amplifying biomarker C08-CANN2
ccctcaacactcattaactcac

SEQ ID NO: 61 - DNA, 21 bp, Artificial Sequence
  Forward primer for amplifying biomarker ANUCS302
aacataaacaccaacaactgc

SEQ ID NO: 62 - DNA, 20 bp, Artificial Sequence
  Reverse primer for amplifying biomarker ANUCS302
atggttgatgttttgatggt

SEQ ID NO: 63 - DNA, 20 bp, Artificial Sequence
  Forward primer for amplifying biomarker CAN0024
gatactggctcacggatctt

SEQ ID NO: 64 - DNA, 20 bp, Artificial Sequence
  Reverse primer for amplifying biomarker CAN0024
cgcctcttcttaacctgtgt

SEQ ID NO: 65 - DNA, 20 bp, Artificial Sequence
  Forward primer for amplifying biomarker CAN0026
aggagcagaacaaaatggag

SEQ ID NO: 66 - DNA, 20 bp, Artificial Sequence
  Reverse primer for amplifying biomarker CAN0026
taaaccgggtcaaccatact

SEQ ID NO: 67 - DNA, 20 bp, Artificial Sequence
  Forward primer for amplifying biomarker CAN0039
gcagccatagtcatggtgta

SEQ ID NO: 68 - DNA, 20 bp, Artificial Sequence
  Reverse primer for amplifying biomarker CAN0039
gtcattggaaagaccagctt

SEQ ID NO: 69 - DNA, 22 bp, Artificial Sequence
  Forward primer for amplifying biomarker CAN0093
cagtctctcagatcagactacc

SEQ ID NO: 70 - DNA, 20 bp, Artificial Sequence
  Reverse primer for amplifying biomarker CAN0093
agcggctagcgtaacagtat

SEQ ID NO: 71 - DNA, 20 bp, Artificial Sequence
  Forward primer for amplifying biomarker CAN0107A
aagtggcgaactggtttatc

SEQ ID NO: 72 - DNA, 20 bp, Artificial Sequence
  Reverse primer for amplifying biomarker CAN0107A
aagccagcgttattatcgac

SEQ ID NO: 73 - DNA, 20 bp, Artificial Sequence
  Forward primer for amplifying biomarker CAN0110
gggtaaagcttacgcaaagt

SEQ ID NO: 74 - DNA, 20 bp, Artificial Sequence
  Reverse primer for amplifying biomarker CAN0110
aacaaacagttggacaccct

SEQ ID NO: 75 - DNA, 20 bp, Artificial Sequence
  Forward primer for amplifying biomarker CAN0585
tcatcatcatccctccctat

SEQ ID NO: 76 - DNA, 20 bp, Artificial Sequence
  Reverse primer for amplifying biomarker CAN0585
ggtccatagttggctgatct

SEQ ID NO: 77 - DNA, 20 bp, Artificial Sequence
  Forward primer for amplifying biomarker CAN0663
catcacagaggacccttttc

SEQ ID NO: 78 - DNA, 20 bp, Artificial Sequence
  Reverse primer for amplifying biomarker CAN0663
gtctccaccaccaatctttc

SEQ ID NO: 79 - DNA, 20 bp, Artificial Sequence
  Forward primer for amplifying biomarker CAN0672
tgcagcaccaacttactttc

SEQ ID NO: 80 - DNA, 20 bp, Artificial Sequence
  Reverse primer for amplifying biomarker CAN0672
gcattgtatttcccttccac

SEQ ID NO: 81 - DNA, 20 bp, Artificial Sequence
  Forward primer for amplifying biomarker CAN0865
cttctctaatccgattccca

SEQ ID NO: 82 - DNA, 20 bp, Artificial Sequence
  Reverse primer for amplifying biomarker CAN0865
gaacgaagaaaacccagaag

SEQ ID NO: 83 - DNA, 20 bp, Artificial Sequence
  Forward primer for amplifying biomarker CAN1001
ctagttacgggggaattcaa

SEQ ID NO: 84 - DNA, 20 bp, Artificial Sequence
  Reverse primer for amplifying biomarker CAN1001
ctttcccaactcgtatttgc

SEQ ID NO: 85 - DNA, 20 bp, Artificial Sequence
  Forward primer for amplifying biomarker CAN1423
ctccacaatctccaatctcc

SEQ ID NO: 86 - DNA, 20 bp, Artificial Sequence
  Reverse primer for amplifying biomarker CAN1423
gctacggtaacggaatcaac

SEQ ID NO: 87 - DNA, 20 bp, Artificial Sequence
  Forward primer for amplifying biomarker CAN1690B
caaacaggggaaaagagaga

SEQ ID NO: 88 - DNA, 20 bp, Artificial Sequence
  Reverse primer for amplifying biomarker CAN1690B
atgaagcgttggtactaggc

SEQ ID NO: 89 - DNA, 20 bp, Artificial Sequence
  Forward primer for amplifying biomarker CAN2167
caaatgggttcctctcaatc

SEQ ID NO: 90 - DNA, 20 bp, Artificial Sequence
  Reverse primer for amplifying biomarker CAN2167
acgaggcgtacctaatgaag

SEQ ID NO: 91 - DNA, 20 bp, Artificial Sequence
  Forward primer for amplifying biomarker CAN2707
taatcagttgtacggaacgc

SEQ ID NO: 92 - DNA, 20 bp, Artificial Sequence
  Reverse primer for amplifying biomarker CAN2707
tgactctgtgaatcagtggg

SEQ ID NO: 93 - DNA, 20 bp, Artificial Sequence
  Forward primer for amplifying biomarker CAN3023
cctctactgggacacacaca

SEQ ID NO: 94 - DNA, 20 bp, Artificial Sequence
  Reverse primer for amplifying biomarker CAN3023
ccttcttcttaacagggcaa

SEQ ID NO: 95 - DNA, 20 bp, Artificial Sequence
  Forward primer for amplifying biomarker CAN3037
cgaactcttttagcattgcc

SEQ ID NO: 96 - DNA, 20 bp, Artificial Sequence
  Reverse primer for amplifying biomarker CAN3037
cgggggattaaaagagaaga

SEQ ID NO: 97 - DNA, 20 bp, Artificial Sequence
  Forward primer for amplifying biomarker CAN3087
accgagaaaaccaactcatc

SEQ ID NO: 98 - DNA, 20 bp, Artificial Sequence
  Reverse primer for amplifying biomarker CAN3087
tctcgtttcggtcagatgta

SEQ ID NO: 99 - DNA, 21 bp, Artificial Sequence
  Forward primer for amplifying biomarker H09-CANN2
cgtacagtgatcgtagttgag

SEQ ID NO: 100 - DNA, 19 bp, Artificial Sequence
  Reverse primer for amplifying biomarker H09-CANN2
acacatacagagagagccc

SEQ ID NO: 101 - DNA, 23 bp, Artificial Sequence
  3′ flanking sequence of biomarker ANUCS501
ggaatctcaatttcttgatctct

SEQ ID NO: 102 - DNA, 19 bp, Artificial Sequence
  3′ flanking sequence of biomarker C11-CANN1
cgccatcgtaaccaattca

SEQ ID NO: 103 - DNA, 22 bp, Artificial Sequence
  3′ flanking sequence of biomarker E07-CANN1
ctacctatacctggctacctac

SEQ ID NO: 104 - DNA, 19 bp, Artificial Sequence
  3′ flanking sequence of biomarker H06-CANN2
ctcgtgtcatcactcacgt

SEQ ID NO: 105 - DNA, 22 bp, Artificial Sequence
  3′ flanking sequence of biomarker B01-CANN1
cttgagtgggataatgctatgg

SEQ ID NO: 106 - DNA, 21 bp, Artificial Sequence
  3′ flanking sequence of biomarker D02-CANN1
ccatcaggaccttggatttct

SEQ ID NO: 107 - DNA, 20 bp, Artificial Sequence
  3′ flanking sequence of biomarker CAN0055
ggacccaattcgagtaacag

SEQ ID NO: 108 - DNA, 20 bp, Artificial Sequence
  3′ flanking sequence of biomarker CAN0111
acaagttctgatcccaccag

SEQ ID NO: 109 - DNA, 20 bp, Artificial Sequence
  3′ flanking sequence of biomarker CAN0509
acctgcaacagcaacagtta

SEQ ID NO: 110 - DNA, 20 bp, Artificial Sequence
  3′ flanking sequence of biomarker CAN0717
agtccctcaaaaacatgagc

SEQ ID NO: 111 - DNA, 20 bp, Artificial Sequence
  3′ flanking sequence of biomarker CAN2550
caccactttcaggcctaatc

SEQ ID NO: 112 - DNA, 20 bp, Artificial Sequence
  3′ flanking sequence of biomarker CAN0009
tctcaactctcatggcttga

SEQ ID NO: 113 - DNA, 20 bp, Artificial Sequence
  3′ flanking sequence of biomarker ANUCS301
accctcacgaaactttgtta

SEQ ID NO: 114 - DNA, 20 bp, Artificial Sequence
  3′ flanking sequence of biomarker ANUCS303
tatcgtcgaggaccttaatc

SEQ ID NO: 115 - DNA, 18 bp, Artificial Sequence
  3′ flanking sequence of biomarker ANUCS304
acgagtcccgcttaaaga

SEQ ID NO: 116 - DNA, 19 bp, Artificial Sequence
  3′ flanking sequence of biomarker B02-CANN2
gggtgcagtgaagaaaaca

SEQ ID NO: 117 - DNA, 21 bp, Artificial Sequence
  3′ flanking sequence of biomarker B05-CANN1
gggttgagattgagattgggg

SEQ ID NO: 118 - DNA, 20 bp, Artificial Sequence
  3′ flanking sequence of biomarker CAN0126
ggggttttctgttacacagg

SEQ ID NO: 119 - DNA, 20 bp, Artificial Sequence
  3′ flanking sequence of biomarker CAN1347
acacttgctttacctttgcc

SEQ ID NO: 120 - DNA, 20 bp, Artificial Sequence
  3′ flanking sequence of biomarker CAN1482
agcctaaccccttctcaaat

SEQ ID NO: 121 - DNA, 20 bp, Artificial Sequence
  3′ flanking sequence of biomarker CAN2913
aagctcaaggtagatgaccg

SEQ ID NO: 122 - DNA, 23 bp, Artificial Sequence
  3′ flanking sequence of biomarker NM101
cgttctcactcgtctcatcatta

SEQ ID NO: 123 - DNA, 20 bp, Artificial Sequence
  3′ flanking sequence of biomarker ANUCS305
tgttgtcgaaagttcctagg

SEQ ID NO: 124 - DNA, 20 bp, Artificial Sequence
  Forward primer for amplifying biomarker CAN0006
agcaagagggtaagagggat

SEQ ID NO: 125 - DNA, 20 bp, Artificial Sequence
  Reverse primer for amplifying biomarker CAN0006
tcatcatcatcctcatcagc

SEQ ID NO: 126 - DNA, 20 bp, Artificial Sequence
  Forward primer for amplifying biomarker CAN0051
aacccaaaagagctgagaga

SEQ ID NO: 127 - DNA, 20 bp, Artificial Sequence
  Reverse primer for amplifying biomarker CAN0051
ctcagcaaggtgagtacacg

SEQ ID NO: 128 - DNA, 20 bp, Artificial Sequence
  Forward primer for amplifying biomarker CAN0061
ggggtgagagaaagtgagaa

SEQ ID NO: 129 - DNA, 20 bp, Artificial Sequence
  Reverse primer for amplifying biomarker CAN0061
ctctgcacactcctgatttg

SEQ ID NO: 130 - DNA, 20 bp, Artificial Sequence
  Forward primer for amplifying biomarker CAN0118
gggtggttttgtgaagagat

SEQ ID NO: 131 - DNA, 20 bp, Artificial Sequence
  Reverse primer for amplifying biomarker CAN0118
ggagaagcattgttcgttct

SEQ ID NO: 132 - DNA, 20 bp, Artificial Sequence
  Forward primer for amplifying biomarker CAN0189
cagagctcaaagaaagggaa

SEQ ID NO: 133 - DNA, 20 bp, Artificial Sequence
  Reverse primer for amplifying biomarker CAN0189
aatagctggctcatcaggtc

SEQ ID NO: 134 - DNA, 20 bp, Artificial Sequence
  Forward primer for amplifying biomarker CAN0590
agggagagagagaatgtccc

SEQ ID NO: 135 - DNA, 20 bp, Artificial Sequence
  Reverse primer for amplifying biomarker CAN0590
tcttctgcaccacaggtaga

SEQ ID NO: 136 - DNA, 20 bp, Artificial Sequence
  Forward primer for amplifying biomarker CAN0716
tcacctcaaatggtgatgtc

SEQ ID NO: 137 - DNA, 20 bp, Artificial Sequence
  Reverse primer for amplifying biomarker CAN0716
gaaaccgaagaagaagtgga

SEQ ID NO: 138 - DNA, 20 bp, Artificial Sequence
  Forward primer for amplifying biomarker CAN0795
agaggagtgttgatgaagcc

SEQ ID NO: 139 - DNA, 20 bp, Artificial Sequence
  Reverse primer for amplifying biomarker CAN0795
catctgagctggatttcaca

SEQ ID NO: 140 - DNA, 20 bp, Artificial Sequence
  Forward primer for amplifying biomarker CAN1455
aagtccaaccacctcacaat

SEQ ID NO: 141 - DNA, 20 bp, Artificial Sequence
  Reverse primer for amplifying biomarker CAN1455
cttaaccaatccccaagatg

SEQ ID NO: 142 - DNA, 20 bp, Artificial Sequence
  Forward primer for amplifying biomarker CAN1481
ttccccaactatccaatcac

SEQ ID NO: 143 - DNA, 20 bp, Artificial Sequence
  Reverse primer for amplifying biomarker CAN1481
atttgagaaggggttaggct

SEQ ID NO: 144 - DNA, 20 bp, Artificial Sequence
  Forward primer for amplifying biomarker CAN1832
gttagaatcccatttggctg

SEQ ID NO: 145 - DNA, 20 bp, Artificial Sequence
  Reverse primer for amplifying biomarker CAN1832
gtttcgaaagttgaaggctc

SEQ ID NO: 146 - DNA, 20 bp, Artificial Sequence
  Forward primer for amplifying biomarker CAN2166
caaatgggttcctctcaatc

SEQ ID NO: 147 - DNA, 20 bp, Artificial Sequence
  Reverse primer for amplifying biomarker CAN2166
acgaggcgtacctaatgaag

SEQ ID NO: 148 - DNA, 20 bp, Artificial Sequence
  Forward primer for amplifying biomarker CAN3105
cttcatcatcaaaccactgc

SEQ ID NO: 149 - DNA, 20 bp, Artificial Sequence
  Reverse primer for amplifying biomarker CAN3105
tgtccatatgaggtcactgc

SEQ ID NO: 150 - DNA, 20 bp, Artificial Sequence
  Forward primer for amplifying biomarker CAN3321
agagtacggcctagttacgg

SEQ ID NO: 151 - DNA, 20 bp, Artificial Sequence
  Reverse primer for amplifying biomarker CAN3321
ataggtgaaaacttggcctg

SEQ ID NO: 152 - DNA, 20 bp, Artificial Sequence
  3′ flanking sequence of biomarker CAN0006
gctgatgaggatgatgatga

SEQ ID NO: 153 - DNA, 20 bp, Artificial Sequence
  3′ flanking sequence of biomarker CAN0051
cgtgtactcaccttgctgag

SEQ ID NO: 154 - DNA, 20 bp, Artificial Sequence
  3′ flanking sequence of biomarker CAN0061
caaatcaggagtgtgcagag

SEQ ID NO: 155 - DNA, 20 bp, Artificial Sequence
  3′ flanking sequence of biomarker CAN0118
agaacgaacaatgcttctcc

SEQ ID NO: 156 - DNA, 20 bp, Artificial Sequence
  3′ flanking sequence of biomarker CAN0189
gacctgatgagccagctatt

SEQ ID NO: 157 - DNA, 20 bp, Artificial Sequence
  3′ flanking sequence of biomarker CAN0590
tctacctgtggtgcagaaga

SEQ ID NO: 158 - DNA, 20 bp, Artificial Sequence
  3′ flanking sequence of biomarker CAN0716
tccacttcttcttcggtttc

SEQ ID NO: 159 - DNA, 20 bp, Artificial Sequence
  3′ flanking sequence of biomarker CAN0795
tgtgaaatccagctcagatg

SEQ ID NO: 160 - DNA, 20 bp, Artificial Sequence
  3′ flanking sequence of biomarker CAN1455
catcttggggattggttaag

SEQ ID NO: 161 - DNA, 20 bp, Artificial Sequence
  3′ flanking sequence of biomarker CAN1481
agcctaaccccttctcaaat

SEQ ID NO: 162 - DNA, 20 bp, Artificial Sequence
  3′ flanking sequence of biomarker CAN1832
gagccttcaactttcgaaac

SEQ ID NO: 163 - DNA, 20 bp, Artificial Sequence
  3′ flanking sequence of biomarker CAN2166
cttcattaggtacgcctcgt

SEQ ID NO: 164 - DNA, 20 bp, Artificial Sequence
  3′ flanking sequence of biomarker CAN3105
gcagtgacctcatatggaca

SEQ ID NO: 165 - DNA, 20 bp, Artificial Sequence
  3′ flanking sequence of biomarker CAN3321
caggccaagttttcacctat

SEQ ID NO: 166 - DNA, 22 bp, Artificial Sequence
  Forward primer for amplifying biomarker ANUCS304ana
tgagaagatgaaaacgtgtgac

SEQ ID NO: 167 - DNA, 21 bp, Artificial Sequence
  Reverse primer for amplifying biomarker ANUCS304ana
aaagaaggacctcccatatcc

SEQ ID NO: 168 - DNA, 22 bp, Artificial Sequence
  Forward primer for amplifying biomarker ANUCS501ana and ANUCS501ana2
gtgagtagcaataatggagtga

SEQ ID NO: 169 - DNA, 23 bp, Artificial Sequence
  Reverse primer for amplifying biomarker ANUCS501ana
caagaaattgagattcccaatgg

SEQ ID NO: 170 - DNA, 20 bp, Artificial Sequence
  Forward primer for amplifying biomarker C11-CANN1ana2
tatgattctggtgatggcgg

SEQ ID NO: 171 - DNA, 24 bp, Artificial Sequence
  Reverse primer for amplifying biomarker C11-CANN1ana2
atgggcgtccttgaattggttacg

SEQ ID NO: 172 - DNA, 22 bp, Artificial Sequence
  Forward primer for amplifying biomarker CAN0009ana2
tctaagctcatctgaaattctc

SEQ ID NO: 173 - DNA, 24 bp, Artificial Sequence
  Reverse primer for amplifying biomarker CAN0009ana2
tccgaatagaaaaaaaaaattggg

SEQ ID NO: 174 - DNA, 22 bp, Artificial Sequence
  Forward primer for amplifying biomarker CAN0055ana4
tactgggtaataagattccacc

SEQ ID NO: 175 - DNA, 24 bp, Artificial Sequence
  Reverse primer for amplifying biomarker CAN0055ana4
cttaatctaacctctaacgaccgc

SEQ ID NO: 176 - DNA, 27 bp, Artificial Sequence
  Forward primer for amplifying biomarker CAN0057ana
gacttgtttgtcagatatttgactgac

SEQ ID NO: 177 - DNA, 29 bp, Artificial Sequence
  Reverse primer for amplifying biomarker CAN0057ana
gaatttttgttgcatttaactcactttag

SEQ ID NO: 178 - DNA, 21 bp, Artificial Sequence
  Forward primer for amplifying biomarker CAN0075ana
aaagctctgtacgacatgtcg

SEQ ID NO: 179 - DNA, 20 bp, Artificial Sequence
  Reverse primer for amplifying biomarker CAN0075ana
ttcttttctggggctctctc

SEQ ID NO: 180 - DNA, 24 bp, Artificial Sequence
  Forward primer for amplifying biomarker CAN0089ana
ctaaaaacattacagctgtgaggc

SEQ ID NO: 181 - DNA, 24 bp, Artificial Sequence
  Reverse primer for amplifying biomarker CAN0089ana
cgccaacatttactcactaaactc

SEQ ID NO: 182 - DNA, 22 bp, Artificial Sequence
  Forward primer for amplifying biomarker CAN0103ana
caaaaacactctagactcctcc

SEQ ID NO: 183 - DNA, 22 bp, Artificial Sequence
  Reverse primer for amplifying biomarker CAN0103ana
ggagcttctttttgagagtctg

SEQ ID NO: 184 - DNA, 23 bp, Artificial Sequence
  Forward primer for amplifying biomarker CAN0124ana
cttacaaaaaggacactagccac

SEQ ID NO: 185 - DNA, 27 bp, Artificial Sequence
  Reverse primer for amplifying biomarker CAN0124ana
ataaacacaaggaagttgatgatgaag

SEQ ID NO: 186 - DNA, 21 bp, Artificial Sequence
  Forward primer for amplifying biomarker CAN0138ana2
gccaaacagaaatgactggtg

SEQ ID NO: 187 - DNA, 26 bp, Artificial Sequence
  Reverse primer for amplifying biomarker CAN0138ana2
ataataagaaattaatggtgttgttg

SEQ ID NO: 188 - DNA, 22 bp, Artificial Sequence
  Forward primer for amplifying biomarker CAN0156ana
gagtctttgcagcaaacacacc

SEQ ID NO: 189 - DNA, 27 bp, Artificial Sequence
  Reverse primer for amplifying biomarker CAN0156ana
ttgctaaataactcagtacaaaacaga

SEQ ID NO: 190 - DNA, 24 bp, Artificial Sequence
  Forward primer for amplifying biomarker CAN0180ana
ctttttcaaactctcaatccttgc

SEQ ID NO: 191 - DNA, 24 bp, Artificial Sequence
  Reverse primer for amplifying biomarker CAN0180ana
agacgataggactaaaatctaatc

SEQ ID NO: 192 - DNA, 20 bp, Artificial Sequence
  Forward primer for amplifying biomarker CAN0202ana and CAN0202ana2
atcgggaagacacatgcttc

SEQ ID NO: 193 - DNA, 20 bp, Artificial Sequence
  Reverse primer for amplifying biomarker CAN0202ana
actacctacaacggttcctg

SEQ ID NO: 194 - DNA, 22 bp, Artificial Sequence
  Forward primer for amplifying biomarker CAN0244ana
ggaacagcaaaatccaataccc

SEQ ID NO: 195 - DNA, 22 bp, Artificial Sequence
  Reverse primer for amplifying biomarker CAN0244ana
aactcagttcaaggaaaaaggg

SEQ ID NO: 196 - DNA, 20 bp, Artificial Sequence
  Forward primer for amplifying biomarker CAN0271ana
aagaggcaagagagggtgag

SEQ ID NO: 197 - DNA, 20 bp, Artificial Sequence
  Reverse primer for amplifying biomarker CAN0271ana
acgacgatgatgatgatggc

SEQ ID NO: 198 - DNA, 24 bp, Artificial Sequence
  Forward primer for amplifying biomarker CAN0278ana
cgaaacacagatgaattcttattc

SEQ ID NO: 199 - DNA, 20 bp, Artificial Sequence
  Reverse primer for amplifying biomarker CAN0278ana
aagtgggcaacgacttgtgg

SEQ ID NO: 200 - DNA, 23 bp, Artificial Sequence
  Forward primer for amplifying biomarker CAN0291ana
atctgagttttggatttggagtg

SEQ ID NO: 201 - DNA, 20 bp, Artificial Sequence
  Reverse primer for amplifying biomarker CAN0291ana
agaagaccggtggttacctg

SEQ ID NO: 202 - DNA, 24 bp, Artificial Sequence
  Forward primer for amplifying biomarker CAN0298ana
caaaatatttctataacgggcagc

SEQ ID NO: 203 - DNA, 23 bp, Artificial Sequence
  Reverse primer for amplifying biomarker CAN0298ana
aaaccctcattagatgtgttcac

SEQ ID NO: 204 - DNA, 22 bp, Artificial Sequence
  Forward primer for amplifying biomarker CAN0308ana
tagctttactcctactcaaacc

SEQ ID NO: 205 - DNA, 21 bp, Artificial Sequence
  Reverse primer for amplifying biomarker CAN0308ana
ttcttgaacatgtcttggcac

SEQ ID NO: 206 - DNA, 24 bp, Artificial Sequence
  Forward primer for amplifying biomarker CAN0338-339ana
acttctactactcaacaatccaac

SEQ ID NO: 207 - DNA, 21 bp, Artificial Sequence
  Reverse primer for amplifying biomarker CAN0338-339ana
tcaatggtggcaagtagcaac

SEQ ID NO: 208 - DNA, 23 bp, Artificial Sequence
  Forward primer for amplifying biomarker CAN0442ana
tcaaaataggaaacaccagaacc

SEQ ID NO: 209 - DNA, 20 bp, Artificial Sequence
  Reverse primer for amplifying biomarker CAN0442ana
ttgttcttctctgagctggg

SEQ ID NO: 210 - DNA, 22 bp, Artificial Sequence
  Forward primer for amplifying biomarker CAN0509ana
tcagtcgtcatctattcaacag

SEQ ID NO: 211 - DNA, 22 bp, Artificial Sequence
  Reverse primer for amplifying biomarker CAN0509ana
ttgaaacgttattttgcggagc

SEQ ID NO: 212 - DNA, 24 bp, Artificial Sequence
  Forward primer for amplifying biomarker CAN0577ana
cctccttattgagaccaaccagtc

SEQ ID NO: 213 - DNA, 26 bp, Artificial Sequence
  Reverse primer for amplifying biomarker CAN0577ana
ctcttttgtttgttctgttggacttg

SEQ ID NO: 214 - DNA, 20 bp, Artificial Sequence
  Forward primer for amplifying biomarker CAN0716ana
tcgatggcaccataaacggc

SEQ ID NO: 215 - DNA, 23 bp, Artificial Sequence
  Reverse primer for amplifying biomarker CAN0716ana
aagtgatgagcagtttgagatac

SEQ ID NO: 216 - DNA, 22 bp, Artificial Sequence
  Forward primer for amplifying biomarker CAN0717ana
gagtcttttcttccggaaattc

SEQ ID NO: 217 - DNA, 23 bp, Artificial Sequence
  Reverse primer for amplifying biomarker CAN0717ana
gtccactatacaaaaactgaagc

SEQ ID NO: 218 - DNA, 22 bp, Artificial Sequence
  Forward primer for amplifying biomarker CAN0802ana
gattatggggctgcttcaaaac

SEQ ID NO: 219 - DNA, 27 bp, Artificial Sequence
  Reverse primer for amplifying biomarker CAN0802ana
aattagaatgatgtcagttttcactgg

SEQ ID NO: 220 - DNA, 25 bp, Artificial Sequence
  Forward primer for amplifying biomarker CAN0907ana
tgtactaaagttggttcaattcgac

SEQ ID NO: 221 - DNA, 24 bp, Artificial Sequence
  Reverse primer for amplifying biomarker CAN0907ana
cactttgactagtgatccaaacag

SEQ ID NO: 222 - DNA, 24 bp, Artificial Sequence
  Forward primer for amplifying biomarker CAN1409ana
ccttacttttcaacttgccatccc

SEQ ID NO: 223 - DNA, 26 bp, Artificial Sequence
  Reverse primer for amplifying biomarker CAN1409ana
atgggttttattcaacaaccggttac

SEQ ID NO: 224 - DNA, 22 bp, Artificial Sequence
  Forward primer for amplifying biomarker CAN1539ana and CAN1539ana2
gagacttgccaaatccaacttc

SEQ ID NO: 225 - DNA, 25 bp, Artificial Sequence
  Reverse primer for amplifying biomarker CAN1539ana
ctgtatgcgtctttgtaactatttg

SEQ ID NO: 226 - DNA, 24 bp, Artificial Sequence
  Forward primer for amplifying biomarker CAN1542ana
gtcggatttatctaaaggttagag

SEQ ID NO: 227 - DNA, 21 bp, Artificial Sequence
  Reverse primer for amplifying biomarker CAN1542ana
gagaagtacaggaaccaagag

SEQ ID NO: 228 - DNA, 21 bp, Artificial Sequence
  Forward primer for amplifying biomarker CAN1832ana
ggtccttacaaaacttcccac

SEQ ID NO: 229 - DNA, 24 bp, Artificial Sequence
  Reverse primer for amplifying biomarker CAN1832ana
tcattcctgagaatataaagcttg

SEQ ID NO: 230 - DNA, 27 bp, Artificial Sequence
  Forward primer for amplifying biomarker CAN2039ana
taatttattttacagttgggggaagtg

SEQ ID NO: 231 - DNA, 25 bp, Artificial Sequence
  Reverse primer for amplifying biomarker CAN2039ana
cagtagtacaagatccaaggaagtg

SEQ ID NO: 232 - DNA, 26 bp, Artificial Sequence
  Forward primer for amplifying biomarker CAN2418ana
ttgttgtactgatgaactcgttgttg

SEQ ID NO: 233 - DNA, 27 bp, Artificial Sequence
  Reverse primer for amplifying biomarker CAN2418ana
agcttaactcaaccaagatacttttgc

SEQ ID NO: 234 - DNA, 24 bp, Artificial Sequence
  Forward primer for amplifying biomarker CAN2550ana2
tcatatgtgagattgtgattagct

SEQ ID NO: 235 - DNA, 22 bp, Artificial Sequence
  Reverse primer for amplifying biomarker CAN2550ana2
ggtggtcgtattattcgcatag

SEQ ID NO: 236 - DNA, 20 bp, Artificial Sequence
  Forward primer for amplifying biomarker CAN2829ana
ttgcaaactagcgagggagc

SEQ ID NO: 237 - DNA, 22 bp, Artificial Sequence
  Reverse primer for amplifying biomarker CAN2829ana
tccggttcccatacaattcttg

SEQ ID NO: 238 - DNA, 22 bp, Artificial Sequence
  Forward primer for amplifying biomarker CAN2846ana
cctcaagatggcaaaaatgggg

SEQ ID NO: 239 - DNA, 20 bp, Artificial Sequence
  Reverse primer for amplifying biomarker CAN2846ana
gcagggattggacccatttg

SEQ ID NO: 240 - DNA, 21 bp, Artificial Sequence
  Forward primer for amplifying biomarker CAN2902ana
tctcctagaagtggcaatccc

SEQ ID NO: 241 - DNA, 24 bp, Artificial Sequence
  Reverse primer for amplifying biomarker CAN2902ana
ccaactgcacgtacagaaacaaaa

SEQ ID NO: 242 - DNA, 20 bp, Artificial Sequence
  Forward primer for amplifying biomarker CAN2913ana
gaaagcgagaggcagaacag

SEQ ID NO: 243 - DNA, 20 bp, Artificial Sequence
  Forward primer for amplifying biomarker CAN3008ana
tggtcttgacgccattctcg

SEQ ID NO: 244 - DNA, 21 bp, Artificial Sequence
  Reverse primer for amplifying biomarker CAN3008ana
aaggaagctttgaggtccagc

SEQ ID NO: 245 - DNA, 20 bp, Artificial Sequence
  Reverse primer for amplifying biomarker CAN3008ana
gctcctccggtggtctaaac

SEQ ID NO: 246 - DNA, 23 bp, Artificial Sequence
  Forward primer for amplifying biomarker D02-CANN1ana
gaaccccaaatgttgtattgaag

SEQ ID NO: 247 - DNA, 25 bp, Artificial Sequence
  Reverse primer for amplifying biomarker D02-CANN1ana
ttttaaatttaggtgtgtactttgg

SEQ ID NO: 248 - DNA, 18 bp, Artificial Sequence
  Forward primer for amplifying biomarker E07-CANN1ana2
tccaccaccatcatcagc

SEQ ID NO: 249 - DNA, 25 bp, Artificial Sequence
  Reverse primer for amplifying biomarker E07-CANN1ana2
tatatgtgggtgtgttagaattttc

SEQ ID NO: 250 - DNA, 20 bp, Artificial Sequence
  Forward primer for amplifying biomarker H06-CANN2ana
agaggagctggtttcagtgg

SEQ ID NO: 251 - DNA, 20 bp, Artificial Sequence
  Reverse primer for amplifying biomarker H06-CANN2ana
ttccctgtcgtggttgcttg

SEQ ID NO: 252 - DNA, 21 bp, Artificial Sequence
  3′ flanking sequence of biomarker ANUCS304ana
ggatatgggaggtccttcttt

SEQ ID NO: 253 - DNA, 23 bp, Artificial Sequence
  3′ flanking sequence of biomarker ANUCS501ana
ccattgggaatctcaatttcttg

SEQ ID NO: 254 - DNA, 24 bp, Artificial Sequence
  3′ flanking sequence of biomarker C11-CANN1ana2
cgtaaccaattcaaggacgcccat

SEQ ID NO: 255 - DNA, 24 bp, Artificial Sequence
  3′ flanking sequence of biomarker CAN0009ana2
cccaattttttttttctattcgga

SEQ ID NO: 256 - DNA, 24 bp, Artificial Sequence
  3′ flanking sequence of biomarker CAN0055ana4
gcggtcgttagaggttagattaag

SEQ ID NO: 257 - DNA, 29 bp, Artificial Sequence
  3′ flanking sequence of biomarker CAN0057ana
ctaaagtgagttaaatgcaacaaaaattc

SEQ ID NO: 258 - DNA, 20 bp, Artificial Sequence
  3′ flanking sequence of biomarker CAN0075ana
gagagagccccagaaaagaa

SEQ ID NO: 259 - DNA, 24 bp, Artificial Sequence
  3′ flanking sequence of biomarker CAN0089ana
gagtttagtgagtaaatgttggcg

SEQ ID NO: 260 - DNA, 22 bp, Artificial Sequence
  3′ flanking sequence of biomarker CAN0103ana
cagactctcaaaaagaagctcc

SEQ ID NO: 261 - DNA, 27 bp, Artificial Sequence
  3′ flanking sequence of biomarker CAN0124ana
cttcatcatcaacttccttgtgtttat

SEQ ID NO: 262 - DNA, 26 bp, Artificial Sequence
  3′ flanking sequence of biomarker CAN0138ana2
caacaacaccattaatttcttattat

SEQ ID NO: 263 - DNA, 27 bp, Artificial Sequence
  3′ flanking sequence of biomarker CAN0156ana
tctgttttgtactgagttatttagcaa

SEQ ID NO: 264 - DNA, 24 bp, Artificial Sequence
  3′ flanking sequence of biomarker CAN0180ana
gattagattttagtcctatcgtct

SEQ ID NO: 265 - DNA, 20 bp, Artificial Sequence
  3′ flanking sequence of biomarker CAN0202ana
caggaaccgttgtaggtagt

SEQ ID NO: 266 - DNA, 22 bp, Artificial Sequence
  3′ flanking sequence of biomarker CAN0244ana
ccctttttccttgaactgagtt

SEQ ID NO: 267 - DNA, 20 bp, Artificial Sequence
  3′ flanking sequence of biomarker CAN0271ana
gccatcatcatcatcgtcgt

SEQ ID NO: 268 - DNA, 20 bp, Artificial Sequence
  3′ flanking sequence of biomarker CAN0278ana
ccacaagtcgttgcccactt

SEQ ID NO: 269 - DNA, 20 bp, Artificial Sequence
  3′ flanking sequence of biomarker CAN0291ana
caggtaaccaccggtcttct

SEQ ID NO: 270 - DNA, 23 bp, Artificial Sequence
  3′ flanking sequence of biomarker CAN0298ana
gtgaacacatctaatgagggttt

SEQ ID NO: 271 - DNA, 21 bp, Artificial Sequence
  3′ flanking sequence of biomarker CAN0308ana
gtgccaagacatgttcaagaa

SEQ ID NO: 272 - DNA, 21 bp, Artificial Sequence
  3′ flanking sequence of biomarker CAN0338-339ana
gttgctacttgccaccattga

SEQ ID NO: 273 - DNA, 20 bp, Artificial Sequence
  3′ flanking sequence of biomarker CAN0442ana
cccagctcagagaagaacaa

SEQ ID NO: 274 - DNA, 22 bp, Artificial Sequence
  3′ flanking sequence of biomarker CAN0509ana
gctccgcaaaataacgtttcaa

SEQ ID NO: 275 - DNA, 26 bp, Artificial Sequence
  3′ flanking sequence of biomarker CAN0577ana
caagtccaacagaacaaacaaaagag

SEQ ID NO: 276 - DNA, 23 bp, Artificial Sequence
  3′ flanking sequence of biomarker CAN0716ana
gtatctcaaactgctcatcactt

SEQ ID NO: 277 - DNA, 23 bp, Artificial Sequence
  3′ flanking sequence of biomarker CAN0717ana
gcttcagtttttgtatagtggac

SEQ ID NO: 278 - DNA, 27 bp, Artificial Sequence
  3′ flanking sequence of biomarker CAN0802ana
ccagtgaaaactgacatcattctaatt

SEQ ID NO: 279 - DNA, 24 bp, Artificial Sequence
  3′ flanking sequence of biomarker CAN0907ana
ctgtttggatcactagtcaaagtg

SEQ ID NO: 280 - DNA, 26 bp, Artificial Sequence
  3′ flanking sequence of biomarker CAN1409ana
gtaaccggttgttgaataaaacccat

SEQ ID NO: 281 - DNA, 25 bp, Artificial Sequence
  3′ flanking sequence of biomarker CAN1539ana
caaatagttacaaagacgcatacag

SEQ ID NO: 282 - DNA, 21 bp, Artificial Sequence
  3′ flanking sequence of biomarker CAN1542ana
ctcttggttcctgtacttctc

SEQ ID NO: 283 - DNA, 24 bp, Artificial Sequence
  3′ flanking sequence of biomarker CAN1832ana
caagctttatattctcaggaatga

SEQ ID NO: 284 - DNA, 25 bp, Artificial Sequence
  3′ flanking sequence of biomarker CAN2039ana
cacttccttggatcttgtactactg

SEQ ID NO: 285 - DNA, 27 bp, Artificial Sequence
  3′ flanking sequence of biomarker CAN2418ana
gcaaaagtatcttggttgagttaagct

SEQ ID NO: 286 - DNA, 22 bp, Artificial Sequence
  3′ flanking sequence of biomarker CAN2550ana2
ctatgcgaataatacgaccacc

SEQ ID NO: 287 - DNA, 22 bp, Artificial Sequence
  3′ flanking sequence of biomarker CAN2829ana
caagaattgtatgggaaccgga

SEQ ID NO: 288 - DNA, 20 bp, Artificial Sequence
  3′ flanking sequence of biomarker CAN2846ana
caaatgggtccaatccctgc

SEQ ID NO: 289 - DNA, 24 bp, Artificial Sequence
  3′ flanking sequence of biomarker CAN2902ana
ttttgtttctgtacgtgcagttgg

SEQ ID NO: 290 - DNA, 20 bp, Artificial Sequence
  3′ flanking sequence of biomarker CAN2913ana
cgagaatggcgtcaagacca

SEQ ID NO: 291 - DNA, 20 bp, Artificial Sequence
  3′ flanking sequence of biomarker CAN3008ana
gtttagaccaccggaggagc

SEQ ID NO: 292 - DNA, 25 bp, Artificial Sequence
  3′ flanking sequence of biomarker D02-CANN1ana
ccaaagtacacacctaaatttaaaa

SEQ ID NO: 293 - DNA, 25 bp, Artificial Sequence
  3′ flanking sequence of biomarker E07-CANN1ana2
gaaaattctaacacacccacatata

SEQ ID NO: 294 - DNA, 20 bp, Artificial Sequence
  3′ flanking sequence of biomarker H06-CANN2ana
caagcaaccacgacagggaa

SEQ ID NO: 295 - DNA, 21 bp, Artificial Sequence
  Reverse primer for amplifying biomarker ANUCS501ana2
tgtcactcactgccatctctc

SEQ ID NO: 296 - DNA, 25 bp, Artificial Sequence
  Forward primer for amplifying biomarker CAN0067ana
cctttgctctattcccacttacaac

SEQ ID NO: 297 - DNA, 26 bp, Artificial Sequence
  Reverse primer for amplifying biomarker CAN0067ana
tttagacaaaccacacaaccgaaaac

SEQ ID NO: 298 - DNA, 25 bp, Artificial Sequence
  Forward primer for amplifying biomarker CAN0097ana
tcactactttcaccaacgttagctc

SEQ ID NO: 299 - DNA, 24 bp, Artificial Sequence
  Reverse primer for amplifying biomarker CAN0097ana
agtaaagcctctgcgagtatcctc

SEQ ID NO: 300 - DNA, 23 bp, Artificial Sequence
  Reverse primer for amplifying biomarker CAN0202ana2 and CAN0202ana3
aaccttgtacgctttccacttgc

SEQ ID NO: 301 - DNA, 24 bp, Artificial Sequence
  Forward primer for amplifying biomarker CAN0250ana
gcacaattcaaaggtagccatagc

SEQ ID NO: 302 - DNA, 25 bp, Artificial Sequence
  Reverse primer for amplifying biomarker CAN0250ana
cagggtaacaatcacatgaaactac

SEQ ID NO: 303 - DNA, 24 bp, Artificial Sequence
  Forward primer for amplifying biomarker CAN0504ana2
cacaagcatagctccacctgtaac

SEQ ID NO: 304 - DNA, 27 bp, Artificial Sequence
  Reverse primer for amplifying biomarker CAN0504ana2
aaggaaatcaactttggatcatctcag

SEQ ID NO: 305 - DNA, 22 bp, Artificial Sequence
  Reverse primer for amplifying biomarker CAN1539ana2
gagtctgctgtacatttggacg

SEQ ID NO: 306 - DNA, 26 bp, Artificial Sequence
  Forward primer for amplifying biomarker E07-CANN1ana4
cattgtgatggagttgatgaaagtac

SEQ ID NO: 307 - DNA, 26 bp, Artificial Sequence
  Forward primer for amplifying biomarker CAN0067ana2
ccctttgctctattcccacttacaac

SEQ ID NO: 308 - DNA, 27 bp, Artificial Sequence
  Reverse primer for amplifying biomarker CAN0067ana2
ttagacaaaccacacaaccgaaaacag

SEQ ID NO: 309 - DNA, 27 bp, Artificial Sequence
  Forward primer for amplifying biomarker CAN0202ana3
atcgggaagacacatgcttcatactac

SEQ ID NO: 310 - DNA, 28 bp, Artificial Sequence
  Forward primer for amplifying biomarker CAN0802ana2
tgggcagagaaactccccctaatgaaag

SEQ ID NO: 311 - DNA, 27 bp, Artificial Sequence
  Reverse primer for amplifying biomarker CAN0802ana2
ttttggtgtgtggaagggaacttgacg

SEQ ID NO: 312 - DNA, 30 bp, Artificial Sequence
  Forward primer for amplifying biomarker CAN0055ana5
gtaataagattccaccttctggctcttatg

SEQ ID NO: 313 - DNA, 28 bp, Artificial Sequence
  Reverse primer for amplifying biomarker CAN0055ana5
ccaaaacttaatctaacctctaacgacc

SEQ ID NO: 314 - DNA, 21 bp, Artificial Sequence
  3′ flanking sequence of biomarker ANUCS501ana2
gagagatggcagtgagtgaca

SEQ ID NO: 315 - DNA, 26 bp, Artificial Sequence
  3′ flanking sequence of biomarker CAN0067ana
gttttcggttgtgtggtttgtctaaa

SEQ ID NO: 316 - DNA, 24 bp, Artificial Sequence
  3′ flanking sequence of biomarker CAN0097ana
gaggatactcgcagaggctttact

SEQ ID NO: 317 - DNA, 23 bp, Artificial Sequence
  3′ flanking sequence of biomarker CAN0202ana2
gcaagtggaaagcgtacaaggtt

SEQ ID NO: 318 - DNA, 25 bp, Artificial Sequence
  3′ flanking sequence of biomarker CAN0250ana
gtagtttcatgtgattgttaccctg

SEQ ID NO: 319 - DNA, 27 bp, Artificial Sequence
  3′ flanking sequence of biomarker CAN0504ana2
ctgagatgatccaaagttgatttcctt

SEQ ID NO: 320 - DNA, 22 bp, Artificial Sequence
  3′ flanking sequence of biomarker CAN1539ana2
cgtccaaatgtacagcagactc

SEQ ID NO: 321 - DNA, 27 bp, Artificial Sequence
  3′ flanking sequence of biomarker CAN0067ana2
ctgttttcggttgtgtggtttgtctaa

SEQ ID NO: 322 - DNA, 27 bp, Artificial Sequence
  3′ flanking sequence of biomarker CAN0802ana2
cgtcaagttcccttccacacaccaaaa

SEQ ID NO: 323 - DNA, 28 bp, Artificial Sequence
  3′ flanking sequence of biomarker CAN0055ana5
ggtcgttagaggttagattaagttttgg

(19) United States
(12) Patent Application Publication
(10) Pub. No.: US 2023/0407414 A1
(43) Pub. Date: Dec. 21, 2023

(54) METHODS OF GENOTYPING CANNABIS

(71) Applicant: 1769474 ALBERTA LTD., Edmonton (CA)

(72) Inventor: Erin Gilchrist, Vancouver (CA)

(73) Assignee: 1769474 ALBERTA LTD., Edmonton (CA)

(21) Appl. No.: 18/030,731

(22) PCT Filed: Oct. 6, 2021
(86) PCT No.: PCT/CA2021/051401

§ 371 Date: Apr. 6, 2023

Related U.S. Application Data
(60) Provisional application No. 63/088,314, filed on Oct. 6, 2020.

(30) Foreign Application Priority Data
Aug. 31, 2021 (CA) 3129537

(51) Int. Cl. C12Q 1/6895; A01H 1/04; A01H 6/28

(52) U.S. Cl. CPC C12Q 1/6895; A01H 1/045; C12Q 2600/16; C12Q 2600/156; A01H 6/28

(57) ABSTRACT

This disclosure relates to methods and compositions for determining the alleles present at polymorphic simple sequence repeat (SSR) sites in the Cannabis genome to produce a genetic profile for Cannabis plants.

### FIELD OF THE DISCLOSURE

This disclosure relates to the field of genetic markers for genotyping Cannabis plants. More specifically, this disclosure relates to methods and compositions useful for determining the alleles present at polymorphic simple sequence repeat (SSR) sites in the Cannabis genome and using the precise combination of alleles at these sites to produce a genetic profile for Cannabis plants.

### BACKGROUND OF THE DISCLOSURE

Genotyping is the process of determining the genetic makeup (or “genotype”) of an organism, such as a Cannabis plant, by examining the DNA in the cells of that organism. Being able to identify the genetic makeup of a particular Cannabis plant may be desirable or necessary for several reasons and add value to a product being marketed.

First, there is a current lack of consistency in naming varieties in the Cannabis industry. Distinct varieties may be marketed under the same name. Alternatively, a single variety may be marketed under several different names. Thus, effective genotyping of individual Cannabis plants can be used to distinguish varieties in the first place, and reduce confusion in the marketplace.

Second, genotyping may also be used for quality assurance. For example, genotyping may also be used confirm genetic provenance, whether or not a particular Cannabis variety or value-added product is indeed of the variety as marketed, to monitor batch-to-batch consistency of a plant or plant product or seed batches, or to confirm that putative clones are identical to each other or to a specific mother plant. Genotyping of progeny can also be used to determine whether a particular line is sufficiently inbred.

Third, genotyping can be used to determine the genetic make-up of an individual plant relative to a parent or parents, and to allow for the early selection of plants carryings specific traits of interest for breeding or production.

Fourth, genotyping can be used to determine that the sample is of the genus Cannabis for forensic purposes.

It is now possible to directly sequence the genomes of individual plants in order to determine their genotypes. However, sequencing the genomes of individual plants remains expensive on a large scale, requires a significant amount of time and resources, and provides unnecessarily large amounts of data to analyze for many purposes. Accordingly, there remains a need for methods of determining the genotype of Cannabis plants accurately and consistently, inexpensively, with a short turnaround time, and with output data that is easily understood by individuals in need of such genotype information.

### SUMMARY OF THE INVENTION

The present disclosure pertains to the utility of a selection of Cannabis SSR markers for genotyping Cannabis plants.

Other aspects and features of the present invention will become apparent to those ordinarily skilled in the art upon review of the following description of specific embodiments of the invention.

Aspect of the disclosure relate to a method for constructing a genetic profile for a Cannabis plant, the method comprising testing a deoxyribonucleic acid (DNA) sample from the plant to determine the length of at least two polymorphic target sequences, thereby constructing the genetic profile of the plant, wherein each polymorphic target sequence comprises a simple sequence repeat (SSR) sequence, wherein the at least two polymorphic target sequences comprise two or more of: a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 7 and SEQ ID NO: 8; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 15 and SEQ ID NO: 16; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 21 and SEQ ID NO: 22; a target sequence amplifiable by a primer pair having the sequences of SEQ ID NO: 23 and SEQ ID NO: 24; a target sequence amplifiable by a primer pair having the sequences of SEQ ID NO: 25 and SEQ ID NO: 26; a target sequence amplifiable by a primer pair having the sequences of SEQ ID NO: 27 and SEQ ID NO: 28; a target sequence amplifiable by a primer pair having the sequences of SEQ ID NO: 29 and SEQ ID NO: 30; a target sequence amplifiable by a primer pair having the sequences of SEQ ID NO: 31 and SEQ ID NO: 32; a target sequence amplifiable by a primer pair having the sequences of SEQ ID NO: 33 and SEQ ID NO: 34; a target sequence amplifiable by a primer pair having the sequences of SEQ ID NO: 35 and SEQ ID NO: 36; a target sequence amplifiable by a primer pair having the sequences of SEQ ID NO: 37 and SEQ ID NO: 38; a target sequence amplifiable by a primer pair having the sequences of SEQ ID NO: 39 and SEQ ID NO: 40; a target sequence amplifiable by a primer pair having the sequences of SEQ ID NO: 41 and SEQ ID NO: 42; a target sequence amplifiable by a primer pair having the sequences of SEQ ID NO: 43 and SEQ ID NO: 44; a target sequence amplifiable by a primer pair having the sequences of SEQ ID NO: 45 and SEQ ID NO: 46; a target sequence amplifiable by a primer pair having the sequences of SEQ ID NO: 124 and SEQ ID NO: 125; a target sequence amplifiable by a primer pair having the sequences of SEQ ID NO: 126 and SEQ ID NO: 127; a target sequence amplifiable by a primer pair having the sequences of SEQ ID NO: 128 and SEQ ID NO: 129; a target sequence amplifiable by a primer pair having the sequences of SEQ ID NO: 130 and SEQ ID NO: 131; a target sequence amplifiable by a primer pair having the sequences of SEQ ID NO: 132 and SEQ ID NO: 133; a target sequence amplifiable by a primer pair having the sequences of SEQ ID NO: 134 and SEQ ID NO: 135; a target sequence amplifiable by a primer pair having the sequences of SEQ ID NO: 136 and SEQ ID NO: 137; a target sequence amplifiable by a primer pair having the sequences of SEQ ID NO: 138 and SEQ ID NO: 139; a target sequence amplifiable by a primer pair having the sequences of SEQ ID NO: 140 and SEQ ID NO: 141; a target sequence amplifiable by a primer pair having the sequences of SEQ ID NO: 142 and SEQ ID NO: 143; a target sequence amplifiable by a primer pair having the sequences of SEQ ID NO: 144 and SEQ ID NO: 145; a target sequence amplifiable by a primer pair having the sequences of SEQ ID NO: 146 and SEQ ID NO: 147; a target sequence amplifiable by a primer pair having the sequences of SEQ ID NO: 148 and SEQ ID NO: 149; a target sequence amplifiable by a primer pair having the sequences of SEQ ID NO: 150 and SEQ ID NO: 151; a target sequence amplifiable by a primer pair having the sequences of SEQ ID NO: 166 and SEQ ID NO: 167; a target sequence amplifiable by a primer pair having the sequences of SEQ ID NO: 168 and SEQ ID NO: 169; a target sequence amplifiable by a primer pair having the sequences of SEQ ID NO: 170 and SEQ ID NO: 171; a target sequence amplifiable by a primer pair having the sequences of SEQ ID NO: 172 and SEQ ID NO: 173; a target sequence amplifiable by a primer pair having the sequences of SEQ ID NO: 174 and SEQ ID NO: 175; a target sequence amplifiable by a primer pair having the sequences of SEQ ID NO: 176 and SEQ ID NO: 177; a target sequence amplifiable by a primer pair having the sequences of SEQ ID NO: 178 and SEQ ID NO: 179; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 184 and SEQ ID NO: 185; a target sequence amplifiable by a primer pair having the sequences of SEQ ID NO: 186 and SEQ ID NO: 187; a target sequence amplifiable by a primer pair having the sequences of SEQ ID NO: 188 and SEQ ID NO: 189; a target sequence amplifiable by a primer pair having the sequences of SEQ ID NO: 190 and SEQ ID NO: 191; a target sequence amplifiable by a primer pair having the sequences of SEQ ID NO: 192 and SEQ ID NO: 193; a target sequence amplifiable by a primer pair having the sequences of SEQ ID NO: 194 and SEQ ID NO: 195; a target sequence amplifiable by a primer pair having the sequences of SEQ ID NO: 196 and SEQ ID NO: 197; a target sequence amplifiable by a primer pair having the sequences of SEQ ID NO: 198 and SEQ ID NO: 199; a target sequence amplifiable by a primer pair having the sequences of SEQ ID NO: 200 and SEQ ID NO: 201; a target sequence amplifiable by a primer pair having the sequences of SEQ ID NO: 202 and SEQ ID NO: 203; a target sequence amplifiable by a primer pair having the sequences of SEQ ID NO: 204 and SEQ ID NO: 205; a target sequence amplifiable by a primer pair having the sequences of SEQ ID NO: 206 and SEQ ID NO: 207; a target sequence amplifiable by a primer pair having the sequences of SEQ ID NO: 208 and SEQ ID NO: 209; a target sequence amplifiable by a primer pair having the sequences of SEQ ID NO: 210 and SEQ ID NO: 211; a target sequence amplifiable by a primer pair having the sequences of SEQ ID NO: 212 and SEQ ID NO: 213; a target sequence amplifiable by a primer pair having the sequences of SEQ ID NO: 214 and SEQ ID NO: 215; a target sequence amplifiable by a primer pair having the sequences of SEQ ID NO: 216 and SEQ ID NO: 217; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 222 and SEQ ID NO: 223; a target sequence amplifiable by a primer pair having the sequences of SEQ ID NO: 224 and SEQ ID NO: 225; a target sequence amplifiable by a primer pair having the sequences of SEQ ID NO: 226 and SEQ ID NO: 227; a target sequence amplifiable by a primer pair having the sequences of SEQ ID NO: 228 and SEQ ID NO: 229; a target sequence amplifiable by a primer pair having the sequences of SEQ ID NO: 230 and SEQ ID NO: 231; a target sequence amplifiable by a primer pair having the sequences of SEQ ID NO: 232 and SEQ ID NO: 233; a target sequence amplifiable by a primer pair having the sequences of SEQ ID NO: 234 and SEQ ID NO: 235; a target sequence amplifiable by a primer pair having the sequences of SEQ ID NO: 236 and SEQ ID NO: 237; and a target sequence amplifiable by a primer pair having the sequences of SEQ ID NO: 238 and SEQ ID NO: 239; a target sequence amplifiable by a primer pair having the sequences of SEQ ID NO: 240 and SEQ ID NO: 241; a target sequence amplifiable by a primer pair having the sequences of SEQ ID NO: 242 and SEQ ID NO: 243; a target sequence amplifiable by a primer pair having the sequences of SEQ ID NO: 244 and SEQ ID NO: 245; a target sequence amplifiable by a primer pair having the sequences of SEQ ID NO: 246 and SEQ ID NO: 247; a target sequence amplifiable by a primer pair having the sequences of SEQ ID NO: 248 and SEQ ID NO: 249; a target sequence amplifiable by a primer pair having the sequences of SEQ ID NO: 250 and SEQ ID NO: 251a target sequence amplifiable by a primer pair having the sequences SEQ ID NO: 168 and SEQ ID NO: 295; a target sequence amplifiable by a primer pair having the sequences SEQ ID NO: 192 and SEQ ID NO: 300; a target sequence amplifiable by a primer pair having the sequences SEQ ID NO: 216 and SEQ ID NO: 217; a target sequence amplifiable by a primer pair having the sequences SEQ ID NO: 224 and SEQ ID NO: 305; a target sequence amplifiable by a primer pair having the sequences SEQ ID NO: 296 and SEQ ID NO: 297; a target sequence amplifiable by a primer pair having the sequences SEQ ID NO: 298 and SEQ ID NO: 299; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 6 and SEQ ID NO: 306; a target sequence amplifiable by a primer pair having the sequences SEQ ID NO: 307 and SEQ ID NO: 308; a target sequence amplifiable by a primer pair having the sequences SEQ ID NO: 309 and SEQ ID NO: 300; a target sequence amplifiable by a primer pair having the sequences SEQ ID NO: 310 and SEQ ID NO: 311; and a target sequence amplifiable by a primer pair having the sequences SEQ ID NO: 312 and SEQ ID NO: 313.

Aspect of the disclosure relate to a method for constructing a genetic profile for a Cannabis plant, the method comprising testing a deoxyribonucleic acid (DNA) sample from the plant to determine the length of at least two polymorphic target sequences, thereby constructing the genetic profile of the plant, wherein each polymorphic target sequence comprises a simple sequence repeat (SSR) sequence, wherein the at least two polymorphic target sequences comprise two or more of: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; a target sequence flanked by SEQ ID NO: 23 on the 5′ end and SEQ ID NO: 112 on the 3′ end; a target sequence flanked by SEQ ID NO: 25 on the 5′ end and SEQ ID NO: 113 on the 3′ end; a target sequence flanked by SEQ ID NO: 27 on the 5′ end and SEQ ID NO: 114 on the 3′ end; a target sequence flanked by SEQ ID NO: 29 on the 5′ end and SEQ ID NO: 115 on the 3′ end; a target sequence flanked by SEQ ID NO: 31 on the 5′ end and SEQ ID NO: 116 on the 3′ end; a target sequence flanked by SEQ ID NO: 33 on the 5′ end and SEQ ID NO: 117 on the 3′ end; a target sequence flanked by SEQ ID NO: 35 on the 5′ end and SEQ ID NO: 118 on the 3′ end; a target sequence flanked by SEQ ID NO: 37 on the 5′ end and SEQ ID NO: 119 on the 3′ end; a target sequence flanked by SEQ ID NO: 39 on the 5′ end and SEQ ID NO: 120 on the 3′ end; a target sequence flanked by SEQ ID NO: 41 on the 5′ end and SEQ ID NO: 121 on the 3′ end; a target sequence flanked by SEQ ID NO: 43 on the 5′ end and SEQ ID NO: 122 on the 3′ end; a target sequence flanked by SEQ ID NO: 45 on the 5′ end and SEQ ID NO: 123 on the 3′ end; a target sequence flanked by SEQ ID NO: 124 on the 5′ end and SEQ ID NO: 152 on the 3′ end; a target sequence flanked by SEQ ID NO: 126 on the 5′ end and SEQ ID NO: 153 on the 3′ end; a target sequence flanked by SEQ ID NO: 128 on the 5′ end and SEQ ID NO: 154 on the 3′ end; a target sequence flanked by SEQ ID NO: 130 on the 5′ end and SEQ ID NO: 155 on the 3′ end; a target sequence flanked by SEQ ID NO: 132 on the 5′ end and SEQ ID NO: 156 on the 3′ end; a target sequence flanked by SEQ ID NO: 134 on the 5′ end and SEQ ID NO: 157 on the 3′ end; a target sequence flanked by SEQ ID NO: 136 on the 5′ end and SEQ ID NO: 158 on the 3′ end; a target sequence flanked by SEQ ID NO: 138 on the 5′ end and SEQ ID NO: 159 on the 3′ end; a target sequence flanked by SEQ ID NO: 140 on the 5′ end and SEQ ID NO: 160 on the 3′ end; a target sequence flanked by SEQ ID NO: 142 on the 5′ end and SEQ ID NO: 161 on the 3′ end; a target sequence flanked by SEQ ID NO: 144 on the 5′ end and SEQ ID NO: 162 on the 3′ end; a target sequence flanked by SEQ ID NO: 146 on the 5′ end and SEQ ID NO: 163 on the 3′ end; a target sequence flanked by SEQ ID NO: 148 on the 5′ end and SEQ ID NO: 164 on the 3′ end; a target sequence flanked by SEQ ID NO: 150 on the 5′ end and SEQ ID NO: 165 on the 3′ end; a target sequence flanked by SEQ ID NO: 166 on the 5′ end and SEQ ID NO: 252 on the 3′ end; a target sequence flanked by SEQ ID NO: 168 on the 5′ end and SEQ ID NO: 253 on the 3′ end; a target sequence flanked by SEQ ID NO: 170 on the 5′ end and SEQ ID NO: 254 on the 3′ end; a target sequence flanked by SEQ ID NO: 172 on the 5′ end and SEQ ID NO: 255 on the 3′ end; a target sequence flanked by SEQ ID NO: 174 on the 5′ end and SEQ ID NO: 256 on the 3′ end; a target sequence flanked by SEQ ID NO: 176 on the 5′ end and SEQ ID NO: 257 on the 3′ end; a target sequence flanked by SEQ ID NO: 178 on the 5′ end and SEQ ID NO: 258 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 184 on the 5′ end and SEQ ID NO: 261 on the 3′ end; a target sequence flanked by SEQ ID NO: 186 on the 5′ end and SEQ ID NO: 262 on the 3′ end; a target sequence flanked by SEQ ID NO: 188 on the 5′ end and SEQ ID NO: 263 on the 3′ end; a target sequence flanked by SEQ ID NO: 190 on the 5′ end and SEQ ID NO: 264 on the 3′ end; a target sequence flanked by SEQ ID NO: 192 on the 5′ end and SEQ ID NO: 265 on the 3′ end; a target sequence flanked by SEQ ID NO: 194 on the 5′ end and SEQ ID NO: 266 on the 3′ end; a target sequence flanked by SEQ ID NO: 196 on the 5′ end and SEQ ID NO: 267 on the 3′ end; a target sequence flanked by SEQ ID NO: 198 on the 5′ end and SEQ ID NO: 268 on the 3′ end; a target sequence flanked by SEQ ID NO: 200 on the 5′ end and SEQ ID NO: 269 on the 3′ end; a target sequence flanked by SEQ ID NO: 202 on the 5′ end and SEQ ID NO: 270 on the 3′ end; a target sequence flanked by SEQ ID NO: 204 on the 5′ end and SEQ ID NO: 271 on the 3′ end; a target sequence flanked by SEQ ID NO: 206 on the 5′ end and SEQ ID NO: 272 on the 3′ end; a target sequence flanked by SEQ ID NO: 208 on the 5′ end and SEQ ID NO: 273 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 222 on the 5′ end and SEQ ID NO: 280 on the 3′ end; a target sequence flanked by SEQ ID NO: 224 on the 5′ end and SEQ ID NO: 281 on the 3′ end; a target sequence flanked by SEQ ID NO: 226 on the 5′ end and SEQ ID NO: 282 on the 3′ end; a target sequence flanked by SEQ ID NO: 228 on the 5′ end and SEQ ID NO: 283 on the 3′ end; a target sequence flanked by SEQ ID NO: 230 on the 5′ end and SEQ ID NO: 284 on the 3′ end; a target sequence flanked by SEQ ID NO: 232 on the 5′ end and SEQ ID NO: 285 on the 3′ end; a target sequence flanked by SEQ ID NO: 234 on the 5′ end and SEQ ID NO: 286 on the 3′ end; a target sequence flanked by SEQ ID NO: 236 on the 5′ end and SEQ ID NO: 287 on the 3′ end; a target sequence flanked by SEQ ID NO: 238 on the 5′ end and SEQ ID NO: 288 on the 3′ end; a target sequence flanked by SEQ ID NO: 240 on the 5′ end and SEQ ID NO: 289 on the 3′ end; a target sequence flanked by SEQ ID NO: 242 on the 5′ end and SEQ ID NO: 290 on the 3′ end; a target sequence flanked by SEQ ID NO: 244 on the 5′ end and SEQ ID NO: 291 on the 3′ end; a target sequence flanked by SEQ ID NO: 246 on the 5′ end and SEQ ID NO: 292 on the 3′ end; a target sequence flanked by SEQ ID NO: 248 on the 5′ end and SEQ ID NO: 293 on the 3′ end; a target sequence flanked by SEQ ID NO: 168 on the 5′ end and SEQ ID NO: 314 on the 3′ end; a target sequence flanked by SEQ ID NO: 192 on the 5′ end and SEQ ID NO: 317 on the 3′ end; a target sequence flanked by SEQ ID NO: 216 on the 5′ end and SEQ ID NO: 277 on the 3′ end; a target sequence flanked by SEQ ID NO: 224 on the 5′ end and SEQ ID NO: 320 on the 3′ end; a target sequence flanked by SEQ ID NO: 296 on the 5′ end and SEQ ID NO: 315 on the 3′ end; a target sequence flanked by SEQ ID NO: 298 on the 5′ end and SEQ ID NO: 316 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 306 on the 5′ end and SEQ ID NO: 103 on the 3′ end; a target sequence flanked by SEQ ID NO: 307 on the 5′ end and SEQ ID NO: 321 on the 3′ end; a target sequence flanked by SEQ ID NO: 309 on the 5′ end and SEQ ID NO: 317 on the 3′ end; a target sequence flanked by SEQ ID NO: 310 on the 5′ end and SEQ ID NO: 322 on the 3′ end; and a target sequence flanked by SEQ ID NO: 312 on the 5′ end and SEQ ID NO: 323 on the 3′ end.

Aspect of the disclosure relate to a set of primers for constructing a genetic profile for a Cannabis plant, the set comprising a plurality of primer pairs for specifically amplifying at least two polymorphic target sequences, wherein the at least two polymorphic target sequences comprise two or more of: a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 7 and SEQ ID NO: 8; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 15 and SEQ ID NO: 16; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 21 and SEQ ID NO: 22; a target sequence amplifiable by a primer pair having the sequences of SEQ ID NO: 23 and SEQ ID NO: 24; a target sequence amplifiable by a primer pair having the sequences of SEQ ID NO: 25 and SEQ ID NO: 26; a target sequence amplifiable by a primer pair having the sequences of SEQ ID NO: 27 and SEQ ID NO: 28; a target sequence amplifiable by a primer pair having the sequences of SEQ ID NO: 29 and SEQ ID NO: 30; a target sequence amplifiable by a primer pair having the sequences of SEQ ID NO: 31 and SEQ ID NO: 32; a target sequence amplifiable by a primer pair having the sequences of SEQ ID NO: 33 and SEQ ID NO: 34; a target sequence amplifiable by a primer pair having the sequences of SEQ ID NO: 35 and SEQ ID NO: 36; a target sequence amplifiable by a primer pair having the sequences of SEQ ID NO: 37 and SEQ ID NO: 38; a target sequence amplifiable by a primer pair having the sequences of SEQ ID NO: 39 and SEQ ID NO: 40; a target sequence amplifiable by a primer pair having the sequences of SEQ ID NO: 41 and SEQ ID NO: 42; a target sequence amplifiable by a primer pair having the sequences of SEQ ID NO: 43 and SEQ ID NO: 44; a target sequence amplifiable by a primer pair having the sequences of SEQ ID NO: 45 and SEQ ID NO: 46; a target sequence amplifiable by a primer pair having the sequences of SEQ ID NO: 124 and SEQ ID NO: 125; a target sequence amplifiable by a primer pair having the sequences of SEQ ID NO: 126 and SEQ ID NO: 127; a target sequence amplifiable by a primer pair having the sequences of SEQ ID NO: 128 and SEQ ID NO: 129; a target sequence amplifiable by a primer pair having the sequences of SEQ ID NO: 130 and SEQ ID NO: 131; a target sequence amplifiable by a primer pair having the sequences of SEQ ID NO: 132 and SEQ ID NO: 133; a target sequence amplifiable by a primer pair having the sequences of SEQ ID NO: 134 and SEQ ID NO: 135; a target sequence amplifiable by a primer pair having the sequences of SEQ ID NO: 136 and SEQ ID NO: 137; a target sequence amplifiable by a primer pair having the sequences of SEQ ID NO: 138 and SEQ ID NO: 139; a target sequence amplifiable by a primer pair having the sequences of SEQ ID NO: 140 and SEQ ID NO: 141; a target sequence amplifiable by a primer pair having the sequences of SEQ ID NO: 142 and SEQ ID NO: 143; a target sequence amplifiable by a primer pair having the sequences of SEQ ID NO: 144 and SEQ ID NO: 145; a target sequence amplifiable by a primer pair having the sequences of SEQ ID NO: 146 and SEQ ID NO: 147; a target sequence amplifiable by a primer pair having the sequences of SEQ ID NO: 148 and SEQ ID NO: 149; a target sequence amplifiable by a primer pair having the sequences of SEQ ID NO: 150 and SEQ ID NO: 151; a target sequence amplifiable by a primer pair having the sequences of SEQ ID NO: 166 and SEQ ID NO: 167; a target sequence amplifiable by a primer pair having the sequences of SEQ ID NO: 168 and SEQ ID NO: 169; a target sequence amplifiable by a primer pair having the sequences of SEQ ID NO: 170 and SEQ ID NO: 171; a target sequence amplifiable by a primer pair having the sequences of SEQ ID NO: 172 and SEQ ID NO: 173; a target sequence amplifiable by a primer pair having the sequences of SEQ ID NO: 174 and SEQ ID NO: 175; a target sequence amplifiable by a primer pair having the sequences of SEQ ID NO: 176 and SEQ ID NO: 177; a target sequence amplifiable by a primer pair having the sequences of SEQ ID NO: 178 and SEQ ID NO: 179; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 184 and SEQ ID NO: 185; a target sequence amplifiable by a primer pair having the sequences of SEQ ID NO: 186 and SEQ ID NO: 187; a target sequence amplifiable by a primer pair having the sequences of SEQ ID NO: 188 and SEQ ID NO: 189; a target sequence amplifiable by a primer pair having the sequences of SEQ ID NO: 190 and SEQ ID NO: 191; a target sequence amplifiable by a primer pair having the sequences of SEQ ID NO: 192 and SEQ ID NO: 193; a target sequence amplifiable by a primer pair having the sequences of SEQ ID NO: 194 and SEQ ID NO: 195; a target sequence amplifiable by a primer pair having the sequences of SEQ ID NO: 196 and SEQ ID NO: 197; a target sequence amplifiable by a primer pair having the sequences of SEQ ID NO: 198 and SEQ ID NO: 199; a target sequence amplifiable by a primer pair having the sequences of SEQ ID NO: 200 and SEQ ID NO: 201; a target sequence amplifiable by a primer pair having the sequences of SEQ ID NO: 202 and SEQ ID NO: 203; a target sequence amplifiable by a primer pair having the sequences of SEQ ID NO: 204 and SEQ ID NO: 205; a target sequence amplifiable by a primer pair having the sequences of SEQ ID NO: 206 and SEQ ID NO: 207; a target sequence amplifiable by a primer pair having the sequences of SEQ ID NO: 208 and SEQ ID NO: 209; a target sequence amplifiable by a primer pair having the sequences of SEQ ID NO: 210 and SEQ ID NO: 211; a target sequence amplifiable by a primer pair having the sequences of SEQ ID NO: 212 and SEQ ID NO: 213; a target sequence amplifiable by a primer pair having the sequences of SEQ ID NO: 214 and SEQ ID NO: 215; a target sequence amplifiable by a primer pair having the sequences of SEQ ID NO: 216 and SEQ ID NO: 217; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 222 and SEQ ID NO: 223; a target sequence amplifiable by a primer pair having the sequences of SEQ ID NO: 224 and SEQ ID NO: 225; a target sequence amplifiable by a primer pair having the sequences of SEQ ID NO: 226 and SEQ ID NO: 227; a target sequence amplifiable by a primer pair having the sequences of SEQ ID NO: 228 and SEQ ID NO: 229; a target sequence amplifiable by a primer pair having the sequences of SEQ ID NO: 230 and SEQ ID NO: 231; a target sequence amplifiable by a primer pair having the sequences of SEQ ID NO: 232 and SEQ ID NO: 233; a target sequence amplifiable by a primer pair having the sequences of SEQ ID NO: 234 and SEQ ID NO: 235; a target sequence amplifiable by a primer pair having the sequences of SEQ ID NO: 236 and SEQ ID NO: 237; and a target sequence amplifiable by a primer pair having the sequences of SEQ ID NO: 238 and SEQ ID NO: 239; a target sequence amplifiable by a primer pair having the sequences of SEQ ID NO: 240 and SEQ ID NO: 241; a target sequence amplifiable by a primer pair having the sequences of SEQ ID NO: 242 and SEQ ID NO: 243; a target sequence amplifiable by a primer pair having the sequences of SEQ ID NO: 244 and SEQ ID NO: 245; a target sequence amplifiable by a primer pair having the sequences of SEQ ID NO: 246 and SEQ ID NO: 247; a target sequence amplifiable by a primer pair having the sequences of SEQ ID NO: 248 and SEQ ID NO: 249; a target sequence amplifiable by a primer pair having the sequences of SEQ ID NO: 250 and SEQ ID NO: 251; a target sequence amplifiable by a primer pair having the sequences SEQ ID NO: 168 and SEQ ID NO: 295; a target sequence amplifiable by a primer pair having the sequences SEQ ID NO: 192 and SEQ ID NO: 300; a target sequence amplifiable by a primer pair having the sequences SEQ ID NO: 216 and SEQ ID NO: 217; a target sequence amplifiable by a primer pair having the sequences SEQ ID NO: 224 and SEQ ID NO: 305; a target sequence amplifiable by a primer pair having the sequences SEQ ID NO: 296 and SEQ ID NO: 297; a target sequence amplifiable by a primer pair having the sequences SEQ ID NO: 298 and SEQ ID NO: 299; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 6 and SEQ ID NO: 306; a target sequence amplifiable by a primer pair having the sequences SEQ ID NO: 307 and SEQ ID NO: 308; a target sequence amplifiable by a primer pair having the sequences SEQ ID NO: 309 and SEQ ID NO: 300; a target sequence amplifiable by a primer pair having the sequences SEQ ID NO: 310 and SEQ ID NO: 311; and a target sequence amplifiable by a primer pair having the sequences SEQ ID NO: 312 and SEQ ID NO: 313.

Aspect of the disclosure relate to a set of primers for constructing a genetic profile for a Cannabis plant, the set comprising a plurality of primer pairs for specifically amplifying at least two polymorphic target sequences, wherein the at least two polymorphic target sequences comprise two or more of: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; a target sequence flanked by SEQ ID NO: 23 on the 5′ end and SEQ ID NO: 112 on the 3′ end; a target sequence flanked by SEQ ID NO: 25 on the 5′ end and SEQ ID NO: 113 on the 3′ end; a target sequence flanked by SEQ ID NO: 27 on the 5′ end and SEQ ID NO: 114 on the 3′ end; a target sequence flanked by SEQ ID NO: 29 on the 5′ end and SEQ ID NO: 115 on the 3′ end; a target sequence flanked by SEQ ID NO: 31 on the 5′ end and SEQ ID NO: 116 on the 3′ end; a target sequence flanked by SEQ ID NO: 33 on the 5′ end and SEQ ID NO: 117 on the 3′ end; a target sequence flanked by SEQ ID NO: 35 on the 5′ end and SEQ ID NO: 118 on the 3′ end; a target sequence flanked by SEQ ID NO: 37 on the 5′ end and SEQ ID NO: 119 on the 3′ end; a target sequence flanked by SEQ ID NO: 39 on the 5′ end and SEQ ID NO: 120 on the 3′ end; a target sequence flanked by SEQ ID NO: 41 on the 5′ end and SEQ ID NO: 121 on the 3′ end; a target sequence flanked by SEQ ID NO: 43 on the 5′ end and SEQ ID NO: 122 on the 3′ end; a target sequence flanked by SEQ ID NO: 45 on the 5′ end and SEQ ID NO: 123 on the 3′ end; a target sequence flanked by SEQ ID NO: 124 on the 5′ end and SEQ ID NO: 152 on the 3′ end; a target sequence flanked by SEQ ID NO: 126 on the 5′ end and SEQ ID NO: 153 on the 3′ end; a target sequence flanked by SEQ ID NO: 128 on the 5′ end and SEQ ID NO: 154 on the 3′ end; a target sequence flanked by SEQ ID NO: 130 on the 5′ end and SEQ ID NO: 155 on the 3′ end; a target sequence flanked by SEQ ID NO: 132 on the 5′ end and SEQ ID NO: 156 on the 3′ end; a target sequence flanked by SEQ ID NO: 134 on the 5′ end and SEQ ID NO: 157 on the 3′ end; a target sequence flanked by SEQ ID NO: 136 on the 5′ end and SEQ ID NO: 158 on the 3′ end; a target sequence flanked by SEQ ID NO: 138 on the 5′ end and SEQ ID NO: 159 on the 3′ end; a target sequence flanked by SEQ ID NO: 140 on the 5′ end and SEQ ID NO: 160 on the 3′ end; a target sequence flanked by SEQ ID NO: 142 on the 5′ end and SEQ ID NO: 161 on the 3′ end; a target sequence flanked by SEQ ID NO: 144 on the 5′ end and SEQ ID NO: 162 on the 3′ end; a target sequence flanked by SEQ ID NO: 146 on the 5′ end and SEQ ID NO: 163 on the 3′ end; a target sequence flanked by SEQ ID NO: 148 on the 5′ end and SEQ ID NO: 164 on the 3′ end; a target sequence flanked by SEQ ID NO: 150 on the 5′ end and SEQ ID NO: 165 on the 3′ end; a target sequence flanked by SEQ ID NO: 166 on the 5′ end and SEQ ID NO: 252 on the 3′ end; a target sequence flanked by SEQ ID NO: 168 on the 5′ end and SEQ ID NO: 253 on the 3′ end; a target sequence flanked by SEQ ID NO: 170 on the 5′ end and SEQ ID NO: 254 on the 3′ end; a target sequence flanked by SEQ ID NO: 172 on the 5′ end and SEQ ID NO: 255 on the 3′ end; a target sequence flanked by SEQ ID NO: 174 on the 5′ end and SEQ ID NO: 256 on the 3′ end; a target sequence flanked by SEQ ID NO: 176 on the 5′ end and SEQ ID NO: 257 on the 3′ end; a target sequence flanked by SEQ ID NO: 178 on the 5′ end and SEQ ID NO: 258 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 184 on the 5′ end and SEQ ID NO: 261 on the 3′ end; a target sequence flanked by SEQ ID NO: 186 on the 5′ end and SEQ ID NO: 262 on the 3′ end; a target sequence flanked by SEQ ID NO: 188 on the 5′ end and SEQ ID NO: 263 on the 3′ end; a target sequence flanked by SEQ ID NO: 190 on the 5′ end and SEQ ID NO: 264 on the 3′ end; a target sequence flanked by SEQ ID NO: 192 on the 5′ end and SEQ ID NO: 265 on the 3′ end; a target sequence flanked by SEQ ID NO: 194 on the 5′ end and SEQ ID NO: 266 on the 3′ end; a target sequence flanked by SEQ ID NO: 196 on the 5′ end and SEQ ID NO: 267 on the 3′ end; a target sequence flanked by SEQ ID NO: 198 on the 5′ end and SEQ ID NO: 268 on the 3′ end; a target sequence flanked by SEQ ID NO: 200 on the 5′ end and SEQ ID NO: 269 on the 3′ end; a target sequence flanked by SEQ ID NO: 202 on the 5′ end and SEQ ID NO: 270 on the 3′ end; a target sequence flanked by SEQ ID NO: 204 on the 5′ end and SEQ ID NO: 271 on the 3′ end; a target sequence flanked by SEQ ID NO: 206 on the 5′ end and SEQ ID NO: 272 on the 3′ end; a target sequence flanked by SEQ ID NO: 208 on the 5′ end and SEQ ID NO: 273 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 222 on the 5′ end and SEQ ID NO: 280 on the 3′ end; a target sequence flanked by SEQ ID NO: 224 on the 5′ end and SEQ ID NO: 281 on the 3′ end; a target sequence flanked by SEQ ID NO: 226 on the 5′ end and SEQ ID NO: 282 on the 3′ end; a target sequence flanked by SEQ ID NO: 228 on the 5′ end and SEQ ID NO: 283 on the 3′ end; a target sequence flanked by SEQ ID NO: 230 on the 5′ end and SEQ ID NO: 284 on the 3′ end; a target sequence flanked by SEQ ID NO: 232 on the 5′ end and SEQ ID NO: 285 on the 3′ end; a target sequence flanked by SEQ ID NO: 234 on the 5′ end and SEQ ID NO: 286 on the 3′ end; a target sequence flanked by SEQ ID NO: 236 on the 5′ end and SEQ ID NO: 287 on the 3′ end; a target sequence flanked by SEQ ID NO: 238 on the 5′ end and SEQ ID NO: 288 on the 3′ end; a target sequence flanked by SEQ ID NO: 240 on the 5′ end and SEQ ID NO: 289 on the 3′ end; a target sequence flanked by SEQ ID NO: 242 on the 5′ end and SEQ ID NO: 290 on the 3′ end; a target sequence flanked by SEQ ID NO: 244 on the 5′ end and SEQ ID NO: 291 on the 3′ end; a target sequence flanked by SEQ ID NO: 246 on the 5′ end and SEQ ID NO: 292 on the 3′ end; a target sequence flanked by SEQ ID NO: 248 on the 5′ end and SEQ ID NO: 293 on the 3′ end; and a target sequence flanked by SEQ ID NO: 250 on the 5′ end SEQ ID NO: 294 on the 3′ end; a target sequence flanked by SEQ ID NO: 168 on the 5′ end and SEQ ID NO: 314 on the 3′ end; a target sequence flanked by SEQ ID NO: 192 on the 5′ end and SEQ ID NO: 317 on the 3′ end; a target sequence flanked by SEQ ID NO: 216 on the 5′ end and SEQ ID NO: 277 on the 3′ end; a target sequence flanked by SEQ ID NO: 224 on the 5′ end and SEQ ID NO: 320 on the 3′ end; a target sequence flanked by SEQ ID NO: 296 on the 5′ end and SEQ ID NO: 315 on the 3′ end; a target sequence flanked by SEQ ID NO: 298 on the 5′ end and SEQ ID NO: 316 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 306 on the 5′ end and SEQ ID NO: 103 on the 3′ end; a target sequence flanked by SEQ ID NO: 307 on the 5′ end and SEQ ID NO: 321 on the 3′ end; a target sequence flanked by SEQ ID NO: 309 on the 5′ end and SEQ ID NO: 317 on the 3′ end; a target sequence flanked by SEQ ID NO: 310 on the 5′ end and SEQ ID NO: 322 on the 3′ end; and a target sequence flanked by SEQ ID NO: 312 on the 5′ end and SEQ ID NO: 323 on the 3′ end.

Aspect of the disclosure relate to a set of primers for constructing a genetic profile for a Cannabis plant, the set comprising two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; a primer pair having the sequences of SEQ ID NO: 23 and SEQ ID NO: 24; a primer pair having the sequences of SEQ ID NO: 25 and SEQ ID NO: 26; a primer pair having the sequences of SEQ ID NO: 27 and SEQ ID NO: 28; a primer pair having the sequences of SEQ ID NO: 29 and SEQ ID NO: 30; a primer pair having the sequences of SEQ ID NO: 31 and SEQ ID NO: 32; a primer pair having the sequences of SEQ ID NO: 33 and SEQ ID NO: 34; a primer pair having the sequences of SEQ ID NO: 35 and SEQ ID NO: 36; a primer pair having the sequences of SEQ ID NO: 37 and SEQ ID NO: 38; a primer pair having the sequences of SEQ ID NO: 39 and SEQ ID NO: a primer pair having the sequences of SEQ ID NO: 41 and SEQ ID NO: 42; a primer pair having the sequences of SEQ ID NO: 43 and SEQ ID NO: 44; and a primer pair having the sequences of SEQ ID NO: 45 and SEQ ID NO: 46; a primer pair having the sequences of SEQ ID NO: 124 and SEQ ID NO: 125; a primer pair having the sequences of SEQ ID NO: 126 and SEQ ID NO: 127; a primer pair having the sequences of SEQ ID NO: 128 and SEQ ID NO: 129; a primer pair having the sequences of SEQ ID NO: 130 and SEQ ID NO: 131; a primer pair having the sequences of SEQ ID NO: 132 and SEQ ID NO: 133; a primer pair having the sequences of SEQ ID NO: 134 and SEQ ID NO: 135; a primer pair having the sequences of SEQ ID NO: 136 and SEQ ID NO: 137; a primer pair having the sequences of SEQ ID NO: 138 and SEQ ID NO: 139; a primer pair having the sequences of SEQ ID NO: 140 and SEQ ID NO: 141; a primer pair having the sequences of SEQ ID NO: 142 and SEQ ID NO: 143; a primer pair having the sequences of SEQ ID NO: 144 and SEQ ID NO: 145; a primer pair having the sequences of SEQ ID NO: 146 and SEQ ID NO: 147; a primer pair having the sequences of SEQ ID NO: 148 and SEQ ID NO: 149; a primer pair having the sequences of SEQ ID NO: 150 and SEQ ID NO: 151; a primer pair having the sequences of SEQ ID NO: 166 and SEQ ID NO: 167; a primer pair having the sequences of SEQ ID NO: 168 and SEQ ID NO: 169; a primer pair having the sequences of SEQ ID NO: 170 and SEQ ID NO: 171; a primer pair having the sequences of SEQ ID NO: 172 and SEQ ID NO: 173; a primer pair having the sequences of SEQ ID NO: 174 and SEQ ID NO: 175; a primer pair having the sequences of SEQ ID NO: 176 and SEQ ID NO: 177; a primer pair having the sequences of SEQ ID NO: 178 and SEQ ID NO: 179; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 184 and SEQ ID NO: 185; a primer pair having the sequences of SEQ ID NO: 186 and SEQ ID NO: 187; a primer pair having the sequences of SEQ ID NO: 188 and SEQ ID NO: 189; a primer pair having the sequences of SEQ ID NO: 190 and SEQ ID NO: 191; a primer pair having the sequences of SEQ ID NO: 192 and SEQ ID NO: 193; a primer pair having the sequences of SEQ ID NO: 194 and SEQ ID NO: 195; a primer pair having the sequences of SEQ ID NO: 196 and SEQ ID NO: 197; a primer pair having the sequences of SEQ ID NO: 198 and SEQ ID NO: 199; a primer pair having the sequences of SEQ ID NO: 200 and SEQ ID NO: 201; a primer pair having the sequences of SEQ ID NO: 202 and SEQ ID NO: 203; a primer pair having the sequences of SEQ ID NO: 204 and SEQ ID NO: 205; a primer pair having the sequences of SEQ ID NO: 206 and SEQ ID NO: 207; a primer pair having the sequences of SEQ ID NO: 208 and SEQ ID NO: 209; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 216 and SEQ ID NO: 217; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 222 and SEQ ID NO: 223; a primer pair having the sequences of SEQ ID NO: 224 and SEQ ID NO: 225; a primer pair having the sequences of SEQ ID NO: 226 and SEQ ID NO: 227; a primer pair having the sequences of SEQ ID NO: 228 and SEQ ID NO: 229; a primer pair having the sequences of SEQ ID NO: 230 and SEQ ID NO: 231; a primer pair having the sequences of SEQ ID NO: 232 and SEQ ID NO: 233; a primer pair having the sequences of SEQ ID NO: 234 and SEQ ID NO: 235; a primer pair having the sequences of SEQ ID NO: 236 and SEQ ID NO: 237; a primer pair having the sequences of SEQ ID NO: 238 and SEQ ID NO: 239; a primer pair having the sequences of SEQ ID NO: 240 and SEQ ID NO: 241; a primer pair having the sequences of SEQ ID NO: 242 and SEQ ID NO: 243; a primer pair having the sequences of SEQ ID NO: 244 and SEQ ID NO: 245; a primer pair having the sequences of SEQ ID NO: 246 and SEQ ID NO: 247; a primer pair having the sequences of SEQ ID NO: 248 and SEQ ID NO: 249; a primer pair having the sequences of SEQ ID NO: 250 and SEQ ID NO: 251; a primer pair having the sequences of SEQ ID NO: 168 and SEQ ID NO: 295; a primer pair having the sequences of SEQ ID NO: 192 and SEQ ID NO: 300; a primer pair having the sequences of SEQ ID NO: 216 and SEQ ID NO: 217; a primer pair having the sequences of SEQ ID NO: 224 and SEQ ID NO: 305; a primer pair having the sequences of SEQ ID NO: 296 and SEQ ID NO: 297; a primer pair having the sequences of SEQ ID NO: 298 and SEQ ID NO: 299; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 6 and SEQ ID NO: 306; a primer pair having the sequences of SEQ ID NO: 307 and SEQ ID NO: 308; a primer pair having the sequences of SEQ ID NO: 309 and SEQ ID NO: 300; a primer pair having the sequences of SEQ ID NO: 310 and SEQ ID NO: 311; and a primer pair having the sequences of SEQ ID NO: 312 and SEQ ID NO: 313.

Aspect of the disclosure relate to a set of primers for constructing a genetic profile for a Cannabis plant, the set comprising a plurality of primer pairs for specifically amplifying at least five polymorphic target sequences, wherein the at least five polymorphic target sequences comprise: a target sequence flanked by SEQ ID NO: 1 on the 5′ end and SEQ ID NO: 101 on the 3′ end; a target sequence flanked by SEQ ID NO: 7 on the 5′ end and SEQ ID NO: 104 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; and a target sequence flanked by SEQ ID NO: 23 on the 5′ end and SEQ ID NO: 112 on the 3′ end.

Aspect of the disclosure relate to a set of primers for constructing a genetic profile for a Cannabis plant, the set comprising a plurality of primer pairs for specifically amplifying at least five polymorphic target sequences, wherein the at least five polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 7 and SEQ ID NO: 8; a target polymorphic sequence amplifiable by a primer pair having the sequences of SEQ ID NO: 17 and SEQ ID NO: 18; a target polymorphic sequence amplifiable by a primer pair having the sequences of SEQ ID NO: 19 and SEQ ID NO: 20; and a target polymorphic sequence amplifiable by a primer pair having the sequences of SEQ ID NO: 23 and SEQ ID NO: 24.

Aspect of the disclosure relate to a set of primers for constructing a genetic profile for a Cannabis plant, the set comprising a plurality of primer pairs for specifically amplifying at least seven polymorphic target sequences, wherein the at least seven polymorphic target sequences comprise: a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 15 and SEQ ID NO: 16; and a target polymorphic sequence amplifiable by a primer pair having the sequences of SEQ ID NO: 21 and SEQ ID NO: 22.

Aspect of the disclosure relate to a set of primers for constructing a genetic profile for a Cannabis plant, the set comprising a plurality of primer pairs for specifically amplifying at least seven polymorphic target sequences, wherein the at least seven polymorphic target sequences comprise: a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; and a target sequence flanked by SEQ ID NO: 21 on the 5′ end and SEQ ID NO: 111 on the 3′ end.

Aspect of the disclosure relate to a set of primers for constructing a genetic profile for a Cannabis plant, the set comprising: a first subset of primer pairs for specifically amplifying at least five polymorphic target sequences, wherein the at least five polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 7 and SEQ ID NO: 8; a target polymorphic sequence amplifiable by a primer pair having the sequences of SEQ ID NO: 17 and SEQ ID NO: 18; a target polymorphic sequence amplifiable by a primer pair having the sequences of SEQ ID NO: 19 and SEQ ID NO: 20; and a target polymorphic sequence amplifiable by a primer pair having the sequences of SEQ ID NO: 23 and SEQ ID NO: 24, and a second subset of primer pairs for specifically amplifying seven polymorphic target sequences, wherein the at least seven polymorphic target sequences comprise: a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 15 and SEQ ID NO: 16; and a target polymorphic sequence amplifiable by a primer pair having the sequences of SEQ ID NO: 21 and SEQ ID NO: 22.

Aspect of the disclosure relate to a set of primers for constructing a genetic profile for a Cannabis plant, the set comprising: a first subset of primer pairs for specifically amplifying at least five polymorphic target sequences, wherein the at least five polymorphic target sequences comprise: a target sequence flanked by SEQ ID NO: 1 on the 5′ end and SEQ ID NO: 101 on the 3′ end; a target sequence flanked by SEQ ID NO: 7 on the 5′ end and SEQ ID NO: 104 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; and a target sequence flanked by SEQ ID NO: 23 on the 5′ end and SEQ ID NO: 112 on the 3′ end, and a second subset of primer pairs for specifically amplifying seven polymorphic target sequences, wherein the at least seven polymorphic target sequences comprise: a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; and a target sequence flanked by SEQ ID NO: 21 on the 5′ end and SEQ ID NO: 111 on the 3′ end.

Aspect of the disclosure relate to a set of primers for constructing a genetic profile for a Cannabis plant, the set comprising a plurality of primer pairs for specifically amplifying at least six polymorphic target sequences, wherein the at least six polymorphic target sequences comprise: a target polymorphic sequence amplifiable by a primer pair having the sequences of SEQ ID NO: 7 and SEQ ID NO: 8; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 29 and SEQ ID NO: 30; a target polymorphic sequence amplifiable by a primer pair having the sequences of SEQ ID NO: 37 and SEQ ID NO: 38; and a target polymorphic sequence amplifiable by a primer pair having the sequences of SEQ ID NO: 45 and SEQ ID NO: 46.

Aspect of the disclosure relate to a set of primers for constructing a genetic profile for a Cannabis plant, the set comprising a plurality of primer pairs for specifically amplifying at least six polymorphic target sequences, wherein the at least six polymorphic target sequences comprise: a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 29 on the 5′ end and SEQ ID NO: 115 on the 3′ end; a target sequence flanked by SEQ ID NO: 37 on the 5′ end and SEQ ID NO: 119 on the 3′ end; and a target sequence flanked by SEQ ID NO: 45 on the 5′ end and SEQ ID NO: 123 on the 3′ end.

Aspect of the disclosure relate to a set of primers for constructing a genetic profile for a Cannabis plant, the set comprising a plurality of primer pairs for specifically amplifying at least six polymorphic target sequences, wherein the at least six polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 39 and SEQ ID NO: 40; and a target polymorphic sequence amplifiable by a primer pair having the sequences of SEQ ID NO: 43 and SEQ ID NO: 44.

Aspect of the disclosure relate to a set of primers for constructing a genetic profile for a Cannabis plant, the set comprising a plurality of primer pairs for specifically amplifying at least six polymorphic target sequences, wherein the at least six polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 13 on the 5′ end and SEQ ID NO: 107 on the 3′ end; a target sequence flanked by SEQ ID NO: 39 on the 5′ end and SEQ ID NO: 120 on the 3′ end; and a target sequence flanked by SEQ ID NO: 43 on the 5′ end and SEQ ID NO: 122 on the 3′ end.

Aspect of the disclosure relate to a set of primers for constructing a genetic profile for a Cannabis plant, the set comprising: a first subset of primer pairs for specifically amplifying at least six polymorphic target sequences, wherein the at least six polymorphic target sequences comprise: a target polymorphic sequence amplifiable by a primer pair having the sequences of SEQ ID NO: 7 and SEQ ID NO: 8; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 29 and SEQ ID NO: 30; a target polymorphic sequence amplifiable by a primer pair having the sequences of SEQ ID NO: 37 and SEQ ID NO: 38; and a target polymorphic sequence amplifiable by a primer pair having the sequences of SEQ ID NO: 45 and SEQ ID NO: 46, and a second subset of primer pairs for specifically amplifying seven polymorphic target sequences, wherein the at least six polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 39 and SEQ ID NO: 40; and a target polymorphic sequence amplifiable by a primer pair having the sequences of SEQ ID NO: 43 and SEQ ID NO: 44.

Aspect of the disclosure relate to a set of primers for constructing a genetic profile for a Cannabis plant, the set comprising: a first subset of primer pairs for specifically amplifying at least six polymorphic target sequences, wherein the at least six polymorphic target sequences comprise: a target sequence flanked by SEQ ID NO: 7 on the 5′ end and SEQ ID NO: 104 on the 3′ end; a target sequence flan ked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 29 on the 5′ end and SEQ ID NO: 115 on the 3′ end; a target sequence flanked by SEQ ID NO: 37 on the 5′ end and SEQ ID NO: 119 on the 3′ end; and a target sequence flanked by SEQ ID NO: 45 on the 5′ end and SEQ ID NO: 123 on the 3′ end, and a second subset of primer pairs for specifically amplifying seven polymorphic target sequences, wherein the at least six polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 13 on the 5′ end and SEQ ID NO: 107 on the 3′ end; a target sequence flanked by SEQ ID NO: 39 on the 5′ end and SEQ ID NO: 120 on the 3′ end; and a target sequence flanked by SEQ ID NO: 43 on the 5′ end and SEQ ID NO: 122 on the 3′ end.

Aspect of the disclosure relate to a set of primers for constructing a genetic profile for a Cannabis plant, the set comprising a plurality of primer pairs for specifically amplifying at least ten polymorphic target sequences, wherein the at least ten polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; and a target sequence flanked by SEQ ID NO: 23 on the 5′ end and SEQ ID NO: 112 on the 3′ end.

Aspect of the disclosure relate to a set of primers for constructing a genetic profile for a Cannabis plant, the set comprising a plurality of primer pairs for specifically amplifying at least ten polymorphic target sequences, wherein the at least ten polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 17 and SEQ ID NO: 18; a target polymorphic sequence amplifiable by a primer pair having the sequences of SEQ ID NO: 19 and SEQ ID NO: 20; a target polymorphic sequence amplifiable by a primer pair having the sequences of SEQ ID NO: 21 and SEQ ID NO: 22; and a target polymorphic sequence amplifiable by a primer pair having the sequences of SEQ ID NO: 23 and SEQ ID NO: 24.

Aspect of the disclosure relate to a set of primers for constructing a genetic profile for a Cannabis plant, the set comprising a plurality of primer pairs for specifically amplifying at least eleven polymorphic target sequences, wherein the at least eleven polymorphic target sequences comprise: a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

Aspect of the disclosure relate to a set of primers for constructing a genetic profile for a Cannabis plant, the set comprising a plurality of primer pairs for specifically amplifying at least eleven polymorphic target sequences, wherein the at least eleven polymorphic target sequences comprise: a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 220 and SEQ ID NO: 221; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences SEQ ID NO: 248 and SEQ ID NO: 249; and a target polymorphic sequence amplifiable by a primer pair having the sequences of SEQ ID NO: 250 and SEQ ID NO: 251.

Aspect of the disclosure relate to a set of primers for constructing a genetic profile for a Cannabis plant, the set comprising a plurality of primer pairs for specifically amplifying at least twelve polymorphic target sequences, wherein the at least twelve polymorphic target sequences comprise: a target sequence flanked by SEQ ID NO: 170 on the 5′ end and SEQ ID NO: 254 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

Aspect of the disclosure relate to a set of primers for constructing a genetic profile for a Cannabis plant, the set comprising a plurality of primer pairs for specifically amplifying at least twelve polymorphic target sequences, wherein the at least twelve polymorphic target sequences comprise: a target polymorphic sequence amplifiable by a primer pair having the sequences of SEQ ID NO: 170 and SEQ ID NO: 171; a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 220 and SEQ ID NO: 221; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences of SEQ ID NO: 248 and SEQ ID NO: 249; and a target polymorphic sequence amplifiable by a primer pair having the sequences of SEQ ID NO: 250 and SEQ ID NO: 251.

Aspect of the disclosure relate to a set of primers for constructing a genetic profile for a Cannabis plant, the set comprising a plurality of primer pairs for specifically amplifying at least thirteen polymorphic target sequences, wherein the at least thirteen polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

Aspect of the disclosure relate to a set of primers for constructing a genetic profile for a Cannabis plant, the set comprising a plurality of primer pairs for specifically amplifying at least thirteen polymorphic target sequences, wherein the at least thirteen polymorphic target sequences comprise: a target polymorphic sequence amplifiable by a primer pair having the sequences of SEQ ID NO: 170 and SEQ ID NO: 171; a target polymorphic sequence amplifiable by a primer pair having the sequences of SEQ ID NO: 174 and SEQ ID NO: 175; a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 220 and SEQ ID NO: 221; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences of SEQ ID NO: 248 and SEQ ID NO: 249; and a target polymorphic sequence amplifiable by a primer pair having the sequences of SEQ ID NO: 250 and SEQ ID NO: 251.

Aspect of the disclosure relate to a set of primers for constructing a genetic profile for a Cannabis plant, the set comprising a plurality of primer pairs for specifically amplifying at least fourteen polymorphic target sequences, wherein the at least fourteen polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

Aspect of the disclosure relate to a set of primers for constructing a genetic profile for a Cannabis plant, the set comprising a plurality of primer pairs for specifically amplifying at least fourteen polymorphic target sequences, wherein the at least fourteen polymorphic target sequences comprise: a target polymorphic sequence amplifiable by a primer pair having the sequences of SEQ ID NO: 170 and SEQ ID NO: 171; a target polymorphic sequence amplifiable by a primer pair having the sequences of SEQ ID NO: 174 and SEQ ID NO: 175; a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 212 and SEQ ID NO: 213; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 220 and SEQ ID NO: 221; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences of SEQ ID NO: 248 and SEQ ID NO: 249; and a target polymorphic sequence amplifiable by a primer pair having the sequences of SEQ ID NO: 250 and SEQ ID NO: 251.

Aspect of the disclosure relate to a set of primers for constructing a genetic profile for a Cannabis plant, the set comprising a plurality of primer pairs for specifically amplifying at least eighteen polymorphic target sequences, wherein the at least eighteen polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 296 on the 5′ end and SEQ ID NO: 315 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 192 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

Aspect of the disclosure relate to a set of primers for constructing a genetic profile for a Cannabis plant, the set comprising a plurality of primer pairs for specifically amplifying at least eighteen polymorphic target sequences, wherein the at least eighteen polymorphic target sequences comprise: a target polymorphic sequence amplifiable by a primer pair having the sequences SEQ ID NO: 168 and SEQ ID NO: 295; a target polymorphic sequence amplifiable by a primer pair having the sequences SEQ ID NO: 170 and SEQ ID NO: 171; a target polymorphic sequence amplifiable by a primer pair having the sequences SEQ ID NO: 174 and SEQ ID NO: 175; a target polymorphic sequence amplifiable by a primer pair having the sequences SEQ ID NO: 296 and SEQ ID NO: 297; a target polymorphic sequence amplifiable by a primer pair having the sequences SEQ ID NO: 180 and SEQ ID NO: 181; a target polymorphic sequence amplifiable by a primer pair having the sequences SEQ ID NO: 298 and SEQ ID NO: 299; a target polymorphic sequence amplifiable by a primer pair having the sequences SEQ ID NO: 192 and SEQ ID NO: 300; a target polymorphic sequence amplifiable by a primer pair having the sequences SEQ ID NO: 301 and SEQ ID NO: 302; a target polymorphic sequence amplifiable by a primer pair having the sequences SEQ ID NO: 303 and SEQ ID NO: 304; a target polymorphic sequence amplifiable by a primer pair having the sequences SEQ ID NO: 210 and SEQ ID NO: 211; a target polymorphic sequence amplifiable by a primer pair having the sequences SEQ ID NO: 212 and SEQ ID NO: 213; a target polymorphic sequence amplifiable by a primer pair having the sequences SEQ ID NO: 214 and SEQ ID NO: 215; a target polymorphic sequence amplifiable by a primer pair having the sequences SEQ ID NO: 216 and SEQ ID NO: 217; a target polymorphic sequence amplifiable by a primer pair having the sequences SEQ ID NO: 218 and SEQ ID NO: 219; a target polymorphic sequence amplifiable by a primer pair having the sequences SEQ ID NO: 220 and SEQ ID NO: 221; a target polymorphic sequence amplifiable by a primer pair having the sequences SEQ ID NO: 224 and SEQ ID NO: 305; a target polymorphic sequence amplifiable by a primer pair having the sequences SEQ ID NO: 244 and SEQ ID NO: 245; and a target polymorphic sequence amplifiable by a primer pair having the sequences SEQ ID NO: 6 and SEQ ID NO: 306.

Aspect of the disclosure relate to a set of primers for constructing a genetic profile for a Cannabis plant, the set comprising a plurality of primer pairs for specifically amplifying at least eighteen polymorphic target sequences, wherein the at least eighteen polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 312 on the 5′ end and SEQ ID NO: 323 on the 3′ end; a target sequence flanked by SEQ ID NO: 307 on the 5′ end and SEQ ID NO: 321 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 309 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 310 on the 5′ end and SEQ ID NO: 322 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

Aspect of the disclosure relate to a set of primers for constructing a genetic profile for a Cannabis plant, the set comprising a plurality of primer pairs for specifically amplifying at least eighteen polymorphic target sequences, wherein the at least eighteen polymorphic target sequences comprise: a target polymorphic sequence amplifiable by a primer pair having the sequences SEQ ID NO: 168 and SEQ ID NO: 295; a target polymorphic sequence amplifiable by a primer pair having the sequences SEQ ID NO: 170 and SEQ ID NO: 171; a target polymorphic sequence amplifiable by a primer pair having the sequences SEQ ID NO: 312 and SEQ ID NO: 313; a target polymorphic sequence amplifiable by a primer pair having the sequences SEQ ID NO: 307 and SEQ ID NO: 308; a target polymorphic sequence amplifiable by a primer pair having the sequences SEQ ID NO: 180 and SEQ ID NO: 181; a target polymorphic sequence amplifiable by a primer pair having the sequences SEQ ID NO: 298 and SEQ ID NO: 299; a target polymorphic sequence amplifiable by a primer pair having the sequences SEQ ID NO: 309 and SEQ ID NO: 300; a target polymorphic sequence amplifiable by a primer pair having the sequences SEQ ID NO: 301 and SEQ ID NO: 302; a target polymorphic sequence amplifiable by a primer pair having the sequences SEQ ID NO: 303 and SEQ ID NO: 304; a target polymorphic sequence amplifiable by a primer pair having the sequences SEQ ID NO: 210 and SEQ ID NO: 211; a target polymorphic sequence amplifiable by a primer pair having the sequences SEQ ID NO: 212 and SEQ ID NO: 213; a target polymorphic sequence amplifiable by a primer pair having the sequences SEQ ID NO: 214 and SEQ ID NO: 215; a target polymorphic sequence amplifiable by a primer pair having the sequences SEQ ID NO: 216 and SEQ ID NO: 217; a target polymorphic sequence amplifiable by a primer pair having the sequences SEQ ID NO: 310 and SEQ ID NO: 311; a target polymorphic sequence amplifiable by a primer pair having the sequences SEQ ID NO: 220 and SEQ ID NO: 221; a target polymorphic sequence amplifiable by a primer pair having the sequences SEQ ID NO: 224 and SEQ ID NO: 305; a target polymorphic sequence amplifiable by a primer pair having the sequences SEQ ID NO: 244 and SEQ ID NO: 245; and a target polymorphic sequence amplifiable by a primer pair having the sequences SEQ ID NO: 6 and SEQ ID NO: 306.

Aspect of the disclosure relate to a set of primers for constructing a genetic profile for a Cannabis plant, the set comprising two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; a primer pair having the sequences of SEQ ID NO: 23 and SEQ ID NO: 24; a primer pair having the sequences of SEQ ID NO: 27 and SEQ ID NO: 28; a primer pair having the sequences of SEQ ID NO: 29 and SEQ ID NO: a primer pair having the sequences of SEQ ID NO: 37 and SEQ ID NO: 38; a primer pair having the sequences of SEQ ID NO: 39 and SEQ ID NO: 40; a primer pair having the sequences of SEQ ID NO: 43 and SEQ ID NO: 44; a primer pair having the sequences of SEQ ID NO: 45 and SEQ ID NO: 46; a primer pair having the sequences of SEQ ID NO: 124 and SEQ ID NO: 125; a primer pair having the sequences of SEQ ID NO: 126 and SEQ ID NO: 127; a primer pair having the sequences of SEQ ID NO: 130 and SEQ ID NO: 131; a primer pair having the sequences of SEQ ID NO: 132 and SEQ ID NO: 133; a primer pair having the sequences of SEQ ID NO: 134 and SEQ ID NO: 135; a primer pair having the sequences of SEQ ID NO: 136 and SEQ ID NO: 137; a primer pair having the sequences of SEQ ID NO: 138 and SEQ ID NO: 139; a primer pair having the sequences of SEQ ID NO: 140 and SEQ ID NO: 141; a primer pair having the sequences of SEQ ID NO: 142 and SEQ ID NO: 143; a primer pair having the sequences of SEQ ID NO: 144 and SEQ ID NO: 145; a primer pair having the sequences of SEQ ID NO: 146 and SEQ ID NO: 147; a primer pair having the sequences of SEQ ID NO: 148 and SEQ ID NO: 149; a primer pair having the sequences of SEQ ID NO: 150 and SEQ ID NO: 151; a primer pair having the sequences of SEQ ID NO: 166 and SEQ ID NO: 167; a primer pair having the sequences of SEQ ID NO: 168 and SEQ ID NO: 169; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 176 and SEQ ID NO: 177; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 184 and SEQ ID NO: 185; a primer pair having the sequences of SEQ ID NO: 186 and SEQ ID NO: 187; a primer pair having the sequences of SEQ ID NO: 188 and SEQ ID NO: 189; a primer pair having the sequences of SEQ ID NO: 190 and SEQ ID NO: 191; a primer pair having the sequences of SEQ ID NO: 196 and SEQ ID NO: 197; a primer pair having the sequences of SEQ ID NO: 200 and SEQ ID NO: 201; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 222 and SEQ ID NO: 223; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 230 and SEQ ID NO: 231; a primer pair having the sequences of SEQ ID NO: 232 and SEQ ID NO: 233; a primer pair having the sequences of SEQ ID NO: 238 and SEQ ID NO: 239; a primer pair having the sequences of SEQ ID NO: 240 and SEQ ID NO: 241; a primer pair having the sequences of SEQ ID NO: 242 and SEQ ID NO: 243; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251; a primer pair having the sequences of SEQ ID NO: 168 and SEQ ID NO: 295; a primer pair having the sequences of SEQ ID NO: 192 and SEQ ID NO: 300; a primer pair having the sequences of SEQ ID NO: 216 and SEQ ID NO: 217; a primer pair having the sequences of SEQ ID NO: 224 and SEQ ID NO: 305; a primer pair having the sequences of SEQ ID NO: 296 and SEQ ID NO: 297; a primer pair having the sequences of SEQ ID NO: 298 and SEQ ID NO: 299; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 6 and SEQ ID NO: 306; a primer pair having the sequences of SEQ ID NO: 307 and SEQ ID NO: 308; a primer pair having the sequences of SEQ ID NO: 309 and SEQ ID NO: 300; a primer pair having the sequences of SEQ ID NO: 310 and SEQ ID NO: 311; and a primer pair having the sequences of SEQ ID NO: 312 and SEQ ID NO: 313.

Aspect of the disclosure relate to a set of primers for constructing a genetic profile for a Cannabis plant, the set comprising: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24.

Aspect of the disclosure relate to a set of primers for constructing a genetic profile for a Cannabis plant, the set comprising: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

Aspect of the disclosure relate to a set of primers for constructing a genetic profile for a Cannabis plant, the set comprising: a first subset of primer pairs comprising: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24, and a second subset of primer pairs comprising: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

Aspect of the disclosure relate to a set of primers for constructing a genetic profile for a Cannabis plant, the set comprising: a primer pair having the sequences of SEQ ID NO: 7 and SEQ ID NO: 8; a primer pair having the sequences of SEQ ID NO: 9 and SEQ ID NO: a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; and a primer pair having the sequences of SEQ ID NO: 45 and SEQ ID NO: 46.

Aspect of the disclosure relate to a set of primers for constructing a genetic profile for a Cannabis plant, the set comprising: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

Aspect of the disclosure relate to a set of primers for constructing a genetic profile for a Cannabis plant, the set comprising: a first subset of primer pairs comprising: a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; a primer pair having the sequences of SEQ ID NO: 45 and SEQ ID NO: 46, and a second subset of primer pairs comprising: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

Aspect of the disclosure relate to a set of primers for constructing a genetic profile for a Cannabis plant, the set comprising: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 17 and SEQ ID NO: 18; a primer pair having the sequences of SEQ ID NO: 19 and SEQ ID NO: a primer pair having the sequences of SEQ ID NO: 21 and SEQ ID NO: 22; and a primer pair having the sequences of SEQ ID NO: 23 and SEQ ID NO: 24.

Aspect of the disclosure relate to a set of primers for constructing a genetic profile for a Cannabis plant, the set comprising: a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

Aspect of the disclosure relate to a set of primers for constructing a genetic profile for a Cannabis plant, the set comprising: a primer pair having the sequences SEQ ID NO: 170 and SEQ ID NO: 171; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

Aspect of the disclosure relate to a set of primers for constructing a genetic profile for a Cannabis plant, the set comprising: a primer pair having the sequences SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

Aspect of the disclosure relate to a set of primers for constructing a genetic profile for a Cannabis plant, the set comprising: a primer pair having the sequences SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

Aspect of the disclosure relate to a set of primers for constructing a genetic profile for a Cannabis plant, the set comprising: a primer pair having the sequences of SEQ ID NO: 168 and SEQ ID NO: 295; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 296 and SEQ ID NO: 297; a primer pair having the sequences of SEQ ID NO: 180 and SEQ ID NO: 181; a primer pair having the sequences of SEQ ID NO: 298 and SEQ ID NO: 299; a primer pair having the sequences of SEQ ID NO: 192 and SEQ ID NO: 300; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 216 and SEQ ID NO: 217; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 305; a primer pair having the sequences of SEQ ID NO: 244 and SEQ ID NO: 245; and a primer pair having the sequences of SEQ ID NO: 6 and SEQ ID NO: 306.

Aspect of the disclosure relate to a set of primers for constructing a genetic profile for a Cannabis plant, the set comprising: a primer pair having the sequences of SEQ ID NO: 168 and SEQ ID NO: 295; a primer pair having the sequences of SEQ ID NO: 170 and SEQ ID NO: 171; a primer pair having the sequences of SEQ ID NO: 312 and SEQ ID NO: 313; a primer pair having the sequences of SEQ ID NO: 307 and SEQ ID NO: 308; a primer pair having the sequences of SEQ ID NO: 180 and SEQ ID NO: 181; a primer pair having the sequences of SEQ ID NO: 298 and SEQ ID NO: 299; a primer pair having the sequences of SEQ ID NO: 309 and SEQ ID NO: 300; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 216 and SEQ ID NO: 217; a primer pair having the sequences of SEQ ID NO: 310 and SEQ ID NO: 311; a primer pair having the sequences of SEQ ID NO: 220 and SEQ ID NO: 221; a primer pair having the sequences of SEQ ID NO: 224 and SEQ ID NO: 305; a primer pair having the sequences of SEQ ID NO: 244 and SEQ ID NO: 245; and a primer pair having the sequences of SEQ ID NO: 6 and SEQ ID NO: 306.

### Definitions

“Allele” as used herein refers to one of at least two alternative forms of a gene that arise by mutation and are found at the same location on a chromosome.

“Allele set” as used herein refers to the precise combination of alleles identified for a particular set of SSR loci in an individual.

“Flanked” or “flanking” as used herein refers to being on each or on one side of a sequence of interest, e.g. an SSR target sequence.

“Multiplex reaction” as used herein refers a single reaction in which several different DNA sequences are amplified simultaneously in a single reaction mix for simultaneous analysis, as opposed to individual reactions in which each DNA sequence is amplified in isolation.

“Plant material” as used herein refers to any portion or combination of portions of a Cannabis plant including but not limited to seeds, stems, stalks, leaves, flowers, roots, whether fresh or dry, or whether intact, cut, or comminuted.

“Provenance” as used herein refers to the origin of a plant or plant variety.

The term “traditional breeding techniques” as used herein includes crossing, selfing, selection, backcrossing, marker assisted breeding/selection, mutation breeding etc. as known to the breeder (i.e. methods other than genetic modification/transformation/transgenic methods), by which, for example, a genetically heritable trait can be transferred from one Cannabis line or variety to another.

“Backcrossing” as used herein is a traditional breeding technique used to introduce a trait into a plant line or variety. A first parent containing a trait of interest is crossed to a second donor parent to produce progeny plants known as Recurrent Parents (RP). The RP plants which have the trait are then “backcrossed” with the first parent (with the trait of interest) or the donor parent, usually the donor parent. After several generations of backcrossing and/or selfing, the progeny plants typically have the genotype of the donor parent but with the trait of interest from the first parent.

“Simple sequence repeats” (“SSRs”), also referred to as “microsatellites”, “short tandem repeats” (“STRs”), or “variable number tandem repeats” (“VNTRs”), as used herein are stretches of DNA comprising core repeat units of two or more nucleotides in length that are tandemly repeated generally between a half dozen and several dozen times.

### DETAILED DESCRIPTION

### Polymorphisms

The genomes of all organisms undergo spontaneous mutations in the course of their evolution to generate variant forms of progenitor genetic sequences, whether they be substitutions of one nucleotide for another at the polymorphic site, or insertions or deletions of as few as one nucleotide and as many as several thousand nucleotides. In many cases, both progenitor and variant forms survive and co-exist in a species population. The coexistence of multiple forms of a genetic sequence segregating at appreciable frequencies is defined as a “genetic polymorphism” at a “polymorphic site”, which includes single nucleotide polymorphisms (“SNPs”), i.e. single base positions in DNA at which different alleles, or alternative nucleotides, exist in a population.

A fast and efficient method of uniquely genotyping plants involves the use of simple sequence repeat (SSR) markers, or random amplified polymorphic DNA (RAPD). SSR markers, also called microsatellites, are repeat sequences that are typically distributed evenly throughout the genome of plants and animals and are commonly used for genotyping in both the human and agricultural context.

The use of SSR markers for genotyping is based on the existence of SSR loci that are polymorphic. That is, the number of repeats at a particular SSR locus may vary due to duplication or deletion of the repeat units, thereby resulting in variants (i.e. alleles) at the locus which differ in terms of nucleotide length. The number of variants (i.e. alleles) that exist at any one SSR locus may also vary. That is, for a single SSR locus, two or more alleles may exist comprising a range of possible sizes, including differences of as few as 1 base pair (bp), which allows for significant diversity within a single locus. The precise combination of alleles across several polymorphic loci can serve as a unique signature of an individual plant's genotype.

Several thousand Cannabis SSR markers have been identified in the literature, including in Alghanim and Almirall (Analytical and Bioanalytical Chemistry, 376(8), 1225-1233), Gao et al. (PLoS ONE, 9(10): e110638), Gilmore and Peakall (Molecular Ecology Notes, 105-107), Dufresnes et al. (PLOS ONE, 12(1), e0170522), Howard et al. (Application of new DNA markers for forensic examination of Cannabis sativa seizures-Developmental validation of protocols and a genetic database funded by the National Drug Law Enforcement Research Fund, an initiative of the National Drug Strategy available at https://aic.gov.au/sites/default/files/monograph-29.pdf?v=1578542645), Houston et al. (International Journal of Legal Medicine, 130(3), 635-647), Presinszka et al. (2015, Analysis of Microsatellite Markers in Hemp (Cannabis sativa L.). Conference Paper, 434-438); Hsieh et al. (Forensic Science international, 13:53-58), and Gilmore et al. (Forensic Science International, 131: 65-74).

This disclosure pertains to the use of polymorphic loci to uniquely identify the genotype of a Cannabis plant. In particular, this disclosure relates to several polymorphic simple sequence repeat (SSR) loci in the Cannabis genome that can be used in combination to provide a unique genetic signature that identifies a particular plant as being of a particular variety or of a particular provenance. Further, in particular embodiments, the disclosure relates to 18 SSR loci that can be queried together in a single multiplex reaction, and oligonucleotide primers that can be used for such multiplex reactions.

Some of the advantages of the use of SSR markers for genotyping include that it is much faster and less expensive than pan-genomic methods like Next Generation Sequencing (NGS), or Single Nucleotide Polymorphism (SNP) methods. In addition, the results can be easily interpreted by the average person, with no need for complex algorithms or software. SSR genotyping provides results in the form of a simple list of fragment sizes reflecting the alleles for each marker. SSR genotyping has been shown to be as accurate as SNP genotyping if the purpose is to confirm cultivar identity, ascertain parentage, or prove the provenance of genetic material (Garcia et al., 2018). SSR markers are much more polymorphic than SNPs which are typically diallelic while most SSRs have between three to more than 40 possible allelic variants (Barcaccia et al., 2020; Guichoux et al., 2011). While the amount of data generated from SSR genotyping is less than NGS or SNP methods, it provides good genomic coverage, and the disclosed marker set is capable of discriminating between a total of 1.8 septillion (1024) possible different genotypes.

### Identification of Polymorphic SSR Loci Useful for Genotyping Cannabis

The RefSeq assembly for Cannabis sativa (Accession #GCA_900626175) was used to map published SSR repeats to specific chromosome locations on one of the 10 Cannabis chromosomes. This was done by examining the sequences of the repeat units, as well as the published primer sequences used to amplify these regions in the literature. Markers were selected that were distributed more or less evenly throughout the genome, with representation on each of the 10 Cannabis chromosomes (see Tables 40-59).

### Designing Amplicon Primers

For the markers in Table 1 b, primers were designed de novo to amplify the regions containing reported repeats, ensuring that the annealing temperature of the primer sets was consistent and the size of the amplicons was between 80 bp and 600 bp, taking into consideration the variation in the number of repeats for each marker. The primers were designed to ensure that the amplicons within each multiplex reaction combination, would be distinguishable by size differences of at least one base pair. One of four fluorophore labels (6FAM, VIC, PET or NED) was selected for the forward primers of each amplicon in order to optimize the differentiation between markers in the same multiplex reaction combination. In cases where it was not possible to distinguish between two marker amplicons by size alone, using different fluorophore labels for these two amplicons allowed them to be distinguished by the capillary electrophoresis instrument.

Sixty-four (64) Cannabis SSR sites as identified (including di-nucleotide, tri-nucleotide, tetra-nucleotide, penta-nucleotide, and hexanucleotide repeat markers) in the first column of Table 1a were first selected to be tested for their utility in genotyping Cannabis varieties. Thus, sixty four (64) corresponding oligonucleotide primer pairs (identified in the second column of Table 1a) were used in initial polymerase chain reaction (PCR) amplifications from genomic DNA isolated from two Cannabis genotypes (i.e. Purple Kush and Finola) to determine whether amplification products of the loci could be generated, as determined by the presence or absence of one or more bands observed upon agarose gel electrophoresis of the PCR products. Fourty seven (47) Cannabis SSR sites as identified (including tri-nucleotide, tetra-nucleotide, penta-nucleotide, and hexanucleotide repeat markers) in the first column of Table 1 b were then selected to be tested for their utility in genotyping Cannabis varieties. Primers were designed to amplify all 47 of these regions (Table 39b), and eleven, twelve, thirteen, fourteen or eighteen of the primer sets were selected to optimize different combinations of markers for genotyping (see tables 51 to 59).

PCR products could only be generated for a subset of the Cannabis SSR sites tested. Once the subset of the eighty seven (87) Cannabis SSR sites that could be amplified was identified, the PCR amplifications were performed again with the corresponding primer sets labeled with fluorophores, i.e. Beckman Coulter WellRed Dyes D2, D3, D4, or Applied Biosystems custom fluorophores 6-FAM, VIC, NED, PET, although the skilled person understands other fluorophores could be used depending on the instrumentation. Fragment analysis using a capillary electrophoresis instrument was then used to assess whether PCR products could be resolved. In the present study, a Sciex, GenomeLab™ GeXP Dual-Rail Genetic Analysis System, or a Thermo Fisher Scientific Applied Biosystems SeqStudio Genetic Analyzer was used. However, a skilled understands that other fluorophores and capillary electrophoresis instruments could be used.

Resolution on the GeXP capillary electrophoresis instrument was insufficient for consistently size identification of di-nucleotide repeat markers.

Where it was possible to effectively resolve alleles, the sites were mapped to Cannabis chromosomes using BLAST searches with the National Center for Biotechnology Information (NCBI) cannabis genome browser (https://www.ncbi.nlm.nih.gov/genome/gdv/browser/?acc=GCF_900626175.1 &context=genome). ANUCS203, ANUCS205, BO2-CANN2, BO5-CANN1, C08-CANN2, ANUCS302, CAN0024, CAN0093, CAN0107, CAN0110, CAN0865, CAN1001, CAN1423, CAN3087, and H09-CANN2 were not mapped to a chromosome.

PCR amplifications were performed again with the corresponding primer pairs, in various multiplex reaction combinations, for these polymorphic loci on genomic DNA isolated from plants having one hundred and thirty nine (139) separate genotypes in order to determine how many alleles existed at each locus across these genotypes.

This left the following seventy five (75) Cannabis polymorphic sites that each map to a single locus as indicated in the fourth column of Tables 1a and 1 b: and are distributed across the Cannabis genome that can be amplified in multiplex reactions with other loci ANUCS201, ANUCS301; ANUCS303; ANUCS304; ANUCS305; ANUCS501; B01-CANN1; B02-CANN2; C08-CANN2; C11-CANN1; CAN0009; CAN0010; CAN0055; CAN0093; CAN0107A; CAN0110; CAN0111, CAN0126; CAN0509; CAN0585; CAN0717; CAN0865; CAN0878; CAN1347; CAN1482; CAN2550; CAN2717; CAN2913; D02-CANN1; E07-CANN1; H06-CANN2; H09-CANN2; NM101; ANUCS304ana; ANUCS501ana; ANUCS501ana2; C11-CANN1ana2; CAN0055ana4; CAN0055ana5; CAN0057ana; CAN0067ana; CAN0067ana2; CAN0089ana; CAN0097ana; CAN0103ana; CAN0124ana; CAN0138ana2; CAN0156ana; CAN0180ana; CAN0202ana2; CAN0202ana3; CAN0250ana; CAN0271ana; CAN0291ana; CAN0504ana2; CAN0509ana; CAN0577ana; CAN0716ana; CAN0717ana; CAN0802ana; CAN0802ana2; CAN0907ana; CAN1409ana; CAN1539ana; CAN1539ana2; CAN1832ana; CAN2039ana; CAN2418ana; CAN2846ana; CAN2902ana; CAN2913ana; CAN3008ana; E07-CANN1ana2; E07-CANN1ana4; and H06-CANN2ana.

### Multiplex Reactions

Ideally, multiple polymorphic sites should be tested in a single reaction. However, different polymorphic sites may require different conditions for polymerase chain reaction (PCR) amplification or may be incompatible for testing in the same multiplex reactions due to the interactions between primer pairs that prevent or distort amplification. In most other applications, multiplex combinations that work rarely exceeded combinations of 12 to 15 primer sets and usually average between 4 and 10.

Thus, the fluorophore-labelled primer pairs were first multiplexed in at least 33 different ways, as indicated in Tables 2 to 34, to identify the maximum number of primer pairs that can be pooled in a single reaction and to determine which combinations of primer pairs are most compatible. DNA samples from twenty different plants were tested with each set of primer pairs approximately 10 times each to confirm consistency of the results. Of the multiplex reaction combinations 1 to 36 identified in Tables 2 to 34, respectively, multiplex reaction combinations 20, 21, 34, 35 and 36 provided particularly consistent, robust, and clear results. A second set of multiplex reactions were tested using novel primer sets designed in-house to optimize the assay as shown in Tables 40 to 59. Of the multiplex combinations 39 to 91, multiplexes 64, 65, 66, 70 and 91 proved particularly consistent and robust.

Multiplex reaction combinations 20 and 21 together comprise 12 polymorphic loci covering six chromosomes of the Cannabis plant. Multiplex reaction combinations 34 and 35 together comprise 12 polymorphic loci covering all ten chromosomes of the Cannabis plant. Multiplex reaction combination 36 is a single multiplex reaction combination comprising 10 polymorphic loci covering all ten chromosomes of the Cannabis plant. Multiplex reaction combination 64 is a single multiplex reaction combination comprising 11 polymorphic loci covering all ten chromosomes of the Cannabis plant. Multiplex reaction combination 65 is a single multiplex reaction combination comprising 12 polymorphic loci covering all ten chromosomes of the Cannabis plant. Multiplex reaction combination 66 is a single multiplex reaction combination comprising 13 polymorphic loci covering all ten chromosomes of the Cannabis plant. Multiplex reaction combination 70 is a single multiplex reaction combination comprising 14 polymorphic loci covering all ten chromosomes of the Cannabis plant. Multiplex reaction combination 91 is a single multiplex reaction combination comprising 18 polymorphic loci covering all ten chromosomes of the Cannabis plant. Multiplex reaction combination 91b is a single multiplex reaction combination comprising 18 polymorphic loci covering all ten chromosomes of the Cannabis plant.

Pairwise primer pair combinations tested among all of the multiplex reactions described in Tables 2 to 34 and Tables 40 to 59, and the number of multiplex reactions comprising these combinations in which PCR products were successfully produced, are indicated in Tables and 35b. Some combinations of primer pairs were not tested in among the multiplex reactions, and thus the absence of positive results in Table 35a or 35b does not necessarily indicate that a combination does not work. Polymorphic loci involving di-nucleotide repeats were excluded because of inconsistent resolution on the GeXP. “LG” indicates the linkage group (i.e. chromosome) on which the polymorphic locus is located.

Multiplex reaction combinations 20 and 21 were further tested 4 times on 15 different genotypes to confirm that they reliably provided consistent results and could identify differences in each different plant genotype.

Multiplex reaction combinations 20 and 21 were then used to test, in duplicate, 99 Cannabis genomic DNA samples as indicated in Table 36a, as well as DNA from three non-Cannabis plants (radish, basil, and hops) in order to determine if they could be used in combination to provide unique genetic signatures that corresponded with, for example, known clones of the same mother plant (i.e. having the same genotype), plants believed to be of the same variety, etc. A total of 233 samples were genotyped at least once using multiplex reaction combinations 20 and 21.

Multiplex reaction combinations 34 and 35 were tested on 175 individual plant samples, including 135 samples that were also tested with multiplex reaction combinations 20 and 21. Seven of these genotypes were tested in duplicate, and two in triplicate to ensure reproducibility.

Multiplex reaction combination 36 was tested on 35 samples in the laboratory. Multiplex reaction combination 36 was also tested in silico on all of the above samples tested with multiplex reaction combinations 34 and 35 in order to determine whether the 10 markers from multiplex 36 provided equivalent resolution to the 12 markers used in the multiplex 34-combination. That is, the results produced using multiplex reaction combinations 34 and were reconstructed in silico using only the results for the 10 markers of multiplex 36.

Multiplex reaction combinations 64, 65, 66, 70, 91 and 91b were used to test 90, 113, 24, 247, 86 and 27 Cannabis genomic DNA samples, respectively, as indicated in Table 36b to determine if they could be used to provide unique genetic signatures that corresponded with, for example, known clones of the same mother plant (i.e. having the same genotype), plants believed to be of the same variety, etc. A total of 405 samples were genotyped at least once using multiplex reaction combinations 64, 65, 66, 70, 91 and 91b.

The 496 plant samples tested included a wide range of plant material including dry material, fresh material, hemp, “Indica”-like varieties, “Sativa”-like varieties, THC-dominant varieties, CBD-dominant varieties, balanced THC:CBD varieties, outcrossed varieties, Land races, and wild varieties, as shown in Table 36b.

PCR amplifications were performed using the cycling parameters described in Table 37.

Several non-cannabis samples were also tested, and did not produce any genotypes consistent with patterns seen in Cannabis plants. Other species tested include Arabidopsis thaliana, Capsicum annuum (Pepper), Cucurbita pepo (Zucchini), Cupressus (Cypress), Humulus lupulus (Cascade Hop), Humulus lupulus (Citra Hop), Humulus lupulus (Simcoe Hop), Ocimum basilicum (Basil), Pisum sativum (Pea), Poaceae (Grass), Raphanus sativus (Radish), Tracheophyta (Fern), an unknown bacterial culture, and an unknown fungal culture.

One hundred and thirty (130) distinct genotypes were identified from the 233 plant samples tested using multiplex reaction combinations 20 and 21. Ninety-nine of these samples were tested in duplicate with both sets providing comparable results. Two hundred and ten samples were tested using multiplex reaction combinations 34, 35 and 36, including 135 samples that had also been tested using multiplex reaction combinations 20 and 21, and five additional genotypes were identified in the new samples tested with multiplex reaction combinations 34, 35 and 36. See Table 36a for the different cultivars tested. Forty six (46) distinct genotypes were identified from the 405 plant samples tested using multiplex reaction combinations 64, 65, 66, 70 and 91 (Table 36b). In total 176 distinct Cannabis genotypes have been consistently identified using these multiplex combinations.

Two unknown samples 36 and 37 shown in Table 36a were identified as having the Cannatonic genotype, and were subsequently determined to be a clone and its grandmother.

One unknown sample Unknown A shown in Table 36b was a plant sample developed by a breeder that had lost its label. It was determined to be a novel genotype and assigned Genotype Number 19287.

Four known mothers were confirmed to have identical genotypes to the clones grown from them (samples 79 to 86 shown in Table 36a).

Two samples described as of the Chocolope variety (sample 67 and 72 shown in Table 36a), did not have the same genotype as two other individuals described as Chocolope (samples 52 and 155) or another Chocolope sample (169). This difference in genetic identity of the three Chocolope types was consistent with all multiplex reaction combinations tested. Samples 67 and 72 came from a different supplier than samples 52, 155 and 169 and differed from each other at several loci. Thus, samples 67 and 72 are clearly a different variety than samples 52 and 155, and are also different than sample 169 even though all three varieties are described as Chocolope.

Similarly, three samples labelled as Blue Dream (samples 23 and 55 and 161 shown in Table 36a) were also identified as having distinct genotypes. This difference in genetic identity of the three Blue Dream samples was consistent with all multiplex reaction combinations tested. Samples 23 and 161 came from a different supplier than sample 55 and differed at several loci. Thus, samples 23, 55 and 161 are clearly all different varieties even though they were all described as Blue Dream.

Similarly, one sample described as Bubba Kush (sample 10) was found to have a different genotype than three other samples labelled Bubba Kush (samples 20, 227, and 275). These samples came from the same supplier, but sample 10 was reported to have a different phenotype than samples 20, 227 and 275, consistent with the fact that they had different genotypes.

Referring to Table 38a, multiplex reaction combination 36 was used to confirm the genotypes of three samples (samples “1”, “2”, and “3”) suspected to be of the varieties Blue Dream, Luminarium™, and CSC, respectively. As expected, Samples 1, 2, and 3 had the same contingent of alleles at the 10 loci as Blue Dream, Luminarium™, and CSC, respectively, thereby confirming that Sample 1 was of the variety Blue Dream, Sample 2 was of the variety Luminarium™, and Sample 3 was of the variety CSC.

Referring to Table 38b, multiplex reaction combination 70 was used to confirm the genotypes of the same three samples (i.e. Samples 1, 2, and 3). As expected, Samples 1, 2, and 3 had the same contingent of alleles at the 14 loci as Blue Dream, Luminarium™, and CSC, respectively, thereby confirming that Sample 1 was of the variety Blue Dream, Sample 2 was of the variety Luminarium™, and Sample 3 was of the variety CSC.

The size of the alleles as reported in Table 38a and 38b corresponds to the size of the amplification products generated, which include sequences corresponding to the amplification primers. Accordingly, the skilled person understands that while the length of the SSR for a particular allele is fixed, the size of the amplification products generated for the amplification of the allele will depend upon the specific pair of amplification primers used. The skilled person further understands that the perceived size of the amplification product may also depend on the label attached to the primers (e.g. a bigger fluorophores may add several base pairs to the perceived amplification product size as determined by an instrument). The skilled person further understands that the perceived size of any amplification product may vary depending on the enzyme used for amplification and the specific instrument utilized (i.e. the specific fragment separation method and the subsequent analysis method embedded in the software). However, it is within the purview of the skilled person to adjust for primer sequence, fluorophore size, and instrumentation in order to establish the identity the allele(s) present at a specific SSR locus.

Thus, polymorphic SSR loci disclosed herein may be useful in genotyping Cannabis plants. In particular, the 12 polymorphic markers addressed by multiplex reaction combinations 20 and 21 are sufficient to differentiate between Cannabis plants of different varieties, and can be used to confirm stability of genotype between mothers and clones, and between clones, over multiple generations. Advantageously, the identity of the alleles present at these 12 polymorphic loci can be determined using as few as two multiplex reaction combinations.

The 12 polymorphic markers addressed by multiplex reaction combinations 34 and 35 are also sufficient to differentiate between Cannabis plants of different varieties, and/or can also be used to confirm stability of genotype between mothers and clones, and between clones, over multiple generations. Similarly, the identity of the alleles present at these 12 polymorphic loci addressed by multiplex reaction combinations 34 and 35 can be determined using as few as two multiplex reactions. Moreover, the 12 polymorphic markers addressed by multiplex reaction combinations 34 and 35 provide coverage of all 10 Cannabis chromosomes.

The 10 polymorphic markers addressed by multiplex reaction combination 36 are also sufficient to differentiate between Cannabis plants of different varieties, and/or can also be used to confirm stability of genotype between mothers and clones, and between clones, over multiple generations. Advantageously, the identity of the alleles present at these 10 polymorphic loci can be determined using as few as one multiplex reaction. Moreover, the 10 polymorphic markers addressed by multiplex reaction combination 36 provide coverage of all 10 Cannabis chromosomes.

The 11 polymorphic markers addressed by multiplex reaction combinations 64 is also sufficient to differentiate between Cannabis plants of different varieties, and/or can also be used to confirm stability of genotype between mothers and clones, and between clones, over multiple generations. Advantageously, the identity of the alleles present at these 11 polymorphic loci can be determined using as few as one multiplex reaction. Moreover, the 11 polymorphic markers addressed by multiplex reaction combinations 64 provide coverage of all 10 Cannabis chromosomes.

The 12 polymorphic markers addressed by multiplex reaction combination 65 is also sufficient to differentiate between Cannabis plants of different varieties, and/or can also be used to confirm stability of genotype between mothers and clones, and between clones, over multiple generations. Advantageously, the identity of the alleles present at these 12 polymorphic loci can be determined using as few as one multiplex reaction. Moreover, the 12 polymorphic markers addressed by multiplex reaction combination 65 provide coverage of all 10 Cannabis chromosomes.

The 13 polymorphic markers addressed by multiplex reaction combination 66 are also sufficient to differentiate between Cannabis plants of different varieties, and/or can also be used to confirm stability of genotype between mothers and clones, and between clones, over multiple generations. Advantageously, the identity of the alleles present at these 13 polymorphic loci can be determined using as few as one multiplex reaction. Moreover, the 13 polymorphic markers addressed by multiplex reaction combination 66 provide coverage of all 10 Cannabis chromosomes.

The 14 polymorphic markers addressed by multiplex reaction combination 70 are also sufficient to differentiate between Cannabis plants of different varieties, and/or can also be used to confirm stability of genotype between mothers and clones, and between clones, over multiple generations. Advantageously, the identity of the alleles present at these 14 polymorphic loci can be determined using as few as one multiplex reaction. Moreover, the 14 polymorphic markers addressed by multiplex reaction combination 70 provide coverage of all 10 Cannabis chromosomes.

The 18 polymorphic markers addressed by multiplex reaction combination 91 are also sufficient to differentiate between Cannabis plants of different varieties, and/or can also be used to confirm stability of genotype between mothers and clones, and between clones, over multiple generations. Advantageously, the identity of the alleles present at these 18 polymorphic loci can be determined using as few as one multiplex reaction. Moreover, the 18 polymorphic markers addressed by multiplex reaction combination 91 provide coverage of all 10 Cannabis chromosomes.

The 18 polymorphic markers addressed by multiplex reaction combination 91b are also sufficient to differentiate between Cannabis plants of different varieties, and/or can also be used to confirm stability of genotype between mothers and clones, and between clones, over multiple generations. Advantageously, the identity of the alleles present at these 18 polymorphic loci can be determined using as few as one multiplex reaction. Moreover, the 18 polymorphic markers addressed by multiplex reaction combination 91 provide coverage of all 10 Cannabis chromosomes.

### Methods of Constructing a Genetic Profile

Thus, the skilled person understands that polymorphic nature of loci identified in the present disclosure, and particularly ANUCS201, ANUCS301; ANUCS303; ANUCS304; ANUCS305; ANUCS501; B01-CANN1; C08-CANN2; C11-CANN1; CAN0009; CAN0010; CAN0055; CAN0093; CAN0107A; CAN0110; CAN0111, CAN0126; CAN0509; CAN0585; CAN0717; CAN0865; CAN0878; CAN1347; CAN1482; CAN2550; CAN2717; CAN2913; D02-CANN1; D02-CANN2; E07-CANN1; H06-CANN2; H09-CANN2; NM101; ANUCS304ana; ANUCS501ana; ANUCS501ana2; C11-CANN1ana2; CAN0055ana4; CAN0055ana5; CAN0057ana; CAN0067ana; CAN0067ana2; CAN0089ana; CAN0097ana; CAN0103ana; CAN0124ana; CAN0138ana2; CAN0156ana; CAN0180ana; CAN0202ana2; CAN0202ana3; CAN0250ana; CAN0271ana; CAN0291ana; CAN0504ana2; CAN0509ana; CAN0577ana; CAN0716ana; CAN0717ana; CAN0802ana; CAN0802ana2; CAN0907ana; CAN1409ana; CAN1539ana; CAN1539ana2; CAN1832ana; CAN2039ana; CAN2418ana; CAN2846ana; CAN2902ana; CAN2913ana; CAN3008ana; E07-CANN1ana2; E07-CANN1ana4; and H06-CANN2ana may be exploited for methods of constructing a unique genetic profile for a Cannabis plant. Each of these polymorphic target sequences comprises a simple sequence repeat (SSR) sequence that is variable in length. Accordingly, the skilled person understands that each “target sequence” is not a single target sequence of a precise length, but rather encompasses several potential target sequences at a single locus that vary in terms of length (i.e. alleles) but are flanked by common nucleotide sequences.

Once an individual plant is selected to be genotyped, then genetic sequence information may be obtained from the plant for the purpose of determining the identity of the alleles at selected polymorphic SSR loci. Genetic sequence information may be obtained in numerous different ways and may involve the collection of a biological sample that contains genetic material, particularly, genomic DNA containing the sequence or sequences of interest. Any suitable genomic DNA samples from a Cannabis plant may be used, e.g. from seeds, seedlings, leaf tissue, stem tissue, root tissue, flower tissue, tissue cultures or plants (i.e. Cannabis plant material) of any age or stage of development. The Cannabis plant may be a Cannabis sativa plant, a Cannabis indica plant, or a Cannabis ruderalis plant and may be a drug-type variety, hemp, or a wild Cannabis accession.

Many methods are known in the art for collecting biological samples and extracting genetic material from those samples, as surveyed, for example, in WO 2009/124396 and WO2016/197258. As discussed in WO2019/222835, samples may include the leaves of Cannabis seedlings or young plants pressed into a paper matrix, such as a paper matrix (e.g. Whatman™ FTA™ cards) comprising a mixture of chemicals that lyse cells and stabilize nucleic acids on contact for long-term storage at room temperature. Alternatively, commercial DNA preparation kits such as the Qiagen DNeasy Mini Kit, or similar column-based kits, or magnetic bead-based protocols may be used. Alternatively, DNA extracted using conventional protocols such as CTAB extraction may be used. Alternatively, a crude extract of plant material may be used where the sample is simply ground using a beadbeater, and then centrifuged to precipitate the plant material, leaving the DNA solubilized in the supernatant

The ability to use leaf material pressed into a paper matrix, or a crude extract of dried plant material is particularly advantageous because the samples are easily collected, and stored and shipped at room temperature. The ability to use leaf material pressed into a paper matrix has the additional advantage that it may be shipped in a format that is not subject to the same restrictions as handling of controlled or regulated substances because no cannabinoids will be present in the paper matrix. Alternatively, the samples may comprise purified DNA isolated from fresh or dry Cannabis plant material.

Once an individual plant's genomic DNA has been obtained, the identity of the allele at one or more of the polymorphic SSR loci can be determined using any one of several methods known in the art. A variety of methods for determining the sequence at polymorphic sites are discussed in, for example, WO 2009/124396, WO2011/133418, WO2016/197258, and WO2019/222835. For example, a primer pair corresponding to the common nucleotide sequences at the locus that flank the SSR sequence can be used to amplify the SSR, e.g. by polymerase chain reaction (PCR), in order to determine its length(s) (i.e. the allele(s) that is present). The sizes of the amplification products indicate the length of the at least two polymorphic target sequences. At least one primer of each primer pair may include a fluorophore tag, e.g. as indicated for the forward primers identified in Tables 2 to 34 and Tables 40 to 59 to assist in detection and sizing of the resulting PCR amplification products.

Detection or determination of the length of the particular allele(s) present at an SSR locus may be accomplished by any one of a number of methods or assays known in the art. Furthermore, there are numerous methods for detecting the products of each type of reaction (e.g. fluorescence, luminescence, etc.). In specific embodiments exemplified herein, capillary electrophoresis using a capillary electrophoresis instrument, was used to resolve and size the PCR products. In another method discussed in WO2019/222835, High Resolution Melt (HRM) analysis may be conducted to determine the identity of the alleles present at an SSR locus. As double stranded DNA denatures during HRM, the loss in fluorescence from an intercalating fluorescent dye over time provides different denaturation curves depending on which alleles are present at the locus. However, the skilled person understands that direct sequencing may be used to determine the sequence, and thus the identity, of the alleles.

Regardless, the skilled person understands that it is not important how the length of the SSR at the locus is determined, i.e. by amplification and detection or otherwise, so long as it may be reliably determined.

The precise combination of alleles at two or more of these loci, in terms of the length of the SSR, provides a genetic profile of the plant that can serve as a unique genetic signature. The genetic profile may be used to identify the ancestry (i.e. the provenance) of the plant. The genetic profile may be used to identify the variety or cultivar to which the plant belongs. The genetic profile may be used to identify the parentage of the plant. The genetic profile may be used to identify the chemotype of the plant. The genetic profiles may be used in breeding methods to identify specific parents and progeny of interest. The genetic profiles may also be used to establish forensic linkages between source and evidentiary samples. The genetic profile may be used to prove that the sample is of the genus Cannabis.

The skilled person will further understand that it is not necessary to know the particular sizes or sequences of any of the alleles at the polymorphic target sites in order to practice the methods disclosed herein. The skilled person will understand that there are potentially an unlimited number of alleles that may exist at the polymorphic loci that have yet to be discovered. The skilled person understands that it is sufficient to know that multiple alleles exist at these loci and that a genetic profile resulting from the precise combination of alleles identified at these loci may be unique to a particular plant or variety. Accordingly, it is well within the purview of the skilled person reading this disclosure to use the methods disclosed herein to develop genetic profiles that may be used to uniquely identify specific individuals, varieties, etc.

Accordingly, aspects of the disclosure pertains to a method comprising testing a deoxyribonucleic acid (DNA) sample from plant material from a Cannabis plant to determine the length of at least two polymorphic target sequences selected from ANUCS201, ANUCS301; ANUCS303; ANUCS304; ANUCS305; ANUCS501; B01-CANN1; C08-CANN2; C11-CANN1; CAN0009; CAN0010; CAN0055; CAN0093; CAN0107A; CAN0110; CAN0111, CAN0126; CAN0509; CAN0585; CAN0717; CAN0865; CAN0878; CAN1347; CAN1482; CAN2550; CAN2717; CAN2913; D02-CANN1; D02-CANN2; E07-CANN1; H06-CANN2; H09-CANN2; NM101; ANUCS304ana; ANUCS501ana; ANUCS501ana2; C11-CANN1ana2; CAN0055ana4; CAN0055ana5; CAN0057ana; CAN0067ana; CAN0067ana2; CAN0089ana; CAN0097ana; CAN0103ana; CAN0124ana; CAN0138ana2; CAN0156ana; CAN0180ana; CAN0202ana2; CAN0202ana3; CAN0250ana; CAN0271ana; CAN0291ana; CAN0504ana2; CAN0509ana; CAN0577ana; CAN0716ana; CAN0717ana; CAN0802ana; CAN0802ana2; CAN0907ana; CAN1409ana; CAN1539ana; CAN1539ana2; CAN1832ana; CAN2039ana; CAN2418ana; CAN2846ana; CAN2902ana; CAN2913ana; CAN3008ana; E07-CANN1ana2; E07-CANN1ana4; and H06-CANN2ana. Each of these polymorphic target sequences comprises a simple sequence repeat (SSR) sequence that is variable in length. The polymorphic sequences may be defined in terms of primer pairs that can be used to amplify them.

In various embodiments, the at least two polymorphic target sequences may comprise at least 3 polymorphic target sequences, at least 4 polymorphic target sequences, at least 5 polymorphic target sequences, at least 6 polymorphic target sequences, at least 7 polymorphic target sequences, at least 8 polymorphic target sequences, at least 9 polymorphic target sequences, at least 10 polymorphic target sequences, at least 11 polymorphic target sequences, at least 12 polymorphic target sequences, at least 13 polymorphic target sequences, at least 14 polymorphic target sequences, at least 15 polymorphic target sequences, at least 16 polymorphic target sequences, at least 17 polymorphic target sequences, at least 18 polymorphic target sequences, at least 19 polymorphic target sequences, at least 20 polymorphic target sequences, at least 21 polymorphic target sequences, at least 22 polymorphic target sequences, at least 23 polymorphic target sequences, at least 24 polymorphic target sequences, at least 25 polymorphic target sequences, at least 26 polymorphic target sequences, at least 27 polymorphic target sequences, at least 28 polymorphic target sequences, at least 29 polymorphic target sequences, at least 30 polymorphic target sequences, at least 31 polymorphic target sequences, at least 32 polymorphic target sequences, at least 33 polymorphic target sequences, at least 34 polymorphic target sequences, at least 35 polymorphic target sequences, at least 36 polymorphic target sequences, at least 37 polymorphic target sequences, at least 38 polymorphic target sequences, at least 39 polymorphic target sequences, at least 40 polymorphic target sequences, at least 41 polymorphic target sequences, at least 42 polymorphic target sequences, at least 43 polymorphic target sequences, at least 44 polymorphic target sequences, at least 45 polymorphic target sequences, at least 46 polymorphic target sequences, at least 47 polymorphic target sequences, at least 48 polymorphic target sequences, at least 49 polymorphic target sequences, at least 50 polymorphic target sequences, at least 51 polymorphic target sequences, at least 52 polymorphic target sequences, at least 53 polymorphic target sequences, at least 54 polymorphic target sequences, at least 55 polymorphic target sequences, at least 56 polymorphic target sequences, at least 57 polymorphic target sequences, at least 58 polymorphic target sequences, at least 59 polymorphic target sequences, at least 60 polymorphic target sequences, at least 61 polymorphic target sequences, at least 62 polymorphic target sequences, at least 63 polymorphic target sequences, at least 64 polymorphic target sequences, at least 65 polymorphic target sequences, at least 66 polymorphic target sequences, at least 67 polymorphic target sequences, at least 68 polymorphic target sequences, at least 69 polymorphic target sequences, at least 70 polymorphic target sequences, or at least 71 polymorphic target sequences.

In particular embodiments, the two or more polymorphic target sequences comprise at least six polymorphic sequences. In particular embodiments, the two or more polymorphic target sequences comprise at least ten polymorphic sequences. In particular embodiments, the two or more polymorphic target sequences comprise at least eleven polymorphic sequences. In particular embodiments, the two or more polymorphic target sequences comprise at least twelve polymorphic sequences. In particular embodiments, the two or more polymorphic target sequences comprise at least thirteen polymorphic sequences. In particular embodiments, the two or more polymorphic target sequences comprise at least fourteen polymorphic sequences. In particular embodiments, the two or more polymorphic target sequences comprise at least eighteen polymorphic sequences. In particular embodiments, each of the ten chromosomes of the Cannabis genome is represented by at least one of the polymorphic target sequences.

In various embodiments, testing the DNA sample from the plant to determine the length of the polymorphic target sequences comprises amplifying the DNA sample with a plurality of primer pairs. Each of the primer pairs specifically hybridizes to one of the polymorphic target sequences to generate amplification products corresponding to the target sequence. The lengths of the amplification product(s) at each of the at least two polymorphic target sequences are then determined in order to determine the length of the SSR (and thus the allele(s) present) at each of the polymorphic loci. Thus, the precise combination of amplification products (i.e. in terms of length) from all of the loci provides a genetic profile of the plant.

### Target Sequences Defined by Flanking Sequences

Accordingly, the at least two polymorphic target sequences comprise two or more of: a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 7 and SEQ ID NO: 8; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 15 and SEQ ID NO: 16; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 21 and SEQ ID NO: 22; a target sequence amplifiable by a primer pair having the sequences of SEQ ID NO: 23 and SEQ ID NO: 24; a target sequence amplifiable by a primer pair having the sequences of SEQ ID NO: 25 and SEQ ID NO: 26; a target sequence amplifiable by a primer pair having the sequences of SEQ ID NO: 27 and SEQ ID NO: 28; a target sequence amplifiable by a primer pair having the sequences of SEQ ID NO: 29 and SEQ ID NO: 30; a target sequence amplifiable by a primer pair having the sequences of SEQ ID NO: 31 and SEQ ID NO: 32; a target sequence amplifiable by a primer pair having the sequences of SEQ ID NO: 33 and SEQ ID NO: 34; a target sequence amplifiable by a primer pair having the sequences of SEQ ID NO: 35 and SEQ ID NO: 36; a target sequence amplifiable by a primer pair having the sequences of SEQ ID NO: 37 and SEQ ID NO: 38; a target sequence amplifiable by a primer pair having the sequences of SEQ ID NO: 39 and SEQ ID NO: 40; a target sequence amplifiable by a primer pair having the sequences of SEQ ID NO: 41 and SEQ ID NO: 42; a target sequence amplifiable by a primer pair having the sequences of SEQ ID NO: 43 and SEQ ID NO: 44; a target sequence amplifiable by a primer pair having the sequences of SEQ ID NO: 45 and SEQ ID NO: 46; a target sequence amplifiable by a primer pair having the sequences of SEQ ID NO: 124 and SEQ ID NO: 125; a target sequence amplifiable by a primer pair having the sequences of SEQ ID NO: 126 and SEQ ID NO: 127; a target sequence amplifiable by a primer pair having the sequences of SEQ ID NO: 128 and SEQ ID NO: 129; a target sequence amplifiable by a primer pair having the sequences of SEQ ID NO: 130 and SEQ ID NO: 131; a target sequence amplifiable by a primer pair having the sequences of SEQ ID NO: 132 and SEQ ID NO: 133; a target sequence amplifiable by a primer pair having the sequences of SEQ ID NO: 134 and SEQ ID NO: 135; a target sequence amplifiable by a primer pair having the sequences of SEQ ID NO: 136 and SEQ ID NO: 137; a target sequence amplifiable by a primer pair having the sequences of SEQ ID NO: 138 and SEQ ID NO: 139; a target sequence amplifiable by a primer pair having the sequences of SEQ ID NO: 140 and SEQ ID NO: 141; a target sequence amplifiable by a primer pair having the sequences of SEQ ID NO: 142 and SEQ ID NO: 143; a target sequence amplifiable by a primer pair having the sequences of SEQ ID NO: 144 and SEQ ID NO: 145; a target sequence amplifiable by a primer pair having the sequences of SEQ ID NO: 146 and SEQ ID NO: 147; a target sequence amplifiable by a primer pair having the sequences of SEQ ID NO: 148 and SEQ ID NO: 149; a target sequence amplifiable by a primer pair having the sequences of SEQ ID NO: 150 and SEQ ID NO: 151; a target sequence amplifiable by a primer pair having the sequences SEQ ID NO: 166 and SEQ ID NO: 167; a target sequence amplifiable by a primer pair having the sequences SEQ ID NO: 168 and SEQ ID NO: 169; a target sequence amplifiable by a primer pair having the sequences SEQ ID NO: 170 and SEQ ID NO: 171; a target sequence amplifiable by a primer pair having the sequences SEQ ID NO: 174 and SEQ ID NO: 175; a target sequence amplifiable by a primer pair having the sequences SEQ ID NO: 176 and SEQ ID NO: 177; a target sequence amplifiable by a primer pair having the sequences SEQ ID NO: 180 and SEQ ID NO: 181; a target sequence amplifiable by a primer pair having the sequences SEQ ID NO: 182 and SEQ ID NO: 183; a target sequence amplifiable by a primer pair having the sequences SEQ ID NO: 184 and SEQ ID NO: 185; a target sequence amplifiable by a primer pair having the sequences SEQ ID NO: 186 and SEQ ID NO: 187; a target sequence amplifiable by a primer pair having the sequences SEQ ID NO: 188 and SEQ ID NO: 189; a target sequence amplifiable by a primer pair having the sequences SEQ ID NO: 190 and SEQ ID NO: 191; a target sequence amplifiable by a primer pair having the sequences SEQ ID NO: 196 and SEQ ID NO: 197; a target sequence amplifiable by a primer pair having the sequences SEQ ID NO: 200 and SEQ ID NO: 201; a target sequence amplifiable by a primer pair having the sequences SEQ ID NO: 210 and SEQ ID NO: 211; a target sequence amplifiable by a primer pair having the sequences SEQ ID NO: 212 and SEQ ID NO: 213; a target sequence amplifiable by a primer pair having the sequences SEQ ID NO: 214 and SEQ ID NO: 215; a target sequence amplifiable by a primer pair having the sequences SEQ ID NO: 218 and SEQ ID NO: 219; a target sequence amplifiable by a primer pair having the sequences SEQ ID NO: 220 and SEQ ID NO: 221; a target sequence amplifiable by a primer pair having the sequences SEQ ID NO: 222 and SEQ ID NO: 223; a target sequence amplifiable by a primer pair having the sequences SEQ ID NO: 224 and SEQ ID NO: 225; a target sequence amplifiable by a primer pair having the sequences SEQ ID NO: 228 and SEQ ID NO: 229; a target sequence amplifiable by a primer pair having the sequences SEQ ID NO: 230 and SEQ ID NO: 231; a target sequence amplifiable by a primer pair having the sequences SEQ ID NO: 232 and SEQ ID NO: 233; a target sequence amplifiable by a primer pair having the sequences SEQ ID NO: 238 and SEQ ID NO: 239; a target sequence amplifiable by a primer pair having the sequences SEQ ID NO: 240 and SEQ ID NO: 241; a target sequence amplifiable by a primer pair having the sequences SEQ ID NO: 242 and SEQ ID NO: 243; a target sequence amplifiable by a primer pair having the sequences SEQ ID NO: 244 and SEQ ID NO: 245; a target sequence amplifiable by a primer pair having the sequences SEQ ID NO: 248 and SEQ ID NO: 249; a target sequence amplifiable by a primer pair having the sequences SEQ ID NO: 250 and SEQ ID NO: 251; a target sequence amplifiable by a primer pair having the sequences SEQ ID NO: 168 and SEQ ID NO: 295; a target sequence amplifiable by a primer pair having the sequences SEQ ID NO: 192 and SEQ ID NO: 300; a target sequence amplifiable by a primer pair having the sequences SEQ ID NO: 216 and SEQ ID NO: 217; a target sequence amplifiable by a primer pair having the sequences SEQ ID NO: 224 and SEQ ID NO: 305; a target sequence amplifiable by a primer pair having the sequences SEQ ID NO: 296 and SEQ ID NO: 297; a target sequence amplifiable by a primer pair having the sequences SEQ ID NO: 298 and SEQ ID NO: 299; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 6 and SEQ ID NO: 306; a target sequence amplifiable by a primer pair having the sequences SEQ ID NO: 307 and SEQ ID NO: 308; a target sequence amplifiable by a primer pair having the sequences SEQ ID NO: 309 and SEQ ID NO: 300; a target sequence amplifiable by a primer pair having the sequences SEQ ID NO: 310 and SEQ ID NO: 311; and a target sequence amplifiable by a primer pair having the sequences SEQ ID NO: 312 and SEQ ID NO: 313. The precise combination of alleles at two or more of these loci, in terms of the length of the SSR, provides a genetic profile of the Cannabis plant.

In some instances, e.g. depending on the method of detection being employed, it may be desirable to avoid using loci that involve dinucleotide repeats. Accordingly, in some embodiments, the at least two polymorphic sequences comprise two or more of: a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 7 and SEQ ID NO: 8; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 15 and SEQ ID NO: 16; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 21 and SEQ ID NO: 22; a target sequence amplifiable by a primer pair having the sequences of SEQ ID NO: 23 and SEQ ID NO: 24; a target sequence amplifiable by a primer pair having the sequences of SEQ ID NO: 27 and SEQ ID NO: 28; a target sequence amplifiable by a primer pair having the sequences of SEQ ID NO: 29 and SEQ ID NO: 30; a target sequence amplifiable by a primer pair having the sequences of SEQ ID NO: 37 and SEQ ID NO: 38; a target sequence amplifiable by a primer pair having the sequences of SEQ ID NO: 39 and SEQ ID NO: 40; a target sequence amplifiable by a primer pair having the sequences of SEQ ID NO: 43 and SEQ ID NO: 44; a target sequence amplifiable by a primer pair having the sequences of SEQ ID NO: 45 and SEQ ID NO: 46; a target sequence amplifiable by a primer pair having the sequences of SEQ ID NO: 124 and SEQ ID NO: 125; a target sequence amplifiable by a primer pair having the sequences of SEQ ID NO: 126 and SEQ ID NO: 127; a target sequence amplifiable by a primer pair having the sequences of SEQ ID NO: 130 and SEQ ID NO: 131; a target sequence amplifiable by a primer pair having the sequences of SEQ ID NO: 132 and SEQ ID NO: 133; a target sequence amplifiable by a primer pair having the sequences of SEQ ID NO: 134 and SEQ ID NO: 135; a target sequence amplifiable by a primer pair having the sequences of SEQ ID NO: 136 and SEQ ID NO: 137; a target sequence amplifiable by a primer pair having the sequences of SEQ ID NO: 138 and SEQ ID NO: 139; a target sequence amplifiable by a primer pair having the sequences of SEQ ID NO: 140 and SEQ ID NO: 141; a target sequence amplifiable by a primer pair having the sequences of SEQ ID NO: 142 and SEQ ID NO: 143; a target sequence amplifiable by a primer pair having the sequences of SEQ ID NO: 144 and SEQ ID NO: 145; a target sequence amplifiable by a primer pair having the sequences of SEQ ID NO: 146 and SEQ ID NO: 147; a target sequence amplifiable by a primer pair having the sequences of SEQ ID NO: 148 and SEQ ID NO: 149; a target sequence amplifiable by a primer pair having the sequences of SEQ ID NO: 150 and SEQ ID NO: 151; a target sequence amplifiable by a primer pair having the sequences of SEQ ID NO: 166 and SEQ ID NO: 167; a target sequence amplifiable by a primer pair having the sequences of SEQ ID NO: 168 and SEQ ID NO: 169; a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 176 and SEQ ID NO: 177; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 184 and SEQ ID NO: 185; a target sequence amplifiable by a primer pair having the sequences of SEQ ID NO: 186 and SEQ ID NO: 187; a target sequence amplifiable by a primer pair having the sequences of SEQ ID NO: 188 and SEQ ID NO: 189; a target sequence amplifiable by a primer pair having the sequences of SEQ ID NO: 190 and SEQ ID NO: 191; a target sequence amplifiable by a primer pair having the sequences of SEQ ID NO: 196 and SEQ ID NO: 197; a target sequence amplifiable by a primer pair having the sequences of SEQ ID NO: 200 and SEQ ID NO: 201; a target sequence amplifiable by a primer pair having the sequences of SEQ ID NO: 210 and SEQ ID NO: 211; a target sequence amplifiable by a primer pair having the sequences of SEQ ID NO: 212 and SEQ ID NO: 213; a target sequence amplifiable by a primer pair having the sequences of SEQ ID NO: 214 and SEQ ID NO: 215; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 222 and SEQ ID NO: 223; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 230 and SEQ ID NO: 231; a target sequence amplifiable by a primer pair having the sequences of SEQ ID NO: 232 and SEQ ID NO: 233; a target sequence amplifiable by a primer pair having the sequences of SEQ ID NO: 238 and SEQ ID NO: 239; a target sequence amplifiable by a primer pair having the sequences of SEQ ID NO: 240 and SEQ ID NO: 241; a target sequence amplifiable by a primer pair having the sequences of SEQ ID NO: 242 and SEQ ID NO: 243; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; a target sequence amplifiable by a primer pair having the sequences of SEQ ID NO: 250 and SEQ ID NO: 251; a target sequence amplifiable by a primer pair having the sequences SEQ ID NO: 168 and SEQ ID NO: 295; a target sequence amplifiable by a primer pair having the sequences SEQ ID NO: 192 and SEQ ID NO: 300; a target sequence amplifiable by a primer pair having the sequences SEQ ID NO: 216 and SEQ ID NO: 217; a target sequence amplifiable by a primer pair having the sequences SEQ ID NO: 224 and SEQ ID NO: 305; a target sequence amplifiable by a primer pair having the sequences SEQ ID NO: 296 and SEQ ID NO: 297; a target sequence amplifiable by a primer pair having the sequences SEQ ID NO: 298 and SEQ ID NO: 299; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 6 and SEQ ID NO: 306; a target sequence amplifiable by a primer pair having the sequences SEQ ID NO: 307 and SEQ ID NO: 308; a target sequence amplifiable by a primer pair having the sequences SEQ ID NO: 309 and SEQ ID NO: 300; a target sequence amplifiable by a primer pair having the sequences SEQ ID NO: 310 and SEQ ID NO: 311; and a target sequence amplifiable by a primer pair having the sequences SEQ ID NO: 312 and SEQ ID NO: 313.

In particular embodiments, the at least two polymorphic target sequences comprise: a target sequence amplifiable by a primer pair having the sequences of SEQ ID NO: 1 and SEQ ID NO: 2; a target sequence amplifiable by a primer pair having the sequences of SEQ ID NO: 7 and SEQ ID NO: 8; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 23 and SEQ ID NO: 24; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 15 and SEQ ID NO: 16; and a target sequence amplifiable by a primer pair having the sequences of SEQ ID NO: 21 and SEQ ID NO: 22.

In particular embodiments, the at least two polymorphic target sequences comprise: a target sequence amplifiable by a primer pair having the sequences of SEQ ID NO: 7 and SEQ ID NO: 8; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 29 and SEQ ID NO: 30; a target sequence amplifiable by a primer pair having the sequences of SEQ ID NO: 37 and SEQ ID NO: 38; a target sequence amplifiable by a primer pair having the sequences of SEQ ID NO: 45 and SEQ ID NO: 46; a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 13 and SEQ ID NO: 14; a target sequence amplifiable by a primer pair having the sequences of SEQ ID NO: 39 and SEQ ID NO: 40; and a target sequence amplifiable by a primer pair having the sequences of SEQ ID NO: 43 and SEQ ID NO: 44.

In particular embodiments, the at least two polymorphic target sequences comprise: a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 21 and SEQ ID NO: 22; and a target sequence amplifiable by a primer pair having the sequences of SEQ ID NO: 23 and SEQ ID NO: 24.

In particular embodiments, the at least two polymorphic target sequences comprise: a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences of SEQ ID NO: 170 and SEQ ID NO: 171; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 212 and SEQ ID NO: 213; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences SEQ ID NO: 168 and SEQ ID NO: 295; a target sequence amplifiable by a primer pair having the sequences SEQ ID NO: 170 and SEQ ID NO: 171; a target sequence amplifiable by a primer pair having the sequences SEQ ID NO: 174 and SEQ ID NO: 175; a target sequence amplifiable by a primer pair having the sequences SEQ ID NO: 296 and SEQ ID NO: 297; a target sequence amplifiable by a primer pair having the sequences SEQ ID NO: 180 and SEQ ID NO: 181; a target sequence amplifiable by a primer pair having the sequences SEQ ID NO: 298 and SEQ ID NO: 299; a target sequence amplifiable by a primer pair having the sequences SEQ ID NO: 192 and SEQ ID NO: 300; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 210 and SEQ ID NO: 211; a target sequence amplifiable by a primer pair having the sequences SEQ ID NO: 212 and SEQ ID NO: 213; a target sequence amplifiable by a primer pair having the sequences SEQ ID NO: 214 and SEQ ID NO: 215; a target sequence amplifiable by a primer pair having the sequences SEQ ID NO: 216 and SEQ ID NO: 217; a target sequence amplifiable by a primer pair having the sequences SEQ ID NO: 218 and SEQ ID NO: 219; a target sequence amplifiable by a primer pair having the sequences SEQ ID NO: 220 and SEQ ID NO: 221; a target sequence amplifiable by a primer pair having the sequences SEQ ID NO: 224 and SEQ ID NO: 305; a target sequence amplifiable by a primer pair having the sequences SEQ ID NO: 244 and SEQ ID NO: 245; and a target sequence amplifiable by a primer pair having the sequences SEQ ID NO: 6 and SEQ ID NO: 306.

In particular embodiments, the at least two polymorphic target sequences comprise a target sequence amplifiable by a primer pair having the sequences SEQ ID NO: 168 and SEQ ID NO: 295; a target sequence amplifiable by a primer pair having the sequences SEQ ID NO: 170 and SEQ ID NO: 171; a target sequence amplifiable by a primer pair having the sequences SEQ ID NO: 312 and SEQ ID NO: 313; a target sequence amplifiable by a primer pair having the sequences SEQ ID NO: 307 and SEQ ID NO: 308; a target sequence amplifiable by a primer pair having the sequences SEQ ID NO: 180 and SEQ ID NO: 181; a target sequence amplifiable by a primer pair having the sequences SEQ ID NO: 298 and SEQ ID NO: 299; a target sequence amplifiable by a primer pair having the sequences SEQ ID NO: 309 and SEQ ID NO: 300; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 210 and SEQ ID NO: 211; a target sequence amplifiable by a primer pair having the sequences SEQ ID NO: 212 and SEQ ID NO: 213; a target sequence amplifiable by a primer pair having the sequences SEQ ID NO: 214 and SEQ ID NO: 215; a target sequence amplifiable by a primer pair having the sequences SEQ ID NO: 216 and SEQ ID NO: 217; a target sequence amplifiable by a primer pair having the sequences SEQ ID NO: 310 and SEQ ID NO: 311; a target sequence amplifiable by a primer pair having the sequences SEQ ID NO: 220 and SEQ ID NO: 221; a target sequence amplifiable by a primer pair having the sequences SEQ ID NO: 224 and SEQ ID NO: 305; a target sequence amplifiable by a primer pair having the sequences SEQ ID NO: 244 and SEQ ID NO: 245; and a target sequence amplifiable by a primer pair having the sequences SEQ ID NO: 6 and SEQ ID NO: 306.

### Target Sequences Defined by Flanking Sequences

The skilled person further understands that the polymorphic target sequences may alternatively be defined in terms of nucleotide sequences that flank the SSR sequence. Accordingly, in various embodiments, the at least two polymorphic target sequences comprise two or more of: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: on the 5′ end and SEQ ID NO: 108 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; a target sequence flanked by SEQ ID NO: 23 on the 5′ end and SEQ ID NO: 112 on the 3′ end; a target sequence flanked by SEQ ID NO: 25 on the 5′ end and SEQ ID NO: 113 on the 3′ end; a target sequence flanked by SEQ ID NO: 27 on the 5′ end and SEQ ID NO: 114 on the 3′ end; a target sequence flanked by SEQ ID NO: 29 on the 5′ end and SEQ ID NO: 115 on the 3′ end; a target sequence flanked by SEQ ID NO: 31 on the 5′ end and SEQ ID NO: 116 on the 3′ end; a target sequence flanked by SEQ ID NO: 33 on the 5′ end and SEQ ID NO: 117 on the 3′ end; a target sequence flanked by SEQ ID NO: 35 on the 5′ end and SEQ ID NO: 118 on the 3′ end; a target sequence flanked by SEQ ID NO: 37 on the 5′ end and SEQ ID NO: 119 on the 3′ end; a target sequence flanked by SEQ ID NO: 39 on the 5′ end and SEQ ID NO: 120 on the 3′ end; a target sequence flanked by SEQ ID NO: 41 on the 5′ end and SEQ ID NO: 121 on the 3′ end; a target sequence flanked by SEQ ID NO: 43 on the 5′ end and SEQ ID NO: 122 on the 3′ end; a target sequence flanked by SEQ ID NO: 45 on the 5′ end and SEQ ID NO: 123 on the 3′ end; a target sequence flanked by SEQ ID NO: 124 on the 5′ end and SEQ ID NO: 152 on the 3′ end; a target sequence flanked by SEQ ID NO: 126 on the 5′ end and SEQ ID NO: 153 on the 3′ end; a target sequence flanked by SEQ ID NO: 128 on the 5′ end and SEQ ID NO: 154 on the 3′ end; a target sequence flanked by SEQ ID NO: 130 on the 5′ end and SEQ ID NO: 155 on the 3′ end; a target sequence flanked by SEQ ID NO: 132 on the 5′ end and SEQ ID NO: 156 on the 3′ end; a target sequence flanked by SEQ ID NO: 134 on the 5′ end and SEQ ID NO: 157 on the 3′ end; a target sequence flanked by SEQ ID NO: 136 on the 5′ end and SEQ ID NO: 158 on the 3′ end; a target sequence flanked by SEQ ID NO: 138 on the 5′ end and SEQ ID NO: 159 on the 3′ end; a target sequence flanked by SEQ ID NO: 140 on the 5′ end and SEQ ID NO: 160 on the 3′ end; a target sequence flanked by SEQ ID NO: 142 on the 5′ end and SEQ ID NO: 161 on the 3′ end; a target sequence flanked by SEQ ID NO: 144 on the 5′ end and SEQ ID NO: 162 on the 3′ end; a target sequence flanked by SEQ ID NO: 146 on the 5′ end and SEQ ID NO: 163 on the 3′ end; a target sequence flanked by SEQ ID NO: 148 on the 5′ end and SEQ ID NO: 164 on the 3′ end; a target sequence flanked by SEQ ID NO: 150 on the 5′ end and SEQ ID NO: 165 on the 3′ end; a target sequence flanked by SEQ ID NO: 166 on the 5′ end and SEQ ID NO: 252 on the 3′ end; a target sequence flanked by SEQ ID NO: 168 on the 5′ end and SEQ ID NO: 253 on the 3′ end; a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 176 on the 5′ end and SEQ ID NO: 257 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 184 on the 5′ end and SEQ ID NO: 261 on the 3′ end; a target sequence flanked by SEQ ID NO: 186 on the 5′ end and SEQ ID NO: 262 on the 3′ end; a target sequence flanked by SEQ ID NO: 188 on the 5′ end and SEQ ID NO: 263 on the 3′ end; a target sequence flanked by SEQ ID NO: 190 on the 5′ end and SEQ ID NO: 264 on the 3′ end; a target sequence flanked by SEQ ID NO: 196 on the 5′ end and SEQ ID NO: 267 on the 3′ end; a target sequence flanked by SEQ ID NO: 200 on the 5′ end and SEQ ID NO: 269 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 222 on the 5′ end and SEQ ID NO: 280 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 230 on the 5′ end and SEQ ID NO: 284 on the 3′ end; a target sequence flanked by SEQ ID NO: 232 on the 5′ end and SEQ ID NO: 285 on the 3′ end; a target sequence flanked by SEQ ID NO: 238 on the 5′ end and SEQ ID NO: 288 on the 3′ end; a target sequence flanked by SEQ ID NO: 240 on the 5′ end and SEQ ID NO: 289 on the 3′ end; a target sequence flanked by SEQ ID NO: 242 on the 5′ end and SEQ ID NO: 290 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end; a target sequence flanked by SEQ ID NO: 168 on the 5′ end and SEQ ID NO: 314 on the 3′ end; a target sequence flanked by SEQ ID NO: 192 on the 5′ end and SEQ ID NO: 317 on the 3′ end; a target sequence flanked by SEQ ID NO: 216 on the 5′ end and SEQ ID NO: 277 on the 3′ end; a target sequence flanked by SEQ ID NO: 224 on the 5′ end and SEQ ID NO: 320 on the 3′ end; a target sequence flanked by SEQ ID NO: 296 on the 5′ end and SEQ ID NO: 315 on the 3′ end; a target sequence flanked by SEQ ID NO: 298 on the 5′ end and SEQ ID NO: 316 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 306 on the 5′ end and SEQ ID NO: 103 on the 3′ end; a target sequence flanked by SEQ ID NO: 307 on the 5′ end and SEQ ID NO: 321 on the 3′ end; a target sequence flanked by SEQ ID NO: 309 on the 5′ end and SEQ ID NO: 317 on the 3′ end; a target sequence flanked by SEQ ID NO: 310 on the 5′ end and SEQ ID NO: 322 on the 3′ end; and a target sequence flanked by SEQ ID NO: 312 on the 5′ end and SEQ ID NO: 323 on the 3′ end.

DNA is double-stranded, with one strand having a sequence that is the reverse complement of the other strand. Accordingly, the skilled person understands that the polymorphic sequences could also be defined in terms of the reverse complements of the flanking sequences defined herein. For example, “a target sequence flanked by SEQ ID NO: 1 on the 5′ end and SEQ ID NO: 101 on the 3′ end” could alternatively be defined as “a target sequence flanked by SEQ ID NO: 2 on the 5′ end and the reverse complement of SEQ ID NO: 1 on the 3′ end”.

Furthermore, the skilled person understands that it is the variation in the SSR sequence within the portion of the genome bounded by the flanking sequences, and not the flanking sequences themselves, that defines the alleles at the polymorphic loci. Accordingly, the skilled person understands that does not matter if a method involves detecting a sequence longer or shorter than the sequence precisely defined by the recited flanking sequences themselves, provided that the method directly or indirectly involves determining the length of the target SSR sequence located within.

In particular embodiments, the at least two polymorphic target sequences comprise: a target sequence flanked by SEQ ID NO: 1 on the 5′ end and SEQ ID NO: 101 on the 3′ end; a target sequence flanked by SEQ ID NO: 7 on the 5′ end and SEQ ID NO: 104 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 23 on the 5′ end and SEQ ID NO: 112 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; a target sequence flanked by SEQ ID NO: 21 on the 5′ end and SEQ ID NO: 111 on the 3′ end;

In particular embodiments, the at least two polymorphic target sequences comprise: a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 29 on the 5′ end and SEQ ID NO: 115 on the 3′ end; a target sequence flanked by SEQ ID NO: 37 on the 5′ end and SEQ ID NO: 119 on the 3′ end; a target sequence flanked by SEQ ID NO: 45 on the 5′ end and SEQ ID NO: 123 on the 3′ end; a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 13 on the 5′ end and SEQ ID NO: 107 on the 3′ end; a target sequence flanked by SEQ ID NO: 39 on the 5′ end and SEQ ID NO: 120 on the 3′ end; and a target sequence flanked by SEQ ID NO: 43 on the 5′ end and SEQ ID NO: 122 on the 3′ end.

In particular embodiments, the at least two polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; and a target sequence flanked by SEQ ID NO: 23 on the 5′ end and SEQ ID NO: 112 on the 3′ end.

In particular embodiments, the at least two polymorphic target sequences comprise: a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 296 on the 5′ end and SEQ ID NO: 315 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 192 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

In particular embodiments, the at least two polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 312 on the 5′ end and SEQ ID NO: 323 on the 3′ end; a target sequence flanked by SEQ ID NO: 307 on the 5′ end and SEQ ID NO: 321 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 309 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 310 on the 5′ end and SEQ ID NO: 322 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

### Embodiments Utilizing Particular Primer Pairs

In embodiments utilizing PCR amplification to test the genomic DNA samples, the skilled person understands that various primer pairs may be designed to specifically amplify portions of the genome comprising the SSR sequence described herein, and thus be useful for methods of constructing genetic profiles for Cannabis plants. Nevertheless, in particular embodiments of the invention the plurality of primers for testing the DNA samples may comprise two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; a primer pair having the sequences of SEQ ID NO: 23 and SEQ ID NO: 24; a primer pair having the sequences of SEQ ID NO: 25 and SEQ ID NO: 26; a primer pair having the sequences of SEQ ID NO: 27 and SEQ ID NO: 28; a primer pair having the sequences of SEQ ID NO: 29 and SEQ ID NO: 30; a primer pair having the sequences of SEQ ID NO: 31 and SEQ ID NO: 32; a primer pair having the sequences of SEQ ID NO: 33 and SEQ ID NO: 34; a primer pair having the sequences of SEQ ID NO: 35 and SEQ ID NO: 36; a primer pair having the sequences of SEQ ID NO: 37 and SEQ ID NO: 38; a primer pair having the sequences of SEQ ID NO: 39 and SEQ ID NO: 40; a primer pair having the sequences of SEQ ID NO: 41 and SEQ ID NO: 42; a primer pair having the sequences of SEQ ID NO: 43 and SEQ ID NO: 44; a primer pair having the sequences of SEQ ID NO: 45 and SEQ ID NO: 46; a primer pair having the sequences of SEQ ID NO: 124 and SEQ ID NO: 125; a primer pair having the sequences of SEQ ID NO: 126 and SEQ ID NO: 127; a primer pair having the sequences of SEQ ID NO: 128 and SEQ ID NO: 129; a primer pair having the sequences of SEQ ID NO: 130 and SEQ ID NO: 131; a primer pair having the sequences of SEQ ID NO: 132 and SEQ ID NO: 133; a primer pair having the sequences of SEQ ID NO: 134 and SEQ ID NO: 135; a primer pair having the sequences of SEQ ID NO: 136 and SEQ ID NO: 137; a primer pair having the sequences of SEQ ID NO: 138 and SEQ ID NO: 139; a primer pair having the sequences of SEQ ID NO: 140 and SEQ ID NO: 141; a primer pair having the sequences of SEQ ID NO: 142 and SEQ ID NO: 143; a primer pair having the sequences of SEQ ID NO: 144 and SEQ ID NO: 145; a primer pair having the sequences of SEQ ID NO: 146 and SEQ ID NO: 147; a primer pair having the sequences of SEQ ID NO: 148 and SEQ ID NO: 149; a primer pair having the sequences of SEQ ID NO: 150 and SEQ ID NO: 151; a primer pair having the sequences of SEQ ID NO: 166 and SEQ ID NO: 167; a primer pair having the sequences of SEQ ID NO: 168 and SEQ ID NO: 169; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 176 and SEQ ID NO: 177; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 184 and SEQ ID NO: 185; a primer pair having the sequences of SEQ ID NO: 186 and SEQ ID NO: 187; a primer pair having the sequences of SEQ ID NO: 188 and SEQ ID NO: 189; a primer pair having the sequences of SEQ ID NO: 190 and SEQ ID NO: 191; a primer pair having the sequences of SEQ ID NO: 196 and SEQ ID NO: 197; a primer pair having the sequences of SEQ ID NO: 200 and SEQ ID NO: 201; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 222 and SEQ ID NO: 223; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 230 and SEQ ID NO: 231; a primer pair having the sequences of SEQ ID NO: 232 and SEQ ID NO: 233; a primer pair having the sequences of SEQ ID NO: 238 and SEQ ID NO: 239; a primer pair having the sequences of SEQ ID NO: 240 and SEQ ID NO: 241; a primer pair having the sequences of SEQ ID NO: 242 and SEQ ID NO: 243; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251; a primer pair having the sequences of SEQ ID NO: 168 and SEQ ID NO: 295; a primer pair having the sequences of SEQ ID NO: 192 and SEQ ID NO: 300; a primer pair having the sequences of SEQ ID NO: 216 and SEQ ID NO: 217; a primer pair having the sequences of SEQ ID NO: 224 and SEQ ID NO: 305; a primer pair having the sequences of SEQ ID NO: 296 and SEQ ID NO: 297; a primer pair having the sequences of SEQ ID NO: 298 and SEQ ID NO: 299; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 6 and SEQ ID NO: 306; a primer pair having the sequences of SEQ ID NO: 307 and SEQ ID NO: 308; a primer pair having the sequences of SEQ ID NO: 309 and SEQ ID NO: 300; a primer pair having the sequences of SEQ ID NO: 310 and SEQ ID NO: 311; and a primer pair having the sequences of SEQ ID NO: 312 and SEQ ID NO: 313.

In particular embodiments, the plurality of primers for testing the DNA samples may comprise: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 23 and SEQ ID NO: 24; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

In particular embodiments where each chromosome of the ten Cannabis chromosomes is represented by at least one of the polymorphic target sequences, the plurality of primers for testing the DNA samples may comprise: a primer pair having the sequences of SEQ ID NO: 7 and SEQ ID NO: 8; a primer pair having the sequences of SEQ ID NO: 9 and SEQ ID NO: a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; a primer pair having the sequences of SEQ ID NO: 45 and SEQ ID NO: 46; a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

In particular embodiments where each chromosome of the ten Cannabis chromosomes is represented by at least one of the polymorphic target sequences, the plurality of primers for testing the DNA samples may comprise: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 17 and SEQ ID NO: 18; a primer pair having the sequences of SEQ ID NO: 19 and SEQ ID NO: a primer pair having the sequences of SEQ ID NO: 21 and SEQ ID NO: 22; and a primer pair having the sequences of SEQ ID NO: 23 and SEQ ID NO: 24.

In particular embodiments where each chromosome of the ten Cannabis chromosomes is represented by at least one of the polymorphic target sequences, the plurality of primers for testing the DNA samples may comprise: a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251

In particular embodiments where each chromosome of the ten Cannabis chromosomes is represented by at least one of the polymorphic target sequences, the plurality of primers for testing the DNA samples may comprise: a target sequence amplifiable by a primer pair having the sequences of SEQ ID NO: 170 and SEQ ID NO: 171; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments where each chromosome of the ten Cannabis chromosomes is represented by at least one of the polymorphic target sequences, the plurality of primers for testing the DNA samples may comprise: a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments where each chromosome of the ten Cannabis chromosomes is represented by at least one of the polymorphic target sequences, the plurality of primers for testing the DNA samples may comprise: a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 212 and SEQ ID NO: 213; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments where each chromosome of the ten Cannabis chromosomes is represented by at least one of the polymorphic target sequences, the plurality of primers for testing the DNA samples may comprise: a target sequence amplifiable by a primer pair having the sequences SEQ ID NO: 168 and SEQ ID NO: 295; a target sequence amplifiable by a primer pair having the sequences SEQ ID NO: 170 and SEQ ID NO: 171; a target sequence amplifiable by a primer pair having the sequences SEQ ID NO: 174 and SEQ ID NO: 175; a target sequence amplifiable by a primer pair having the sequences SEQ ID NO: 296 and SEQ ID NO: 297; a target sequence amplifiable by a primer pair having the sequences SEQ ID NO: 180 and SEQ ID NO: 181; a target sequence amplifiable by a primer pair having the sequences SEQ ID NO: 298 and SEQ ID NO: 299; a target sequence amplifiable by a primer pair having the sequences SEQ ID NO: 192 and SEQ ID NO: 300; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 210 and SEQ ID NO: 211; a target sequence amplifiable by a primer pair having the sequences SEQ ID NO: 212 and SEQ ID NO: 213; a target sequence amplifiable by a primer pair having the sequences SEQ ID NO: 214 and SEQ ID NO: 215; a target sequence amplifiable by a primer pair having the sequences SEQ ID NO: 216 and SEQ ID NO: 217; a target sequence amplifiable by a primer pair having the sequences SEQ ID NO: 218 and SEQ ID NO: 219; a target sequence amplifiable by a primer pair having the sequences SEQ ID NO: 220 and SEQ ID NO: 221; a target sequence amplifiable by a primer pair having the sequences SEQ ID NO: 224 and SEQ ID NO: 305; a target sequence amplifiable by a primer pair having the sequences SEQ ID NO: 244 and SEQ ID NO: 245; and a target sequence amplifiable by a primer pair having the sequences SEQ ID NO: 6 and SEQ ID NO: 306.

In particular embodiments where each chromosome of the ten Cannabis chromosomes is represented by at least one of the polymorphic target sequences, the plurality of primers for testing the DNA samples may comprise: a target sequence amplifiable by a primer pair having the sequences SEQ ID NO: 168 and SEQ ID NO: 295; a target sequence amplifiable by a primer pair having the sequences SEQ ID NO: 170 and SEQ ID NO: 171; a target sequence amplifiable by a primer pair having the sequences SEQ ID NO: 312 and SEQ ID NO: 313; a target sequence amplifiable by a primer pair having the sequences SEQ ID NO: 307 and SEQ ID NO: 308; a target sequence amplifiable by a primer pair having the sequences SEQ ID NO: 180 and SEQ ID NO: 181; a target sequence amplifiable by a primer pair having the sequences SEQ ID NO: 298 and SEQ ID NO: 299; a target sequence amplifiable by a primer pair having the sequences SEQ ID NO: 309 and SEQ ID NO: 300; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 210 and SEQ ID NO: 211; a target sequence amplifiable by a primer pair having the sequences SEQ ID NO: 212 and SEQ ID NO: 213; a target sequence amplifiable by a primer pair having the sequences SEQ ID NO: 214 and SEQ ID NO: 215; a target sequence amplifiable by a primer pair having the sequences SEQ ID NO: 216 and SEQ ID NO: 217; a target sequence amplifiable by a primer pair having the sequences SEQ ID NO: 310 and SEQ ID NO: 311; a target sequence amplifiable by a primer pair having the sequences SEQ ID NO: 220 and SEQ ID NO: 221; a target sequence amplifiable by a primer pair having the sequences SEQ ID NO: 224 and SEQ ID NO: 305; a target sequence amplifiable by a primer pair having the sequences SEQ ID NO: 244 and SEQ ID NO: 245; and a target sequence amplifiable by a primer pair having the sequences SEQ ID NO: 6 and SEQ ID NO: 306.

### Embodiments Utilizing Multiplex Reactions

The skilled person further understands that, while the length of each polymorphic sequence can be determined using a separate reaction specific for a given polymorphic target sequence, it is preferable to be able to test the DNA samples to determine the lengths of multiple polymorphic target sequences simultaneously by, for example, with multiplex PCR amplifications involving combinations of primer pairs corresponding to different polymorphic target sequences. The use of multiplex reactions provides for advantages in terms of reductions in the time and resources required to construct a genetic profile for a plant or plurality of plants.

A potentially unlimited number of primer pair combinations could be used in multiplex reactions to test genomic Cannabis DNA to construct genetic profiles. Moreover, it is not necessary that primer pairs for all of the polymorphic sequences be tested for the purposes of constructing a genetic profile be included in a single multiplex reaction. That is, multiple multiplex reactions could be performed, each with different combinations primer pairs designed to amplify a subset of the target polymorphic sequences. The results of multiplex reactions can then be combined in order to construct the genetic profile.

Accordingly, various embodiments involving amplification of a genomic Cannabis DNA sample with a plurality of primers to amplify the polymorphic SSR sequences comprise amplifying the DNA sample in at least one multiplex reaction.

In various embodiments, the at least one multiplex reaction comprises two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; a primer pair having the sequences of SEQ ID NO: 23 and SEQ ID NO: 24; a primer pair having the sequences of SEQ ID NO: 25 and SEQ ID NO: 26; a primer pair having the sequences of SEQ ID NO: 27 and SEQ ID NO: 28; a primer pair having the sequences of SEQ ID NO: 29 and SEQ ID NO: 30; a primer pair having the sequences of SEQ ID NO: 31 and SEQ ID NO: 32; a primer pair having the sequences of SEQ ID NO: 33 and SEQ ID NO: 34; a primer pair having the sequences of SEQ ID NO: 35 and SEQ ID NO: 36; a primer pair having the sequences of SEQ ID NO: 37 and SEQ ID NO: 38; a primer pair having the sequences of SEQ ID NO: 39 and SEQ ID NO: 40; a primer pair having the sequences of SEQ ID NO: 41 and SEQ ID NO: 42; a primer pair having the sequences of SEQ ID NO: 43 and SEQ ID NO: 44; a primer pair having the sequences of SEQ ID NO: 45 and SEQ ID NO: 46; a primer pair having the sequences of SEQ ID NO: 124 and SEQ ID NO: 125; a primer pair having the sequences of SEQ ID NO: 126 and SEQ ID NO: 127; a primer pair having the sequences of SEQ ID NO: 128 and SEQ ID NO: 129; a primer pair having the sequences of SEQ ID NO: 130 and SEQ ID NO: 131; a primer pair having the sequences of SEQ ID NO: 132 and SEQ ID NO: 133; a primer pair having the sequences of SEQ ID NO: 134 and SEQ ID NO: 135; a primer pair having the sequences of SEQ ID NO: 136 and SEQ ID NO: 137; a primer pair having the sequences of SEQ ID NO: 138 and SEQ ID NO: 139; a primer pair having the sequences of SEQ ID NO: 140 and SEQ ID NO: 141; a primer pair having the sequences of SEQ ID NO: 142 and SEQ ID NO: 143; a primer pair having the sequences of SEQ ID NO: 144 and SEQ ID NO: 145; a primer pair having the sequences of SEQ ID NO: 146 and SEQ ID NO: 147; a primer pair having the sequences of SEQ ID NO: 148 and SEQ ID NO: 149; a primer pair having the sequences of SEQ ID NO: 150 and SEQ ID NO: 151; a primer pair having the sequences of SEQ ID NO: 166 and SEQ ID NO: 167; a primer pair having the sequences of SEQ ID NO: 168 and SEQ ID NO: 169; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 176 and SEQ ID NO: 177; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 184 and SEQ ID NO: 185; a primer pair having the sequences of SEQ ID NO: 186 and SEQ ID NO: 187; a primer pair having the sequences of SEQ ID NO: 188 and SEQ ID NO: 189; a primer pair having the sequences of SEQ ID NO: 190 and SEQ ID NO: 191; a primer pair having the sequences of SEQ ID NO: 196 and SEQ ID NO: 197; a primer pair having the sequences of SEQ ID NO: 200 and SEQ ID NO: 201; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 222 and SEQ ID NO: 223; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 230 and SEQ ID NO: 231; a primer pair having the sequences of SEQ ID NO: 232 and SEQ ID NO: 233; a primer pair having the sequences of SEQ ID NO: 238 and SEQ ID NO: 239; a primer pair having the sequences of SEQ ID NO: 240 and SEQ ID NO: 241; a primer pair having the sequences of SEQ ID NO: 242 and SEQ ID NO: 243; a primer pair having the sequences of SEQ ID NO: 244 and SEQ ID NO: 245; a primer pair having the sequences of SEQ ID NO: 248 and SEQ ID NO: 249; a primer pair having the sequences of SEQ ID NO: 250 and SEQ ID NO: 251; a primer pair having the sequences of SEQ ID NO: 168 and SEQ ID NO: 295; a primer pair having the sequences of SEQ ID NO: 192 and SEQ ID NO: 300; a primer pair having the sequences of SEQ ID NO: 216 and SEQ ID NO: 217; a primer pair having the sequences of SEQ ID NO: 224 and SEQ ID NO: 305; a primer pair having the sequences of SEQ ID NO: 296 and SEQ ID NO: 297; a primer pair having the sequences of SEQ ID NO: 298 and SEQ ID NO: 299; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 6 and SEQ ID NO: 306; a primer pair having the sequences of SEQ ID NO: 307 and SEQ ID NO: 308; a primer pair having the sequences of SEQ ID NO: 309 and SEQ ID NO: 300; a primer pair having the sequences of SEQ ID NO: 310 and SEQ ID NO: 311; and a primer pair having the sequences of SEQ ID NO: 312 and SEQ ID NO: 313.

In various embodiments, the at least one multiplex reaction comprises two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; a primer pair having the sequences of SEQ ID NO: 23 and SEQ ID NO: 24; a primer pair having the sequences of SEQ ID NO: 29 and SEQ ID NO: 30; a primer pair having the sequences of SEQ ID NO: 37 and SEQ ID NO: 38; a primer pair having the sequences of SEQ ID NO: 39 and SEQ ID NO: 40; a primer pair having the sequences of SEQ ID NO: 43 and SEQ ID NO: 44; a primer pair having the sequences of SEQ ID NO: 45 and SEQ ID NO: 46 a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251; a primer pair having the sequences of SEQ ID NO: 168 and SEQ ID NO: 295; a primer pair having the sequences of SEQ ID NO: 192 and SEQ ID NO: 300; a primer pair having the sequences of SEQ ID NO: 216 and SEQ ID NO: 217; a primer pair having the sequences of SEQ ID NO: 224 and SEQ ID NO: 305; a primer pair having the sequences of SEQ ID NO: 296 and SEQ ID NO: 297; a primer pair having the sequences of SEQ ID NO: 298 and SEQ ID NO: 299; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 6 and SEQ ID NO: 306; a primer pair having the sequences of SEQ ID NO: 307 and SEQ ID NO: 308; a primer pair having the sequences of SEQ ID NO: 309 and SEQ ID NO: 300; a primer pair having the sequences of SEQ ID NO: 310 and SEQ ID NO: 311; and a primer pair having the sequences of SEQ ID NO: 312 and SEQ ID NO: 313.

In various embodiments, the at least one multiplex reaction comprises two or more of: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24.

In various embodiments, the at least one multiplex reaction comprises: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24.

In various embodiments, the at least one multiplex reaction comprises two or more of: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

In various embodiments, the at least one multiplex reaction comprises: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

In various embodiments, the at least one multiplex reaction comprises a first multiplex reaction comprising two or more of: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24. The at least one multiplex reaction comprises a second multiplex reaction comprising two or more of: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

In various embodiments, the at least one multiplex reaction comprises a first multiplex reaction comprising: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24. The at least one multiplex reaction further comprises a second multiplex reaction comprising: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

In various embodiments, the at least one multiplex reaction comprises two or more of: a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; and a primer pair having the sequences of SEQ ID NO: 45 and SEQ ID NO: 46.

In various embodiments, the at least one multiplex reaction comprises: a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; and a primer pair having the sequences of SEQ ID NO: 45 and SEQ ID NO: 46.

In various embodiments, the at least one multiplex reaction comprises two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

In various embodiments, the at least one multiplex reaction comprises: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

In various embodiments, the at least one multiplex reaction comprises a multiplex reaction comprising two or more of: a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; and a primer pair having the sequences of SEQ ID NO: and SEQ ID NO: 46. The at least one multiplex reaction further comprises a second multiplex reaction comprising two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

In various embodiments, the at least one multiplex reaction comprises a multiplex reaction comprising: a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; and a primer pair having the sequences of SEQ ID NO: 45 and SEQ ID NO: 46. The at least one multiplex reaction further comprises a second multiplex reaction comprising: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

In various embodiments, the at least one multiplex reaction comprises a single multiplex reaction comprising two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 17 and SEQ ID NO: 18; a primer pair having the sequences of SEQ ID NO: 19 and SEQ ID NO: a primer pair having the sequences of SEQ ID NO: 21 and SEQ ID NO: 22; and a primer pair having the sequences of SEQ ID NO: 23 and SEQ ID NO: 24.

In various embodiments, the at least one multiplex reaction comprises a single multiplex reaction comprising: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; and a primer pair having the sequences of SEQ ID NO: 23 and SEQ ID NO: 24.

In various embodiments, the at least one multiplex reaction comprises a single multiplex reaction comprising two or more of: a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising: a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising two or more of: a primer pair having the sequences of SEQ ID NO: 170 and SEQ ID NO: 171; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising: a primer pair having the sequences of SEQ ID NO: 170 and SEQ ID NO: 171; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising two or more of: a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising: a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising two or more of: a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising: a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In various embodiments, the at least one multiplex reaction comprises a single multiplex reaction comprising two or more of: a primer pair having the sequences of SEQ ID NO: 168 and SEQ ID NO: 295; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 296 and SEQ ID NO: 297; a primer pair having the sequences of SEQ ID NO: 180 and SEQ ID NO: 181; a primer pair having the sequences of SEQ ID NO: 298 and SEQ ID NO: 299; a primer pair having the sequences of SEQ ID NO: 192 and SEQ ID NO: 300; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 216 and SEQ ID NO: 217; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 305; a primer pair having the sequences of SEQ ID NO: 244 and SEQ ID NO: 245; and a primer pair having the sequences of SEQ ID NO: 6 and SEQ ID NO: 306.

In various embodiments, the at least one multiplex reaction comprises a single multiplex reaction comprising: a primer pair having the sequences of SEQ ID NO: 168 and SEQ ID NO: 295; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 296 and SEQ ID NO: 297; a primer pair having the sequences of SEQ ID NO: 180 and SEQ ID NO: 181; a primer pair having the sequences of SEQ ID NO: 298 and SEQ ID NO: 299; a primer pair having the sequences of SEQ ID NO: 192 and SEQ ID NO: 300; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 216 and SEQ ID NO: 217; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 305; a primer pair having the sequences of SEQ ID NO: 244 and SEQ ID NO: 245; and a primer pair having the sequences of SEQ ID NO: 6 and SEQ ID NO: 306.

In various embodiments, the at least one multiplex reaction comprises a single multiplex reaction comprising two or more of: a primer pair having the sequences of SEQ ID NO: 168 and SEQ ID NO: 295; a primer pair having the sequences of SEQ ID NO: 170 and SEQ ID NO: 171; a primer pair having the sequences of SEQ ID NO: 312 and SEQ ID NO: 313; a primer pair having the sequences of SEQ ID NO: 307 and SEQ ID NO: 308; a primer pair having the sequences of SEQ ID NO: 180 and SEQ ID NO: 181; a primer pair having the sequences of SEQ ID NO: 298 and SEQ ID NO: 299; a primer pair having the sequences of SEQ ID NO: 309 and SEQ ID NO: 300; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 216 and SEQ ID NO: 217; a primer pair having the sequences of SEQ ID NO: 310 and SEQ ID NO: 311; a primer pair having the sequences of SEQ ID NO: 220 and SEQ ID NO: 221; a primer pair having the sequences of SEQ ID NO: 224 and SEQ ID NO: 305; a primer pair having the sequences of SEQ ID NO: 244 and SEQ ID NO: 245; and a primer pair having the sequences of SEQ ID NO: 6 and SEQ ID NO: 306.

In various embodiments, the at least one multiplex reaction comprises a single multiplex reaction comprising: a primer pair having the sequences of SEQ ID NO: 168 and SEQ ID NO: 295; a primer pair having the sequences of SEQ ID NO: 170 and SEQ ID NO: 171; a primer pair having the sequences of SEQ ID NO: 312 and SEQ ID NO: 313; a primer pair having the sequences of SEQ ID NO: 307 and SEQ ID NO: 308; a primer pair having the sequences of SEQ ID NO: 180 and SEQ ID NO: 181; a primer pair having the sequences of SEQ ID NO: 298 and SEQ ID NO: 299; a primer pair having the sequences of SEQ ID NO: 309 and SEQ ID NO: 300; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 216 and SEQ ID NO: 217; a primer pair having the sequences of SEQ ID NO: 310 and SEQ ID NO: 311; a primer pair having the sequences of SEQ ID NO: 220 and SEQ ID NO: 221; a primer pair having the sequences of SEQ ID NO: 224 and SEQ ID NO: 305; a primer pair having the sequences of SEQ ID NO: 244 and SEQ ID NO: 245; and a primer pair having the sequences of SEQ ID NO: 6 and SEQ ID NO: 306.

### Marker Assisted Breeding

Breeding new varieties, lines and hybrids may be achieved using techniques of crossing and selection on a set of parental lines taking advantage of the plant's method of pollination (self-, sib- or cross-pollination). Multiple rounds of crossing and selection may be needed to produce plant varieties with the desired traits. After each round, the breeder selects candidates with the desired traits or markers for the next round of selection.

Molecular markers can also be used during the breeding process for the selection of qualitative or quantitative traits. For example, markers linked to traits can be used to select plants that express the traits of interest during a breeding program. The markers can also be used to select for the allele of the desired trait and against the alleles of the undesired trait in young plants prior to the expression of the trait at the phenotypic level. The use of this procedure can speed up selection of desired progeny plants and reduce the number of back crosses to the desired parent in a backcrossing program.

The skilled person understands that other desired traits (e.g. altered height, specific chemistry, early or late maturity, or disease resistance) can be transferred into selected varieties by selecting a first parent, crossing it with a second parent plant having the desired traits that are linked to a particular genetic profile (or subset of a genetic profile) constructed according to the methods described herein, collecting F₁ seed, growing a F₁ plant which is allowed to self-pollinate and collecting the F₂ seed. The F₂ seed would then be grown, and individual plants that have the genotype of interest could be identified according to the methods herein. The skilled person will be able to backcross the selected individuals to the second parent.

Thus, the skilled person further understands that the presently disclosed methods may be useful in methods of marker-assisted breeding of Cannabis plants.

Accordingly, another aspect of the disclosure relates to methods of producing a Cannabis plant with a desired genetic profile. In a particular embodiment, the method comprising initially selecting a first parent having a first parent genetic profile constructed according to a method as described herein and a second parent having a second parent genetic profile constructed according to a method as described herein. The first parent is then crossed with the second parent to produce progeny plants. The method further includes constructing genetic profiles of a plurality of progeny plants of the cross according to a method described herein. Finally, the method includes identifying a progeny plant from the cross that has the desired genetic profile.

In another embodiment, the method comprises initially selecting a first parent having a first parent genetic profile constructed according to a method as described herein and a second parent having a second parent genetic profile constructed according to a method as described herein. The first parent is then crossed with the second parent to produce F₁ progeny plants. The F₁ individuals may then be treated to induce the formation of plants with both female and male flowers. For example, female F₁ progeny plants may be treated with silver thiosulfate (“STS”), a suppressor of ethylene production in plants, to induce the formation of male flowers. F₁ progeny plants with both female and male flowers may be placed in pollination bags to self-pollinate to produce F₂ seed. Genetic profiles are then constructed for a plurality of the F₂ progeny plants of the cross according to a method as described herein, thereby allowing for the identification of an F₂ progeny plant that has the desired genetic profile.

In another embodiment, the method comprises selecting a parent having a parent genetic profile constructed according to a method as described herein.

The parent may then be treated to induce the formation of both female and male flowers, and placed in pollination bags to self-pollinate to produce progeny plants. Genetic profiles may then be constructed for a plurality of the progeny plants according to a method as described herein, thereby allowing for the identification of a progeny plant that has the desired genetic profile.

The skilled person understands that the methods may further include backcrossing progeny plants having the desired genetic profile to the original parent plants, or plants having genetic profiles identical to an original parent plant. Alternatively, the method may further include crossing progeny plants having the desired genetic profile to other plants of interest in the process of producing additional plants having genetic profiles of interest.

The skilled person further understands that the methods can also be used to accelerate the development of homozygous, inbred or near-isogenic lines, which are a prerequisite to the creation of hybrid plants with improved vigor and increased agronomic advantages.

### Paternity Testing

The presently disclosed methods and molecular markers may be useful in determining whether a first plant is genetically related to a second plant. The genotype of the first plant is compared to the genotype of the second plant, and statistical analysis of the alleles present in both the first and second plants can be used to determine the probability of parentage.

### Polynucleotides and Kits

The skilled person further understands that the disclosure pertains to reagents and kits that are useful or necessary for performing the methods described herein for constructing genetic profiles for Cannabis plants.

Thus, the disclosure further pertains to sets of primers for constructing a genetic profile for a Cannabis plant. In one embodiment, the set comprises a plurality of primer pairs for specifically amplifying at least two polymorphic target sequences. The at least two polymorphic target sequences comprise two or more of: a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 7 and SEQ ID NO: 8; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 15 and SEQ ID NO: 16; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 21 and SEQ ID NO: 22; a target sequence amplifiable by a primer pair having the sequences of SEQ ID NO: 23 and SEQ ID NO: 24; a target sequence amplifiable by a primer pair having the sequences of SEQ ID NO: 25 and SEQ ID NO: 26; a target sequence amplifiable by a primer pair having the sequences of SEQ ID NO: 27 and SEQ ID NO: 28; a target sequence amplifiable by a primer pair having the sequences of SEQ ID NO: 29 and SEQ ID NO: 30; a target sequence amplifiable by a primer pair having the sequences of SEQ ID NO: 31 and SEQ ID NO: 32; a target sequence amplifiable by a primer pair having the sequences of SEQ ID NO: 33 and SEQ ID NO: 34; a target sequence amplifiable by a primer pair having the sequences of SEQ ID NO: 35 and SEQ ID NO: 36; a target sequence amplifiable by a primer pair having the sequences of SEQ ID NO: 37 and SEQ ID NO: 38; a target sequence amplifiable by a primer pair having the sequences of SEQ ID NO: 39 and SEQ ID NO: 40; a target sequence amplifiable by a primer pair having the sequences of SEQ ID NO: 41 and SEQ ID NO: 42; a target sequence amplifiable by a primer pair having the sequences of SEQ ID NO: 43 and SEQ ID NO: 44; a target sequence amplifiable by a primer pair having the sequences of SEQ ID NO: 45 and SEQ ID NO: 46; a target sequence amplifiable by a primer pair having the sequences of SEQ ID NO: 124 and SEQ ID NO: 125; a target sequence amplifiable by a primer pair having the sequences of SEQ ID NO: 126 and SEQ ID NO: 127; a target sequence amplifiable by a primer pair having the sequences of SEQ ID NO: 128 and SEQ ID NO: 129; a target sequence amplifiable by a primer pair having the sequences of SEQ ID NO: 130 and SEQ ID NO: 131; a target sequence amplifiable by a primer pair having the sequences of SEQ ID NO: 132 and SEQ ID NO: 133; a target sequence amplifiable by a primer pair having the sequences of SEQ ID NO: 134 and SEQ ID NO: 135; a target sequence amplifiable by a primer pair having the sequences of SEQ ID NO: 136 and SEQ ID NO: 137; a target sequence amplifiable by a primer pair having the sequences of SEQ ID NO: 138 and SEQ ID NO: 139; a target sequence amplifiable by a primer pair having the sequences of SEQ ID NO: 140 and SEQ ID NO: 141; a target sequence amplifiable by a primer pair having the sequences of SEQ ID NO: 142 and SEQ ID NO: 143; a target sequence amplifiable by a primer pair having the sequences of SEQ ID NO: 144 and SEQ ID NO: 145; a target sequence amplifiable by a primer pair having the sequences of SEQ ID NO: 146 and SEQ ID NO: 147; a target sequence amplifiable by a primer pair having the sequences of SEQ ID NO: 148 and SEQ ID NO: 149; a target sequence amplifiable by a primer pair having the sequences of SEQ ID NO: 150 and SEQ ID NO: 151; a target sequence amplifiable by a primer pair having the sequences of SEQ ID NO: 166 and SEQ ID NO: 167; a target sequence amplifiable by a primer pair having the sequences of SEQ ID NO: 168 and SEQ ID NO: 169; a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 176 and SEQ ID NO: 177; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 184 and SEQ ID NO: 185; a target sequence amplifiable by a primer pair having the sequences of SEQ ID NO: 186 and SEQ ID NO: 187; a target sequence amplifiable by a primer pair having the sequences of SEQ ID NO: 188 and SEQ ID NO: 189; a target sequence amplifiable by a primer pair having the sequences of SEQ ID NO: 190 and SEQ ID NO: 191; a target sequence amplifiable by a primer pair having the sequences of SEQ ID NO: 196 and SEQ ID NO: 197; a target sequence amplifiable by a primer pair having the sequences of SEQ ID NO: 200 and SEQ ID NO: 201; a target sequence amplifiable by a primer pair having the sequences of SEQ ID NO: 210 and SEQ ID NO: 211; a target sequence amplifiable by a primer pair having the sequences of SEQ ID NO: 212 and SEQ ID NO: 213; a target sequence amplifiable by a primer pair having the sequences of SEQ ID NO: 214 and SEQ ID NO: 215; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 222 and SEQ ID NO: 223; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 230 and SEQ ID NO: 231; a target sequence amplifiable by a primer pair having the sequences of SEQ ID NO: 232 and SEQ ID NO: 233; a target sequence amplifiable by a primer pair having the sequences of SEQ ID NO: 238 and SEQ ID NO: 239; a target sequence amplifiable by a primer pair having the sequences of SEQ ID NO: 240 and SEQ ID NO: 241; a target sequence amplifiable by a primer pair having the sequences of SEQ ID NO: 242 and SEQ ID NO: 243; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; a target sequence amplifiable by a primer pair having the sequences of SEQ ID NO: 250 and SEQ ID NO: 251; a target sequence amplifiable by a primer pair having the sequences SEQ ID NO: 168 and SEQ ID NO: 295; a target sequence amplifiable by a primer pair having the sequences SEQ ID NO: 192 and SEQ ID NO: 300; a target sequence amplifiable by a primer pair having the sequences SEQ ID NO: 216 and SEQ ID NO: 217; a target sequence amplifiable by a primer pair having the sequences SEQ ID NO: 224 and SEQ ID NO: 305; a target sequence amplifiable by a primer pair having the sequences SEQ ID NO: 296 and SEQ ID NO: 297; a target sequence amplifiable by a primer pair having the sequences SEQ ID NO: 298 and SEQ ID NO: 299; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 6 and SEQ ID NO: 306; a target sequence amplifiable by a primer pair having the sequences SEQ ID NO: 307 and SEQ ID NO: 308; a target sequence amplifiable by a primer pair having the sequences SEQ ID NO: 309 and SEQ ID NO: 300; a target sequence amplifiable by a primer pair having the sequences SEQ ID NO: 310 and SEQ ID NO: 311; and a target sequence amplifiable by a primer pair having the sequences SEQ ID NO: 312 and SEQ ID NO: 313.

In particular embodiments, the at least two polymorphic target sequences comprise: a target sequence amplifiable by a primer pair having the sequences of SEQ ID NO: 1 and SEQ ID NO: 2; a target sequence amplifiable by a primer pair having the sequences of SEQ ID NO: 7 and SEQ ID NO: 8; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 23 and SEQ ID NO: 24; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 15 and SEQ ID NO: 16; and a target sequence amplifiable by a primer pair having the sequences of SEQ ID NO: 21 and SEQ ID NO: 22.

In particular embodiments, the at least two polymorphic target sequences comprise: a target sequence amplifiable by a primer pair having the sequences of SEQ ID NO: 7 and SEQ ID NO: 8; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 29 and SEQ ID NO: 30; a target sequence amplifiable by a primer pair having the sequences of SEQ ID NO: 37 and SEQ ID NO: 38; a target sequence amplifiable by a primer pair having the sequences of SEQ ID NO: 45 and SEQ ID NO: 46; a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 13 and SEQ ID NO: 14; a target sequence amplifiable by a primer pair having the sequences of SEQ ID NO: 39 and SEQ ID NO: 40; and a target sequence amplifiable by a primer pair having the sequences of SEQ ID NO: 43 and SEQ ID NO: 44.

In particular embodiments, the at least two polymorphic target sequences comprise: a target sequence amplifiable by a primer pair having the sequences of SEQ ID NO: 1 and SEQ ID NO: 2; a target sequence amplifiable by a primer pair having the sequences of SEQ ID NO: 3 and SEQ ID NO: 4; a target sequence amplifiable by a primer pair having the sequences of SEQ ID NO: 5 and SEQ ID NO: 6; a target sequence amplifiable by a primer pair having the sequences of SEQ ID NO: 9 and SEQ ID NO: 10; a target sequence amplifiable by a primer pair having the sequences of SEQ ID NO: 11 and SEQ ID NO: 12; a target sequence amplifiable by a primer pair having the sequences of SEQ ID NO: 13 and SEQ ID NO: 14; a target sequence amplifiable by a primer pair having the sequences of SEQ ID NO: 17 and SEQ ID NO: 18; a target sequence amplifiable by a primer pair having the sequences of SEQ ID NO: 19 and SEQ ID NO: 20; a target sequence amplifiable by a primer pair having the sequences of SEQ ID NO: 21 and SEQ ID NO: 22; and a target sequence amplifiable by a primer pair having the sequences of SEQ ID NO: 23 and SEQ ID NO: 24.

In particular embodiments, the at least two polymorphic target sequences comprise: a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences of SEQ ID NO: 170 and SEQ ID NO: 171; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences of SEQ ID NO: 170 and SEQ ID NO: 171; a target sequence amplifiable by a primer pair having the sequences of SEQ ID NO: 174 and SEQ ID NO: 175; a target sequence amplifiable by a primer pair having the sequences of SEQ ID NO: 180 and SEQ ID NO: 181; a target sequence amplifiable by a primer pair having the sequences of SEQ ID NO: 182 and SEQ ID NO: 183; a target sequence amplifiable by a primer pair having the sequences of SEQ ID NO: 188 and SEQ ID NO: 189; a target sequence amplifiable by a primer pair having the sequences of SEQ ID NO: 210 and SEQ ID NO: 211; a target sequence amplifiable by a primer pair having the sequences of SEQ ID NO: 212 and SEQ ID NO: 213; a target sequence amplifiable by a primer pair having the sequences of SEQ ID NO: 218 and SEQ ID NO: 219; a target sequence amplifiable by a primer pair having the sequences of SEQ ID NO: 220 and SEQ ID NO: 221; a target sequence amplifiable by a primer pair having the sequences of SEQ ID NO: 224 and SEQ ID NO: 225; a target sequence amplifiable by a primer pair having the sequences of SEQ ID NO: 228 and SEQ ID NO: 229; a target sequence amplifiable by a primer pair having the sequences of SEQ ID NO: 244 and SEQ ID NO: 245; a target sequence amplifiable by a primer pair having the sequences of SEQ ID NO: 248 and SEQ ID NO: 249; and a target sequence amplifiable by a primer pair having the sequences of SEQ ID NO: 250 and SEQ ID NO: 251.

In particular embodiments, the at least two polymorphic target sequences comprise: a target sequence amplifiable by a primer pair having the sequences SEQ ID NO: 168 and SEQ ID NO: 295; a target sequence amplifiable by a primer pair having the sequences SEQ ID NO: 170 and SEQ ID NO: 171; a target sequence amplifiable by a primer pair having the sequences SEQ ID NO: 174 and SEQ ID NO: 175; a target sequence amplifiable by a primer pair having the sequences SEQ ID NO: 296 and SEQ ID NO: 297; a target sequence amplifiable by a primer pair having the sequences SEQ ID NO: 180 and SEQ ID NO: 181; a target sequence amplifiable by a primer pair having the sequences SEQ ID NO: 298 and SEQ ID NO: 299; a target sequence amplifiable by a primer pair having the sequences SEQ ID NO: 192 and SEQ ID NO: 300; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 210 and SEQ ID NO: 211; a target sequence amplifiable by a primer pair having the sequences SEQ ID NO: 212 and SEQ ID NO: 213; a target sequence amplifiable by a primer pair having the sequences SEQ ID NO: 214 and SEQ ID NO: 215; a target sequence amplifiable by a primer pair having the sequences SEQ ID NO: 216 and SEQ ID NO: 217; a target sequence amplifiable by a primer pair having the sequences SEQ ID NO: 218 and SEQ ID NO: 219; a target sequence amplifiable by a primer pair having the sequences SEQ ID NO: 220 and SEQ ID NO: 221; a target sequence amplifiable by a primer pair having the sequences SEQ ID NO: 224 and SEQ ID NO: 305; a target sequence amplifiable by a primer pair having the sequences SEQ ID NO: 244 and SEQ ID NO: 245; and a target sequence amplifiable by a primer pair having the sequences SEQ ID NO: 6 and SEQ ID NO: 306.

In particular embodiments, the at least two polymorphic target sequences comprise: a target sequence amplifiable by a primer pair having the sequences SEQ ID NO: 168 and SEQ ID NO: 295; a target sequence amplifiable by a primer pair having the sequences SEQ ID NO: 170 and SEQ ID NO: 171; a target sequence amplifiable by a primer pair having the sequences SEQ ID NO: 312 and SEQ ID NO: 313; a target sequence amplifiable by a primer pair having the sequences SEQ ID NO: 307 and SEQ ID NO: 308; a target sequence amplifiable by a primer pair having the sequences SEQ ID NO: 180 and SEQ ID NO: 181; a target sequence amplifiable by a primer pair having the sequences SEQ ID NO: 298 and SEQ ID NO: 299; a target sequence amplifiable by a primer pair having the sequences SEQ ID NO: 309 and SEQ ID NO: 300; a target sequence amplifiable by a primer pair having the sequences SEQ ID NO: 301 and SEQ ID NO: 302; a target sequence amplifiable by a primer pair having the sequences SEQ ID NO: 303 and SEQ ID NO: 304; a target sequence amplifiable by a primer pair having the sequences SEQ ID NO: 210 and SEQ ID NO: 211; a target sequence amplifiable by a primer pair having the sequences SEQ ID NO: 212 and SEQ ID NO: 213; a target sequence amplifiable by a primer pair having the sequences SEQ ID NO: 214 and SEQ ID NO: 215; a target sequence amplifiable by a primer pair having the sequences SEQ ID NO: 216 and SEQ ID NO: 217; a target sequence amplifiable by a primer pair having the sequences SEQ ID NO: 310 and SEQ ID NO: 311; a target sequence amplifiable by a primer pair having the sequences SEQ ID NO: 220 and SEQ ID NO: 221; a target sequence amplifiable by a primer pair having the sequences SEQ ID NO: 224 and SEQ ID NO: 305; a target sequence amplifiable by a primer pair having the sequences SEQ ID NO: 244 and SEQ ID NO: 245; and a target sequence amplifiable by a primer pair having the sequences SEQ ID NO: 6 and SEQ ID NO: 306.

In another embodiment, the at least two polymorphic target sequences comprise two or more of: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; a target sequence flanked by SEQ ID NO: 23 on the 5′ end and SEQ ID NO: 112 on the 3′ end; a target sequence flanked by SEQ ID NO: 25 on the 5′ end and SEQ ID NO: 113 on the 3′ end; a target sequence flanked by SEQ ID NO: 27 on the 5′ end and SEQ ID NO: 114 on the 3′ end; a target sequence flanked by SEQ ID NO: 29 on the 5′ end and SEQ ID NO: 115 on the 3′ end; a target sequence flanked by SEQ ID NO: 31 on the 5′ end and SEQ ID NO: 116 on the 3′ end; a target sequence flanked by SEQ ID NO: 33 on the 5′ end and SEQ ID NO: 117 on the 3′ end; a target sequence flanked by SEQ ID NO: 35 on the 5′ end and SEQ ID NO: 118 on the 3′ end; a target sequence flanked by SEQ ID NO: 37 on the 5′ end and SEQ ID NO: 119 on the 3′ end; a target sequence flanked by SEQ ID NO: 39 on the 5′ end and SEQ ID NO: 120 on the 3′ end; a target sequence flanked by SEQ ID NO: 41 on the 5′ end and SEQ ID NO: 121 on the 3′ end; a target sequence flanked by SEQ ID NO: 43 on the 5′ end and SEQ ID NO: 122 on the 3′ end; a target sequence flanked by SEQ ID NO: 45 on the 5′ end and SEQ ID NO: 123 on the 3′ end; a target sequence flanked by SEQ ID NO: 124 on the 5′ end and SEQ ID NO: 152 on the 3′ end a target sequence flanked by SEQ ID NO: 126 on the 5′ end and SEQ ID NO: 153 on the 3′ end; a target sequence flanked by SEQ ID NO: 128 on the 5′ end and SEQ ID NO: 154 on the 3′ end; a target sequence flanked by SEQ ID NO: 130 on the 5′ end and SEQ ID NO: 155 on the 3′ end; a target sequence flanked by SEQ ID NO: 132 on the 5′ end and SEQ ID NO: 156 on the 3′ end; a target sequence flanked by SEQ ID NO: 134 on the 5′ end and SEQ ID NO: 157 on the 3′ end; a target sequence flanked by SEQ ID NO: 136 on the 5′ end and SEQ ID NO: 158 on the 3′ end; a target sequence flanked by SEQ ID NO: 138 on the 5′ end and SEQ ID NO: 159 on the 3′ end; a target sequence flanked by SEQ ID NO: 140 on the 5′ end and SEQ ID NO: 160 on the 3′ end; a target sequence flanked by SEQ ID NO: 142 on the 5′ end and SEQ ID NO: 161 on the 3′ end; a target sequence flanked by SEQ ID NO: 144 on the 5′ end and SEQ ID NO: 162 on the 3′ end; a target sequence flanked by SEQ ID NO: 146 on the 5′ end and SEQ ID NO: 163 on the 3′ end; a target sequence flanked by SEQ ID NO: 148 on the 5′ end and SEQ ID NO: 164 on the 3′ end; a target sequence flanked by SEQ ID NO: 150 on the 5′ end and SEQ ID NO: 165 on the 3′ end a target sequence flanked by SEQ ID NO: 166 on the 5′ end and SEQ ID NO: 252 on the 3′ end; a target sequence flanked by SEQ ID NO: 168 on the 5′ end and SEQ ID NO: 253 on the 3′ end; a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 176 on the 5′ end and SEQ ID NO: 257 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 184 on the 5′ end and SEQ ID NO: 261 on the 3′ end; a target sequence flanked by SEQ ID NO: 186 on the 5′ end and SEQ ID NO: 262 on the 3′ end; a target sequence flanked by SEQ ID NO: 188 on the 5′ end and SEQ ID NO: 263 on the 3′ end; a target sequence flanked by SEQ ID NO: 190 on the 5′ end and SEQ ID NO: 264 on the 3′ end; a target sequence flanked by SEQ ID NO: 196 on the 5′ end and SEQ ID NO: 267 on the 3′ end; a target sequence flanked by SEQ ID NO: 200 on the 5′ end and SEQ ID NO: 269 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 222 on the 5′ end and SEQ ID NO: 280 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 230 on the 5′ end and SEQ ID NO: 284 on the 3′ end; a target sequence flanked by SEQ ID NO: 232 on the 5′ end and SEQ ID NO: 285 on the 3′ end; a target sequence flanked by SEQ ID NO: 238 on the 5′ end and SEQ ID NO: 288 on the 3′ end; a target sequence flanked by SEQ ID NO: 240 on the 5′ end and SEQ ID NO: 289 on the 3′ end; a target sequence flanked by SEQ ID NO: 242 on the 5′ end and SEQ ID NO: 290 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; a target sequence flanked by SEQ ID NO: 250 on the 5′ end and SEQ ID NO: 294 on the 3′ end; a target sequence flanked by SEQ ID NO: 168 on the 5′ end and SEQ ID NO: 314 on the 3′ end; a target sequence flanked by SEQ ID NO: 192 on the 5′ end and SEQ ID NO: 317 on the 3′ end; a target sequence flanked by SEQ ID NO: 216 on the 5′ end and SEQ ID NO: 277 on the 3′ end; a target sequence flanked by SEQ ID NO: 224 on the 5′ end and SEQ ID NO: 320 on the 3′ end; a target sequence flanked by SEQ ID NO: 296 on the 5′ end and SEQ ID NO: 315 on the 3′ end; a target sequence flanked by SEQ ID NO: 298 on the 5′ end and SEQ ID NO: 316 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 306 on the 5′ end and SEQ ID NO: 103 on the 3′ end; a target sequence flanked by SEQ ID NO: 307 on the 5′ end and SEQ ID NO: 321 on the 3′ end; a target sequence flanked by SEQ ID NO: 309 on the 5′ end and SEQ ID NO: 317 on the 3′ end; a target sequence flanked by SEQ ID NO: 310 on the 5′ end and SEQ ID NO: 322 on the 3′ end; and a target sequence flanked by SEQ ID NO: 312 on the 5′ end and SEQ ID NO: 323 on the 3′ end.

In a particular embodiment, the at least two polymorphic target sequences comprise: a target sequence flanked by SEQ ID NO: 1 on the 5′ end and SEQ ID NO: 101 on the 3′ end; a target sequence flanked by SEQ ID NO: 7 on the 5′ end and SEQ ID NO: 104 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 23 on the 5′ end and SEQ ID NO: 112 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; a target sequence flanked by SEQ ID NO: 21 on the 5′ end and SEQ ID NO: 111 on the 3′ end;

In particular embodiments, the at least two polymorphic target sequences comprise: a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 29 on the 5′ end and SEQ ID NO: 115 on the 3′ end; a target sequence flanked by SEQ ID NO: 37 on the 5′ end and SEQ ID NO: 119 on the 3′ end; a target sequence flanked by SEQ ID NO: 45 on the 5′ end and SEQ ID NO: 123 on the 3′ end; a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 13 on the 5′ end and SEQ ID NO: 107 on the 3′ end; a target sequence flanked by SEQ ID NO: 39 on the 5′ end and SEQ ID NO: 120 on the 3′ end; and a target sequence flanked by SEQ ID NO: 43 on the 5′ end and SEQ ID NO: 122 on the 3′ end.

In particular embodiments, the at least two polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; and a target sequence flanked by SEQ ID NO: 23 on the 5′ end and SEQ ID NO: 112 on the 3′ end.

In particular embodiments, the at least two polymorphic target sequences comprise: a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end; and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In particular embodiments, the at least two polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 296 on the 5′ end and SEQ ID NO: 315 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 192 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

In particular embodiments, the at least two polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 312 on the 5′ end and SEQ ID NO: 323 on the 3′ end; a target sequence flanked by SEQ ID NO: 307 on the 5′ end and SEQ ID NO: 321 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 309 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 310 on the 5′ end and SEQ ID NO: 322 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

In another embodiment, the disclosure pertains to a set of primers for constructing a genetic profile for a Cannabis plant comprising two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; a primer pair having the sequences of SEQ ID NO: 23 and SEQ ID NO: 24; a primer pair having the sequences of SEQ ID NO: 25 and SEQ ID NO: 26; a primer pair having the sequences of SEQ ID NO: 27 and SEQ ID NO: 28; a primer pair having the sequences of SEQ ID NO: 29 and SEQ ID NO: 30; a primer pair having the sequences of SEQ ID NO: 31 and SEQ ID NO: 32; a primer pair having the sequences of SEQ ID NO: 33 and SEQ ID NO: 34; a primer pair having the sequences of SEQ ID NO: 35 and SEQ ID NO: 36; a primer pair having the sequences of SEQ ID NO: 37 and SEQ ID NO: 38; a primer pair having the sequences of SEQ ID NO: 39 and SEQ ID NO: 40; a primer pair having the sequences of SEQ ID NO: 41 and SEQ ID NO: 42; a primer pair having the sequences of SEQ ID NO: 43 and SEQ ID NO: 44; a primer pair having the sequences of SEQ ID NO: 45 and SEQ ID NO: 46; a primer pair having the sequences of SEQ ID NO: 124 and SEQ ID NO: 125; a primer pair having the sequences of SEQ ID NO: 126 and SEQ ID NO: 127; a primer pair having the sequences of SEQ ID NO: 128 and SEQ ID NO: 129; a primer pair having the sequences of SEQ ID NO: 130 and SEQ ID NO: 131; a primer pair having the sequences of SEQ ID NO: 132 and SEQ ID NO: 133; a primer pair having the sequences of SEQ ID NO: 134 and SEQ ID NO: 135; a primer pair having the sequences of SEQ ID NO: 136 and SEQ ID NO: 137; a primer pair having the sequences of SEQ ID NO: 138 and SEQ ID NO: 139; a primer pair having the sequences of SEQ ID NO: 140 and SEQ ID NO: 141; a primer pair having the sequences of SEQ ID NO: 142 and SEQ ID NO: 143; a primer pair having the sequences of SEQ ID NO: 144 and SEQ ID NO: 145; a primer pair having the sequences of SEQ ID NO: 146 and SEQ ID NO: 147; a primer pair having the sequences of SEQ ID NO: 148 and SEQ ID NO: 149; a primer pair having the sequences of SEQ ID NO: 150 and SEQ ID NO: 151 a primer pair having the sequences of SEQ ID NO: 166 and SEQ ID NO: 167; a primer pair having the sequences of SEQ ID NO: 168 and SEQ ID NO: 169; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 176 and SEQ ID NO: 177; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 184 and SEQ ID NO: 185; a primer pair having the sequences of SEQ ID NO: 186 and SEQ ID NO: 187; a primer pair having the sequences of SEQ ID NO: 188 and SEQ ID NO: 189; a primer pair having the sequences of SEQ ID NO: 190 and SEQ ID NO: 191; a primer pair having the sequences of SEQ ID NO: 196 and SEQ ID NO: 197; a primer pair having the sequences of SEQ ID NO: 200 and SEQ ID NO: 201; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 214 and SEQ ID NO: 215; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 222 and SEQ ID NO: 223; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 230 and SEQ ID NO: 231; a primer pair having the sequences of SEQ ID NO: 232 and SEQ ID NO: 233; a primer pair having the sequences of SEQ ID NO: 238 and SEQ ID NO: 239; a primer pair having the sequences of SEQ ID NO: 240 and SEQ ID NO: 241; a primer pair having the sequences of SEQ ID NO: 242 and SEQ ID NO: 243; a primer pair having the sequences of SEQ ID NO: 244 and SEQ ID NO: 245; a primer pair having the sequences of SEQ ID NO: 248 and SEQ ID NO: 249; a primer pair having the sequences of SEQ ID NO: 250 and SEQ ID NO: 251; a primer pair having the sequences of SEQ ID NO: 168 and SEQ ID NO: 295; a primer pair having the sequences of SEQ ID NO: 192 and SEQ ID NO: 300; a primer pair having the sequences of SEQ ID NO: 216 and SEQ ID NO: 217; a primer pair having the sequences of SEQ ID NO: 224 and SEQ ID NO: 305; a primer pair having the sequences of SEQ ID NO: 296 and SEQ ID NO: 297; a primer pair having the sequences of SEQ ID NO: 298 and SEQ ID NO: 299; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 6 and SEQ ID NO: 306; a primer pair having the sequences of SEQ ID NO: 307 and SEQ ID NO: 308; a primer pair having the sequences of SEQ ID NO: 309 and SEQ ID NO: 300; a primer pair having the sequences of SEQ ID NO: 310 and SEQ ID NO: 311; and a primer pair having the sequences of SEQ ID NO: 312 and SEQ ID NO: 313.

In another embodiment, the disclosure pertains to a set of primers for constructing a genetic profile for a Cannabis plant. The set comprises a plurality of primer pairs for specifically amplifying at least five polymorphic target sequences. The at least five polymorphic target sequences comprise: a target sequence flanked by SEQ ID NO: 1 on the 5′ end and SEQ ID NO: 101 on the 3′ end; a target sequence flanked by SEQ ID NO: 7 on the 5′ end and SEQ ID NO: 104 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; and a target sequence flanked by SEQ ID NO: 23 on the 5′ end and SEQ ID NO: 112 on the 3′ end.

In another embodiment, the disclosure pertains to a set of primers for constructing a genetic profile for a Cannabis plant. The set comprises a plurality of primer pairs for specifically amplifying at least five polymorphic target sequences. The at least five polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 7 and SEQ ID NO: 8; a target polymorphic sequence amplifiable by a primer pair having the sequences of SEQ ID NO: 17 and SEQ ID NO: 18; a target polymorphic sequence amplifiable by a primer pair having the sequences of SEQ ID NO: 19 and SEQ ID NO: 20; and a target polymorphic sequence amplifiable by a primer pair having the sequences of SEQ ID NO: 23 and SEQ ID NO: 24.

In another embodiment, the disclosure pertains to a set of primers for constructing a genetic profile for a Cannabis plant. The set comprises a plurality of primer pairs for specifically amplifying at least seven polymorphic target sequences. The at least seven polymorphic target sequences comprise: a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 15 and SEQ ID NO: 16; and a target polymorphic sequence amplifiable by a primer pair having the sequences of SEQ ID NO: 21 and SEQ ID NO: 22.

In another embodiment, the disclosure pertains to a set of primers for constructing a genetic profile for a Cannabis plant. The set comprises a plurality of primer pairs for specifically amplifying at least seven polymorphic target sequences. The at least seven polymorphic target sequences comprise: a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; and a target sequence flanked by SEQ ID NO: 21 on the 5′ end and SEQ ID NO: 111 on the 3′ end.

In another embodiment, the disclosure pertains to a set of primers for constructing a genetic profile for a Cannabis plant. The set comprises a first subset of primer pairs for specifically amplifying at least five polymorphic target sequences, wherein the at least five polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 7 and SEQ ID NO: 8; a target polymorphic sequence amplifiable by a primer pair having the sequences of SEQ ID NO: 17 and SEQ ID NO: 18; a target polymorphic sequence amplifiable by a primer pair having the sequences of SEQ ID NO: 19 and SEQ ID NO: 20; and a target polymorphic sequence amplifiable by a primer pair having the sequences of SEQ ID NO: 23 and SEQ ID NO: 24. The set further comprises a second subset of primer pairs for specifically amplifying seven polymorphic target sequences, wherein the at least seven polymorphic target sequences: a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 15 and SEQ ID NO: 16; and a target polymorphic sequence amplifiable by a primer pair having the sequences of SEQ ID NO: 21 and SEQ ID NO: 22.

In another embodiment, the disclosure pertains to a set of primers for constructing a genetic profile for a Cannabis plant. The set comprises a first subset of primer pairs for specifically amplifying at least five polymorphic target sequences, wherein the at least five polymorphic target sequences comprise: a target sequence flanked by SEQ ID NO: 1 on the 5′ end and SEQ ID NO: 101 on the 3′ end; a target sequence flanked by SEQ ID NO: 7 on the 5′ end and SEQ ID NO: 104 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; and a target sequence flanked by SEQ ID NO: 23 on the 5′ end and SEQ ID NO: 112 on the 3′ end. The set further comprises a second subset of primer pairs for specifically amplifying seven polymorphic target sequences, wherein the at least seven polymorphic target sequences comprise: a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 15 on the 5′ end and SEQ ID NO: 108 on the 3′ end; and a target sequence flanked by SEQ ID NO: 21 on the 5′ end and SEQ ID NO: 111 on the 3′ end.

In another embodiment, the disclosure pertains to a set of primers for constructing a genetic profile for a Cannabis plant. The set comprises a plurality of primer pairs for specifically amplifying at least six polymorphic target sequences. The at least six polymorphic target sequences comprise: a target polymorphic sequence amplifiable by a primer pair having the sequences of SEQ ID NO: 7 and SEQ ID NO: 8; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 29 and SEQ ID NO: 30; a target polymorphic sequence amplifiable by a primer pair having the sequences of SEQ ID NO: 37 and SEQ ID NO: 38; and a target polymorphic sequence amplifiable by a primer pair having the sequences of SEQ ID NO: 45 and SEQ ID NO: 46.

In another embodiment, the disclosure pertains to a set of primers for constructing a genetic profile for a Cannabis plant. The set comprises a plurality of primer pairs for specifically amplifying at least six polymorphic target sequences. The at least six polymorphic target sequences comprise: a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 29 on the 5′ end and SEQ ID NO: 115 on the 3′ end; a target sequence flanked by SEQ ID NO: 37 on the 5′ end and SEQ ID NO: 119 on the 3′ end; and a target sequence flanked by SEQ ID NO: 45 on the 5′ end and SEQ ID NO: 123 on the 3′ end.

In another embodiment, the disclosure pertains to a set of primers for constructing a genetic profile for a Cannabis plant. The set comprises a plurality of primer pairs for specifically amplifying at least six polymorphic target sequences. The at least six polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 39 and SEQ ID NO: 40; and a target polymorphic sequence amplifiable by a primer pair having the sequences of SEQ ID NO: 43 and SEQ ID NO: 44.

In another embodiment, the disclosure pertains to a set of primers for constructing a genetic profile for a Cannabis plant. The set comprises a plurality of primer pairs for specifically amplifying at least six polymorphic target sequences. The at least six polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 13 on the 5′ end and SEQ ID NO: 107 on the 3′ end; a target sequence flanked by SEQ ID NO: 39 on the 5′ end and SEQ ID NO: 120 on the 3′ end; and a target sequence flanked by SEQ ID NO: 43 on the 5′ end and SEQ ID NO: 122 on the 3′ end.

In another embodiment, the disclosure pertains to a set of primers for constructing a genetic profile for a Cannabis plant. The set comprises a first subset of primer pairs for specifically amplifying at least six polymorphic target sequences, wherein the at least six polymorphic target sequences comprise: a target polymorphic sequence amplifiable by a primer pair having the sequences of SEQ ID NO: 7 and SEQ ID NO: 8; a target polymorphic sequence amplifiable by a primer pair having the sequences of SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 29 and SEQ ID NO: 30; a target polymorphic sequence amplifiable by a primer pair having the sequences of SEQ ID NO: 37 and SEQ ID NO: 38; and a target polymorphic sequence amplifiable by a primer pair having the sequences of SEQ ID NO: 45 and SEQ ID NO: 46. The set further comprises a second subset of primer pairs for specifically amplifying seven polymorphic target sequences, wherein the at least six polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 39 and SEQ ID NO: 40; and a target polymorphic sequence amplifiable by a primer pair having the sequences of SEQ ID NO: 43 and SEQ ID NO: 44.

In another embodiment, the disclosure pertains to a set of primers for constructing a genetic profile for a Cannabis plant. The set comprises a first subset of primer pairs for specifically amplifying at least six polymorphic target sequences. The at least six polymorphic target sequences comprise: a target sequence flanked by SEQ ID NO: 7 on the 5′ end and SEQ ID NO: 104 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 29 on the 5′ end and SEQ ID NO: 115 on the 3′ end; a target sequence flanked by SEQ ID NO: 37 on the 5′ end and SEQ ID NO: 119 on the 3′ end; and a target sequence flanked by SEQ ID NO: 45 on the 5′ end and SEQ ID NO: 123 on the 3′ end. The set further comprises a second subset of primer pairs for specifically amplifying seven polymorphic target sequences, wherein the at least six polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 13 on the 5′ end and SEQ ID NO: 107 on the 3′ end; a target sequence flanked by SEQ ID NO: 39 on the 5′ end and SEQ ID NO: 120 on the 3′ end; and a target sequence flanked by SEQ ID NO: 43 on the 5′ end and SEQ ID NO: 122 on the 3′ end.

In another embodiment, the disclosure pertains to a set of primers for constructing a genetic profile for a Cannabis plant. The set comprises a plurality of primer pairs for specifically amplifying at least ten polymorphic target sequences. The at least ten polymorphic target sequences comprise: a target sequence flanked by SEQ ID NO: 1 on the 5′ end and SEQ ID NO: 101 on the 3′ end; a target sequence flanked by SEQ ID NO: 3 on the 5′ end and SEQ ID NO: 102 on the 3′ end; a target sequence flanked by SEQ ID NO: 5 on the 5′ end and SEQ ID NO: 103 on the 3′ end; a target sequence flanked by SEQ ID NO: 9 on the 5′ end and SEQ ID NO: 105 on the 3′ end; a target sequence flanked by SEQ ID NO: 11 on the 5′ end and SEQ ID NO: 106 on the 3′ end; a target sequence flanked by SEQ ID NO: 13 on the 5′ end and SEQ ID NO: 107 on the 3′ end; a target sequence flanked by SEQ ID NO: 17 on the 5′ end and SEQ ID NO: 109 on the 3′ end; a target sequence flanked by SEQ ID NO: 19 on the 5′ end and SEQ ID NO: 110 on the 3′ end; a target sequence flanked by SEQ ID NO: 21 on the 5′ end and SEQ ID NO: 111 on the 3′ end; and a target sequence flanked by SEQ ID NO: 23 on the 5′ end and SEQ ID NO: 112 on the 3′ end.

In another embodiment, the disclosure pertains to a set of primers for constructing a genetic profile for a Cannabis plant. The set comprises a plurality of primer pairs for specifically amplifying at least ten polymorphic target sequences. The at least ten polymorphic target sequences comprise: a target polymorphic sequence amplifiable by a primer pair having the sequences of SEQ ID NO: 1 and SEQ ID NO: 2; a target polymorphic sequence amplifiable by a primer pair having the sequences of SEQ ID NO: 3 and SEQ ID NO: 4; a target polymorphic sequence amplifiable by a primer pair having the sequences of SEQ ID NO: 5 and SEQ ID NO: 6; a target polymorphic sequence amplifiable by a primer pair having the sequences of S SEQ ID NO: 9 and SEQ ID NO: 10; a target polymorphic sequence amplifiable by a primer pair having the sequences of SEQ ID NO: 11 and SEQ ID NO: 12; a target polymorphic sequence amplifiable by a primer pair having the sequences of SEQ ID NO: 13 and SEQ ID NO: 14; a target polymorphic sequence amplifiable by a primer pair having the sequences of SEQ ID NO: 17 and SEQ ID NO: 18; a target polymorphic sequence amplifiable by a primer pair having the sequences of SEQ ID NO: 19 and SEQ ID NO: 20; a target polymorphic sequence amplifiable by a primer pair having the sequences of SEQ ID NO: 21 and SEQ ID NO: 22; and a target polymorphic sequence amplifiable by a primer pair having the sequences of SEQ ID NO: 23 and SEQ ID NO: 24.

In another embodiment, the disclosure pertains to a set of primers for constructing a genetic profile for a Cannabis plant. The set comprises a plurality of primer pairs for specifically amplifying at least eleven polymorphic target sequences. The at least eleven polymorphic target sequences comprise: a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In another embodiment, the disclosure pertains to a set of primers for constructing a genetic profile for a Cannabis plant. The set comprises a plurality of primer pairs for specifically amplifying at least eleven polymorphic target sequences. The at least eleven polymorphic target sequences comprise: a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 214 and SEQ ID NO: 215; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences SEQ ID NO: 248 and SEQ ID NO: 249; and a target polymorphic sequence amplifiable by a primer pair having the sequences of SEQ ID NO: 250 and SEQ ID NO: 251.

In another embodiment, the disclosure pertains to a set of primers for constructing a genetic profile for a Cannabis plant. The set comprises a plurality of primer pairs for specifically amplifying at least twelve polymorphic target sequences. The at least twelve polymorphic target sequences comprise: a target sequence flanked by SEQ ID NO: 170 on the 5′ end and SEQ ID NO: 254 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In another embodiment, the disclosure pertains to a set of primers for constructing a genetic profile for a Cannabis plant. The set comprises a plurality of primer pairs for specifically amplifying at least twelve polymorphic target sequences. The at least twelve polymorphic target sequences comprise: a target polymorphic sequence amplifiable by a primer pair having the sequences of SEQ ID NO: 170 and SEQ ID NO: 171; a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 220 and SEQ ID NO: 221; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences of SEQ ID NO: 248 and SEQ ID NO: 249; and a target polymorphic sequence amplifiable by a primer pair having the sequences of SEQ ID NO: 250 and SEQ ID NO: 251.

In another embodiment, the disclosure pertains to a set of primers for constructing a genetic profile for a Cannabis plant. The set comprises a plurality of primer pairs for specifically amplifying at least thirteen polymorphic target sequences. The at least thirteen polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In another embodiment, the disclosure pertains to a set of primers for constructing a genetic profile for a Cannabis plant. The set comprises a plurality of primer pairs for specifically amplifying at least thirteen polymorphic target sequences. The at least thirteen polymorphic target sequences comprise: a target polymorphic sequence amplifiable by a primer pair having the sequences of SEQ ID NO: 170 and SEQ ID NO: 171; a target polymorphic sequence amplifiable by a primer pair having the sequences of SEQ ID NO: 174 and SEQ ID NO: 175; a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 220 and SEQ ID NO: 221; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences of SEQ ID NO: 248 and SEQ ID NO: 249; and a target polymorphic sequence amplifiable by a primer pair having the sequences of SEQ ID NO: 250 and SEQ ID NO: 251.

In another embodiment, the disclosure pertains to a set of primers for constructing a genetic profile for a Cannabis plant. The set comprises a plurality of primer pairs for specifically amplifying at least fourteen polymorphic target sequences. The at least fourteen polymorphic target sequences comprise: a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 180 on the 5′ end and SEQ ID NO: 259 on the 3′ end; a target sequence flanked by SEQ ID NO: 182 on the 5′ end and SEQ ID NO: 260 on the 3′ end; a target sequence flanked by SEQ ID NO: 188 on the 5′ end and SEQ ID NO: 263 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 281 on the 3′ end; a target sequence flanked by SEQ ID NO: 228 on the 5′ end and SEQ ID NO: 283 on the 3′ end; a target sequence flanked by SEQ ID NO: 244 on the 5′ end and SEQ ID NO: 291 on the 3′ end; a target sequence flanked by SEQ ID NO: 248 on the 5′ end and SEQ ID NO: 293 on the 3′ end; and a target sequence flanked by SEQ ID NO: 250 on the 5′ end and SEQ ID NO: 294 on the 3′ end.

In another embodiment, the disclosure pertains to a set of primers for constructing a genetic profile for a Cannabis plant. The set comprises a plurality of primer pairs for specifically amplifying at least fourteen polymorphic target sequences. The at least fourteen polymorphic target sequences comprise: a target polymorphic sequence amplifiable by a primer pair having the sequences of SEQ ID NO: 170 and SEQ ID NO: 171; a target polymorphic sequence amplifiable by a primer pair having the sequences of SEQ ID NO: 174 and SEQ ID NO: 175; a target polymorphic sequence amplifiable by a primer pair having the sequences of SEQ ID NO: 180 and SEQ ID NO: 181; a target polymorphic sequence amplifiable by a primer pair having the sequences of SEQ ID NO: 182 and SEQ ID NO: 183; a target polymorphic sequence amplifiable by a primer pair having the sequences of SEQ ID NO: 188 and SEQ ID NO: 189; a target polymorphic sequence amplifiable by a primer pair having the sequences of SEQ ID NO: 210 and SEQ ID NO: 211; a target polymorphic sequence amplifiable by a primer pair having the sequences of SEQ ID NO: 212 and SEQ ID NO: 213; a target polymorphic sequence amplifiable by a primer pair having the sequences of SEQ ID NO: 218 and SEQ ID NO: 219; a target polymorphic sequence amplifiable by a primer pair having the sequences of SEQ ID NO: 220 and SEQ ID NO: 221; a target polymorphic sequence amplifiable by a primer pair having the sequences of SEQ ID NO: 224 and SEQ ID NO: 225; a target polymorphic sequence amplifiable by a primer pair having the sequences of SEQ ID NO: 228 and SEQ ID NO: 229; a target polymorphic sequence amplifiable by a primer pair having the sequences of SEQ ID NO: 244 and SEQ ID NO: 245; a target polymorphic sequence amplifiable by a primer pair having the sequences of SEQ ID NO: 248 and SEQ ID NO: 249; and a target polymorphic sequence amplifiable by a primer pair having the sequences of SEQ ID NO: 250 and SEQ ID NO: 251.

In another embodiment, the disclosure pertains to a set of primers for constructing a genetic profile for a Cannabis plant. The set comprises a plurality of primer pairs for specifically amplifying at least eighteen polymorphic target sequences. The at least eighteen polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 174 on the 5′ end and SEQ ID NO: 256 on the 3′ end; a target sequence flanked by SEQ ID NO: 296 on the 5′ end and SEQ ID NO: 315 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 192 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 218 on the 5′ end and SEQ ID NO: 278 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

In another embodiment, the disclosure pertains to a set of primers for constructing a genetic profile for a Cannabis plant. The set comprises a plurality of primer pairs for specifically amplifying at least eighteen polymorphic target sequences. The at least eighteen polymorphic target sequences comprise: a target polymorphic sequence amplifiable by a primer pair having the sequences SEQ ID NO: 168 and SEQ ID NO: 295; a target polymorphic sequence amplifiable by a primer pair having the sequences SEQ ID NO: 170 and SEQ ID NO: 171; a target polymorphic sequence amplifiable by a primer pair having the sequences SEQ ID NO: 174 and SEQ ID NO: 175; a target polymorphic sequence amplifiable by a primer pair having the sequences SEQ ID NO: 296 and SEQ ID NO: 297; a target polymorphic sequence amplifiable by a primer pair having the sequences SEQ ID NO: 180 and SEQ ID NO: 181; a target polymorphic sequence amplifiable by a primer pair having the sequences SEQ ID NO: 298 and SEQ ID NO: 299; a target polymorphic sequence amplifiable by a primer pair having the sequences SEQ ID NO: 192 and SEQ ID NO: 300; a target polymorphic sequence amplifiable by a primer pair having the sequences SEQ ID NO: 301 and SEQ ID NO: 302; a target polymorphic sequence amplifiable by a primer pair having the sequences SEQ ID NO: 303 and SEQ ID NO: 304; a target polymorphic sequence amplifiable by a primer pair having the sequences SEQ ID NO: 210 and SEQ ID NO: 211; a target polymorphic sequence amplifiable by a primer pair having the sequences SEQ ID NO: 212 and SEQ ID NO: 213; a target polymorphic sequence amplifiable by a primer pair having the sequences SEQ ID NO: 214 and SEQ ID NO: 215; a target polymorphic sequence amplifiable by a primer pair having the sequences SEQ ID NO: 216 and SEQ ID NO: 217; a target polymorphic sequence amplifiable by a primer pair having the sequences SEQ ID NO: 218 and SEQ ID NO: 219; a target polymorphic sequence amplifiable by a primer pair having the sequences SEQ ID NO: 220 and SEQ ID NO: 221; a target polymorphic sequence amplifiable by a primer pair having the sequences SEQ ID NO: 224 and SEQ ID NO: 305; a target polymorphic sequence amplifiable by a primer pair having the sequences SEQ ID NO: 244 and SEQ ID NO: 245; and a target polymorphic sequence amplifiable by a primer pair having the sequences SEQ ID NO: 6 and SEQ ID NO: 306.

In another embodiment, the disclosure pertains to a set of primers for constructing a genetic profile for a Cannabis plant. The set comprises a plurality of primer pairs for specifically amplifying at least eighteen polymorphic target sequences. The at least eighteen polymorphic target sequences comprise: a target sequence flanked by SEQ ID NO: 168 on the 5′ end and SEQ ID NO: 314 on the 3′ end; a target sequence flanked by SEQ ID NO: 170 on the 5′ end and SEQ ID NO: 254 on the 3′ end; a target sequence flanked by SEQ ID NO: 312 on the 5′ end and SEQ ID NO: 323 on the 3′ end; a target sequence flanked by SEQ ID NO: 307 on the 5′ end and SEQ ID NO: 321 on the 3′ end; a target sequence flanked by SEQ ID NO: 180 on the 5′ end and SEQ ID NO: 259 on the 3′ end; a target sequence flanked by SEQ ID NO: 298 on the 5′ end and SEQ ID NO: 316 on the 3′ end; a target sequence flanked by SEQ ID NO: 309 on the 5′ end and SEQ ID NO: 317 on the 3′ end; a target sequence flanked by SEQ ID NO: 301 on the 5′ end and SEQ ID NO: 318 on the 3′ end; a target sequence flanked by SEQ ID NO: 303 on the 5′ end and SEQ ID NO: 319 on the 3′ end; a target sequence flanked by SEQ ID NO: 210 on the 5′ end and SEQ ID NO: 274 on the 3′ end; a target sequence flanked by SEQ ID NO: 212 on the 5′ end and SEQ ID NO: 275 on the 3′ end; a target sequence flanked by SEQ ID NO: 214 on the 5′ end and SEQ ID NO: 276 on the 3′ end; a target sequence flanked by SEQ ID NO: 216 on the 5′ end and SEQ ID NO: 277 on the 3′ end; a target sequence flanked by SEQ ID NO: 310 on the 5′ end and SEQ ID NO: 322 on the 3′ end; a target sequence flanked by SEQ ID NO: 220 on the 5′ end and SEQ ID NO: 279 on the 3′ end; a target sequence flanked by SEQ ID NO: 224 on the 5′ end and SEQ ID NO: 320 on the 3′ end; a target sequence flanked by SEQ ID NO: 244 on the 5′ end and SEQ ID NO: 291 on the 3′ end; and a target sequence flanked by SEQ ID NO: 306 on the 5′ end and SEQ ID NO: 103 on the 3′ end.

In another embodiment, the disclosure pertains to a set of primers for constructing a genetic profile for a Cannabis plant. The set comprises a plurality of primer pairs for specifically amplifying at least eighteen polymorphic target sequences. The at least eighteen polymorphic target sequences comprise: a target polymorphic sequence amplifiable by a primer pair having the sequences SEQ ID NO: 168 and SEQ ID NO: 295; a target polymorphic sequence amplifiable by a primer pair having the sequences SEQ ID NO: 170 and SEQ ID NO: 171; a target polymorphic sequence amplifiable by a primer pair having the sequences SEQ ID NO: 312 and SEQ ID NO: 313; a target polymorphic sequence amplifiable by a primer pair having the sequences SEQ ID NO: 307 and SEQ ID NO: 308; a target polymorphic sequence amplifiable by a primer pair having the sequences SEQ ID NO: 180 and SEQ ID NO: 181; a target polymorphic sequence amplifiable by a primer pair having the sequences SEQ ID NO: 298 and SEQ ID NO: 299; a target polymorphic sequence amplifiable by a primer pair having the sequences SEQ ID NO: 309 and SEQ ID NO: 300; a target polymorphic sequence amplifiable by a primer pair having the sequences SEQ ID NO: 301 and SEQ ID NO: 302; a target polymorphic sequence amplifiable by a primer pair having the sequences SEQ ID NO: 303 and SEQ ID NO: 304; a target polymorphic sequence amplifiable by a primer pair having the sequences SEQ ID NO: 210 and SEQ ID NO: 211; a target polymorphic sequence amplifiable by a primer pair having the sequences SEQ ID NO: 212 and SEQ ID NO: 213; a target polymorphic sequence amplifiable by a primer pair having the sequences SEQ ID NO: 214 and SEQ ID NO: 215; a target polymorphic sequence amplifiable by a primer pair having the sequences SEQ ID NO: 216 and SEQ ID NO: 217; a target polymorphic sequence amplifiable by a primer pair having the sequences SEQ ID NO: 310 and SEQ ID NO: 311; a target polymorphic sequence amplifiable by a primer pair having the sequences SEQ ID NO: 220 and SEQ ID NO: 221; a target polymorphic sequence amplifiable by a primer pair having the sequences SEQ ID NO: 224 and SEQ ID NO: 305; a target polymorphic sequence amplifiable by a primer pair having the sequences SEQ ID NO: 244 and SEQ ID NO: 245; and a target polymorphic sequence amplifiable by a primer pair having the sequences SEQ ID NO: 6 and SEQ ID NO: 306.

In another embodiment, the disclosure pertains to a set of primers for constructing a genetic profile for a Cannabis plant. The set comprises two or more of: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; a primer pair having the sequences of SEQ ID NO: 17 and SEQ ID NO: 18; a primer pair having the sequences of SEQ ID NO: 19 and SEQ ID NO: 20; a primer pair having the sequences of SEQ ID NO: 21 and SEQ ID NO: 22; a primer pair having the sequences of SEQ ID NO: 23 and SEQ ID NO: 24. a primer pair having the sequences of SEQ ID NO: 29 and SEQ ID NO: 30; a primer pair having the sequences of SEQ ID NO: 37 and SEQ ID NO: 38; a primer pair having the sequences of SEQ ID NO: 39 and SEQ ID NO: a primer pair having the sequences of SEQ ID NO: 43 and SEQ ID NO: 44; a primer pair having the sequences of SEQ ID NO: 45 and SEQ ID NO: 46; a primer pair having the sequences of SEQ ID NO: 124 and SEQ ID NO: 125; a primer pair having the sequences of SEQ ID NO: 126 and SEQ ID NO: 127; a primer pair having the sequences of SEQ ID NO: 130 and SEQ ID NO: 131; a primer pair having the sequences of SEQ ID NO: 132 and SEQ ID NO: 133; a primer pair having the sequences of SEQ ID NO: 134 and SEQ ID NO: 135; a primer pair having the sequences of SEQ ID NO: 136 and SEQ ID NO: 137; a primer pair having the sequences of SEQ ID NO: 138 and SEQ ID NO: 139; a primer pair having the sequences of SEQ ID NO: 140 and SEQ ID NO: 141; a primer pair having the sequences of SEQ ID NO: 142 and SEQ ID NO: 143; a primer pair having the sequences of SEQ ID NO: 144 and SEQ ID NO: 145; a primer pair having the sequences of SEQ ID NO: 146 and SEQ ID NO: 147; a primer pair having the sequences of SEQ ID NO: 148 and SEQ ID NO: 149; a primer pair having the sequences of SEQ ID NO: 150 and SEQ ID NO: 151; a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; a primer pair having the sequences of SEQ ID NO: 250 and SEQ ID NO: 251; a primer pair having the sequences of SEQ ID NO: 168 and SEQ ID NO: 295; a primer pair having the sequences of SEQ ID NO: 192 and SEQ ID NO: 300; a primer pair having the sequences of SEQ ID NO: 216 and SEQ ID NO: 217; a primer pair having the sequences of SEQ ID NO: 224 and SEQ ID NO: 305; a primer pair having the sequences of SEQ ID NO: 296 and SEQ ID NO: 297; a primer pair having the sequences of SEQ ID NO: 298 and SEQ ID NO: 299; a primer pair having the sequences of SEQ ID NO: 301 and SEQ ID NO: 302; a primer pair having the sequences of SEQ ID NO: 303 and SEQ ID NO: 304; a primer pair having the sequences of SEQ ID NO: 6 and SEQ ID NO: 306; a primer pair having the sequences of SEQ ID NO: 307 and SEQ ID NO: 308; a primer pair having the sequences of SEQ ID NO: 309 and SEQ ID NO: 300; a primer pair having the sequences of SEQ ID NO: 310 and SEQ ID NO: 311; and a primer pair having the sequences of SEQ ID NO: 312 and SEQ ID NO: 313.

In another embodiment, the disclosure pertains to a set of primers for constructing a genetic profile for a Cannabis plant. The set comprises: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24.

In another embodiment, the disclosure pertains to a set of primers for constructing a genetic profile for a Cannabis plant. The set comprises: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

In another embodiment, the disclosure pertains to a set of primers for constructing a genetic profile for a Cannabis plant. The set comprises a first subset of primer pairs comprising: a primer pair having the sequences of SEQ ID NO: 1 and SEQ ID NO: 2; a primer pair having the sequences of SEQ ID NO: 7 and SEQ ID NO: 8; a primer pair having the sequences of SEQ ID NO: 17 and SEQ ID NO: 18; a primer pair having the sequences of SEQ ID NO: 19 and SEQ ID NO: 20; and a primer pair having the sequences of SEQ ID NO: 23 and SEQ ID NO: 24. The set further comprises a second subset of primer pairs comprising: a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 15 and SEQ ID NO: 16; and a primer pair having the sequences of SEQ ID NO: 21 and SEQ ID NO: 22.

In another embodiment, the disclosure pertains to a set of primers for constructing a genetic profile for a Cannabis plant. The set comprises: a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; and a primer pair having the sequences of SEQ ID NO: 45 and SEQ ID NO: 46.

In another embodiment, the disclosure pertains to a set of primers for constructing a genetic profile for a Cannabis plant. The set comprises: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

In another embodiment, the disclosure pertains to a set of primers for constructing a genetic profile for a Cannabis plant. The set comprises a first subset of primer pairs comprising: a primer pair having the sequences of SEQ ID NO: 7 and SEQ ID NO: 8; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 29 and SEQ ID NO: 30; a primer pair having the sequences of SEQ ID NO: 37 and SEQ ID NO: 38; a primer pair having the sequences of SEQ ID NO: 45 and SEQ ID NO: 46. The set further comprises a second subset of primer pairs comprising: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 13 and SEQ ID NO: 14; a primer pair having the sequences of SEQ ID NO: 39 and SEQ ID NO: 40; and a primer pair having the sequences of SEQ ID NO: 43 and SEQ ID NO: 44.

In another embodiment, the disclosure pertains to a set of primers for constructing a genetic profile for a Cannabis plant. The set comprises: a primer pair having the sequences of SEQ ID NO: 1 and SEQ ID NO: 2; a primer pair having the sequences of SEQ ID NO: 3 and SEQ ID NO: 4; a primer pair having the sequences of SEQ ID NO: 5 and SEQ ID NO: 6; a primer pair having the sequences of SEQ ID NO: 9 and SEQ ID NO: 10; a primer pair having the sequences of SEQ ID NO: 11 and SEQ ID NO: 12; a primer pair having the sequences of SEQ ID NO: 13 and SEQ ID NO: 14; a primer pair having the sequences of SEQ ID NO: 17 and SEQ ID NO: 18; a primer pair having the sequences of SEQ ID NO: 19 and SEQ ID NO: a primer pair having the sequences of SEQ ID NO: 21 and SEQ ID NO: 22; and a primer pair having the sequences of SEQ ID NO: 23 and SEQ ID NO: 24.

In another embodiment, the disclosure pertains to a set of primers for constructing a genetic profile for a Cannabis plant. The set comprises: a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251

In another embodiment, the disclosure pertains to a set of primers for constructing a genetic profile for a Cannabis plant. The set comprises: a primer pair having the sequences of SEQ ID NO: 170 and SEQ ID NO: 171; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences off SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In another embodiment, the disclosure pertains to a set of primers for constructing a genetic profile for a Cannabis plant. The set comprises: a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of .lo9 SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In another embodiment, the disclosure pertains to a set of primers for constructing a genetic profile for a Cannabis plant. The set comprises: a primer pair having the sequences of SEQ ID NO: 170 and SEQ ID NO: 171; a primer pair having the sequences of SEQ ID NO: 174 and SEQ ID NO: 175; a primer pair having the sequences of SEQ ID NO: 180 and SEQ ID NO: 181; a primer pair having the sequences of SEQ ID NO: 182 and SEQ ID NO: 183; a primer pair having the sequences of SEQ ID NO: 188 and SEQ ID NO: 189; a primer pair having the sequences of SEQ ID NO: 210 and SEQ ID NO: 211; a primer pair having the sequences of SEQ ID NO: 212 and SEQ ID NO: 213; a primer pair having the sequences of SEQ ID NO: 218 and SEQ ID NO: 219; a primer pair having the sequences of SEQ ID NO: 220 and SEQ ID NO: 221; a primer pair having the sequences of SEQ ID NO: 224 and SEQ ID NO: 225; a primer pair having the sequences of SEQ ID NO: 228 and SEQ ID NO: 229; a primer pair having the sequences of SEQ ID NO: 244 and SEQ ID NO: 245; a primer pair having the sequences of SEQ ID NO: 248 and SEQ ID NO: 249; and a primer pair having the sequences of SEQ ID NO: 250 and SEQ ID NO: 251.

In another embodiment, the disclosure pertains to a set of primers for constructing a genetic profile for a Cannabis plant. The set comprises: a primer pair having the sequences of SEQ ID NO: 168 and SEQ ID NO: 295; SEQ ID NO: 170 and SEQ ID NO: 171; SEQ ID NO: 174 and SEQ ID NO: 175; SEQ ID NO: 296 and SEQ ID NO: 297; SEQ ID NO: 180 and SEQ ID NO: 181; SEQ ID NO: 298 and SEQ ID NO: 299; SEQ ID NO: 192 and SEQ ID NO: 300; SEQ ID NO: 301 and SEQ ID NO: 302; SEQ ID NO: 303 and SEQ ID NO: 304; SEQ ID NO: 210 and SEQ ID NO: 211; SEQ ID NO: 212 and SEQ ID NO: 213; SEQ ID NO: 214 and SEQ ID NO: 215; SEQ ID NO: 216 and SEQ ID NO: 217; SEQ ID NO: 218 and SEQ ID NO: 219; SEQ ID NO: 220 and SEQ ID NO: 221; SEQ ID NO: 224 and SEQ ID NO: 305; SEQ ID NO: 244 and SEQ ID NO: 245; and SEQ ID NO: 6 and SEQ ID NO: 306.

In another embodiment, the disclosure pertains to a set of primers for constructing a genetic profile for a Cannabis plant. The set comprises: a primer pair having the sequences of SEQ ID NO: 168 and SEQ ID NO: 295; SEQ ID NO: 170 and SEQ ID NO: 171; SEQ ID NO: 312 and SEQ ID NO: 313; SEQ ID NO: 307 and SEQ ID NO: 308; SEQ ID NO: 180 and SEQ ID NO: 181; SEQ ID NO: 298 and SEQ ID NO: 299; SEQ ID NO: 309 and SEQ ID NO: 300; SEQ ID NO: 301 and SEQ ID NO: 302; SEQ ID NO: 303 and SEQ ID NO: 304; SEQ ID NO: 210 and SEQ ID NO: 211; SEQ ID NO: 212 and SEQ ID NO: 213; SEQ ID NO: 214 and SEQ ID NO: 215; SEQ ID NO: 216 and SEQ ID NO: 217; SEQ ID NO: 310 and SEQ ID NO: 311; SEQ ID NO: 220 and SEQ ID NO: 221; SEQ ID NO: 224 and SEQ ID NO: 305; SEQ ID NO: 244 and SEQ ID NO: 245; and SEQ ID NO: 6 and SEQ ID NO: 306.

In various embodiments of the sets of primers described above, at least one of each primer pair comprises sequences comprise a primer tag, a capture tag, a sequencing tag, a unique molecular identifier tag, or a combination thereof.

The disclosure further pertains to kits for use in the methods described above. The kits comprise a set of primers as described above and at least one further component, e.g. a buffer, deoxynucleotide triphosphates (dNTPs), an amplification primer pair, an enzyme, or any combination thereof. The enzyme may be a polymerase or a ligase.

In certain embodiments, the kits are provided to laboratories, whereby a breeder collects biological samples from individual plants and submits the samples to the laboratory for testing using the kits to determine the genotype at one or more polymorphic sites disclosed herein, and to provide the results to the breeder e.g. in the form of a report. Alternatively, the kits include reagents for performing the testing of the samples by the breeders themselves.

The skilled person further understands that the methods disclosed herein could be automated and implemented with the use of computers. The results of a test to determine the identify of an allele at a polymorphic site may be presented as a “report”, that can be generated as part of a testing process, which can be provided to the breeder or any other intended recipient in physical or electronic form. Reports may include the alleles that the individual plant carries at various polymorphic sites.

### Plants Having a Desired Genetic Profile

The skilled person further understands that aspects of this disclosure pertain to novel plants having desired genetic profiles.

In one aspect, the disclosure pertains to a Cannabis plant or plant cell produced according to a method as described herein in the section entitled “Marker-assisted Breeding”.

In various aspects, the disclosure pertains to seed produced by a Cannabis plant or plant cell as described above. In various aspects, the disclosure pertains to a method of producing seeds comprising growing a Cannabis plant as described above, allowing the Cannabis plant to flower, be pollinated, and set seed, and harvesting the seed from the plant.

In various aspects, the disclosure pertains to dried flower from a Cannabis plant as described above, or comprising a Cannabis plant cell as described above.

In various aspects, the disclosure relates to a crop comprising a plurality of Cannabis plants as described above.

In various aspects, the disclosure relates to use of a plant or plant cell as described above for the production of cannabinoids and/or terpenes.

### Plants Having a Genetic Profile of Interest

The skilled person further understands that aspects of this disclosure pertain to novel plants having a particular genetic profile of interest.

In one aspect, the disclosure pertains to a Cannabis plant or plant cell generated according to a method as described above in the section entitled “Marker-assisted Breeding”.

In various aspects, the disclosure pertains to seed produced by a Cannabis plant or plant cell as described above. In various aspects, the disclosure pertains to a method of producing seeds comprising growing a Cannabis plant as described above, allowing the Cannabis plant to flower, be pollinated, and set seed, and harvesting the seed from the plant.

In various aspects, the disclosure pertains to dried flower from a Cannabis plant as described above, or comprising a Cannabis plant cell as described above.

In various aspects, the disclosure relates to a crop comprising a plurality of Cannabis plants as described above.

In various aspects, the disclosure relates to use of a plant or plant cell as described above for the production of cannabinoids and/or terpenes.

### Value Added Products

The skilled person understands that the disclosure further pertains to cannabinoids and/or terpenes derived from a plant or plant cell as described above, or extracted from dried flower as described above. Thus, the disclosure further pertains to methods of producing cannabinoids and/or terpenes comprising extracting cannabinoids and/or terpenes from flowers harvested from plants as described above.

The skilled person further understands that the plants and plant cells described above could further be used to derive extracts, concentrates, isolates, and oils containing cannabinoids and/or terpenes, which may be consumed or optionally used in the production of a variety of food items. The food items may be edible oils. The food items may be snack foods, e.g. candy. The candy may be chocolate, gummies, mints, lozenges, lollipops, or chewing gums. The food items may be baked goods, including, but not limited to, cookies, brownies, cakes, or breads. The food items may be beverages including, but not limited to, soft drinks, energy drinks, teas, coffees, juices, or waters.

The skilled person further understands that the plants and plant cells described above could further be used to derive extracts, concentrates, isolates, and oils containing cannabinoids and/or terpenes, which may be used in the production of cosmetics and/or topicals. The topicals may be massage creams, massage oils, bath oils, body oils, cosmetic oils, oils for toiletry purposes, skin creams, skin lotions, lip care preparations, face and body lotions, bath additives, soaps for personal use, beauty creams for body care, non-medicated skin preparations, or medicated skin preparations.

The disclosure further pertains to extracts, concentrates, isolates, and oils containing cannabinoids and/or terpenes derived from a cannabis plant or plant cell, or dried flower, as described above, and pharmaceutical compositions comprising such extracts, concentrates, isolates, and oils, wherein the extracts, concentrates, isolates, and oils contain cannabinoids and/or terpenes.

The disclosure further pertains to methods of producing oils containing cannabinoids and/or terpenes, the methods comprising crushing seeds produced by a plant described above.

The disclosure further pertains to methods of producing oils, extracts, and concentrates containing cannabinoids and/or terpenes, the methods comprising extracting the oils, extracts, and concentrates from a plant or plant cell, or dried flower, as described above.

The disclosure further pertains to methods of producing isolates comprising a cannabinoid or terpene, the methods comprising isolating the cannabinoid or terpene from a plant or plant cell, or dried flower, as described above.

While specific embodiments of the invention have been described and illustrated, such embodiments should be considered illustrative of the invention only and not as limiting the invention as construed in accordance with the accompanying claims.

All applications and publications referred to herein are incorporated by reference in their entirety.

TABLE 1a
Cannabis SSR Markers, SSR-specific primers, and numbers of associated
alleles, motif, and allele sizes for multiplexes 1-36.

| | | SEQ | | | | | |
|---|---|---|---|---|---|---|---|
| | Primer Name/ | ID | LG | | | Range | |
| Marker | Primer Sequence | NO | (NCBI) | alleles | Motif | (bp) | Reference |
| ANUCS501 | EG220f/ | — | NC_ | 4 | (TTGTG)4 | 78-111 | Gilmore et |
| | AGCAATAATGGAGTGAGTG | | 044370.1 | | | | al, 2003 |
| | AAC | | | | | | |
| ANUCS501 | EG221r/ | 2 | NC_ | 4 | (TTGTG)4 | 78-111 | Gilmore et |
| | AGAGATCAAGAAATTGAGA | | 044370.1 | | | | al, 2003 |
| | TTCC | | | | | | |
| C11- | EG224f/ | 3 | NC_ | 7 | (GAT)8 | 144-177 | Alghanim, |
| CANN1 | GTGGTGGTGATGATGATAA | | 044378.1 | | (GGT)7 | | 2003 |
| | TGG | | | | | | |
| C11- | EG225r/ | 4 | NC_ | 7 | (GAT)8 | 144-177 | Alghanim, |
| CANN1 | TGAATTGGTTACGATGGCG | | 044378.1 | | (GGT)7 | | 2003 |
| E07- | EG226f/ | 5 | NC_ | 4 | (CTA)9 | 108-117 | Alghanim, |
| CANN1 | CAAATGCCACACCACCTTC | | 044374.1 | | | | 2003 |
| E07- | EG227r/ | 6 | NC_ | 4 | (CTA)9 | 108-117 | Alghanim, |
| CANN1 | GTAGGTAGCCAGGTATAG | | 044374.1 | | | | 2003 |
| | GTAG | | | | | | |
| H06- | EG228f/ | 7 | NC_ | 3 | (ACG)7 | 268-277 | Alghanim, |
| CANN2 | TGGTTTCAGTGGTCCTCTC | | 044373.1 | | | | 2003 |
| H06- | EG229r/ | 8 | NC_ | 3 | (ACG)7 | 268-277 | Alghanim, |
| CANN2 | ACGTGAGTGATGACACGA | | 044373.1 | | | | 2003 |
| | G | | | | | | |
| B01- | EG232f/ | 9 | NC_ | 6 | (GAA)13 | 312-339 | Alghanim, |
| CANN1 | TGGAGTCAAATGAAAGGG | | 044373.1 | | (A)(GAA)3 | | 2003 |
| | AAC | | | | | | |
| B01- | EG233r/ | 10 | NC_ | 6 | (GAA)13 | 312-339 | Alghanim, |
| CANN1 | CCATAGCATTATCCCACTC | | 044373.1 | | (A)(GAA)3 | | 2003 |
| | AAG | | | | | | |
| D02- | EG238f/ | 11 | NC_ | 4 | (GTT)7 | 110-119 | Alghanim, |
| CANN1 | GGTTGGGATGTTGTTGTTG | | 044375.1 | | | | 2003 |
| | TG | | | | | | |
| D02- | EG239r/ | 12 | NC_ | 4 | (GTT)7 | 110-119 | Alghanim, |
| CANN1 | AGAAATCCAAGGTCCTGAT | | 044375.1 | | | | 2003 |
| | GG | | | | | | |
| CAN0055 | EG266f/ | 13 | NC_ | 2 | (AATAA)4 | 273-282 | Gao, 2014 |
| | CACCTTCTGGCTCTTATGG | | 044379.1 | | | | |
| | T | | | | | | |
| CAN0055 | EG267r/ | 14 | NC_ | 2 | (AATAA)4 | 273-282 | Gao, 2014 |
| | CTGTTACTCGAATTGGGTC | | 044379.1 | | | | |
| | C | | | | | | |
| CAN0111 | EG300f/ | 15 | NC_ | 2 | (ATC)6 | 295-305 | Gao, 2014 |
| | AAGAGCTGGAACGAACAA | | 044370.1 | | | | |
| | AG | | | | | | |
| CAN0111 | EG301r/ | 16 | NC_ | 2 | (ATC)6 | 295-305 | Gao, 2014 |
| | CTGGTGGGATCAGAACTT | | 044370.1 | | | | |
| | GT | | | | | | |
| CAN0509 | EG306f/ | 17 | NC_ | 6 | (CAG)6 | 292-309 | Gao, 2014 |
| | CAGCAGAATCAGTCGTCAT | | 044371.1 | | | | |
| | C | | | | | | |
| CAN0509 | EG307r/ | 18 | NC_ | 6 | (CAG)6 | 292-309 | Gao, 2014 |
| | TAACTGTTGCTGTTGCAGG | | 044371.1 | | | | |
| | T | | | | | | |
| CAN0717 | EG316f/ | 19 | NC_ | 4 | (TCT)6 | 301-310 | Gao, 2014 |
| | ACGCACACAGGTGTATTCT | | 044372.1 | | | | |
| | G | | | | | | |
| CAN0717 | EG317r/ | 20 | NC_ | 4 | (TCT)6 | 301-310 | Gao, 2014 |
| | GCTCATGTTTTTGAGGGAC | | 044372.1 | | | | |
| | T | | | | | | |
| CAN2550 | EG340f/ | 21 | NC_ | 6 | (CTCAAA)3 | 301-325 | Gao, 2014 |
| | GAGCCCATTTCAGTTTCAG | | 044376.1 | | | | |
| | T | | | | | | |
| CAN2550 | EG341r/ | 22 | NC_ | 6 | (CTCAAA)3 | 301-325 | Gao, 2014 |
| | GATTAGGCCTGAAAGTGGT | | 044376.1 | | | | |
| | G | | | | | | |
| CAN0009 | EG352f/ | 23 | NC_ | 10 | (TCT)6 | 116-145 | Gao, 2014 |
| | CTCCAAACCCAACAACACA | | 044377.1 | | | | |
| CAN0009 | EG353r/ | 24 | NC_ | 10 | (TCT)6 | 116-145 | Gao, 2014 |
| | TCAAGCCATGAGAGTTGAG | | 044377.1 | | | | |
| | A | | | | | | |
| ANUCS301 | EG210f/ | 25 | NC_ | 4 | (TTA)15 | | Gilmore et |
| | ATATGGTTGAAATCCATTG | | 044370.1 | | | | al, 2003 |
| | C | | | | | | |
| ANUCS301 | EG211r/ | 26 | NC_ | 4 | (TTA)15 | | Gilmore et |
| | TAACAAAGTTTCGTGAGGG | | 044370.1 | | | | al, 2003 |
| | T | | | | | | |
| ANUCS303 | EG214f/ | 27 | NC_044 | 4 | (CAA)4 | | Gilmore et |
| | TAATCAACAATGACAATGG | | 374.1 | | catcagtagca | | al, 2003 |
| | C | | | | (TGT)7 | | |
| ANUCS303 | EG215r/ | 28 | NC_ | 4 | (CAA)4 | | Gilmore et |
| | GATTAAGGTCCTCGACGAT | | 044374.1 | | catcagtagca | | al, 2003 |
| | A | | | | (TGT)7 | | |
| ANUCS304 | EG216f/ | 29 | NC_ | 4 | (TCT)8 | 143-210 | Gilmore et |
| | TCTTCACTCACCTCCTCTC | | 044379.1 | | TCA(TCT)7 | | al, 2003 |
| | T | | | | | | |
| ANUCS304 | EG217r/ | 30 | NC_ | 4 | (TCT)8 | 143-210 | Gilmore et |
| | TCTTTAAGCGGGACTCGT | | 044379.1 | | TCA(TCT)7 | | al, 2003 |
| B02- | EG234f/ | 31 | | 4 | (AAG)10 | | Alghanim, |
| CANN2 | CAACCAAATGAGAATGCAA | | | | | | 2003 |
| | CC | | | | | | |
| B02- | EG235r/ | 32 | | 4 | (AAG)10 | | Alghanim, |
| CANN2 | TGTTTTCTTCACTGCACCC | | | | | | 2003 |
| B05- | EG222f/ | 33 | | | (TTG)9 | | Alghanim, |
| CANN1 | TTGATGGTGGTGAAACGG | | | | | | 2003 |
| | C | | | | | | |
| B05- | EG223r/ | 34 | | | (TTG)9 | | Alghanim, |
| CANN1 | CCCCAATCTCAATCTCAAC | | | | | | 2003 |
| | CC | | | | | | |
| CAN0126 | EG252f/ | 35 | NC_ | | (AATACC)3 | | Gao, 2014 |
| | GAGTAAGAGAAGGCGAAC | | 044378.1 | | (CAG)6 | | |
| | CA | | | | | | |
| CAN0126 | EG253r/ | 36 | NC_ | | (AATACC)3 | | Gao, 2014 |
| | CCTGTGTAACAGAAAACCC | | 044378.1 | | (CAG)6 | | |
| | C | | | | | | |
| CAN1347 | EG256f/ | 37 | NC_ | | (AAC)6 | | Gao, 2014 |
| | TGTTTCTAAGGCTCAGTCC | | 044378.1 | | (ATC)7 | | |
| | C | | | | | | |
| CAN1347 | EG257r/ | 38 | NC_ | | (AAC)6 | | Gao, 2014 |
| | GGCAAAGGTAAAGCAAGT | | 304478.1 | | (ATC)7 | | |
| | GT | | | | | | |
| CAN1482 | EG288f/ | 39 | NC_ | | (CTT)7 | | Gao, 2014 |
| | TTCCCCAACTATCCAATCA | | 044370.1 | | | | |
| | C | | | | | | |
| CAN1482 | EG289r/ | 40 | NC_ | | (CTT)7 | | Gao, 2014 |
| | ATTTGAGAAGGGGTTAGG | | 044370.1 | | | | |
| | CT | | | | | | |
| CAN2913 | EG260f/ | 41 | NC_ | | (AAG)7 | | Gao, 2014 |
| | AGGAACACTTTGAAAGCGA | | 044373.1 | | | | |
| | G | | | | | | |
| CAN2913 | EG261r/ | 42 | NC_ | | (AAG)7 | | Gao, 2014 |
| | CGGTCATCTACCTTGAGCT | | 044373.1 | | | | |
| | T | | | | | | |
| NM101 | EG292f/ | 43 | NC_ | | CACCAT | | Hseih, |
| | AAGCAACTCCAATTCCAGC | | 044375.1 | | | | 2003 |
| | C | | | | | | |
| NM101 | EG293r/ | 44 | NC_ | | CACCAT | | Hseih, |
| | TAATGATGAGACGAGTGAG | | 044375.1 | | | | 2003 |
| | AACG | | | | | | |
| ANUCS305 | EG218f/ | 45 | NC_ | 3 | (TGG)10 | | Gilmore et |
| | AAAGTTGGTCTGAGAAGCA | | 044375.1 | | | | al, 2003 |
| | AT | | | | | | |
| ANUCS305 | EG219r/ | 46 | NC_ | 3 | (TGG)10 | | Gilmore et |
| | CCTAGGAACTTTCGACAAC | | 044375.1 | | | | al, 2003 |
| | A | | | | | | |
| CAN0010 | EG354f/ | 47 | NC_ | | (GTG)6 | | Gao, 2014 |
| | TCCAAACGTTCTCTCTCTC | | 044377.1 | | | | |
| | C | | | | | | |
| CAN0010 | EG355r/ | 48 | NC_ | | (GTG)6 | | Gao, 2014 |
| | CTACTAACCCAATCAGACC | | 044377.1 | | | | |
| | CA | | | | | | |
| CAN0878 | EG320f/ | 49 | NC_ | | (ATA)6 | | Gao, 2014 |
| | GATGAACATGAAACAGCAG | | 044371.1 | | | | |
| | C | | | | | | |
| CAN0878 | EG321r/ | 50 | NC_ | | (ATA)6 | | Gao, 2014 |
| | ATATGAGCCATGACCGAAG | | 044371.1 | | | | |
| | T | | | | | | |
| CAN2717 | EG344f/ | 51 | NC_ | | (GGT)6 | | Gao, 2014 |
| | TGATGACAGAGTGATAGCA | | 044372.1 | | | | |
| | GC | | | | | | |
| CAN2717 | EG345r/ | 52 | NC_ | | (GGT)6 | | Gao, 2014 |
| | CTAATCAATGCGGTAAACC | | 044372.1 | | | | |
| | C | | | | | | |
| ANUCS201 | EG208f/ | 53 | NC_ | | (GA)26 | | Gilmore et |
| | GGTTCAATGGAGATTCTCG | | 044371 | | | | al, 2003 |
| | T | | | | | | |
| ANUCS201 | EG209r/ | 54 | NC_ | | (GA)26 | | Gilmore et |
| | CCACTAAACCAAAAGTACT | | 044371 | | | | al, 2003 |
| | CTTC | | | | | | |
| ANUCS203 | EG242f/ | 55 | | | (CT)50 | | Gilmore et |
| | GCTCTTCTTATTAATTCCTC | | | | | | al, 2003 |
| | CTT | | | | | | |
| ANUCS203 | EG243r/ | 56 | | | (CT)50 | | Gilmore et |
| | GAATATGATAAGACACAAC | | | | | | al, 2003 |
| | TTCATT | | | | | | |
| ANUCS205 | EG244f/ | 57 | | | (CT)21 | | Gilmore et |
| | TTGACTAACCGGCAAAGAT | | | | | | al, 2003 |
| | A | | | | | | |
| ANUCS205 | EG245r/ | 58 | | | (CT)21 | | Gilmore et |
| | AAATTCAAAACCGATTCTC | | | | | | al, 2003 |
| | AG | | | | | | |
| C08- | EG236f/ | 59 | | | (GA)21 | | Alghanim, |
| CANN2 | GCAAGAAGTAGAGAGACA | | | | | | 2003 |
| | ATCC | | | | | | |
| C08- | EG237r/ | 60 | | | (GA)21 | | Alghanim, |
| CANN2 | CCCTCAACACTCATTAACT | | | | | | 2003 |
| | CAC | | | | | | |
| ANUCS302 | EG212f/ | 61 | | | (CAA)7- | | Gilmore et |
| | AACATAAACACCAACAACT | | | | (CAA)4 | | al, 2003 |
| | GC | | | | | | |
| ANUCS302 | EG213r/ | 62 | | | (CAA)7- | | Gilmore et |
| | ATGGTTGATGTTTTGATGG | | | | (CAA)4 | | al, 2003 |
| | T | | | | | | |
| CAN0024 | EG262f/ | 63 | | | (AGA)6 | | Gao, 2014 |
| | GATACTGGCTCACGGATCT | | | | | | |
| | T | | | | | | |
| CAN0024 | EG263r/ | 64 | | | (AGA)6 | | Gao, 2014 |
| | CGCCTCTTCTTAACCTGTG | | | | | | |
| | T | | | | | | |
| CAN0026 | EG264f/ | 65 | NC_ | | (CAA)6 | | Gao, 2014 |
| | AGGAGCAGAACAAAATGG | | 044378.1 | | | | |
| | AG | | | | | | |
| CAN0026 | EG265r/ | 66 | NC_ | | (CAA)6 | | Gao, 2014 |
| | TAAACCGGGTCAACCATAC | | 044378.1 | | | | |
| | T | | | | | | |
| CAN0039 | EG246f/ | 67 | NC_ | | (CAT)8 | | Gao, 2014 |
| | GCAGCCATAGTCATGGTGT | | 044379.1 | | | | |
| | A | | | | | | |
| CAN0039 | EG247r/ | 68 | NC_ | | (CAT)8 | | Gao, 2014 |
| | GTCATTGGAAAGACCAGCT | | 044379.1 | | | | |
| | T | | | | | | |
| CAN0093 | EG248f/ | 69 | | | (GA)11 | | Gao, 2014 |
| | CAGTCTCTCAGATCAGACT | | | | | | |
| | ACC | | | | | | |
| CAN0093 | EG249r/ | 70 | | | (GA)11 | | Gao, 2014 |
| | AGCGGCTAGCGTAACAGT | | | | | | |
| | AT | | | | | | |
| CAN0107A | EG268f/ | 71 | | | (AGAAGGAGG) | | Gao, 2014 |
| | AAGTGGCGAACTGGTTTAT | | | | 2(AGG)6 | | |
| | C | | | | | | |
| CAN0107A | EG269r/ | 72 | | | (AGAAGGAGG) | | Gao, 2014 |
| | AAGCCAGCGTTATTATCGA | | | | 2(AGG)6 | | |
| | C | | | | | | |
| CAN0110 | EG250f/ | 73 | | | (AT)10 | | Gao, 2014 |
| | GGGTAAAGCTTACGCAAA | | | | | | |
| | GT | | | | | | |
| CAN0110 | EG251r/ | 74 | | | (AT)10 | | Gao, 2014 |
| | AACAAACAGTTGGACACCC | | | | | | |
| | T | | | | | | |
| CAN0585 | EG254f/ | 75 | NC_ | | (ACTTCTATT) | | Gao, 2014 |
| | TCATCATCATCCCTCCCTA | | 044374.1 | | 2t(CAAAAC)3 | | |
| | T | | | | | | |
| CAN0585 | EG255r/ | 76 | NC_ | | (ACTTCTATT) | | Gao, 2014 |
| | GGTCCATAGTTGGCTGATC | | 044374.1 | | 2t(CAAAAC)3 | | |
| | T | | | | | | |
| CAN0663 | EG272f/ | 77 | NC_ | | (TGG)8 | | Gao, 2014 |
| | CATCACAGAGGACCCTTTT | | 044371.1 | | | | |
| | C | | | | | | |
| CAN0663 | EG273r/ | 78 | NC_ | | (TGG)8 | | Gao, 2014 |
| | GTCTCCACCACCAATCTTT | | 044371.1 | | | | |
| | C | | | | | | |
| CAN0672 | EG284f/ | 79 | NC_ | | (ATA)7 | | Gao, 2014 |
| | TGCAGCACCAACTTACTTT | | 044378 | | | | |
| | C | | | | | | |
| CAN0672 | EG285r/ | 80 | NC_ | | (ATA)7 | | Gao, 2014 |
| | GCATTGTATTTCCCTTCCA | | 044378 | | | | |
| | C | | | | | | |
| CAN0865 | EG270f/ | 81 | | | (TCTCTCTTC) | | Gao, 2014 |
| | CTTCTCTAATCCGATTCCC | | | | 2(TC)9* | | |
| | A | | | | | | |
| CAN0865 | EG271r/ | 82 | | | (TCTCTCTTC) | | Gao, 2014 |
| | GAACGAAGAAAACCCAGA | | | | 2(TC)9* | | |
| | AG | | | | | | |
| CAN1001 | EG274f/ | 83 | | | (AAAG)5 | | Gao, 2014 |
| | CTAGTTACGGGGGAATTCA | | | | | | |
| | A | | | | | | |
| CAN1001 | EG275r/ | 84 | | | (AAAG)5 | | Gao, 2014 |
| | CTTTCCCAACTCGTATTTG | | | | | | |
| | C | | | | | | |
| CAN1423 | EG286f/ | 85 | | | (GAA)7 | | Gao, 2014 |
| | CTCCACAATCTCCAATCTC | | | | | | |
| | C | | | | | | |
| CAN1423 | EG287r/ | 86 | | | (GAA)7 | | Gao, 2014 |
| | GCTACGGTAACGGAATCAA | | | | | | |
| | C | | | | | | |
| CAN1690B | EG258f/ | 87 | NC_ | | (CAA)6(CATCA | | Gao, 2014 |
| | CAAACAGGGGAAAAGAGA | | 044374.1 | | TAAT)2 | | |
| | GA | | | | | | |
| CAN1690B | EG259r/ | 88 | NC_ | | (CAA)6(CATCA | | Gao, 2014 |
| | ATGAAGCGTTGGTACTAGG | | 044374.1 | | TAAT)2 | | |
| | C | | | | | | |
| CAN2167 | EG276f/ | 89 | NC_ | | (AGA)8 | | Gao, 2014 |
| | CAAATGGGTTCCTCTCAAT | | 044379.1 | | | | |
| | C | | | | | | |
| CAN2167 | EG277r/ | 90 | NC_ | | (AGA)8 | | Gao, 2014 |
| | ACGAGGCGTACCTAATGAA | | 044379.1 | | | | |
| | G | | | | | | |
| CAN2707 | EG278f/ | 91 | NC_ | | (TCAACC)5 | | Gao, 2014 |
| | TAATCAGTTGTACGGAACG | | 044370.1 | | | | |
| | C | | | | | | |
| CAN2707 | EG279r/ | 92 | NC_ | | (TCAACC)5 | | Gao, 2014 |
| | TGACTCTGTGAATCAGTGG | | 044370.1 | | | | |
| | G | | | | | | |
| CAN3023 | EG280f/ | 93 | NC_ | | (CAACAG)3 | | Gao, 2014 |
| | CCTCTACTGGGACACACAC | | 044377 | | | | |
| | A | | | | | | |
| CAN3023 | EG281r/ | 94 | NC_ | | (CAACAG)3 | | Gao, 2014 |
| | CCTTCTTCTTAACAGGGCA | | 044377 | | | | |
| | A | | | | | | |
| CAN3037 | EG282f/ | 95 | NC_ | | (ATC)9 | | Gao, 2014 |
| | CGAACTCTTTTAGCATTGC | | 304472.1 | | | | |
| | C | | | | | | |
| CAN3037 | EG283r/ | 96 | NC_ | | (ATC)9 | | Gao, 2014 |
| | CGGGGGATTAAAAGAGAA | | 044372.1 | | | | |
| | GA | | | | | | |
| CAN3087 | EG290f/ | 97 | | | (CACCGG)3 | | Gao, 2014 |
| | ACCGAGAAAACCAACTCAT | | | | | | |
| | C | | | | | | |
| CAN3087 | EG291r/ | 98 | | | (CACCGG)3 | | Gao, 2014 |
| | TCTCGTTTCGGTCAGATGT | | | | | | |
| | A | | | | | | |
| H09- | EG230f/ | 99 | | | (GA)15 | | Alghanim, |
| CANN2 | CGTACAGTGATCGTAGTTG | | | | | | 2003 |
| | AG | | | | | | |
| H09- | EG231r/ | 100 | | | (GA)15 | | Alghanim, |
| CANN2 | ACACATACAGAGAGAGCC | | | | | | 2003 |
| | C | | | | | | |
| CAN0006 | EG294f/ | 124 | NC_ | 2 | (TCT)7 | 298- | Gao, 2014 |
| | AGCAAGAGGGTAAGAGGG | | 044377.1 | | | | |
| | AT | | | | | | |
| CAN0006 | EG295r/ | 125 | NC_ | 2 | (TCT)7 | 298- | Gao, 2014 |
| | TCATCATCATCCTCATCAG | | 044377.1 | | | | |
| | C | | | | | | |
| CAN0051 | EG356f/ | 126 | NC_ | 3 | (TCA)6 | 297-308 | Gao, 2014 |
| | AACCCAAAAGAGCTGAGA | | 044377.1 | | | | |
| | GA | | | | | | |
| CAN0051 | EG357r/ | 127 | NC_ | 3 | (TCA)6 | 297-308 | Gao, 2014 |
| | CTCAGCAAGGTGAGTACA | | 044377.1 | | | | |
| | CG | | | | | | |
| CAN0061 | EG298f/ | 128 | NC_ | 2 | (AG)14 | 299- | Gao, 2014 |
| | GGGGTGAGAGAAAGTGAG | | 044371.1 | | | | |
| | AA | | | | | | |
| CAN0061 | EG299r/ | 129 | NC_ | 2 | (AG)14 | 299- | Gao, 2014 |
| | CTCTGCACACTCCTGATTT | | 044371.1 | | | | |
| | G | | | | | | |
| CAN0118 | EG302f/ | 130 | NC_ | | (AACTCA)3 | 300- | Gao, 2014 |
| | GGGTGGTTTTGTGAAGAG | | 044374.1 | | | | |
| | AT | | | | | | |
| CAN0118 | EG303r/ | 131 | NC_ | | (AACTCA)3 | 300- | Gao, 2014 |
| | GGAGAAGCATTGTTCGTTC | | 044374.1 | | | | |
| | T | | | | | | |
| CAN0189 | EG304f/ | 132 | NC_ | | (ATA)7 | 300- | Gao, 2014 |
| | CAGAGCTCAAAGAAAGGG | | 044376.1 | | | | |
| | AA | | | | | | |
| CAN0189 | EG305r/ | 133 | NC_ | | (ATA)7 | 300- | Gao, 2014 |
| | AATAGCTGGCTCATCAGGT | | 044376.1 | | | | |
| | C | | | | | | |
| CAN0590 | EG308f/ | 134 | NC_ | | (AAT)6 | 300- | Gao, 2014 |
| | AGGGAGAGAGAGAATGTC | | 044379.1 | | | | |
| | CC | | | | | | |
| CAN0590 | EG309r/ | 135 | NC_ | | (AAT)6 | 300- | Gao, 2014 |
| | TCTTCTGCACCACAGGTAG | | 304479.1 | | | | |
| | A | | | | | | |
| CAN0716 | EG314f/ | 136 | NC_ | 2 | (ATGGGG)3 | 283-296 | Gao, 2014 |
| | TCACCTCAAATGGTGATGT | | 044372.1 | | (ATGGGA)5 | | |
| | C | | | | | | |
| CAN0716 | EG315r/ | 137 | NC_ | 2 | (ATGGGG)3 | 283-296 | Gao, 2014 |
| | GAAACCGAAGAAGAAGTG | | 044372.1 | | (ATGGGA)5 | | |
| | GA | | | | | | |
| CAN0795 | EG318f/ | 138 | NC_ | | (AAAGAG)3 | 283-296 | Gao, 2014 |
| | AGAGGAGTGTTGATGAAG | | 044379.1 | | | | |
| | CC | | | | | | |
| CAN0795 | EG319r/ | 139 | NC_ | | (AAAGAG)3 | 283-296 | Gao, 2014 |
| | CATCTGAGCTGGATTTCAC | | 044379.1 | | | | |
| | A | | | | | | |
| CAN1455 | EG326f/ | 140 | NC_ | | (AAG)7 | 304- | Gao, 2014 |
| | AAGTCCAACCACCTCACAA | | 044373.1 | | | | |
| | T | | | | | | |
| CAN1455 | EG327r/ | 141 | NC_ | | (AAG)7 | 304- | Gao, 2014 |
| | CTTAACCAATCCCCAAGAT | | 044373.1 | | | | |
| | G | | | | | | |
| CAN1481 | EG328f/ | 142 | NC_ | | (TTC)6 | 279-307 | Gao, 2014 |
| | TTCCCCAACTATCCAATCA | | 044370.1 | | | | |
| | C | | | | | | |
| CAN1481 | EG329r/ | 143 | NC_ | | (TTC)6 | 279-307 | Gao, 2014 |
| | ATTTGAGAAGGGGTTAGG | | 044370.1 | | | | |
| | CT | | | | | | |
| CAN1832 | EG334f/ | 144 | NC_ | | (AGGAGA)3 | 304- | Gao, 2014 |
| | GTTAGAATCCCATTTGGCT | | 044371.1 | | | | |
| | G | | | | | | |
| CAN1832 | EG335r/ | 145 | NC_ | | (AGGAGA)3 | 304- | Gao, 2014 |
| | GTTTCGAAAGTTGAAGGCT | | 044371.1 | | | | |
| | C | | | | | | |
| CAN2166 | EG336f/ | 146 | NC_ | | (AAC)6 | 300- | Gao, 2014 |
| | CAAATGGGTTCCTCTCAAT | | 044379.1 | | | | |
| | C | | | | | | |
| CAN2166 | EG337r/ | 147 | NC_ | | (AAC)6 | 300- | Gao, 2014 |
| | ACGAGGCGTACCTAATGAA | | 044379.1 | | | | |
| | G | | | | | | |
| CAN3105 | EG348f/ | 148 | NC_ | | (TTC)6 | 305- | Gao, 2014 |
| | CTTCATCATCAAACCACTG | | 044373.1 | | | | |
| | C | | | | | | |
| CAN3105 | EG349r/ | 149 | NC_ | | (TTC)6 | 305- | Gao, 2014 |
| | TGTCCATATGAGGTCACTG | | 044373.1 | | | | |
| | C | | | | | | |
| CAN3321 | EG350f/ | 150 | NC_ | | (TCA)6 | 300- | Gao, 2014 |
| | AGAGTACGGCCTAGTTAC | | 044379.1 | | | | |
| | GG | | | | | | |
| CAN3321 | EG351r/ | 151 | NC_ | | (TCA)6 | 300- | Gao, 2014 |
| | ATAGGTGAAAACTTGGCCT | | 044379.1 | | | | |
| | G | | | | | | |

TABLE 1b
Cannabis SSR Markers, SSR-specific primers, and numbers of associated
alleles, motif, and allele sizes for multiplexes 39-91. All primers were designed in-house.

| | | | SEQ | | | | |
|---|---|---|---|---|---|---|---|
| | Primer | | ID | LG | # | | Range |
| Marker | Name | Sequence | NO | (NCBI) | alleles | Motif | (bp) |
| ANUCS304ana | EG496f | TGAGAAGATGAAAA | 166 | NC_ | 4 | (TCT)8TCA | 345-423 |
| | | CGTGTGAC | | 044379 | | (TCT)7 | |
| ANUCS304ana | EG497r | AAAGAAGGACCTCC | 167 | NC_ | 4 | (TCT)8TCA | 345-423 |
| | | CATATCC | | 044379 | | (TCT)7 | |
| ANUCS501ana | EG426f | GTGAGTAGCAATAA | 168 | NC_ | 4 | (TTGTG)4 | 83-103 |
| | | TGGAGTGA | | 044370 | | | |
| ANUCS501ana | EG425r | CAAGAAATTGAGATT | 169 | NC_ | 4 | (TTGTG)4 | 83-103 |
| | | CCCAATGG | | 044370 | | | |
| C11- | EG429f | TATGATTCTGGTGAT | 170 | NC_ | 7 | (GAT)8(GGT) | 187-211 |
| CANN1ana2 | | GGCGG | | 044378 | | 7 | |
| C11- | EG537r | ATGGGCGTCCTTGA | 171 | NC_0443 | 7 | (GAT)8(GGT) | 187-211 |
| CANN1ana2 | | ATTGGTTACG | | 78 | | 7 | |
| CAN0009ana2 | EG409f | TCTAAGCTCATCTGA | 172 | NC_ | 6 | (TCT)6 | 111-140 |
| | | AATTCTC | | 044377 | | | |
| CAN0009ana2 | EG467r | TCCGAATAGAAAAA | 173 | NC_ | 6 | (TCT)6 | 111-140 |
| | | AAAAATTGGG | | 044377 | | | |
| CAN0055ana4 | EG430f | TACTGGGTAATAAG | 174 | NC_ | 4 | (AATAA)4 | 110-115 |
| | | ATTCCACC | | 044379 | | | |
| CAN0055ana4 | EG538r | CTTAATCTAACCTCT | 175 | NC_ | 4 | (AATAA)4 | 110-115 |
| | | AACGACCGC | | 044379 | | | |
| CAN0057ana | EG543f | GACTTGTTTGTCAG | 176 | NC_ | 7 | (CAA)7 | 391-415 |
| | | ATATTTGACTGAC | | 044374 | | | |
| CAN0057ana | EG544r | GAATTTTTGTTGCAT | 177 | NC_ | 7 | (CAA)7 | 391-415 |
| | | TTAACTCACTTTAG | | 044374 | | | |
| CAN0075ana | EG490f | AAAGCTCTGTACGA | 178 | NC_ | 7 | (TCT)7 | 418-421 |
| | | CATGTCG | | 044379 | | | |
| CAN0075ana | EG491r | TTCTTTTCTGGGGCT | 179 | NC_ | 7 | (TCT)7 | 418-421 |
| | | CTCTC | | 044379 | | | |
| CAN0089ana | EG517f | CTAAAAACATTACAG | 180 | NC_ | 5 | (AAAGA)5 | 299-313 |
| | | CTGTGAGGC | | 044376 | | | |
| CAN0089ana | EG518r | CGCCAACATTTACT | 181 | NC_ | 5 | (AAAGA)5 | 299-313 |
| | | CACTAAACTC | | 044376 | | | |
| CAN0103ana | EG523f | CAAAAACACTCTAG | 182 | NC_ | 7 | (TTC)7 | 230-251 |
| | | ACTCCTCC | | 044372 | | | |
| CAN0103ana | EG524r | GGAGCTTCTTTTTGA | 183 | NC_ | 7 | (TTC)7 | 230-251 |
| | | GAGTCTG | | 044372 | | | |
| CAN0124ana | EG509f | CTTACAAAAAGGAC | 184 | NC_ | 7 | (AATAACAAC)2 | 160-193 |
| | | ACTAGCCAC | | 044371 | | (AAT)7 | |
| CAN0124ana | EG510r | ATAAACACAAGGAA | 185 | NC_ | 7 | (AATAACAAC)2 | 160-193 |
| | | GTTGATGATGAAG | | 044371 | | (AAT)7 | |
| CAN0138ana2 | EG498f | GCCAAACAGAAATG | 186 | NC_ | 5 | (ACCAG)5 | 180-200 |
| | | ACTGGTG | | 044375 | | | |
| CAN0138ana2 | EG471r | ATAATAAGAAATTAA | 187 | NC_ | 5 | (ACCAG)5 | 180-200 |
| | | TGGTGTTGTTG | | 044375 | | | |
| CAN0156ana | EG513f | GAGTCTTTGCAGCA | 188 | NC_ | 5 | (CCTT)5 | 424-444 |
| | | AACACACC | | 044370 | | | |
| CAN0156ana | EG514r | TTGCTAAATAACTCA | 189 | NC_ | 5 | (CCTT)5 | 424-444 |
| | | GTACAAAACAGA | | 044370 | | | |
| CAN0180ana | EG486f | CTTTTTCAAACTCTC | 190 | NC_ | 5 | (AAAT)5 | 290-306 |
| | | AATCCTTGC | | 044376 | | | |
| CAN0180ana | EG487r | AGACGATAGGACTA | 191 | NC_ | 5 | (AAAT)5 | 290-306 |
| | | AAATCTAATC | | 044376 | | | |
| CAN0202ana | EG476f | ATCGGGAAGACACA | 192 | NC_ | 7 | (TTC)7 | 456-474 |
| | | TGCTTC | | 044377 | | | |
| CAN0202ana | EG477r | ACTACCTACAACGG | 193 | NC_ | 7 | (TTC)7 | 456-474 |
| | | TTCCTG | | 044377 | | | |
| CAN0244ana | EG480f | GGAACAGCAAAATC | 194 | NC_ | 4 | (AAACCT)4 | 269-273 |
| | | CAATACCC | | 044378 | | | |
| CAN0244ana | EG481r | AACTCAGTTCAAGG | 195 | NC_ | 4 | (AAACCT)4 | 269-273 |
| | | AAAAAGGG | | 044378 | | | |
| CAN0271ana | EG472f | AAGAGGCAAGAGAG | 196 | NC_ | 4 | (TCTTT)4 | 180-280 |
| | | GGTGAG | | 044375 | | | |
| CAN0271ana | EG473r | ACGACGATGATGAT | 197 | NC_ | 4 | (TCTTT)4 | 180-280 |
| | | GATGGC | | 044375 | | | |
| CAN0278ana | EG492f | CGAAACACAGATGA | 198 | NC_ | 8 | (ATC)8 | 369-372 |
| | | ATTCTTATTC | | 044379 | | | |
| CAN0278ana | EG493r | AAGTGGGCAACGAC | 199 | NC_ | 8 | (ATC)8 | 369-372 |
| | | TTGTGG | | 044379 | | | |
| CAN0291ana | EG482f | ATCTGAGTTTTGGAT | 200 | NC_ | 7 | (AGA)7 | 329-345 |
| | | TTGGAGTG | | 044378 | | | |
| CAN0291ana | EG483r | AGAAGACCGGTGGT | 201 | NC_ | 7 | (AGA)7 | 329-345 |
| | | TACCTG | | 044378 | | | |
| CAN0298ana | EG484f | CAAAATATTTCTATA | 202 | NC_ | 9 | (CTA)9 | 170-179 |
| | | ACGGGCAGC | | 044378 | | | |
| CAN0298ana | EG485r | AAACCCTCATTAGAT | 203 | NC_ | 9 | (CTA)9 | 170-179 |
| | | GTGTTCAC | | 044378 | | | |
| CAN0308ana | EG488f | TAGCTTTACTCCTAC | 204 | NC_ | 7 | (TCT)7 | 339-342 |
| | | TCAAACC | | 044378 | | | |
| CAN0308ana | EG489r | TTCTTGAACATGTCT | 205 | NC_ | 7 | (TCT)7 | 339-342 |
| | | TGGCAC | | 044378 | | | |
| CAN0338- | EG503f | ACTTCTACTACTCAA | 206 | NC_ | 6 | (CACTAC)3... | 451-478 |
| 339ana | | CAATCCAAC | | 044374 | | (TCA)6 | |
| CAN0338- | EG504r | TCAATGGTGGCAAG | 207 | NC_ | 6 | (CACTAC)3... | 451-478 |
| 339ana | | TAGCAAC | | 044374 | | (TCA)6 | |
| CAN0442ana | EG478f | TCAAAATAGGAAAC | 208 | NC_ | 6 | (AAAT)6 | 137-143 |
| | | ACCAGAACC | | 044377 | | | |
| CAN0442ana | EG479r | TTGTTCTTCTCTGAG | 209 | NC_ | 6 | (AAAT)6 | 137-143 |
| | | CTGGG | | 044377 | | | |
| CAN0509ana | EG432f | TCAGTCGTCATCTAT | 210 | NC_ | 6 | (CAG)6 | 302-322 |
| | | TCAACAG | | 044371 | | | |
| CAN0509ana | EG431r | TTGAAACGTTATTTT | 211 | NC_ | 6 | (CAG)6 | 302-322 |
| | | GCGGAGC | | 044371 | | | |
| CAN0577ana | EG550f | CCTCCTTATTGAGA | 212 | NC_ | 6 | (CAAC)6 | 474-490 |
| | | CCAACCAGTC | | 044374 | | | |
| CAN0577ana | EG551r | CTCTTTTGTTTGTTC | 213 | NC_ | 6 | (CAAC)6 | 474-490 |
| | | TGTTGGACTTG | | 044374 | | | |
| CAN0716ana | EG499f | TCGATGGCACCATA | 214 | NC_ | 2 | (ATGGGG)3 | 232-264 |
| | | AACGGC | | 044372 | | (ATGGGA)5 | |
| CAN0716ana | EG500r | AAGTGATGAGCAGT | 215 | NC_ | 2 | (ATGGGG)3 | 232-264 |
| | | TTGAGATAC | | 044372 | | (ATGGGA)5 | |
| CAN0717ana | EG433f | GAGTCTTTTCTTCCG | 216 | NC_ | 6 | (CAC)6 | 243-252 |
| | | GAAATTC | | 044372 | | | |
| CAN0717ana | EG424r | GTCCACTATACAAAA | 217 | NC_ | 6 | (CAC)6 | 243-252 |
| | | ACTGAAGC | | 044372 | | | |
| CAN0802ana | EG521f | GATTATGGGGCTGC | 218 | NC_ | 9 | (TTC)9 | 324-353 |
| | | TTCAAAAC | | 044378 | | | |
| CAN0802ana | EG522r | AATTAGAATGATGTC | 219 | NC_ | 9 | (TTC)9 | 324-353 |
| | | AGTTTTCACTGG | | 044378 | | | |
| CAN0907ana | EG515f | TGTACTAAAGTTGGT | 220 | NC_ | 9 | (TGA)9 | 223-235 |
| | | TCAATTCGAC | | 044375 | | | |
| CAN0907ana | EG516r | CACTTTGACTAGTG | 221 | NC_ | 9 | (TGA)9 | 223-235 |
| | | ATCCAAACAG | | 044375 | | | |
| CAN1409ana | EG548f | CCTTACTTTTCAACT | 222 | NC_ | 11 | (ATG)11 | 155-188 |
| | | TGCCATCCC | | 044374 | | | |
| CAN1409ana | EG549r | ATGGGTTTTATTCAA | 223 | NC_ | 11 | (ATG)11 | 155-188 |
| | | CAACCGGTTAC | | 044374 | | | |
| CAN1539ana | EG507f | GAGACTTGCCAAAT | 224 | NC_ | 9 | (ATC)9 | 491-520 |
| | | CCAACTTC | | 704437 | | | |
| CAN1539ana | EG508r | CTGTATGCGTCTTT | 225 | NC_ | 9 | (ATC)9 | 491-520 |
| | | GTAACTATTTG | | 044377 | | | |
| CAN1542ana | EG474f | GTCGGATTTATCTAA | 226 | NC_ | 9 | (TAT)9 | 154-178 |
| | | AGGTTAGAG | | 044374 | | | |
| CAN1542ana | EG475r | GAGAAGTACAGGAA | 227 | NC_ | 9 | (TAT)9 | 154-178 |
| | | CCAAGAG | | 044374 | | | |
| CAN1832ana | EG501f | GGTCCTTACAAAAC | 228 | NC_ | 3 | (AGGAGA)3 | 127-133 |
| | | TTCCCAC | | 044371 | | | |
| CAN1832ana | EG502r | TCATTCCTGAGAATA | 229 | NC_ | 3 | (AGGAGA)3 | 127-133 |
| | | TAAAGCTTG | | 044371 | | | |
| CAN2039ana | EG541f | TAATTTATTTTACAG | 230 | NC_ | 12 | (CAA)12 | 372-408 |
| | | TTGGGGGAAGTG | | 044374 | | | |
| CAN2039ana | EG542r | CAGTAGTACAAGAT | 231 | NC_ | 12 | (CAA)12 | 372-408 |
| | | CCAAGGAAGTG | | 044374 | | | |
| CAN2418ana | EG546f | TTGTTGTACTGATGA | 232 | NC_ | 13 | (CAA)13 | 450-489 |
| | | ACTCGTTGTTG | | 044374 | | | |
| CAN2418ana | EG547r | AGCTTAACTCAACC | 233 | NC_ | 13 | (CAA)13 | 450-489 |
| | | AAGATACTTTTGC | | 044374 | | | |
| CAN2550ana2 | EG434f | TCATATGTGAGATTG | 234 | NC_ | 3 | (CTT)3... | |
| | | TGATTAGCT | | 044374 | | (CTCAAA)6 | |
| CAN2550ana2 | EG415r | GGTGGTCGTATTAT | 235 | NC_ | 3 | (CTT)3... | |
| | | TCGCATAG | | 044374 | | (CTCAAA)6 | |
| CAN2829ana | EG525f | TTGCAAACTAGCGA | 236 | NC_ | 8 | (GAA)8 | 510-534 |
| | | GGGAGC | | 044377 | | | |
| CAN2829ana | EG526r | TCCGGTTCCCATAC | 237 | NC_ | 8 | (GAA)8 | 510-534 |
| | | AATTCTTG | | 044377 | | | |
| CAN2846ana | EG533f | CCTCAAGATGGCAA | 238 | NC_ | 9 | (GGT)9 | 281-308 |
| | | AAATGGGG | | 044376 | | | |
| CAN2846ana | EG534r | GCAGGGATTGGACC | 239 | NC_ | 9 | (GGT)9 | 281-308 |
| | | CATTTG | | 044376 | | | |
| CAN2902ana | EG529f | TCTCCTAGAAGTGG | 240 | NC_ | 7 | (TTC)7 | 486-507 |
| | | CAATCCC | | 044374 | | | |
| CAN2902ana | EG530r | CCAACTGCACGTAC | 241 | NC_ | 7 | (TTC)7 | 486-507 |
| | | AGAAACAAAA | | 044374 | | | |
| CAN2913ana | EG527f | GAAAGCGAGAGGCA | 242 | NC_ | 7 | (AAG)7 | 130-180 |
| | | GAACAG | | 044373 | | | |
| CAN2913ana | EG528r | TGGTCTTGACGCCA | 243 | NC_ | 7 | (AAG)7 | 130-180 |
| | | TTCTCG | | 044373 | | | |
| CAN3008ana | EG531f | AAGGAAGCTTTGAG | 244 | NC_ | 7 | (AGA)7 | 353-373 |
| | | GTCCAGC | | 044379 | | | |
| CAN3008ana | EG532r | GCTCCTCCGGTGGT | 245 | NC_ | 7 | (AGA)7 | 353-373 |
| | | CTAAAC | | 044379 | | | |
| D02- | EG418f | GAACCCCAAATGTT | 246 | NC_ | 3 | (GTT)7 | 338-359 |
| CANN1ana | | GTATTGAAG | | 044375 | | | |
| D02- | EG413r | TTTTAAATTTAGGTG | 247 | NC_ | 3 | (GTT)7 | 338-359 |
| CANN1ana | | TGTACTTTGG | | 044375 | | | |
| E07- | EG469f | TCCACCACCATCAT | 248 | NC_ | 3 | (CTA)9 | 273-282 |
| CANN1ana2 | | CAGC | | 044374 | | | |
| E07- | EG420r | TATATGTGGGTGTG | 249 | NC_ | 3 | (CTA)9 | 273-282 |
| CANN1ana2 | | TTAGAATTTTC | | 044374 | | | |
| H06- | EG494f | AGAGGAGCTGGTTT | 250 | NC_ | 7 | (ACG)7 | 151-157 |
| CANN2ana | | CAGTGG | | 044373 | | | |
| H06- | EG495r | TTCCCTGTCGTGGT | 251 | NC_ | 7 | (ACG)7 | 151-157 |
| CANN2ana | | TGCTTG | | 044373 | | | |
| ANUCS501ana2 | EG417r | TGTCACTCACTGCC | 295 | NC_ | 4 | (TTGTG)4 | 137-157 |
| | | ATCTCTC | | 044370 | | | |
| CAN0067ana | EG657f | CCTTTGCTCTATTCC | 296 | NC_ | 7 | (TTC)7 | 544-567 |
| | | CACTTACAAC | | 704433 | | | |
| CAN0067ana | EG658r | TTTAGACAAACCACA | 297 | NC_ | 7 | (TTC)7 | 544-567 |
| | | CAACCGAAAAC | | 044373 | | | |
| CAN0097ana | EG685f | TCACTACTTTCACCA | 298 | NC_ | 6 | (CAT)6 | 157-178 |
| | | ACGTTAGCTC | | 044373 | | | |
| CAN0097ana | EG686r | AGTAAAGCCTCTGC | 299 | NC_ | 6 | (CAT)6 | 157-178 |
| | | GAGTATCCTC | | 044373 | | | |
| CAN0202ana2 | EG591r | AACCTTGTACGCTTT | 300 | NC_ | 7 | (TTC)7 | 434-457 |
| | | CCACTTGC | | 044377 | | | |
| CAN0250ana | EG581f | GCACAATTCAAAGG | 301 | NC_ | 6 | (TTC)6 | 403-424 |
| | | TAGCCATAGC | | 044371 | | | |
| CAN0250ana | EG582r | CAGGGTAACAATCA | 302 | NC_ | 6 | (TTC)6 | 403-424 |
| | | CATGAAACTAC | | 044371 | | | |
| CAN0504ana2 | EG602f | CACAAGCATAGCTC | 303 | NC_ | 8 | (ACA)7a | 170-197 |
| | | CACCTGTAAC | | 044375 | | (GAT)8 | |
| CAN0504ana2 | EG683r | AAGGAAATCAACTTT | 304 | NC_ | 8 | (ACA)7a | 170-197 |
| | | GGATCATCTCAG | | 044375 | | (GAT)8 | |
| CAN1539ana2 | EG588r | GAGTCTGCTGTACA | 305 | NC_ | 9 | (ATC)9 | 279-303 |
| | | TTTGGACG | | 044377 | | | |
| E07- | EG587f | CATTGTGATGGAGT | 306 | NC_ | 9 | (CTA)9 | 479-512 |
| CANN1ana4 | | TGATGAAAGTAC | | 044374 | | | |
| CAN0067ana2 | EG693f | CCCTTTGCTCTATTC | 307 | NC_ | 7 | (TTC)7 | 544-567 |
| | | CCACTTACAAC | | 044373 | | | |
| CAN0067ana2 | EG694r | TTAGACAAACCACA | 308 | NC_ | 7 | (TTC)7 | 544-567 |
| | | CAACCGAAAACAG | | 044373 | | | |
| CAN0202ana3 | EG692f | ATCGGGAAGACACA | 309 | NC_ | 7 | (TTC)7 | 434-457 |
| | | TGCTTCATACTAC | | 044377 | | | |
| CAN0802ana2 | EG690f | TGGGCAGAGAAACT | 310 | NC_ | 9 | (TTC)9 | 324-353 |
| | | CCCCCTAATGAAAG | | 044378 | | | |
| CAN0802ana2 | EG691r | TTTTGGTGTGTGGA | 311 | NC_ | 9 | (TTC)9 | 324-353 |
| | | AGGGAACTTGACG | | 044378 | | | |
| CAN0055ana5 | EG688f | GTAATAAGATTCCAC | 312 | NC_ | 4 | (AATAA)4 | 110-115 |
| | | CTTCTGGCTCTTATG | | 044379 | | | |
| CAN0055ana5 | EG689r | CCAAAACTTAATCTA | 313 | NC_ | 4 | (AATAA)4 | 110-115 |
| | | ACCTCTAACGACC | | 044379 | | | |

TABLE 2
Multiplex 1

| Marker | Label | Small frag | Large frag | Primer Name |
|---|---|---|---|---|
| ANUCS302 | D2 | 140 | 160 | EG212f-213r |
| ANUCS305 | D3 | 142 | 167 | EG218f-219r |
| ANUCS501 | D2 | 78 | 98 | EG220f-221r |
| B01-CANN1 | D4 | 311 | 371 | EG232f-233r |
| B02-CANN2 | D2 | 164 | 173 | EG234f-235r |
| C08-CANN2 | D4 | 171 | 203 | EG236f-237r |
| CAN0093 | D3 | 205 | 250 | EG248f-249r |
| CAN0110 | D4 | 100 | 130 | EG250f-251r |
| CAN1347 | D2 | 210 | 260 | EG256f-257r |
| D02-CANN1 | D2 | 105 | 111 | EG238f-239r |
| E07-CANN1 | D3 | 105 | 132 | EG226f-227r |
| H06-CANN2 | D3 | 266 | 273 | EG228f-229r |
| H09-CANN2 | D4 | 204 | 300 | EG230f-231r |

TABLE 3
Multiplex 2

| Marker | Label | Small frag | Large frag | Primer Name |
|---|---|---|---|---|
| ANUCS303 | D3 | 139 | 163 | EG214f-15r |
| ANUCS304 | D4 | 141 | 222 | EG216f-217r |
| ANUCS501 | D2 | 78 | 98 | EG220f-221r |
| B01-CANN1 | D4 | 311 | 371 | EG232f-233r |
| CAN0055 | D4 | 225 | 250 | EG266f-267r |
| CAN0110 | D4 | 100 | 130 | EG250f-251r |
| CAN0126 | D2 | 160 | 180 | EG252f-253r |
| CAN0865 | D3 | 175 | 230 | EG270f-271r |
| CAN1347 | D2 | 210 | 260 | EG256f-257r |
| CAN2913 | D2 | 100 | 150 | EG260f-261r |
| E07-CANN1 | D3 | 105 | 132 | EG226f-227r |
| H06-CANN2 | D3 | 266 | 273 | EG228f-229r |

TABLE 4
Multiplex 3

| Marker | Label | Small frag | Large frag | Primer Name |
|---|---|---|---|---|
| ANUCS303 | D3 | 139 | 163 | EG214f-15r |
| ANUCS304 | D4 | 141 | 222 | EG216f-217r |
| ANUCS501 | D2 | 78 | 98 | EG220f-221r |
| B01-CANN1 | D4 | 311 | 371 | EG232f-233r |
| B05-CANN1 | D3 | 227 | 245 | EG222f-223r |
| CAN0055 | D4 | 225 | 250 | EG266f-267r |
| CAN0110 | D4 | 100 | 130 | EG250f-251r |
| D02-CANN1 | D2 | 105 | 111 | EG238f-239r |
| E07-CANN1 | D3 | 105 | 132 | EG226f-227r |
| H06-CANN2 | D3 | 266 | 273 | EG228f-229r |
| NM101 | D2 | 134 | 356 | EG292f-293r |

TABLE 5
Multiplex 4

| Marker | Label | Small frag | Large frag | Primer Name |
|---|---|---|---|---|
| ANUCS201 | D4 | 155 | 227 | EG208f-209r |
| ANUCS302 | D2 | 140 | 160 | EG212f-213r |
| ANUCS305 | D3 | 142 | 167 | EG218f-219r |
| ANUCS501 | D2 | 78 | 98 | EG220f-221r |
| B01-CANN1 | D4 | 311 | 371 | EG232f-233r |
| B02-CANN2 | D2 | 164 | 173 | EG234f-235r |
| CAN0055 | D4 | 225 | 250 | EG266f-267r |
| CAN0110 | D4 | 100 | 130 | EG250f-251r |
| CAN0865 | D3 | 175 | 230 | EG270f-271r |
| CAN1347 | D2 | 210 | 260 | EG256f-257r |
| D02-CANN1 | D2 | 105 | 111 | EG238f-239r |
| E07-CANN1 | D3 | 105 | 132 | EG226f-227r |
| H06-CANN2 | D3 | 266 | 273 | EG228f-229r |

TABLE 6
Multiplex 5

| Marker | Label | Small frag | Large frag | Primer Name |
|---|---|---|---|---|
| ANUCS305 | D3 | 142 | 167 | EG218f-219r |
| ANUCS501 | D2 | 78 | 98 | EG220f-221r |
| B01-CANN1 | D4 | 311 | 371 | EG232f-233r |
| C08-CANN2 | D4 | 171 | 203 | EG236f-237r |
| CAN0110 | D4 | 100 | 130 | EG250f-251r |
| CAN0865 | D3 | 175 | 230 | EG270f-271r |
| E07-CANN1 | D3 | 105 | 132 | EG226f-227r |
| H06-CANN2 | D3 | 266 | 273 | EG228f-229r |
| H09-CANN2 | D4 | 204 | 300 | EG230f-231r |
| NM101 | D2 | 134 | 356 | EG292f-293r |

TABLE 7
Multiplex 6

| Marker | Label | Small frag | Large frag | Primer Name |
|---|---|---|---|---|
| ANUCS304 | D4 | 141 | 222 | EG216f-217r |
| ANUCS501 | D2 | 78 | 98 | EG220f-221r |
| B01-CANN1 | D4 | 311 | 371 | EG232f-233r |
| C11-CANN1 | D3 | 150 | 175 | EG224f-225r |
| CAN0055 | D4 | 225 | 250 | EG266f-267r |
| CAN0093 | D3 | 205 | 250 | EG248f-249r |
| CAN0110 | D4 | 100 | 130 | EG250f-251r |
| CAN0126 | D2 | 160 | 180 | EG252f-253r |
| CAN1347 | D2 | 210 | 260 | EG256f-257r |
| CAN2913 | D2 | 100 | 150 | EG260f-261r |
| E07-CANN1 | D3 | 105 | 132 | EG226f-227r |
| H06-CANN2 | D3 | 266 | 273 | EG228f-229r |

TABLE 8
Multiplex 7

| Marker | Label | Small frag | Large frag | Primer Name |
|---|---|---|---|---|
| ANUCS201 | D4 | 155 | 227 | EG208f-209r |
| ANUCS302 | D2 | 140 | 160 | EG212f-213r |
| ANUCS501 | D2 | 78 | 98 | EG220f-221r |
| B01-CANN1 | D4 | 311 | 371 | EG232f-233r |
| B02-CANN2 | D2 | 164 | 173 | EG234f-235r |
| B05-CANN1 | D3 | 227 | 245 | EG222f-223r |
| C11-CANN1 | D3 | 150 | 175 | EG224f-225r |
| CAN0110 | D4 | 100 | 130 | EG250f-251r |
| CAN1347 | D2 | 210 | 260 | EG256f-257r |
| D02-CANN1 | D2 | 105 | 111 | EG238f-239r |
| E07-CANN1 | D3 | 105 | 132 | EG226f-227r |
| H06-CANN2 | D3 | 266 | 273 | EG228f-229r |

TABLE 9
Multiplex 8

| Marker | Label | Small frag | Large frag | Primer Name |
|---|---|---|---|---|
| ANUCS303 | D3 | 139 | 163 | EG214f-15r |
| ANUCS304 | D4 | 141 | 222 | EG216f-217r |
| ANUCS501 | D2 | 78 | 98 | EG220f-221r |
| B01-CANN1 | D4 | 311 | 371 | EG232f-233r |
| CAN0055 | D4 | 225 | 250 | EG266f-267r |
| CAN0110 | D4 | 100 | 130 | EG250f-251r |
| CAN0865 | D3 | 175 | 230 | EG270f-271r |
| E07-CANN1 | D3 | 105 | 132 | EG226f-227r |
| H06-CANN2 | D3 | 266 | 273 | EG228f-229r |
| NM101 | D2 | 134 | 356 | EG292f-293r |

TABLE 10
Multiplex 11

| | | Small | | |
|---|---|---|---|---|
| Marker | Label | frag | Large frag | Primer Name |
| ANUCS201_D4 | D4 | 152 | 228 | EG208f_D4-209r |
| ANUCS501_D2 | D2 | 78 | 108 | EG220f_D2-221r |
| CAN0093_D3 | D3 | 181 | 232 | EG248f_D3-249r |
| CAN0110_D4 | D4 | 96 | 134 | EG250f_D4-251r |
| CAN1482_D3 | D3 | 270 | 309 | EG288f_D3-289r |
| E07-CANN1_D3 | D3 | 105 | 117 | EG226f_D3-227r |
| NM101_D2 | D2 | 131 | 335 | EG292f_D2-293r |

TABLE 11
Multiplex 12

| | | Small | | |
|---|---|---|---|---|
| Marker | Label | frag | Large frag | Primer Name |
| D02-CANN1_D2 | D2 | 110 | 119 | EG238f_D2-239r |
| CAN1347_D2 | D2 | 202 | 229 | EG256f_D2-257r |
| ANUCS305_D3 | D3 | 141 | 168 | EG218f_D3-219r |
| CAN0865_D3 | D3 | 175 | 230 | EG270f_D3-271r |
| H06-CANN2_D3 | D3 | 268 | 277 | EG228f_D3-229r |
| C08-CANN2_D4 | D4 | 147 | 205 | EG236f_D4-237r |
| H09-CANN2_D4 | D4 | 196 | 224 | EG230f_D4-231r |

TABLE 12
Multiplex 13

| Marker | Label | Small frag | Large frag | Primer Name |
|---|---|---|---|---|
| ANUCS301_D4 | D4 | 205 | 276 | EG210f_D4-211r |
| ANUCS305_D3 | D3 | 141 | 168 | EG218f_D3-219r |
| ANUCS501_D3* | D3* | 78 | 108 | EG220f_D3-221r |
| B01-CANN1_D4 | D4 | 311 | 371 | EG232f_D4-233r |
| CAN0055_D2* | D2* | 250 | 275 | EG266f_D2-267r |
| CAN1001_D2 | D2 | 300 | 350 | EG274f_D2-275r |
| CAN1347_D2 | D2 | 202 | 229 | EG256f_D2-257r |
| H06-CANN2_D3 | D3 | 268 | 277 | EG228f_D3-229r |

TABLE 13
Multiplex 14

| Marker | Label | Small frag | Large frag | Primer Name |
|---|---|---|---|---|
| ANUCS304_D4 | D4 | 141 | 222 | EG216f_D4-217r |
| C11-CANN1_D3 | D3 | 150 | 175 | EG224f_D3-225r |
| CAN0055_D4 | D4 | 250 | 280 | EG266f_D4-267r |
| CAN1482_D3 | D3 | 270 | 309 | EG288f_D3-289r |
| D02-CANN1_D2 | D2 | 110 | 119 | EG238f_D2-239r |
| E07-CANN1_D3 | D3 | 105 | 117 | EG226f_D3-227r |
| NM101_D2 | D2 | 131 | 335 | EG292f_D2-293r |

TABLE 14
Multiplex 15

| Marker | Label | Small frag | Large frag | Primers |
|---|---|---|---|---|
| ANUCS301_D4 | D4 | 205 | 276 | EG210f_D4-211r |
| ANUCS304_D4 | D4 | 141 | 222 | EG216f_D4-217r |
| ANUCS305_D3 | D3 | 141 | 168 | EG218f_D3-219r |
| ANUCS501_D2 | D2 | 78 | 108 | EG220f_D2-221r |
| CAN1482_D3 | D3 | 270 | 309 | EG288f_D3-289r |
| D02-CANN1_D2 | D2 | 110 | 119 | EG238f_D2-239r |
| E07-CANN1_D3 | D3 | 105 | 117 | EG226f_D3-227r |
| NM_101_D2 | D2 | 131 | 335 | EG292f_D2-293r |

TABLE 15
Multiplex 16

| Marker | Label | Small frag | Large frag | Primers |
|---|---|---|---|---|
| ANUCS304_D4 | D4 | 141 | 222 | EG216f_D4-217r |
| ANUCS501_D2 | D2 | 78 | 108 | EG220f_D2-221r |
| B01-CANN1_D4 | D4 | 311 | 371 | EG232f_D4-233r |
| C11-CANN1_D3 | D3 | 150 | 175 | EG224f_D3-225r |
| CAN0055_D4 | D4 | 250 | 280 | EG266f_D4-267r |
| E07-CANN1_D3 | D3 | 105 | 117 | EG226f_D3-227r |
| H06-CANN2_D3 | D3 | 268 | 277 | EG228f_D3-229r |

TABLE 16
Multiplex 17

| Marker | Label | Small frag | Large frag | Primers |
|---|---|---|---|---|
| B01-CANN1_D4 | D4 | 311 | 371 | EG232f_D4-233r |
| C11-CANN1_D3 | D3 | 150 | 175 | EG224f_D3-225r |
| CAN0055_D4 | D4 | 250 | 280 | EG266f_D4-267r |
| CAN1482_D3 | D3 | 270 | 309 | EG288f_D3-289r |
| D02-CANN1_D2 | D2 | 110 | 119 | EG238f_D2-239r |
| E07-CANN1_D3 | D3 | 105 | 117 | EG226f_D3-227r |
| NM101_D2 | D2 | 131 | 335 | EG292f_D2-293r |

TABLE 17
Multiplex 18

| Marker | Label | Small frag | Large frag | Primers |
|---|---|---|---|---|
| ANUCS301_D4 | D4 | 205 | 276 | EG210f_D4-211r |
| ANUCS304_D2* | D2 | 141 | 222 | EG216f_D2-217r |
| B01-CANN1_D4 | D4 | 311 | 371 | EG232f_D4-233r |
| C11-CANN1_D3 | D3 | 150 | 175 | EG224f_D3-225r |
| D02-CANN1_D2 | D2 | 110 | 119 | EG238f_D2-239r |
| E07-CANN1_D3 | D3 | 105 | 117 | EG226f_D3-227r |
| H06-CANN2_D3 | D3 | 268 | 277 | EG228f_D3-229r |

TABLE 18
Multiplex 19

| Marker | Label | Small frag | Large frag | Primers |
|---|---|---|---|---|
| ANUCS304_D4 | D4 | 141 | 222 | EG216f_D4-217r |
| ANUCS305_D3 | D3 | 141 | 168 | EG218f_D3-219r |
| ANUCS501_D2 | D2 | 78 | 108 | EG220f_D2-221r |
| CAN0055_D4 | D4 | 250 | 280 | EG266f_D4-267r |
| E07-CANN1_D3 | D3 | 105 | 117 | EG226f_D3-227r |
| H06-CANN2_D3 | D3 | 268 | 277 | EG228f_D3-229r |
| NM101_D2 | D2 | 131 | 335 | EG292f_D2-293r |

TABLE 19
Multiplex 20

| Marker | Label | Small frag | Large frag | Primers |
|---|---|---|---|---|
| ANUCS304_D4 | D4 | 141 | 222 | EG216f_D4-217r |
| ANUCS305_D3 | D3 | 141 | 168 | EG218f_D3-219r |
| B01-CANN1_D4 | D4 | 312 | 366 | EG232f_D4-233r |
| CAN1347_D2 | D2 | 202 229 | 229 | EG256f_D2-257r |
| D02-CANN1_D2 | D2 | 110 | 119 | EG238f_D2-239r |
| H06-CANN2_D3 | D3 | 268 | 277 | EG228f_D3-229r |

TABLE 20
Multiplex 21

| Marker | Label | Small frag | Large frag | Primers |
|---|---|---|---|---|
| ANUCS501_D2 | D2 | 74 | 109 | EG220f_D2-221r |
| C11-CANN1_D3 | D3 | 144 | 165 | EG224f_D3-225r |
| CAN0055_D4 | D4 | 268 | 288 | EG266f_D4-267r |
| CAN1482_D3 | D3 | 270 | 309 | EG288f_D3-289r |
| E07-CANN1_D3 | D3 | 105 | 117 | EG226f_D3-227r |
| NM101_D2 | D2 | 131 | 335 | EG292f_D2-293r |

TABLE 21
Multiplex 22

| Marker | Label | Small frag | Large frag | Primers |
|---|---|---|---|---|
| ANUCS305_D3 | D3 | 141 | 168 | EG218f_D3-219r |
| ANUCS501_D2 | D2 | 74 | 109 | EG220f_D2-221r |
| B01-CANN1_D4 | D4 | 312 | 366 | EG232f_D4-233r |
| CAN0055_D4 | D4 | 268 | 288 | EG266f_D4-267r |
| H06-CANN2_D3 | D3 | 268 | 277 | EG228f_D3-229r |
| NM101_D2 | D2 | 131 | 335 | EG292f_D2-293r |

TABLE 22
Multiplex 23

| Marker | Label | Small frag | Large frag | Primers |
|---|---|---|---|---|
| ANUCS304_D4 | D4 | 141 | 222 | EG216f_D4-217r |
| C11-CANN1_D3 | D3 | 144 | 165 | EG224f_D3-225r |
| CAN1347_D2 | D2 | 202 | 229 | EG256f_D2-257r |
| CAN1482_D3 | D3 | 270 | 309 | EG288f_D3-289r |
| D02-CANN1_D2 | D2 | 110 | 119 | EG238f_D2-239r |
| E07-CANN1_D3 | D3 | 105 | 117 | EG226f_D3-227r |

TABLE 23
Multiplex 24

| Marker | Label | Small frag | Large frag | Primers |
|---|---|---|---|---|
| ANUCS501_D2 | D2 | 74 | 109 | EG220f_D2-221r |
| CAN1347_D2 | D2 | 202 229 | 229 | EG256f_D2-257r |
| ANUCS305_D3 | D3 | 141 | 168 | EG218f_D3-219r |
| CAN1482_D3 | D3 | 270 | 309 | EG288f_D3-289r |
| ANUCS304_D4 | D4 | 141 | 222 | EG216f_D4-217r |
| B01-CANN1_D4 | D4 | 312 | 366 | EG232f_D4-233r |

TABLE 24
Multiplex 25

| Marker | Label | Small frag | Large frag | Primers |
|---|---|---|---|---|
| C11-CANN1_D3 | D3 | 144 | 165 | EG224f_D3-225r |
| CAN0055_D4 | D4 | 268 | 288 | EG266f_D4-267r |
| D02-CANN1_D2 | D2 | 110 | 119 | EG238f_D2-239r |
| E07-CANN1_D3 | D3 | 105 | 117 | EG226f_D3-227r |
| H06-CANN2_D3 | D3 | 268 | 277 | EG228f_D3-229r |
| NM101_D2 | D2 | 131 | 335 | EG292f_D2-293r |

TABLE 25
Multiplex 26

| | | Small | Large | |
|---|---|---|---|---|
| Marker | Label | frag | frag | Primers |
| ANUCS501_D2 | D2 | 110 | 119 | EG220f_D2-221r |
| CAN0009_D4 | D4 | 289 | 312 | EG352f_D4-353r |
| CAN0509_D2 | D2 | 271 | 277 | EG306f_D2-307r |
| CAN0717_D3 | D3 | 301 | 310 | EG316f_D3-317r |
| H06-CANN2_D3 | D3 | 116 | 145 | EG228f_D3-229r |

TABLE 26
Multiplex 27

| | | Small | Large | |
|---|---|---|---|---|
| Marker | Label | frag | frag | Primers |
| B01-CANN1_D4 | D4 | 83 | 103 | EG232f_D4-233r |
| C11-CANN1_D3 | D3 | 131 | 335 | EG224f_D3-225r |
| CAN0055_D4 | D4 | 108 | 117 | EG266f_D4-267r |
| CAN0111_D3 | D3 | 153 | 178 | EG300f_D3-301r |
| CAN2550_D2 | D2 | 278 | 283 | EG340f_D2-341r |
| D02-CANN1_D2 | D2 | 315 | 370 | EG238f_D2-239r |
| E07-CANN1_D3 | D3 | 83 | 103 | EG226f_D3-227r |

TABLE 27
Multiplex 28

| | | Small | Large | |
|---|---|---|---|---|
| Marker | Label | frag | frag | Primers |
| CAN0009_D4 | D4 | 116 | 145 | EG352f_D4-353r |
| CAN0010_D2 | D2 | 268 | 281 | EG354f_D2-355r |
| CAN0717_D3 | D3 | 301 | 310 | EG316f_D3-317r |
| CAN2550_D4 | D4 | 301 | 325 | EG340f_D4-341r |
| D02-CANN1_D2 | D2 | 110 | 119 | EG238f_D2-239r |
| H06-CANN2_D3 | D3 | 271 | 277 | EG228f_D3-229r |

TABLE 28
Multiplex 29

| Marker | Label | Small frag | Large frag | Primers |
|---|---|---|---|---|
| ANUCS501_D2 | D2 | 83 | 103 | EG220f_D2-221r |
| C11-CANN1_D3 | D3 | 153 | 178 | EG224f_D3-225r |
| CAN0055_D4 | D4 | 278 | 283 | EG266f_D4-267r |
| CAN0878_D4 | D4 | 299 | 324 | EG320f_D4-321r |
| CAN2717_D2 | D2 | 296 | 308 | EG344f_D2-345r |
| E07-CANN1_D3 | D3 | 108 | 117 | EG226f_D3-227r |

TABLE 29
Multiplex 30

| Marker | Label | Small frag | Large frag | Primers |
|---|---|---|---|---|
| ANUCS501_D2 | D2 | 83 | 103 | EG220f_D2-221r |
| CAN0009_D4 | D4 | 116 | 145 | EG352f_D4-353r |
| CAN0509_D2 | D2 | 289 | 312 | EG306f_D2-307r |
| CAN0717_D3 | D3 | 301 | 310 | EG316f_D3-317r |
| CAN2550_D4 | D4 | 301 | 325 | EG340f_D4-341r |
| H06-CANN2_D3 | D3 | 271 | 277 | EG228f_D3-229r |

TABLE 30
Multiplex 32

| Marker | Label | Small frag | Large frag | Primers |
|---|---|---|---|---|
| NM101_D2 | D2 | 131 | 335 | EG292f_D2-293r |
| E07-CANN1_D3 | D3 | 108 | 117 | EG226f_D3-227r |
| C11-CANN1_D3 | D3 | 153 | 178 | EG224f_D3-225r |
| CAN0111_D3 | D3 | 291 | 310 | EG300f_D3-301r |
| CAN0055_D4 | D4 | 278 | 283 | EG266f_D4-267r |
| B01-CANN1_D4 | D4 | 315 | 370 | EG232f_D4-233r |

TABLE 31
Multiple 33

| Marker | Label | Small frag | Large frag | Primers |
|---|---|---|---|---|
| D02-CANN1_D2 | D2 | 110 | 119 | EG238f_D2-239r |
| E07-CANN1_D3 | D3 | 108 | 117 | EG226f_D3-227r |
| C11-CANN1_D3 | D3 | 153 | 178 | EG224f_D3-225r |
| CAN0111_D3 | D3 | 291 | 310 | EG300f_D3-301r |
| CAN0055_D4 | D4 | 278 | 283 | EG266f_D4-267r |
| B01-CANN1_D4 | D4 | 315 | 370 | EG232f_D4-233r |

TABLE 32
Multiplex 34

| Marker | Label | Small frag | Large frag | Primers |
|---|---|---|---|---|
| ANUCS501_D2 | D2 | 83 | 103 | EG220f_D2-221r |
| CAN0009_D4 | D4 | 116 | 145 | EG352f_D4-353r |
| CAN0509_D2 | D2 | 289 | 312 | EG306f_D2-307r |
| CAN0717_D3 | D3 | 301 | 310 | EG316f_D3-317r |
| H06-CANN2_D3 | D3 | 271 | 277 | EG228f_D3-229r |

TABLE 33
Multiplex 35

| Marker | Label | Small frag | Large frag | Primers |
|---|---|---|---|---|
| D02-CANN1_D2 | D2 | 110 | 119 | EG238f_D2-239r |
| CAN2550_D2 | D2 | 301 | 325 | EG340f_D2-341r |
| E07-CANN1_D3 | D3 | 105 | 117 | EG226f_D3-227r |
| C11-CANN1_D3 | D3 | 153 | 178 | EG224f_D3-225r |
| CAN0111_D3 | D3 | 291 | 310 | EG300f_D3-301r |
| CAN0055_D4 | D4 | 278 | 283 | EG266f_D4-267r |
| B01-CANN1_D4 | D4 | 315 | 370 | EG232f_D4-233r |

TABLE 34
Multiple 36

| Marker | Label | Small frag | Large frag | Primers |
|---|---|---|---|---|
| ANUCS501_D2 | D2 | 83 | 103 | EG220f_D2-221r |
| B01-CANN1_D4 | D4 | 315 | 370 | EG232f_D4-233r |
| C11-CANN1_D3 | D3 | 153 | 178 | EG224f_D3-225r |
| CAN0009_D4 | D4 | 116 | 145 | EG352f_D4-353r |
| CAN0055_D4 | D4 | 278 | 283 | EG266f_D4-267r |
| CAN0509_D4 | D4 | 289 | 312 | EG306f_D4-307r |
| CAN0717_D3 | D3 | 301 | 310 | EG316f_D3-317r |
| CAN2550_D2 | D2 | 301 | 325 | EG340f_D2-341r |
| D02-CANN1_D2 | D2 | 110 | 119 | EG238f_D2-239r |
| E07-CANN1_D3 | D3 | 108 | 117 | EG226f_D3-227r |

TABLE 35a
Results of pairwise primer pair combinations tested among multiplex reactions 1 to 36.

| | ANUCS301 | ANUCS303 | ANUCS304 | ANUCS305 | ANUCS501 | B01-CANN1 | B02-CANN2 | B05-CANN1 | C11-CANN1 |
|---|---|---|---|---|---|---|---|---|---|
| ANUCS301 | | | | | | | | | |
| ANUCS303 | | | | | | | | | |
| ANUCS304 | | 1 | | | | | | | |
| ANUCS305 | | | | | | | | | |
| ANUCS501 | 1 | 1 | 5 | 3 | | | | | |
| B01-CANN1 | | 1 | 4 | 4 | 9 | | | | |
| B02-CANN2 | | | | | 2 | 2 | | | |
| B05-CANN1 | | | | | 1 | 1 | 1 | | |
| C11-CANN1 | | | 1 | | 5 | 9 | 1 | 1 | |
| CAN0009 | | | | | 3 | | | | |
| CAN0010 | | | | | | | | | |
| CAN0055 | | | 4 | | 8 | 12 | 2 | | 10 |
| CAN0111 | | | | | | 2 | | | 2 |
| CAN0126 | | | 1 | | 2 | 1 | | | |
| CAN0509 | | | | | 3 | 1 | | | |
| CAN0717 | | | | | 3 | 2 | | | 2 |
| CAN0878 | | | | | 1 | | | | 1 |
| CAN1347 | | | 1 | 3 | 3 | 4 | 2 | 1 | 1 |
| CAN1482 | | | | | 1 | 1 | | | 2 |
| CAN2550 | | | | | 2 | 2 | | | 2 |
| CAN2717 | | | | | 1 | | | | 1 |
| CAN2913 | | | 1 | | 2 | 1 | | | |
| D02-CANN1 | | 1 | 1 | 4 | 4 | 9 | 2 | 1 | 4 |
| E07-CANN1 | | 1 | 4 | 3 | 12 | 15 | 2 | 1 | 10 |
| H06-CANN2 | | 1 | 4 | 5 | 11 | 10 | 2 | 1 | 2 |
| NM101 | | | 2 | 2 | 5 | 6 | | | 6 |
| LG | X | 5 | 8 | 2 | X | 4 | | | 7 |

| | CAN0009 | CAN0010 | CAN0055 | CAN0111 | CAN0126 | CAN0509 | CAN0717 | CAN0878 | CAN1347 |
|---|---|---|---|---|---|---|---|---|---|
| ANUCS301 | | | | | | | | | |
| ANUCS303 | | | | | | | | | |
| ANUCS304 | | | | | | | | | |
| ANUCS305 | | | | | | | | | |
| ANUCS501 | | | | | | | | | |
| B01-CANN1 | | | | | | | | | |
| B02-CANN2 | | | | | | | | | |
| B05-CANN1 | | | | | | | | | |
| C11-CANN1 | | | | | | | | | |
| CAN0009 | | | | | | | | | |
| CAN0010 | | | | | | | | | |
| CAN0055 | | | | | | | | | |
| CAN0111 | | | 2 | | | | | | |
| CAN0126 | | | | | | | | | |
| CAN0509 | 4 | | | | | | | | |
| CAN0717 | 5 | 1 | 2 | | | 4 | | | |
| CAN0878 | | | 1 | | | | | | |
| CAN1347 | | | 2 | | 1 | | | | |
| CAN1482 | | | 2 | | | | | | |
| CAN2550 | 4 | 1 | 2 | | | 3 | 4 | | |
| CAN2717 | | | 1 | | | | | 1 | |
| CAN2913 | | | | | 2 | | | | 1 |
| D02-CANN1 | 3 | 1 | 4 | 1 | | 2 | 3 | | 5 |
| E07-CANN1 | 1 | | 11 | 1 | 1 | 1 | 2 | 1 | 2 |
| H06-CANN2 | 1 | 1 | 5 | | 1 | 2 | 3 | | 8 |
| NM101 | | | 7 | | | | 1 | | |
| LG | 6 | 6 | 8 | X | 7 | 1 | 3 | 1 | 7 |

| | CAN1482 | CAN2550 | CAN2717 | CAN2913 | D02-CANN1 | E07-CANN1 | H06-CANN2 | NM101 | LG |
|---|---|---|---|---|---|---|---|---|---|
| ANUCS301 | | | | | | | | | X |
| ANUCS303 | | | | | | | | | 5 |
| ANUCS304 | | | | | | | | | 8 |
| ANUCS305 | | | | | | | | | 2 |
| ANUCS501 | | | | | | | | | X |
| B01-CANN1 | | | | | | | | | 4 |
| B02-CANN2 | | | | | | | | | |
| B05-CANN1 | | | | | | | | | |
| C11-CANN1 | | | | | | | | | 7 |
| CAN0009 | | | | | | | | | 6 |
| CAN0010 | | | | | | | | | 6 |
| CAN0055 | | | | | | | | | 8 |
| CAN0111 | | | | | | | | | X |
| CAN0126 | | | | | | | | | 7 |
| CAN0509 | | | | | | | | | 1 |
| CAN0717 | | | | | | | | | 3 |
| CAN0878 | | | | | | | | | 1 |
| CAN1347 | | | | | | | | | 7 |
| CAN1482 | | | | | | | | | X |
| CAN2550 | | | | | | | | | 9 |
| CAN2717 | | | | | | | | | 3 |
| CAN2913 | | | | | | | | | 4 |
| D02-CANN1 | | 4 | | | | | | | 2 |
| E07-CANN1 | 3 | 2 | 1 | 1 | 7 | | | | 5 |
| H06-CANN2 | | 3 | | 1 | 7 | 10 | | | 4 |
| NM101 | 3 | | | | 1 | 9 | 4 | | 2 |
| LG | X | 9 | 3 | 4 | 2 | 5 | 4 | 2 | |

TABLE 35b
Results of pairwise primer pair combinations tested among all of the multiplex reactions with new primer sets (Multiplexes 39-91).

| | ANUCS304ana | ANUCS501ana | ANUCS501ana2 | C11-CANN1ana2 | CAN0032ana | CAN0055ana4 | CAN0067ana |
|---|---|---|---|---|---|---|---|
| ANUCS304ana | | | | | | | |
| ANUCS501ana | 3 | | | | | | |
| ANUCS501ana2 | | | | | | | |
| C11-CANN1ana2 | | | >10 | | | | |
| CAN0032ana | | | 1 | 1 | | | |
| CAN0055ana4 | | | >10 | >10 | 1 | | |
| CAN0067ana | | | 9 | 9 | | 9 | |
| CAN0085ana2 | | | 1 | 1 | | 1 | |
| CAN0089ana | | 1 | >10 | >10 | 1 | >10 | 9 |
| CAN0097ana | | | 5 | 5 | | 5 | 4 |
| CAN0103ana | | 1 | | 8 | | 7 | |
| CAN0111ana | | | 7 | 3 | 1 | 7 | |
| CAN0111ana2 | | | 5 | 5 | | 5 | 4 |
| CAN0124ana | | 1 | | | | | |
| CAN0138ana2 | 1 | 1 | | | | | |
| CAN0156ana | | | 1 | 9 | | 8 | |
| CAN0180ana | 1 | 2 | | | | | |
| CAN0202ana2 | | | >10 | >10 | 1 | >10 | 8 |
| CAN0250ana | | | >10 | >10 | 1 | >10 | 8 |
| CAN0271ana | | 1 | | | | | |
| CAN0291ana | 2 | 3 | | | | | |
| CAN0338-339ana | 1 | 1 | | | | | |
| CAN0504ana | | | 6 | 8 | | 6 | 2 |
| CAN0504ana2 | | | 7 | 7 | | 7 | 6 |
| CAN0509ana | 1 | 1 | >10 | >10 | 1 | >10 | 8 |
| CAN0577ana | | | >10 | >10 | 1 | >10 | 8 |
| CAN0716ana | 1 | 2 | >10 | 10 | 1 | >10 | 5 |
| CAN0716ana2 | | | 3 | 3 | | 3 | 3 |
| CAN0717ana | | | >10 | >10 | 1 | >10 | 4 |
| CAN0802ana | | 1 | >10 | >10 | 1 | >10 | 8 |
| CAN0907ana | | 1 | >10 | >10 | 1 | >10 | 8 |
| CAN1409ana | | | | 1 | | 1 | |
| CAN1539ana | 1 | 2 | | 8 | | 7 | |
| CAN1539ana2 | | | >10 | >10 | 1 | >10 | 8 |
| CAN1832ana | | 1 | | 8 | | 7 | |
| CAN2418ana | | | | 1 | | 1 | |
| CAN2550ana2 | 1 | 1 | | | | | |
| CAN2829ana | | 1 | | | | | |
| CAN2846ana | | 1 | | | | | |
| CAN2902ana | | | | | | | |
| CAN3008ana | | 1 | >10 | >10 | 1 | >10 | 8 |
| DO2-CANN1ana | | | 3 | | | 3 | |
| E07-CANN1ana2 | 1 | 1 | 4 | 8 | | >10 | |
| E07-CANN1ana4 | | | >10 | >10 | 1 | >10 | 8 |
| H06-CANN2ana | 1 | 1 | 6 | >10 | 1 | >10 | |
| LG | 8 | X | X | 7 | 2 | 8 | 4 |

| | CAN0085ana2 | CAN0089ana | CAN0097ana | CAN0103ana | CAN0111ana | CAN0111ana2 | CAN0124ana |
|---|---|---|---|---|---|---|---|
| ANUCS304ana | | | | | | | |
| ANUCS501ana | | | | | | | |
| ANUCS501ana2 | | | | | | | |
| C11-CANN1ana2 | | | | | | | |
| CAN0032ana | | | | | | | |
| CAN0055ana4 | | | | | | | |
| CAN0067ana | | | | | | | |
| CAN0085ana2 | | | | | | | |
| CAN0089ana | 1 | | | | | | |
| CAN0097ana | | 4 | | | | | |
| CAN0103ana | | >10 | | | | | |
| CAN0111ana | | 7 | | | | | |
| CAN0111ana2 | | 4 | 3 | | | | |
| CAN0124ana | | 2 | | 1 | | | |
| CAN0138ana2 | | | | | | | |
| CAN0156ana | | >10 | 1 | >10 | | | 1 |
| CAN0180ana | | | | | | | |
| CAN0202ana2 | 1 | >10 | 5 | | 7 | 5 | |
| CAN0250ana | 1 | >10 | 5 | | 7 | 5 | |
| CAN0271ana | | 3 | | 3 | | | 1 |
| CAN0291ana | | | | | | | |
| CAN0338-339ana | | | | | | | |
| CAN0504ana | | 6 | | | 2 | 2 | |
| CAN0504ana2 | 1 | 5 | 5 | | | 3 | |
| CAN0509ana | 1 | >10 | 5 | >10 | 7 | 5 | |
| CAN0577ana | 1 | >10 | 5 | | 7 | 5 | |
| CAN0716ana | | >10 | 5 | | 7 | 4 | 1 |
| CAN0716ana2 | 1 | 1 | 1 | | | 1 | |
| CAN0717ana | | >10 | 3 | | 5 | 3 | |
| CAN0802ana | 1 | >10 | 5 | >10 | 7 | 5 | 2 |
| CAN0907ana | 1 | >10 | 5 | >10 | 7 | 5 | |
| CAN1409ana | | 1 | | | | | |
| CAN1539ana | | >10 | | >10 | | | 2 |
| CAN1539ana2 | 1 | >10 | 5 | | 7 | 5 | |
| CAN1832ana | | >10 | | >10 | | | |
| CAN2418ana | | 1 | | | | | |
| CAN2550ana2 | | | | | | | |
| CAN2829ana | | 1 | | 1 | | | |
| CAN2846ana | | | | 4 | | | |
| CAN2902ana | | | | | | | |
| CAN3008ana | 1 | >10 | 5 | >10 | 7 | 5 | 1 |
| DO2-CANN1ana | | 2 | | | 3 | | |
| E07-CANN1ana2 | | >10 | | >10 | 4 | | |
| E07-CANN1ana4 | 1 | >10 | 5 | | 3 | 5 | |
| H06-CANN2ana | | >10 | | >10 | 6 | | 1 |
| LG | 4 | 9 | 4 | 3 | X | X | 1 |
| | CAN0138ana2 | CAN0156ana | CAN0180ana | CAN0202ana2 | CAN0250ana | CAN0271ana | CAN0291ana |
| ANUCS304ana | | | | | | | |
| ANUCS501ana | | | | | | | |
| ANUCS501ana2 | | | | | | | |
| C11-CANN1ana2 | | | | | | | |
| CAN0032ana | | | | | | | |
| CAN0055ana4 | | | | | | | |
| CAN0067ana | | | | | | | |
| CAN0085ana2 | | | | | | | |
| CAN0089ana | | | | | | | |
| CAN0097ana | | | | | | | |
| CAN0103ana | | | | | | | |
| CAN0111ana | | | | | | | |
| CAN0111ana2 | | | | | | | |
| CAN0124ana | | | | | | | |
| CAN0138ana2 | | | | | | | |
| CAN0156ana | | | | | | | |
| CAN0180ana | | | | | | | |
| CAN0202ana2 | | 1 | | | | | |
| CAN0250ana | | 1 | | >10 | | | |
| CAN0271ana | | 2 | | | | | |
| CAN0291ana | 1 | | 1 | | | | |
| CAN0338-339ana | 1 | | | | | | 1 |
| CAN0504ana | | | | 6 | 6 | | |
| CAN0504ana2 | | 1 | | 7 | 7 | | |
| CAN0509ana | 1 | >10 | | >10 | >10 | 1 | 1 |
| CAN0577ana | | 2 | | >10 | >10 | | |
| CAN0716ana | 2 | 3 | | >10 | >10 | 1 | 1 |
| CAN0716ana2 | | | | 3 | 3 | | |
| CAN0717ana | | 1 | | >10 | >10 | | |
| CAN0802ana | | >10 | | >10 | >10 | 3 | |
| CAN0907ana | | >10 | | >10 | >10 | | |
| CAN1409ana | | 1 | | | | | |
| CAN1539ana | 2 | >10 | | | | 2 | 3 |
| CAN1539ana2 | | 1 | | >10 | >10 | | |
| CAN1832ana | 1 | >10 | | | | 2 | |
| CAN2418ana | | 1 | | | | | |
| CAN2550ana2 | 1 | | | | | | 1 |
| CAN2829ana | | 1 | | | | 1 | |
| CAN2846ana | | 6 | | | | | |
| CAN2902ana | | 1 | | | | | 1 |
| CAN3008ana | | >10 | | >10 | >10 | 2 | |
| DO2-CANN1ana | | | | 3 | 3 | | |
| E07-CANN1ana2 | | >10 | 1 | 4 | 4 | 1 | 1 |
| E07-CANN1ana4 | | 1 | | >10 | >10 | | |
| H06-CANN2ana | 2 | >10 | | 6 | 6 | 1 | 2 |
| LG | 2 | X | 9 | 6 | 1 | 2 | 7 |

| | CAN0338-339ana | CAN0504ana | CAN0504ana2 | CAN0509ana | CAN0577ana | CAN0716ana |
|---|---|---|---|---|---|---|
| ANUCS304ana | | | | | | |
| ANUCS501ana | | | | | | |
| ANUCS501ana2 | | | | | | |
| C11-CANN1ana2 | | | | | | |
| CAN0032ana | | | | | | |
| CAN0055ana4 | | | | | | |
| CAN0067ana | | | | | | |
| CAN0085ana2 | | | | | | |
| CAN0089ana | | | | | | |
| CAN0097ana | | | | | | |
| CAN0103ana | | | | | | |
| CAN0111ana | | | | | | |
| CAN0111ana2 | | | | | | |
| CAN0124ana | | | | | | |
| CAN0138ana2 | | | | | | |
| CAN0156ana | | | | | | |
| CAN0180ana | | | | | | |
| CAN0202ana2 | | | | | | |
| CAN0250ana | | | | | | |
| CAN0271ana | | | | | | |
| CAN0291ana | | | | | | |
| CAN0338-339ana | | | | | | |
| CAN0504ana | | | | | | |
| CAN0504ana2 | | | | | | |
| CAN0509ana | 1 | 6 | 7 | | | |
| CAN0577ana | | 6 | 7 | >10 | | |
| CAN0716ana | 1 | 6 | 4 | >10 | >10 | |
| CAN0716ana2 | | | 3 | 3 | 3 | |
| CAN0717ana | | 6 | 3 | >10 | >10 | >10 |
| CAN0802ana | | 6 | 7 | >10 | >10 | >10 |
| CAN0907ana | | 6 | 7 | >10 | >10 | >10 |
| CAN1409ana | | | | 1 | | |
| CAN1539ana | 1 | | | >10 | 1 | 6 |
| CAN1539ana2 | | 6 | 7 | >10 | >10 | >10 |
| CAN1832ana | | | | >10 | 1 | 4 |
| CAN2418ana | | | | 1 | | |
| CAN2550ana2 | 1 | | | 1 | | 1 |
| CAN2829ana | | | | | | 1 |
| CAN2846ana | | | | 2 | | 2 |
| CAN2902ana | | | | | | 1 |
| CAN3008ana | | 6 | 7 | >10 | >10 | >10 |
| DO2-CANN1ana | | | | 3 | 3 | 4 |
| E07-CANN1ana2 | | | | >10 | 5 | 6 |
| E07-CANN1ana4 | | 6 | 7 | >10 | >10 | >10 |
| H06-CANN2ana | 1 | 2 | | >10 | 7 | >10 |
| LG | 5 | 2 | 2 | 1 | 5 | 3 |

| | CAN0716ana2 | CAN0717ana | CAN0802ana | CAN0907ana | CAN1409ana | CAN1539ana |
|---|---|---|---|---|---|---|
| ANUCS304ana | | | | | | |
| ANUCS501ana | | | | | | |
| ANUCS501ana2 | | | | | | |
| C11-CANN1ana2 | | | | | | |
| CAN0032ana | | | | | | |
| CAN0055ana4 | | | | | | |
| CAN0067ana | | | | | | |
| CAN0085ana2 | | | | | | |
| CAN0089ana | | | | | | |
| CAN0097ana | | | | | | |
| CAN0103ana | | | | | | |
| CAN0111ana | | | | | | |
| CAN0111ana2 | | | | | | |
| CAN0124ana | | | | | | |
| CAN0138ana2 | | | | | | |
| CAN0156ana | | | | | | |
| CAN0180ana | | | | | | |
| CAN0202ana2 | | | | | | |
| CAN0250ana | | | | | | |
| CAN0271ana | | | | | | |
| CAN0291ana | | | | | | |
| CAN0338-339ana | | | | | | |
| CAN0504ana | | | | | | |
| CAN0504ana2 | | | | | | |
| CAN0509ana | | | | | | |
| CAN0577ana | | | | | | |
| CAN0716ana | | | | | | |
| CAN0716ana2 | | | | | | |
| CAN0717ana | | | | | | |
| CAN0802ana | 4 | >10 | | | | |
| CAN0907ana | 4 | >10 | >10 | | | |
| CAN1409ana | | | 1 | 1 | | |
| CAN1539ana | | | >10 | >10 | 1 | |
| CAN1539ana2 | 4 | >10 | >10 | >10 | | |
| CAN1832ana | | | >10 | >10 | 1 | >10 |
| CAN2418ana | | | 1 | | | |
| CAN2550ana2 | | | | | | 1 |
| CAN2829ana | | | 1 | 1 | | |
| CAN2846ana | | | 5 | 7 | | 5 |
| CAN2902ana | | | | 1 | | 1 |
| CAN3008ana | 4 | >10 | >10 | >10 | 1 | >10 |
| DO2-CANN1ana | | 1 | 3 | 3 | | |
| E07-CANN1ana2 | 1 | 2 | >10 | >10 | 1 | >10 |
| E07-CANN1ana4 | 4 | 10 | >10 | >10 | | |
| H06-CANN2ana | | 5 | >10 | >10 | 1 | >10 |
| LG | 3 | 3 | 7 | 2 | 5 | 6 |
| | CAN1539ana2 | CAN1832ana | CAN2418ana | CAN2550ana2 | CAN2829ana | CAN2846ana |
| ANUCS304ana | | | | | | |
| ANUCS501ana | | | | | | |
| ANUCS501ana2 | | | | | | |
| C11-CANN1ana2 | | | | | | |
| CAN0032ana | | | | | | |
| CAN0055ana4 | | | | | | |
| CAN0067ana | | | | | | |
| CAN0085ana2 | | | | | | |
| CAN0089ana | | | | | | |
| CAN0097ana | | | | | | |
| CAN0103ana | | | | | | |
| CAN0111ana | | | | | | |
| CAN0111ana2 | | | | | | |
| CAN0124ana | | | | | | |
| CAN0138ana2 | | | | | | |
| CAN0156ana | | | | | | |
| CAN0180ana | | | | | | |
| CAN0202ana2 | | | | | | |
| CAN0250ana | | | | | | |
| CAN0271ana | | | | | | |
| CAN0291ana | | | | | | |
| CAN0338-339ana | | | | | | |
| CAN0504ana | | | | | | |
| CAN0504ana2 | | | | | | |
| CAN0509ana | | | | | | |
| CAN0577ana | | | | | | |
| CAN0716ana | | | | | | |
| CAN0716ana2 | | | | | | |
| CAN0717ana | | | | | | |
| CAN0802ana | | | | | | |
| CAN0907ana | | | | | | |
| CAN1409ana | | | | | | |
| CAN1539ana | | | | | | |
| CAN1539ana2 | | | | | | |
| CAN1832ana | | | | | | |
| CAN2418ana | | | | | | |
| CAN2550ana2 | | | | | | |
| CAN2829ana | | 2 | | | | |
| CAN2846ana | | 6 | | | 1 | |
| CAN2902ana | | | | | | 1 |
| CAN3008ana | >10 | >10 | | | | 7 |
| DO2-CANN1ana | 3 | | | | | |
| E07-CANN1ana2 | 4 | >10 | | | | 2 |
| E07-CANN1ana4 | >10 | | | | | |
| H06-CANN2ana | 5 | >10 | | 1 | 1 | 2 |
| LG | 6 | 1 | 5 | 9 | 6 | 9 |

| | | | DO2- | E07- | E07- | H06- | |
|---|---|---|---|---|---|---|---|
| | CAN2902ana | CAN3008ana | CANN1ana | CANN1ana2 | CANN1ana4 | CANN2ana | LG |
| ANUCS304ana | | | | | | | 8 |
| ANUCS501ana | | | | | | | X |
| ANUCS501ana2 | | | | | | | X |
| C11-CANN1ana2 | | | | | | | 7 |
| CAN0032ana | | | | | | | 2 |
| CAN0055ana4 | | | | | | | 8 |
| CAN0067ana | | | | | | | 4 |
| CAN0085ana2 | | | | | | | 4 |
| CAN0089ana | | | | | | | 9 |
| CAN0097ana | | | | | | | 4 |
| CAN0103ana | | | | | | | 3 |
| CAN0111ana | | | | | | | X |
| CAN0111ana2 | | | | | | | X |
| CAN0124ana | | | | | | | 1 |
| CAN0138ana2 | | | | | | | 2 |
| CAN0156ana | | | | | | | X |
| CAN0180ana | | | | | | | 9 |
| CAN0202ana2 | | | | | | | 6 |
| CAN0250ana | | | | | | | 1 |
| CAN0271ana | | | | | | | 2 |
| CAN0291ana | | | | | | | 7 |
| CAN0338-339ana | | | | | | | 5 |
| CAN0504ana | | | | | | | 2 |
| CAN0504ana2 | | | | | | | 2 |
| CAN0509ana | | | | | | | 1 |
| CAN0577ana | | | | | | | 5 |
| CAN0716ana | | | | | | | 3 |
| CAN0716ana2 | | | | | | | 3 |
| CAN0717ana | | | | | | | 3 |
| CAN0802ana | | | | | | | 7 |
| CAN0907ana | | | | | | | 2 |
| CAN1409ana | | | | | | | 5 |
| CAN1539ana | | | | | | | 6 |
| CAN1539ana2 | | | | | | | 6 |
| CAN1832ana | | | | | | | 1 |
| CAN2418ana | | | | | | | 5 |
| CAN2550ana2 | | | | | | | 9 |
| CAN2829ana | | | | | | | 6 |
| CAN2846ana | | | | | | | 9 |
| CAN2902ana | | | | | | | 5 |
| CAN3008ana | | | | | | | 8 |
| DO2-CANN1ana | | 3 | | | | | 2 |
| E07-CANN1ana2 | | >10 | 2 | | | | 5 |
| E07-CANN1ana4 | | >10 | | | | | 5 |
| H06-CANN2ana | 1 | >10 | 2 | >10 | 3 | | 4 |
| LG | 5 | 8 | 2 | 5 | 5 | 4 | |

TABLE 36a
Cannabis Genomic DNA Samples Tested with multiplex primer
pair combinations 20 and 21, 34 and 35 or 36.

| Plant | | | | |
|---|---|---|---|---|
| Sample | | | | Genotype |
| Number | Name | Type | Multiplex | Number |
| 1 | Hash Passion | High THC | MPLX20-21, | 19255 |
| 2 | Guatemala | Land Race | MPLX20-21 | 19256 |
| 3 | Malawi Gold | Land Race | MPLX20-21 | 19257 |
| 4 | Hindu Kush | High THC | MPLX20-21 | 19258 |
| 5 | African Buzz | High THC | MPLX20-21, MPLX34-35 | 19235 |
| 6 | Wild Thailand | Land Race | MPLX20-21, MPLX34-35 | 19251 |
| 7 | South African Sativa | Land Race | MPLX20-21, MPLX34-35 | 19250 |
| 8 | Ruderalis Indica | Land Race | MPLX20-21, MPLX34-35 | 19259 |
| 9 | Agent Orange | High THC | MPLX20-21 | 19254 |
| 10 | Bubba Kush | High THC | MPLX20-21 | 19278 |
| 11 | Neville's Haze | High THC | MPLX20-21 | 19279 |
| 12 | Sweet Skunk | High THC | MPLX20-21 | 19280 |
| 13 | Enemy of the State | Balanced 1:1 | MPLX20-21 | 19265 |
| 14 | CBG Shiatzu | High THC | MPLX20-21 | 19233 |
| 15 | Tangerine Dream | High THC | MPLX20-21 | 19165 |
| 16 | Kosher Kush | High THC | MPLX20-21 | 19113 |
| 17 | Otto | High CBD | MPLX20-21 | 19229 |
| 18 | Nuken | High THC | MPLX20-21 | 19166 |
| 19 | Gods Green Crack | High THC | MPLX20-21 | 19234 |
| 20 | Bubba Kush | High THC | MPLX20-21 | 19224 |
| 21 | Purple Kush | High THC | MPLX20-21, MPLX34-35, | 19108 |
| | | | MPLX36 | |
| 22 | Lemon Skunk | High THC | MPLX20-21 | 19281 |
| 23 | Blue Dream | High THC | MPLX20-21 | 19282 |
| 24 | Finola | Hemp | MPLX20-21, MPLX34-35 | 19237 |
| 25 | Relevo | High THC | MPLX62 | 19108 |
| 26 | Tranquillum | High THC | MPLX62 | 19108 |
| 27 | Potentia | High THC | MPLX62 | 19247 |
| 28 | Socialis | High THC | MPLX62 | 19247 |
| 29 | Agent Orange | High THC | MPLX34-35, MPLX36 | 19254 |
| 30 | Agent Orange | High THC | MPLX36 | 19254 |
| 31 | Piccolo | Hemp | MPLX20-21, MPLX34-35 | 19261 |
| 32 | Banana Split | High THC | MPLX62 | 19111 |
| 33 | Zombie Kush | High THC | MPLX62 | 19111 |
| 34 | 2709-061 | Outcross | MPLX20-21 | 19283 |
| 35 | Avidekel | High CBD | MPLX20-21 | 19204 |
| 36 | Cannatonic | High CBD | MPLX20-21 | 19101 |
| 37 | Cannatonic | High CBD | MPLX20-21 | 19101 |
| 38 | 1212-001 | Hemp | MPLX20-21, MPLX34-35 | 19236 |
| 39 | 1212-005 | Hemp | MPLX20-21 | 19267 |
| 40 | Tropicana | High THC | MPLX20-21 | 19224 |
| 41 | 1402-002 | High CBD | MPLX20-21 | 19276 |
| 42 | 1402-003 | High CBD | MPLX20-21 | 19277 |
| 43 | Cannatonic | High CBD | MPLX20-21 | 19101 |
| 44 | 2111-001 | Hemp | MPLX20-21 | 19268 |
| 45 | 2111-002 | Hemp | MPLX20-21 | 19269 |
| 46 | Starting material | Hemp | MPLX20-21 | 19270 |
| 47 | LA Confidential | High THC | MPLX20-21 | 19102 |
| 48 | MK Ultra | High THC | MPLX20-21 | 19106 |
| 49 | Kosher Kush | High THC | MPLX20-21 | 19113 |
| 50 | LA Confidential | High THC | MPLX20-21 | 19102 |
| 51 | Krypt OG Melon | High THC | MPLX20-21 | 19112 |
| 52 | Chocolope | High THC | MPLX20-21 | 19173 |
| 53 | 19-004 | Balanced 1:1 | MPLX20-21 | 19220 |
| 54 | Mk Ultra | High THC | MPLX20-21 | 19106 |
| 55 | Blue Dream | High THC | MPLX20-21 | 19208 |
| 56 | Banana Split | High THC | MPLX20-21 | 19111 |
| 57 | Equiposa | Balanced 1:1 | MPLX20-21 | 19114 |
| 58 | D190822A | High THC | MPLX20-21 | 19164 |
| 59 | L190819A | High THC | MPLX20-21 | 19128 |
| 60 | LA Confidential | High THC | MPLX20-21 | 19102 |
| 61 | LA Confidential | High THC | MPLX20-21 | 19102 |
| 62 | 20000027 | High CBD | MPLX20-21 | 19101 |
| 63 | Sour Tangie | High THC | MPLX20-21 | 19170 |
| 64 | F190904A | High THC | MPLX20-21 | 19107 |
| 65 | 1807-002 | Hemp | MPLX20-21 | 19271 |
| 66 | Zombie Kush | High THC | MPLX20-21 | 19105 |
| 67 | Chocolope | High THC | MPLX20-21 | 19104 |
| 68 | LA Confidential | High THC | MPLX20-21 | 19102 |
| 69 | LA Confidential | High THC | MPLX20-21 | 19102 |
| 70 | LA Confidential | High THC | MPLX20-21 | 19102 |
| 71 | Trutiva | High CBD | MPLX20-21 | 19187 |
| 72 | Chocolope | High THC | MPLX20-21, MPLX34-35 | 19104 |
| 73 | Zombie Kush | High THC | MPLX20-21, MPLX34-35 | 19105 |
| 74 | LA Confidential | High THC | MPLX20-21 | 19102 |
| 75 | Ghost Train Haze | High THC | MPLX20-21 | 19103 |
| 76 | C190918A | Balanced 1:1 | MPLX20-21 | 19107 |
| 77 | Chase | Hemp | MPLX20-21 | 19272 |
| 78 | Alan | Hemp | MPLX20-21 | 19273 |
| 79 | Tower - Mother Tissue | High CBD | MPLX20-21 | 19101 |
| 80 | Tower - Flowering Plant | High CBD | MPLX20-21 | 19101 |
| | Tissue | | | |
| 81 | Cannatonic - Mother | High CBD | MPLX20-21 | 19101 |
| | Tissue | | | |
| 82 | Cannatonic - Flowering | High CBD | MPLX20-21 | 19101 |
| | Plant Tissue | | | |
| 83 | Snow Dome - Mother | High THC | MPLX20-21 | 19102 |
| | Tissue | | | |
| 84 | Snow Dome - Flowering | High THC | MPLX20-21 | 19102 |
| | Plant Tissue | | | |
| 85 | Thor - Mother Tissue | High THC | MPLX20-21 | 19103 |
| 86 | Thor - Flowering Plant | High THC | MPLX20-21 | 19103 |
| | Tissue | | | |
| 87 | CBD Shark | Balanced 1:1 | MPLX20-21 | 19167 |
| 88 | Grease Monkey | High THC | MPLX20-21 | 19178 |
| 89 | Critical Haze | High THC | MPLX20-21 | 19188 |
| 90 | Acapulco Gold | High THC | MPLX20-21 | 19160 |
| 91 | B190925A | High THC | MPLX20-21 | 19129 |
| 92 | LA Confidential | High THC | MPLX20-21 | 19102 |
| 93 | LA Confidential | High THC | MPLX20-21 | 19102 |
| 94 | LA Confidential | High THC | MPLX20-21 | 19102 |
| 95 | Banana Split | High THC | MPLX20-21 | 19111 |
| 96 | Equiposa | Balanced 1:1 | MPLX20-21 | 19114 |
| 97 | LA Confidential | High THC | MPLX20-21 | 19102 |
| 98 | php-19-02M | Hemp | MPLX20-21 | 19115 |
| 99 | php-18-16M | Hemp | MPLX20-21 | 19115 |
| 100 | Chase-Finola | Hemp | MPLX20-21 | 19274 |
| 10 | Alan-X59 | Hemp | MPLX20-21 | 19275 |
| 102 | Voluptas | High THC | MPLX20-21 | 19176 |
| 103 | Mk Ultra | High THC | MPLX20-21 | 19106 |
| 104 | Krypt OG Melon | High THC | MPLX20-21 | 19112 |
| 105 | CBD Skunk | Balanced 1:1 | MPLX20-21 | 19266 |
| 106 | 1207-001 | High THC | MPLX20-21, MPLX34-35 | 19133 |
| 107 | 1207-002 | High THC | MPLX20-21, MPLX34-35 | 19132 |
| 108 | 1207-003 | High THC | MPLX20-21, MPLX34-35 | 19135 |
| 109 | 1207-004 | High THC | MPLX20-21, MPLX34-35 | 19130 |
| 110 | 1207-005 | Balanced 1:1 | MPLX20-21, MPLX34-35 | 19126 |
| 111 | 1207-006 | High THC | MPLX20-21, MPLX34-35 | 19135 |
| 112 | LA Confidential | High THC | MPLX20-21 | 19102 |
| 113 | CSCJ | High THC | MPLX20-21 | 19134 |
| 114 | CSC | High THC | MPLX20-21 | 19131 |
| 115 | CSCL | High THC | MPLX20-21 | 19128 |
| 116 | CSCB | High THC | MPLX20-21 | 19129 |
| 117 | CSCH | High THC | MPLX20-21 | 19107 |
| 118 | CSCK | High THC | MPLX20-21 | 19164 |
| 119 | 007 Self #7 | Self cross | MPLX20-21, MPLX34-35, | 19138 |
| | | | MPLX36 | |
| 120 | 17 × 5 #2 | Outcross | MPLX20-21, MPLX34-35, | 19139 |
| | | | MPLX36 | |
| 121 | 8 × 3-3 #2 | Outcross | MPLX20-21, MPLX34-35, | 19140 |
| | | | MPLX36 | |
| 122 | 8 × 3-3 #4 | Outcross | MPLX20-21, MPLX34-35, | 19141 |
| | | | MPLX36 | |
| 123 | EF3 self #10 | Self cross | MPLX20-21, MPLX34-35 | 19142 |
| 124 | EF3 self #13 | Self cross | MPLX20-21, MPLX34-35 | 19143 |
| 125 | EF3 self #15 | Self cross | MPLX20-21, MPLX34-35 | 19209 |
| 126 | EF3 self #16 | Self cross | MPLX20-21, MPLX34-35 | 19144 |
| 127 | EF3 self #2 | Self cross | MPLX20-21, MPLX34-35 | 19145 |
| 128 | EF3 self #4 | Self cross | MPLX20-21, MPLX34-35 | 19210 |
| 129 | EF3 self #8 | Self cross | MPLX20-21, MPLX34-35 | 19146 |
| 130 | LC-009 | Unknown | MPLX20-21, MPLX34-35 | 19147 |
| 131 | LC-010 | Unknown | MPLX20-21, MPLX34-35 | 19148 |
| 132 | MS-002 | Unknown | MPLX20-21, MPLX34-35 | 19149 |
| 133 | MS17-388 | Unknown | MPLX20-21, MPLX34-35 | 19150 |
| 134 | MS9-934 | Unknown | MPLX20-21, MPLX34-35 | 19151 |
| 135 | PK | High THC | MPLX20-21, MPLX34-35 | 19108 |
| 136 | Purple Kush | High THC | MPLX20-21, MPLX34-35 | 19152 |
| 137 | S48-018 | Unknown | MPLX20-21, MPLX34-35 | 19153 |
| 138 | S9-003 | Unknown | MPLX20-21, MPLX34-35 | 19154 |
| 139 | S9-007 | Unknown | MPLX20-21, MPLX34-35 | 19147 |
| 140 | Shark | High CBD | MPLX20-21, MPLX34-35 | 19155 |
| 141 | Stem | Hemp | MPLX20-21, MPLX34-35 | 19156 |
| 142 | USO | Hemp | MPLX20-21, MPLX34-35 | 19157 |
| 143 | Med1-1 | Balanced 1:1 | MPLX20-21, MPLX34-35 | 19186 |
| 144 | OG Skunk | High THC | MPLX20-21, MPLX34-35 | 19181 |
| 145 | Acapulco Gold | High THC | MPLX20-21, MPLX34-35 | 19160 |
| 146 | Alaska | High THC | MPLX20-21, MPLX34-35 | 19174 |
| 147 | Alien Chemdawg | High THC | MPLX20-21, MPLX34-35 | 19132 |
| 148 | Amnesia | Balanced 1:1 | MPLX20-21, MPLX34-35 | 19114 |
| 149 | AURORA1 | High THC | MPLX20-21, MPLX34-35 | 19102 |
| 150 | AURORA23 | High THC | MPLX20-21, MPLX34-35 | 19103 |
| 151 | AURORA288 | High THC | MPLX20-21, MPLX34-35 | 19106 |
| 152 | Sour Tangie | High THC | MPLX20-21, MPLX34-35 | 19170 |
| 153 | Banana Split | High THC | MPLX20-21, MPLX34-35 | 19111 |
| 154 | AURORA43 | High THC | MPLX20-21, MPLX34-35 | 19168 |
| 155 | Chocolope | High THC | MPLX20-21, MPLX34-35 | 19173 |
| 156 | AURORA44 | High THC | MPLX20-21, MPLX34-35 | 19190 |
| 157 | AURORA68 | High CBD | MPLX20-21, MPLX34-35 | 19101 |
| 158 | AURORA78 | High THC | MPLX20-21, MPLX34-35 | 19208 |
| 159 | Avidekel | High CBD | MPLX20-21, MPLX34-35 | 19204 |
| 160 | Blue Cheese | High THC | MPLX20-21, MPLX34-35 | 19188 |
| 161 | Blue Dream | High THC | MPLX20-21, MPLX34-35 | 19205 |
| 162 | BlueJackCity-30 | High THC | MPLX20-21, MPLX34-35 | 19206 |
| 163 | BlueJackCity-32 | High THC | MPLX20-21, MPLX34-35 | 19201 |
| 164 | BlueJackCity-35 | High THC | MPLX20-21, MPLX34-35 | 19162 |
| 165 | Blue Lime Pie | High THC | MPLX20-21, MPLX34-35 | 19200 |
| 166 | Buddha's | High CBD | MPLX20-21, MPLX34-35 | 19180 |
| 167 | Northern Tonic | Balanced 1:1 | MPLX20-21, MPLX34-35 | 19169 |
| 168 | CBD Shark | Balanced 1:1 | MPLX20-21, MPLX34-35 | 19167 |
| 169 | Chocolope | High THC | MPLX20-21, MPLX34-35 | 19171 |
| 170 | Critical Haze | High THC | MPLX20-21, MPLX34-35 | 19188 |
| 171 | Critical Kush | High THC | MPLX20-21, MPLX34-35 | 19177 |
| 172 | CSC | High THC | MPLX20-21, MPLX34-35, | 19131 |
| | | | MPLX36 | |
| 173 | CSCB | High THC | MPLX20-21, MPLX34-35 | 19129 |
| 174 | CSCH | High THC | MPLX20-21, MPLX34-35 | 19107 |
| 175 | CSCJ | High THC | MPLX20-21, MPLX34-35, | 19134 |
| | | | MPLX36 | |
| 176 | CSCK | High THC | MPLX20-21, MPLX34-35 | 19164 |
| 177 | CSCL | High THC | MPLX20-21, MPLX34-35 | 19128 |
| 178 | Luminarium ™ | High THC | MPLX20-21, MPLX34-35 | 19191 |
| 179 | EranAlmog-1 | High THC | MPLX20-21, MPLX34-35 | 19183 |
| 180 | God'sGreenCrack-D | High THC | MPLX20-21, MPLX34-35 | 19199 |
| 181 | God'sGreenCrack-F | High THC | MPLX20-21, MPLX34-35 | 19161 |
| 182 | God'sGreenCrack-I | High THC | MPLX20-21, MPLX34-35 | 19202 |
| 183 | Grease Monkey | High THC | MPLX20-21, MPLX34-35 | 19178 |
| 184 | Hindu Kush | High THC | MPLX20-21, MPLX34-35 | 19108 |
| 185 | IslandSweetSkunk-1 | High THC | MPLX20-21, MPLX34-35 | 19176 |
| 186 | Juanita Lagrimosa | High THC | MPLX20-21, MPLX34-35 | 19192 |
| 187 | Kosher Kush | High THC | MPLX20-21, MPLX34-35 | 19158 |
| 188 | Lavender-23 | High THC | MPLX20-21, MPLX34-35 | 19175 |
| 189 | LSD-1 | High THC | MPLX20-21, MPLX34-35 | 19203 |
| 190 | Med12-4 | High THC | MPLX20-21, MPLX34-35 | 19163 |
| 191 | Med14-4 | High THC | MPLX20-21, MPLX34-35 | 19193 |
| 192 | Med1-5 | High CBD | MPLX20-21, MPLX34-35 | 19185 |
| 193 | Med3-9 | High CBD | MPLX20-21, MPLX34-35 | 19194 |
| 194 | Midnight-1 | Balanced 1:1 | MPLX20-21, MPLX34-35 | 19195 |
| 195 | Nuken | High THC | MPLX20-21, MPLX34-35 | 19166 |
| 196 | PackCheese-6 | High THC | MPLX20-21, MPLX34-35 | 19182 |
| 197 | Pink Kush | High THC | MPLX20-21, MPLX34-35 | 19159 |
| 198 | Romi-1 | Unknown | MPLX20-21, MPLX34-35 | 19179 |
| 199 | Skywalker | High THC | MPLX20-21, MPLX34-35 | 19184 |
| 200 | Strawberry Short | High THC | MPLX20-21, MPLX34-35 | 19172 |
| | Cookies | | | |
| 201 | SugarBlackRose-1 | High THC | MPLX20-21, MPLX34-35 | 19189 |
| 202 | Tangerine Dream | High THC | MPLX20-21, MPLX34-35 | 19165 |
| 203 | Tangerine Dream | High THC | MPLX20-21, MPLX34-35 | 19196 |
| 204 | Trutiva | High CBD | MPLX20-21, MPLX34-35 | 19187 |
| 205 | UKCheese-1 | High THC | MPLX20-21, MPLX34-35 | 19197 |
| 206 | White Widow | High THC | MPLX20-21, MPLX34-35 | 19188 |
| 207 | Yakusha-10 | High THC | MPLX20-21, MPLX34-35 | 19198 |
| 208 | Yakusha-11 | High THC | MPLX20-21, MPLX34-35 | 19198 |
| 209 | LA Confidential | High THC | MPLX20-21, MPLX36 | 19102 |
| 210 | LA Confidential | High THC | MPLX20-21 | 19102 |
| 211 | LA Confidential | High THC | MPLX20-21 | 19102 |
| 212 | Hurkle | Balanced 1:1 | MPLX20-21, MPLX34-35 | 19211 |
| 213 | Sour Diesel/Odin | High THC | MPLX20-21, MPLX34-35 | 19212 |
| 214 | OG Grape Krypt | High THC | MPLX20-21, MPLX34-35 | 19213 |
| 215 | OG Grape Krypt | High THC | MPLX20-21, MPLX34-35 | 19214 |
| 216 | Blackwater | High THC | MPLX20-21, MPLX34-35 | 19215 |
| 217 | Querkle | High THC | MPLX20-21, MPLX34-35 | 19216 |
| 218 | Zombie Kush | High THC | MPLX20-21, MPLX34-35 | 19111 |
| 219 | Zombie Kush | High THC | MPLX20-21, MPLX34-35 | 19217 |
| 220 | Zombie Kush | High THC | MPLX20-21, MPLX34-35 | 19218 |
| 221 | Dairy Queen | High THC | MPLX20-21, MPLX34-35 | 19219 |
| 222 | Cannatonic 1:1 | Balanced 1:1 | MPLX20-21, MPLX34-35 | 19220 |
| 223 | Grand Daddy Purp | High THC | MPLX20-21, MPLX34-35 | 19221 |
| 224 | Pennywise | Balanced 1:1 | MPLX20-21, MPLX34-35 | 19222 |
| 225 | Heisenberg Kush | High THC | MPLX20-21, MPLX34-35 | 19223 |
| 226 | BC Rockstar | High THC | MPLX20-21, MPLX34-35 | 19224 |
| 227 | Bubba Kush | High THC | MPLX20-21, MPLX34-35 | 19224 |
| 228 | CBG Shiatzu | High THC | MPLX20-21, MPLX34-35 | 19233 |
| 229 | Dancehall | High CBD | MPLX20-21, MPLX34-35 | 19226 |
| 230 | Devil Fruit | High THC | MPLX20-21, MPLX34-35 | 19227 |
| 231 | God's Green Crack | High THC | MPLX20-21, MPLX34-35 | 19234 |
| 232 | Gorilla Glue | High THC | MPLX20-21, MPLX34-35 | 19228 |
| 233 | Kosher Kush | High THC | MPLX20-21, MPLX34-35 | 19113 |
| 234 | Otto | High CBD | MPLX20-21, MPLX34-35 | 19229 |
| 235 | Romulan Diesel | High THC | MPLX20-21, MPLX34-35 | 19230 |
| 236 | Sour Jack | High THC | MPLX20-21, MPLX34-35 | 19231 |
| 237 | Blueberry Lambsbread | High THC | MPLX20-21, MPLX34-35 | 19232 |
| 238 | LA Confidential | High THC | MPLX20-21, MPLX34-35 | 19102 |
| 239 | LA Confidential | High THC | MPLX20-21 | 19102 |
| 240 | LA Confidential | High THC | MPLX20-21, MPLX34-35 | 19102 |
| 241 | LA Confidential | High THC | MPLX20-21, MPLX34-35 | 19102 |
| 242 | Kanteris | High THC | MPLX34-35 | 19238 |
| 243 | Bellis | High THC | MPLX34-35 | 19239 |
| 244 | Cerebri | High THC | MPLX34-35 | 19240 |
| 245 | Dorit | High THC | MPLX34-35 | 19241 |
| 246 | Erez | High THC | MPLX34-35 | 19241 |
| 247 | Girl Scout Cookies | High THC | MPLX34-35 | 19242 |
| 248 | Remissio | High THC | MPLX34-35 | 19243 |
| 249 | Operari | High THC | MPLX34-35 | 19108 |
| 250 | Relevo | High THC | MPLX34-35 | 19108 |
| 251 | Tranquillum | High THC | MPLX34-35 | 19108 |
| 252 | Claritas | High THC | MPLX34-35 | 19245 |
| 253 | Or | High THC | MPLX34-35 | 19246 |
| 254 | Potentia | High THC | MPLX34-35 | 19247 |
| 255 | Durabilis | High THC | MPLX34-35 | 19248 |
| 256 | 2913-015 | Unknown | MPLX34-35 | 19249 |
| 257 | Socialis | High THC | MPLX34-35 | 19247 |
| 258 | LA Confidential | High THC | MPLX34-35 | 19102 |
| 259 | Ghost Train Haze | High THC | MPLX34-35 | 19103 |
| 260 | Ghost Train Haze | High THC | MPLX34-35 | 19103 |
| 261 | Banana Split | High THC | MPLX34-35 | 19111 |
| 262 | Banana Split | High THC | MPLX34-35 | 19111 |
| 263 | Banana Split | High THC | MPLX34-35 | 19111 |
| 264 | Cannatonic | High CBD | MPLX34-35 | 19101 |
| 265 | CSCJ | High THC | MPLX34-35 | 19134 |
| 266 | CSC | High THC | MPLX34-35 | 19131 |
| 267 | CSC | High THC | MPLX34-35 | 19131 |
| 268 | CSCJ | High THC | MPLX34-35 | 19134 |
| 269 | CSCJ | High THC | MPLX34-35 | 19134 |
| 270 | CSCL | High THC | MPLX34-35 | 19128 |
| 271 | CSC | High THC | MPLX34-35 | 19131 |
| 272 | CSC | High THC | MPLX34-35 | 19131 |
| 273 | LA Confidential | High THC | MPLX34-35, MPLX36 | 19102 |
| 274 | BC Rockstar | High THC | MPLX34-35 | 19224 |
| 275 | Bubba Kush | High THC | MPLX34-35 | 19224 |
| 276 | CBD Shark | Balanced 1:1 | MPLX34-35 | 19167 |
| 277 | Grapefruit | High THC | MPLX34-35 | 19252 |
| 278 | Medical Glue | High THC | MPLX34-35 | 19253 |
| 279 | Zombie Kush | High THC | MPLX34-35 | 19111 |
| 280 | Banana Split | High THC | MPLX34-35 | 19111 |
| 281 | CSCJ | High THC | MPLX36 | 19134 |
| 282 | CSC | High THC | MPLX36 | 19131 |
| 283 | CSC | High THC | MPLX36 | 19131 |
| 284 | CSCJ | High THC | MPLX36 | 19134 |
| 285 | CSC | High THC | MPLX36 | 19131 |
| 286 | Cannatonic | High CBD | MPLX36 | 19101 |
| 287 | Cannatonic | High CBD | MPLX36 | 19101 |
| 288 | Cannatonic | High CBD | MPLX36 | 19101 |
| 289 | Ghost train Haze | High THC | MPLX36 | 19103 |
| 290 | Ghost train Haze | High THC | MPLX36 | 19103 |
| 291 | 1209-006 | High THC | MPLX36 | 19260 |
| 292 | 1209-007 | High THC | MPLX36 | 19260 |
| 293 | 1209-008 | High THC | MPLX36 | 19260 |
| 294 | Banana Split | High THC | MPLX36 | 19111 |
| 295 | Banana Split | High THC | MPLX36 | 19111 |
| 296 | Banana Split | High THC | MPLX36 | 19111 |
| 297 | Banana Split | High THC | MPLX36 | 19111 |
| 298 | Banana Split | High THC | MPLX36 | 19111 |
| 299 | Pink Kush | High THC | MPLX36 | 19159 |
| 300 | LA Confidential | High THC | MPLX36 | 19102 |
| 301 | LA Confidential | High THC | MPLX36 | 19102 |
| 302 | LA Confidential | High THC | MPLX36 | 19102 |
| 303 | LA Confidential | High THC | MPLX36 | 19102 |
| 304 | LA Confidential | High THC | MPLX36 | 19102 |

TABLE 36b
Cannabis Genomic DNA Samples Tested with multiplex
primer pair combinations 64, 65, 66, 70, and 91.

| Plant | | | | |
|---|---|---|---|---|
| Sample | | | | Geno |
| Number | Name | Type | Multiplex | Number |
| 1 | Hash Passion | High THC | MPLX20-21, MPLX70 | 19255 |
| 2 | Guatemala | Land Race | MPLX20-21, MPLX70 | 19256 |
| 3 | Malawi Gold | Land Race | MPLX20-21, MPLX62, MPLX70, | 19257 |
| | | | MPLX91 | |
| 4 | Hindu Kush | High THC | MPLX20-21, MPLX70 | 19258 |
| 5 | African Buzz | High THC | MPLX20-21, MPLX34-35, | 19235 |
| | | | MPLX70 | |
| 6 | Wild Thailand | Land Race | MPLX20-21, MPLX34-35, | 19251 |
| | | | MPLX70 | |
| 7 | S.A.S. (South African | Land Race | MPLX20-21, MPLX34-35, | 19250 |
| | Sativa) | | MPLX70 | |
| 8 | Ruderalis Indica | Land Race | MPLX20-21, MPLX34-35, | 19259 |
| | | | MPLX62, MPLX70 | |
| 9 | Agent Orange | High THC | MPLX20-21, MPLX70 | 19254 |
| 10 | C10-Bubba Kush | High THC | MPLX20-21, MPLX70 | 19278 |
| 11 | Neville's Haze | High THC | MPLX20-21, MPLX70 | 19279 |
| 12 | Sweet Skunk | High THC | MPLX20-21, MPLX70 | 19280 |
| 13 | Enemy of the State | Balanced | MPLX20-21, MPLX70 | 19265 |
| 14 | Purple Kush | High THC | MPLX20-21, MPLX34-35, | 19108 |
| | | | MPLX36, MPLX62, MPLX64, | |
| | | | MPLX70, MPLX88, MPLX91 | |
| 15 | Lemon Skunk | High THC | MPLX20-21, MPLX70 | 19281 |
| 16 | Blue Dream | High THC | MPLX20-21 | 19282 |
| 17 | Finola | Hemp | MPLX20-21, MPLX34-35, | 19237 |
| | | | MPLX62, MPLX70, MPLX88, | |
| | | | MPLX91 | |
| 18 | Nepal Jam | High THC | MPLX70 | 19238 |
| 19 | Relevo | High THC | MPLX62 | 19108 |
| 20 | Tranquillum | High THC | MPLX62 | 19108 |
| 21 | Potentia | High THC | MPLX62 | 19247 |
| 22 | Socialis | High THC | MPLX62 | 19247 |
| 23 | Agent Orange | High THC | MPLX34-35, MPLX36 | 19254 |
| 24 | Piccolo | Hemp | MPLX20-21, MPLX34-35, | 19261 |
| | | | MPLX62, MPLX64, MPLX70 | |
| 25 | Banana Split | High THC | MPLX62, MPLX70 | 19111 |
| 26 | Zombie Kush | High THC | MPLX62, MPLX70 | 19111 |
| 27 | CONE-01-SC | Hemp | MPLX20-21, MPLX34-35 | 19236 |
| 28 | Chocolope | High THC | MPLX20-21, MPLX34-35 | 19104 |
| 29 | Zombie Kush | High THC | MPLX20-21, MPLX34-35 | 19105 |
| 30 | Cannatonic | High CBD | MPLX88, MPLX91 | 19101 |
| 31 | Cannatonic | High CBD | MPLX20-21, MPLX34-35, | 19101 |
| | | | MPLX64 | |
| 32 | Ghost Train Haze | High THC | MPLX88, MPLX91 | 19103 |
| 33 | Grace | High THC | MPLX20-21, MPLX34-35, | 19133 |
| | | | MPLX70 | |
| 34 | Gems | High THC | MPLX20-21, MPLX34-35 | 19132 |
| 35 | Eldo | High THC | MPLX20-21, MPLX34-35, | 19135 |
| | | | MPLX70 | |
| 36 | 50 | High THC | MPLX20-21, MPLX34-35, | 19130 |
| | | | MPLX70 | |
| 37 | Moon | Balanced | MPLX20-21, MPLX34-35, | 19126 |
| | | | MPLX70 | |
| 38 | Meridian | High THC | MPLX20-21, MPLX34-35 | 19135 |
| 39 | LA Confidential | High THC | MPLX20-21 | 19102 |
| 40 | CSCJ | High CBD | MPLX20-21 | 19134 |
| 41 | CSC | High THC | MPLX20-21 | 19131 |
| 42 | CSCL | High THC | MPLX20-21 | 19128 |
| 43 | CSCB | High THC | MPLX20-21 | 19129 |
| 44 | CSCH | High THC | MPLX20-21 | 19107 |
| 45 | CSCK | High THC | MPLX20-21, MPLX91 | 19164 |
| 46 | 007 Self #7 | Self cross | MPLX20-21, MPLX34-35, | 19138 |
| | | | MPLX36 | |
| 47 | 17 × 5 #2 | Outcross | MPLX20-21, MPLX34-35, | 19139 |
| | | | MPLX36 | |
| 48 | 8 × 3-3 #2 | Outcross | MPLX20-21, MPLX34-35, | 19140 |
| | | | MPLX36 | |
| 49 | 8 × 3-3 #4 | Outcross | MPLX20-21, MPLX34-35, | 19141 |
| | | | MPLX36 | |
| 50 | EF3 self #10 | Self cross | MPLX20-21, MPLX34-35 | 19142 |
| 51 | EF3 self #13 | Self cross | MPLX20-21, MPLX34-35 | 19143 |
| 52 | EF3 self #15 | Self cross | MPLX20-21, MPLX34-35 | 19209 |
| 53 | EF3 self #16 | Self cross | MPLX20-21, MPLX34-35 | 19144 |
| 54 | EF3 self #2 | Self cross | MPLX20-21, MPLX34-35 | 19145 |
| 55 | EF3 self #4 | Self cross | MPLX20-21, MPLX34-35 | 19210 |
| 56 | EF3 self #8 | Self cross | MPLX20-21, MPLX34-35 | 19146 |
| 57 | LC-009 | Unknown | MPLX20-21, MPLX34-35 | 19147 |
| 58 | LC-010 | Unknown | MPLX20-21, MPLX34-35 | 19148 |
| 59 | MS-002 | Unknown | MPLX20-21, MPLX34-35 | 19149 |
| 60 | MS17-388 | Unknown | MPLX20-21, MPLX34-35 | 19150 |
| 61 | MS9-934 | Unknown | MPLX20-21, MPLX34-35 | 19151 |
| 62 | Purple Kush | High THC | MPLX20-21, MPLX34-35 | 19108 |
| 63 | Purple Kush_other | High THC | MPLX20-21, MPLX34-35 | 19152 |
| 64 | S48-018 | Unknown | MPLX20-21, MPLX34-35 | 19153 |
| 65 | S9-003 | Unknown | MPLX20-21, MPLX34-35 | 19154 |
| 66 | S9-007 | Unknown | MPLX20-21, MPLX34-35 | 19147 |
| 67 | Shark | High CBD | MPLX20-21, MPLX34-35, | 19155 |
| | | | MPLX64 | |
| 68 | Stem 240-3 | Hemp | MPLX20-21, MPLX34-35 | 19156 |
| 69 | hemp | Hemp | MPLX20-21, MPLX34-35 | 19157 |
| 70 | Vespera | Balanced | MPLX20-21, MPLX34-35, | 19186 |
| | | | MPLX70, MPLX91-91b | |
| 71 | OG Skunk | High THC | MPLX20-21, MPLX34-35, | 19181 |
| | | | MPLX70, MPLX91 | |
| 72 | Acapulco Gold | High THC | MPLX20-21, MPLX34-35, | 19160 |
| | | | MPLX70, MPLX91 | |
| 73 | Alaska | High THC | MPLX20-21, MPLX34-35, | 19174 |
| | | | MPLX64, MPLX70, MPLX91-91b | |
| 74 | Elevare/Peyto | High THC | MPLX20-21, MPLX34-35, | 19132 |
| | | | MPLX70, MPLX91-91b | |
| 75 | Equiposa | Balanced | MPLX20-21, MPLX34-35, | 19114 |
| | | | MPLX64, MPLX70, MPLX91-91b | |
| 76 | LA Confidential | High THC | MPLX20-21, MPLX34-35, | 19102 |
| | | | MPLX62, MPLX64, MPLX70, | |
| | | | MPLX91-91b | |
| 77 | Ghost Train Haze | High THC | MPLX20-21, MPLX34-35, | 19103 |
| | | | MPLX62, MPLX64, MPLX70, | |
| | | | MPLX91-91b | |
| 78 | MK Ultra | High THC | MPLX20-21, MPLX34-35, | 19106 |
| | | | MPLX70, MPLX91-91b | |
| 79 | Sour Tangie | High THC | MPLX20-21, MPLX34-35, | 19170 |
| | | | MPLX66, MPLX70, MPLX91 | |
| 80 | Banana Split | High THC | MPLX20-21, MPLX34-35, | 19111 |
| | | | MPLX64, MPLX66, MPLX70, | |
| | | | MPLX91 | |
| 81 | 91 Krypt OG-Melon | High THC | MPLX20-21, MPLX34-35, | 19168 |
| | | | MPLX62, MPLX64, MPLX66, | |
| | | | MPLX70, MPLX91 | |
| 82 | Chocolope | High THC | MPLX20-21, MPLX34-35, | 19173 |
| | | | MPLX62, MPLX64, MPLX66, | |
| | | | MPLX70, MPLX91 | |
| 83 | 91 Krypt OG - | High THC | MPLX20-21, MPLX34-35, | 19190 |
| | Chemdawg/Warwick# | | MPLX70, MPLX91 | |
| | 2 | | | |
| 84 | Cannatonic | High CBD | MPLX20-21, MPLX34-35, | 19101 |
| | | | MPLX62, MPLX66, MPLX70, | |
| | | | MPLX91 | |
| 85 | Blue Dream | High THC | MPLX20-21, MPLX34-35, | 19208 |
| | | | MPLX62, MPLX70, MPLX91-91b | |
| 86 | Avidekel | High CBD | MPLX20-21, MPLX34-35, | 19204 |
| | | | MPLX64, MPLX70, MPLX91-91b | |
| 87 | Blue Cheese | High THC | MPLX20-21, MPLX34-35, | 19188 |
| | | | MPLX70, MPLX91-91b | |
| 88 | Hollio | High THC | MPLX20-21, MPLX34-35, | 19205 |
| | | | MPLX70, MPLX91 | |
| 89 | BlueJackCity 30 | High THC | MPLX20-21, MPLX34-35, | 19206 |
| | | | MPLX70, MPLX91-91b | |
| 90 | BlueJackCity 32 | High THC | MPLX20-21, MPLX34-35, | 19201 |
| | | | MPLX70, MPLX91-91b | |
| 91 | Blue Jack City 35 | High THC | MPLX20-21, MPLX34-35, | 19162 |
| | | | MPLX70, MPLX91-91b | |
| 92 | BlueLimePie 21 | High THC | MPLX20-21, MPLX34-35, | 19200 |
| | | | MPLX70, MPLX91 | |
| 93 | Nollia/Campfire | High CBD | MPLX20-21, MPLX34-35, | 19180 |
| | | | MPLX66, MPLX70, MPLX91 | |
| 94 | Northern Tonic | Balanced | MPLX20-21, MPLX34-35, | 19169 |
| | | | MPLX70, MPLX91 | |
| 95 | CBD Shark | Balanced | MPLX20-21, MPLX34-35, | 19167 |
| | | | MPLX66, MPLX70, MPLX91 | |
| 96 | Chocolope | High THC | MPLX20-21, MPLX34-35, | 19171 |
| | | | MPLX62, MPLX64, MPLX70, | |
| | | | MPLX91 | |
| 97 | Critical Haze | High THC | MPLX20-21, MPLX34-35, | 19188 |
| | | | MPLX91 | |
| 98 | Integrius | High THC | MPLX20-21, MPLX34-35, | 19177 |
| | | | MPLX70, MPLX91 | |
| 99 | CSC | High THC | MPLX20-21, MPLX34-35, | 19131 |
| | | | MPLX36, MPLX62, MPLX70, | |
| | | | MPLX91-91b | |
| 100 | CSCB | High THC | MPLX20-21, MPLX34-35, | 19129 |
| | | | MPLX70, MPLX91-91b | |
| 101 | CSCH | High THC | MPLX20-21, MPLX34-35, | 19107 |
| | | | MPLX70 | |
| 102 | CSCJ | High CBD | MPLX20-21, MPLX34-35, | 19134 |
| | | | MPLX36, MPLX64, MPLX70, | |
| | | | MPLX91-91b | |
| 103 | CSCK | High THC | MPLX20-21, MPLX34-35, | 19164 |
| | | | MPLX64 | |
| 104 | CSCL | High THC | MPLX20-21, MPLX34-35, | 19128 |
| | | | MPLX64, MPLX70 | |
| 105 | Luminarium | High THC | MPLX20-21, MPLX34-35, | 19191 |
| | | | MPLX62, MPLX64, MPLX66, | |
| | | | MPLX70, MPLX91 | |
| 106 | Eran Almog | High THC | MPLX20-21, MPLX34-35, | 19183 |
| | | | MPLX64, MPLX66, MPLX70, | |
| | | | MPLX91-91b | |
| 107 | God'sGreenCrack-D | High THC | MPLX20-21, MPLX34-35, | 19199 |
| | | | MPLX64, MPLX70, MPLX91-91b | |
| 108 | God'sGreenCrack-F | High THC | MPLX20-21, MPLX34-35, | 19161 |
| | | | MPLX64, MPLX70, MPLX91-91b | |
| 109 | God'sGreenCrack-I | High THC | MPLX20-21, MPLX34-35, | 19202 |
| | | | MPLX64, MPLX70, MPLX91-91b | |
| 110 | Grease Monkey | High THC | MPLX20-21, MPLX34-35, | 19178 |
| | | | MPLX64, MPLX70 | |
| 111 | Hindu Kush | High THC | MPLX20-21, MPLX34-35, | 19108 |
| | | | MPLX64, MPLX70 | |
| 112 | Voluptas | High THC | MPLX20-21, MPLX34-35, | 19176 |
| | | | MPLX64, MPLX66, MPLX70 | |
| 113 | Juanita La Lagrimosa | High THC | MPLX20-21, MPLX34-35, | 19192 |
| | | | MPLX64, MPLX70 | |
| 114 | Stellio | High THC | MPLX20-21, MPLX34-35, | 19158 |
| | | | MPLX64, MPLX65, MPLX66, | |
| | | | MPLX70, MPLX91-91b | |
| 115 | Salinca | High THC | MPLX20-21, MPLX34-35, | 19175 |
| | | | MPLX64, MPLX70 | |
| 116 | LSD | High THC | MPLX20-21, MPLX34-35, | 19203 |
| | | | MPLX64, MPLX70 | |
| 117 | Med12-4 | High THC | MPLX20-21, MPLX34-35, | 19163 |
| | | | MPLX64, MPLX70 | |
| 118 | Med14-4 | High THC | MPLX20-21, MPLX34-35, | 19193 |
| | | | MPLX64, MPLX70 | |
| 119 | Med1-5 | High CBD | MPLX20-21, MPLX34-35, | 19185 |
| | | | MPLX64, MPLX65, MPLX70 | |
| 120 | Orellium | High CBD | MPLX20-21, MPLX34-35, | 19194 |
| | | | MPLX64, MPLX70, MPLX91 | |
| 121 | Midnight | Balanced | MPLX20-21, MPLX34-35, | 19195 |
| | | | MPLX64, MPLX65, MPLX70 | |
| 122 | Nuken | High THC | MPLX20-21, MPLX34-35, | 19166 |
| | | | MPLX64, MPLX65, MPLX66, | |
| | | | MPLX70 | |
| 123 | Purple Chitral | High THC | MPLX20-21, MPLX34-35, | 19182 |
| | | | MPLX65, MPLX66, MPLX70 | |
| 124 | Sedamen/Pink Kush | High THC | MPLX20-21, MPLX34-35, | 19159 |
| | | | MPLX62, MPLX64, MPLX65, | |
| | | | MPLX66, MPLX70, MPLX91-91b | |
| 125 | Romi | Unknown | MPLX20-21, MPLX34-35, | 19179 |
| | | | MPLX70 | |
| 126 | Skywalker OG Kush | High THC | MPLX20-21, MPLX34-35, | 19184 |
| | | | MPLX70 | |
| 127 | Strawberry Short | High THC | MPLX20-21, MPLX34-35, | 19172 |
| | Cookies | | MPLX65, MPLX70 | |
| 128 | Sugar Black Rose | High THC | MPLX20-21, MPLX34-35, | 19189 |
| | | | MPLX70 | |
| 129 | Tangerine Dream | High THC | MPLX20-21, MPLX34-35, | 19165 |
| | | | MPLX62, MPLX65, MPLX70 | |
| 130 | Tangerine Dream | High THC | MPLX20-21, MPLX34-35, | 19196 |
| | | | MPLX62, MPLX64, MPLX65, | |
| | | | MPLX66, MPLX70, MPLX91 | |
| 131 | Trutiva | High CBD | MPLX20-21, MPLX34-35, | 19187 |
| | | | MPLX65, MPLX70 | |
| 132 | Cognitiva | High THC | MPLX20-21, MPLX34-35, | 19197 |
| | | | MPLX70 | |
| 133 | White Widow | High THC | MPLX20-21, MPLX34-35, | 19188 |
| | | | MPLX65, MPLX70 | |
| 134 | Yakusha-10 | High THC | MPLX20-21, MPLX34-35, | 19198 |
| | | | MPLX62, MPLX64, MPLX70 | |
| 135 | Yakusha-11 | High THC | MPLX20-21, MPLX34-35, | 19198 |
| | | | MPLX64, MPLX70 | |
| 136 | LA Confidential | High THC | MPLX20-21, MPLX36, MPLX91- | 19102 |
| | | | 91b | |
| 137 | LA Confidential | High THC | MPLX20-21 | 19102 |
| 138 | LA Confidential | High THC | MPLX20-21 | 19102 |
| 139 | Hurkle | Balanced | MPLX20-21, MPLX34-35, | 19211 |
| | | | MPLX70 | |
| 140 | Sour Diesel/Odin | High THC | MPLX20-21, MPLX34-35, | 19212 |
| | | | MPLX70 | |
| 141 | OG Grape Krypt | High THC | MPLX20-21, MPLX34-35, | 19213 |
| | | | MPLX70 | |
| 142 | OG Grape Krypt | High THC | MPLX20-21, MPLX34-35, | 19214 |
| | | | MPLX70 | |
| 143 | Blackwater | High THC | MPLX20-21, MPLX34-35, | 19215 |
| | | | MPLX70 | |
| 144 | Querkle | High THC | MPLX20-21, MPLX34-35, | 19216 |
| | | | MPLX70 | |
| 145 | Zombie Kush | High THC | MPLX20-21, MPLX34-35, | 19111 |
| | | | MPLX64, MPLX65, MPLX66, | |
| | | | MPLX70, MPLX91 | |
| 146 | Zombie Kush | High THC | MPLX20-21, MPLX34-35, | 19217 |
| | | | MPLX70, MPLX91 | |
| 147 | Zombie Kush | High THC | MPLX20-21, MPLX34-35, | 19218 |
| | | | MPLX70, MPLX91 | |
| 148 | Dairy Queen | High THC | MPLX20-21, MPLX34-35, | 19219 |
| | | | MPLX70, MPLX91 | |
| 149 | Cannatonic 1:1 | Balanced | MPLX20-21, MPLX34-35, | 19220 |
| | | | MPLX70, MPLX91 | |
| 150 | Grand Daddy Purp | High THC | MPLX20-21, MPLX34-35, | 19221 |
| | | | MPLX70, MPLX91 | |
| 151 | Pennywise | Balanced | MPLX20-21, MPLX34-35, | 19222 |
| | | | MPLX70, MPLX91 | |
| 152 | Heisenberg Kush | High THC | MPLX20-21, MPLX34-35, | 19223 |
| | | | MPLX70, MPLX91 | |
| 153 | BC Rockstar | High THC | MPLX20-21, MPLX34-35, | 19224 |
| | | | MPLX65, MPLX70, MPLX91 | |
| 154 | Bubba Kush | High THC | MPLX20-21, MPLX34-35, | 19224 |
| | | | MPLX64, MPLX65, MPLX66, | |
| | | | MPLX70, MPLX91-91b | |
| 155 | CBG Shiatsu Kush | High THC | MPLX20-21, MPLX34-35, | 19233 |
| | | | MPLX70, MPLX91 | |
| 156 | Dancehall | Balanced | MPLX20-21, MPLX34-35, | 19226 |
| | | | MPLX64, MPLX70, MPLX91 | |
| 157 | Devil Fruit | High THC | MPLX20-21, MPLX34-35, | 19227 |
| | | | MPLX64, MPLX70, MPLX91 | |
| 158 | God's Green Crack | High THC | MPLX20-21, MPLX34-35, | 19234 |
| | | | MPLX70, MPLX91 | |
| 159 | Gorilla Glue | High THC | MPLX20-21, MPLX34-35, | 19228 |
| | | | MPLX70, MPLX91 | |
| 160 | Kosher Kush | High THC | MPLX20-21, MPLX34-35, | 19113 |
| | | | MPLX64, MPLX65, MPLX70, | |
| | | | MPLX91 | |
| 161 | Otto | High CBD | MPLX20-21, MPLX34-35, | 19229 |
| | | | MPLX70 | |
| 162 | Romulan Diesel | High THC | MPLX20-21, MPLX34-35, | 19230 |
| | | | MPLX70 | |
| 163 | Sour Jack | High THC | MPLX20-21, MPLX34-35, | 19231 |
| | | | MPLX65, MPLX70 | |
| 164 | Blueberry | High THC | MPLX20-21, MPLX34-35, | 19232 |
| | Lambsbread | | MPLX64, MPLX70 | |
| 165 | LA Confidential | High THC | MPLX20-21, MPLX34-35 | 19102 |
| 166 | LA Confidential | High THC | MPLX20-21 | 19102 |
| 167 | LA Confidential | High THC | MPLX20-21, MPLX34-35 | 19102 |
| 168 | LA Confidential | High THC | MPLX20-21, MPLX34-35 | 19102 |
| 169 | Unknown | High THC | MPLX34-35, MPLX70 | 19238 |
| 170 | Bellis | High THC | MPLX34-35, MPLX70 | 19239 |
| 171 | Cerebri | High THC | MPLX34-35, MPLX70 | 19240 |
| 172 | Dorit | High THC | MPLX34-35, MPLX70 | 19241 |
| 173 | Erez | High THC | MPLX34-35, MPLX70 | 19241 |
| 174 | Girl Scout Cookies | High THC | MPLX34-35, MPLX70 | 19242 |
| 175 | Remissio | High THC | MPLX34-35, MPLX70 | 19243 |
| 176 | Operari | High THC | MPLX34-35, MPLX70 | 19108 |
| 177 | Relevo | High THC | MPLX34-35, MPLX70 | 19108 |
| 178 | Tranquillum | High THC | MPLX34-35, MPLX70 | 19108 |
| 179 | Claritas | High THC | MPLX34-35, MPLX70 | 19245 |
| 180 | Or | High THC | MPLX34-35, MPLX70 | 19246 |
| 181 | Potentia | High THC | MPLX34-35, MPLX70 | 19247 |
| 182 | Durabilis | High THC | MPLX34-35, MPLX70, MPLX91 | 19248 |
| 183 | Articulus | Unknown | MPLX34-35, MPLX70, MPLX91 | 19249 |
| 184 | Socialis | High THC | MPLX34-35 | 19247 |
| 185 | LA Confidential | High THC | MPLX34-35 | 19102 |
| 186 | Ghost Train Haze | High THC | MPLX34-35 | 19103 |
| 187 | Ghost Train Haze | High THC | MPLX34-35 | 19103 |
| 188 | Banana Split | High THC | MPLX34-35 | 19111 |
| 189 | Banana Split | High THC | MPLX34-35 | 19111 |
| 190 | Banana Split | High THC | MPLX34-35 | 19111 |
| 191 | Cannatonic | High CBD | MPLX34-35 | 19101 |
| 192 | CSCJ | High CBD | MPLX34-35 | 19134 |
| 193 | CSC | High THC | MPLX34-35 | 19131 |
| 194 | CSC | High THC | MPLX34-35 | 19131 |
| 195 | CSCJ | High CBD | MPLX34-35 | 19134 |
| 196 | CSCJ | High CBD | MPLX34-35 | 19134 |
| 197 | CSCL | High THC | MPLX34-35 | 19128 |
| 198 | CSC | High THC | MPLX34-35 | 19131 |
| 199 | CSC | High THC | MPLX34-35 | 19131 |
| 200 | LA Confidential | High THC | MPLX34-35, MPLX36 | 19102 |
| 201 | BC Rockstar | High THC | MPLX34-35, MPLX70 | 19224 |
| 202 | Bubba Kush | High THC | MPLX34-35 | 19224 |
| 203 | CBD Shark | Balanced | MPLX34-35, MPLX70 | 19167 |
| 204 | Grapefruit | High THC | MPLX34-35, MPLX70 | 19252 |
| 205 | Medical Glue | High THC | MPLX34-35, MPLX70 | 19253 |
| 206 | Zombie Kush | High THC | MPLX34-35 | 19111 |
| 207 | Banana Split | High THC | MPLX34-35 | 19111 |
| 208 | CSCJ | High CBD | MPLX36 | 19134 |
| 209 | CSC | High THC | MPLX36 | 19131 |
| 210 | CSC | High THC | MPLX36 | 19131 |
| 211 | CSCJ | High CBD | MPLX36 | 19134 |
| 212 | CSC | High THC | MPLX36 | 19131 |
| 213 | Cannatonic | High CBD | MPLX36 | 19101 |
| 214 | Cannatonic | High CBD | MPLX36 | 19101 |
| 215 | Cannatonic | High CBD | MPLX36 | 19101 |
| 216 | Ghost Train Haze | High THC | MPLX36 | 19103 |
| 217 | Ghost Train Haze | High THC | MPLX36 | 19103 |
| 218 | Shishkaberry | High THC | MPLX36 | 19260 |
| 219 | Shishkaberry | High THC | MPLX36 | 19260 |
| 220 | Shishkaberry | High THC | MPLX36 | 19260 |
| 221 | Banana Split | High THC | MPLX36 | 19111 |
| 222 | Banana Split | High THC | MPLX36 | 19111 |
| 223 | Banana Split | High THC | MPLX36 | 19111 |
| 224 | Banana Split | High THC | MPLX36 | 19111 |
| 225 | Banana Split | High THC | MPLX36 | 19111 |
| 226 | Sedamen/Pink Kush | High THC | MPLX36 | 19159 |
| 227 | LA Confidential | High THC | MPLX36 | 19102 |
| 228 | LA Confidential | High THC | MPLX36 | 19102 |
| 229 | LA Confidential | High THC | MPLX36 | 19102 |
| 230 | LA Confidential | High THC | MPLX36 | 19102 |
| 231 | LA Confidential | High THC | MPLX36 | 19102 |
| 232 | CSC | High THC | MPLX34-35, MPLX64 | 19131 |
| 233 | CSCJ | High CBD | MPLX34-35, MPLX64 | 19134 |
| 234 | CSCJ | High CBD | MPLX34-35, MPLX64 | 19134 |
| 235 | CSC | High THC | MPLX34-35, MPLX64 | 19131 |
| 236 | CSCJ | High CBD | MPLX34-35, MPLX64 | 19134 |
| 237 | CSC | High THC | MPLX34-35, MPLX64 | 19131 |
| 238 | Chocolope | High THC | MPLX34-35, MPLX64 | 19173 |
| 239 | Chocolope | High THC | MPLX34-35, MPLX64 | 19173 |
| 240 | Chocolope | High THC | MPLX34-35, MPLX64 | 19173 |
| 241 | Chocolope | High THC | MPLX34-35, MPLX64 | 19173 |
| 242 | CSCJ | High CBD | MPLX34-35, MPLX64 | 19134 |
| 243 | CSC | High THC | MPLX34-35, MPLX64 | 19131 |
| 244 | CSC | High THC | MPLX34-35, MPLX64 | 19131 |
| 245 | LA Confidential | High THC | MPLX34-35, MPLX64 | 19102 |
| 246 | LA Confidential | High THC | MPLX34-35, MPLX64 | 19102 |
| 247 | Tangerine Dream | High THC | MPLX34-35, MPLX64 | 19196 |
| 248 | Tangerine Dream | High THC | MPLX34-35, MPLX64 | 19196 |
| 249 | Afghan Kush | High THC | MPLX34-35, MPLX64, MPLX70 | 19262 |
| 250 | Island Honey | High THC | MPLX34-35, MPLX64, MPLX70 | 19263 |
| 251 | Afghan Kush | High THC | MPLX34-35, MPLX64 | 19262 |
| 252 | CSCL | High THC | MPLX34-35, MPLX64 | 19128 |
| 253 | Luminarium | High THC | MPLX34-35, MPLX64 | 19191 |
| 254 | Equiposa | Balanced | MPLX34-35, MPLX64 | 19114 |
| 255 | LA Confidential | High THC | MPLX34-35, MPLX64 | 19102 |
| 256 | LA Confidential | High THC | MPLX34-35, MPLX64 | 19102 |
| 257 | 91 Krypt OG-Melon | High THC | MPLX34-35, MPLX64 | 19168 |
| 258 | Blue Dream | High THC | MPLX34-35, MPLX64 | 19208 |
| 259 | Blue Dream | High THC | MPLX34-35, MPLX64 | 19208 |
| 260 | White Rhino | High THC | MPLX70, MPLX91 | 19264 |
| 261 | LA Confidential | High THC | MPLX34-35, MPLX64 | 19102 |
| 262 | LA Confidential | High THC | MPLX34-35, MPLX64 | 19102 |
| 263 | LA Confidential | High THC | MPLX34-35, MPLX64 | 19102 |
| 264 | Sedamen/Pink Kush | High THC | MPLX34-35, MPLX64 | 19159 |
| 265 | Sedamen/Pink Kush | High THC | MPLX34-35, MPLX64 | 19159 |
| 266 | Ghost Train Haze | High THC | MPLX34-35, MPLX64 | 19103 |
| 267 | Ghost Train Haze | High THC | MPLX34-35, MPLX64 | 19103 |
| 268 | Alaska | High THC | MPLX34-35, MPLX64 | 19174 |
| 269 | Eran Almog | High THC | MPLX34-35, MPLX64 | 19183 |
| 270 | Stellio | High THC | MPLX34-35, MPLX64 | 19158 |
| 271 | CSCJ | High CBD | MPLX34-35, MPLX64 | 19134 |
| 272 | LA Confidential | High THC | MPLX34-35, MPLX64 | 19102 |
| 273 | LA Confidential | High THC | MPLX34-35, MPLX64 | 19102 |
| 274 | Sedamen/Pink Kush | High THC | MPLX34-35, MPLX64 | 19159 |
| 275 | Luminarium | High THC | MPLX34-35, MPLX64 | 19191 |
| 276 | Luminarium | High THC | MPLX34-35, MPLX64 | 19191 |
| 277 | Sedamen/Pink Kush | High THC | MPLX34-35, MPLX64 | 19159 |
| 278 | Tangerine Dream | High THC | MPLX34-35, MPLX64 | 19196 |
| 279 | Indica-1 | High THC | MPLX70 | 19302 |
| 280 | Sunday Special | High THC | MPLX70 | 19296 |
| 281 | Subway Scientist | High THC | MPLX70 | 19297 |
| 282 | Raider Kush | High THC | MPLX70 | 19290 |
| 283 | Sativa-2 | High THC | MPLX70 | 19301 |
| 284 | Quadra | High THC | MPLX70 | 19291 |
| 285 | Hybrid-1 | High THC | MPLX70 | 19298 |
| 286 | Savary | High THC | MPLX70 | 19224 |
| 287 | Sedamen/Pink Kush | High THC | MPLX65 | 19159 |
| 288 | Purple Chitral | High THC | MPLX65 | 19182 |
| 289 | Ghost Train Haze | High THC | MPLX65 | 19103 |
| 290 | Ghost Train Haze | High THC | MPLX65 | 19103 |
| 291 | MK Ultra | High THC | MPLX65 | 19106 |
| 292 | CSCJ | High CBD | MPLX65 | 19134 |
| 293 | Sour Jack | High THC | MPLX65 | 19231 |
| 294 | Sour Jack | High THC | MPLX65 | 19231 |
| 295 | Nuken | High THC | MPLX65 | 19166 |
| 296 | Nuken | High THC | MPLX65 | 19166 |
| 297 | Nuken | High THC | MPLX65 | 19166 |
| 298 | Blue Cheese | High THC | MPLX65 | 19188 |
| 299 | Blue Cheese | High THC | MPLX65 | 19188 |
| 300 | 91 Krypt OG-Melon | High THC | MPLX65 | 19168 |
| 301 | LA Confidential | High THC | MPLX65 | 19102 |
| 302 | LA Confidential | High THC | MPLX65 | 19102 |
| 303 | 91 Krypt OG-Melon | High THC | MPLX65 | 19168 |
| 304 | 91 Krypt OG-Melon | High THC | MPLX65 | 19168 |
| 305 | Sativa-2 | High THC | MPLX70 | 19301 |
| 306 | Quadra | High THC | MPLX70 | 19291 |
| 307 | Sativa-1 | High THC | MPLX70 | 19288 |
| 308 | Indica-1 | High THC | MPLX70 | 19302 |
| 309 | Gather | High THC | MPLX70 | 19299 |
| 310 | Sunday Special | High THC | MPLX70 | 19296 |
| 311 | Alaska | High THC | MPLX65 | 19174 |
| 312 | Eran Almog | High THC | MPLX65 | 19183 |
| 313 | Eran Almog | High THC | MPLX65 | 19183 |
| 314 | Eran Almog | High THC | MPLX65 | 19183 |
| 315 | Luminarium | High THC | MPLX65 | 19191 |
| 316 | Luminarium | High THC | MPLX65 | 19191 |
| 317 | Luminarium | High THC | MPLX65 | 19191 |
| 318 | Voluptas | High THC | MPLX65 | 19176 |
| 319 | Luminarium | High THC | MPLX65 | 19191 |
| 320 | Luminarium | High THC | MPLX65 | 19191 |
| 321 | Stellio | High THC | MPLX65 | 19158 |
| 322 | Stellio | High THC | MPLX65 | 19158 |
| 323 | Equiposa | Balanced | MPLX65 | 19114 |
| 324 | Equiposa | Balanced | MPLX65 | 19114 |
| 325 | Equiposa | Balanced | MPLX65 | 19114 |
| 326 | Equiposa | Balanced | MPLX65 | 19114 |
| 327 | Equiposa | Balanced | MPLX65 | 19114 |
| 328 | Equiposa | Balanced | MPLX65 | 19114 |
| 329 | Equiposa | Balanced | MPLX65 | 19114 |
| 330 | Equiposa | Balanced | MPLX65 | 19114 |
| 331 | Chocolope | High THC | MPLX65 | 19173 |
| 332 | Chocolope | High THC | MPLX65 | 19173 |
| 333 | Chocolope | High THC | MPLX65 | 19173 |
| 334 | LA Confidential | High THC | MPLX65 | 19102 |
| 335 | Blue Dream | High THC | MPLX65 | 19208 |
| 336 | Sedamen/Pink Kush | High THC | MPLX65 | 19159 |
| 337 | Luminarium | High THC | MPLX65 | 19191 |
| 338 | 91 Krypt OG-Melon | High THC | MPLX65 | 19168 |
| 339 | LA Confidential | High THC | MPLX65 | 19102 |
| 340 | 91 Krypt OG-Melon | High THC | MPLX65 | 19168 |
| 34 | MK Ultra | High THC | MPLX65 | 19106 |
| 342 | MK Ultra | High THC | MPLX65 | 19106 |
| 343 | LA Confidential | High THC | MPLX65 | 19102 |
| 344 | LA Confidential | High THC | MPLX65 | 19102 |
| 345 | LA Confidential | High THC | MPLX65 | 19102 |
| 346 | Sedamen/Pink Kush | High THC | MPLX65 | 19159 |
| 347 | 91 Krypt OG-Melon | High THC | MPLX65 | 19168 |
| 348 | Ghost Train Haze | High THC | MPLX65 | 19103 |
| 349 | Sedamen/Pink Kush | High THC | MPLX65, MPLX66 | 19159 |
| 350 | Eran Almog | High THC | MPLX65, MPLX66 | 19183 |
| 35' | Eran Almog | High THC | MPLX65, MPLX66 | 19183 |
| 352 | Sedamen/Pink Kush | High THC | MPLX65, MPLX66 | 19159 |
| 353 | Sour Tangie | High THC | MPLX65, MPLX66 | 19170 |
| 354 | 91 Krypt OG-Melon | High THC | MPLX65, MPLX66 | 19168 |
| 355 | Sedamen/Pink Kush | High THC | MPLX65, MPLX66 | 19159 |
| 356 | 91 Krypt OG-Melon | High THC | MPLX65 | 19168 |
| 357 | 91 Krypt OG-Melon | High THC | MPLX65 | 19168 |
| 358 | 91 Krypt OG-Melon | High THC | MPLX65 | 19168 |
| 359 | Blue Dream | High THC | MPLX70 | 19289 |
| 360 | Sativa | High THC | MPLX70 | 19299 |
| 361 | Subway Scientist | High THC | MPLX70 | 19297 |
| 362 | Raider Kush | High THC | MPLX70 | 19290 |
| 363 | Quadra | High THC | MPLX70 | 19291 |
| 364 | Savary | High THC | MPLX70 | 19224 |
| 365 | Tangerine Dream | High THC | MPLX65 | 19196 |
| 366 | Sedamen/Pink Kush | High THC | MPLX65 | 19159 |
| 367 | Avidekel | High CBD | MPLX65 | 19204 |
| 368 | Ghost Train Haze | High THC | MPLX65 | 19103 |
| 369 | Ghost Train Haze | High THC | MPLX65 | 19103 |
| 370 | Ghost Train Haze | High THC | MPLX65 | 19103 |
| 371 | Ghost Train Haze | High THC | MPLX65 | 19103 |
| 372 | MK Ultra | High THC | MPLX65 | 19106 |
| 373 | MK Ultra | High THC | MPLX65 | 19106 |
| 374 | 91 Krypt OG-Melon | High THC | MPLX65 | 19168 |
| 375 | Strawberry Short | High THC | MPLX65 | 19172 |
| | Cookies | | | |
| 376 | Kosher Kush | High THC | MPLX65 | 19113 |
| 377 | Chocolope | High THC | MPLX65 | 19171 |
| 378 | Tangerine Dream | High THC | MPLX65 | 19165 |
| 379 | Chocolope | High THC | MPLX65 | 19171 |
| 380 | Blue Jack City 35 | High THC | MPLX65 | 19162 |
| 381 | BC Rockstar | High THC | MPLX65 | 19224 |
| 382 | White Widow | High THC | MPLX65 | 19188 |
| 383 | Nuken | High THC | MPLX65 | 19166 |
| 384 | Voluptas | High THC | MPLX65 | 19176 |
| 385 | Sedamen/Pink Kush | High THC | MPLX65 | 19159 |
| 386 | Sedamen/Pink Kush | High THC | MPLX65 | 19159 |
| 387 | Voluptas | High THC | MPLX65 | 19176 |
| 388 | Indica-2 | High THC | MPLX70 | 19108 |
| 389 | Saturna | High THC | MPLX70 | 19292 |
| 390 | Lagoon | High THC | MPLX70 | 19293 |
| 391 | Chemdog | High THC | MPLX70 | 19294 |
| 392 | Headband | High THC | MPLX70 | 19295 |
| 393 | Sunday Special | High THC | MPLX70 | 19296 |
| 394 | Subway Scientist | High THC | MPLX70 | 19297 |
| 395 | Hybrid-1 | High THC | MPLX70 | 19298 |
| 396 | Sativa | High THC | MPLX70 | 19299 |
| 397 | Indica | High THC | MPLX70 | 19300 |
| 398 | Sativa-2 | High THC | MPLX70, MPLX91 | 19301 |
| 399 | Indica-1 | High THC | MPLX70 | 19302 |
| 400 | LA Confidential | High THC | MPLX65, MPLX70 | 19102 |
| 401 | LA Confidential | High THC | MPLX65, MPLX70 | 19102 |
| 402 | Luminarium | High THC | MPLX65, MPLX70 | 19191 |
| 403 | Luminarium | High THC | MPLX65, MPLX70 | 19191 |
| 404 | LA Confidential | High THC | MPLX65, MPLX70 | 19102 |
| 405 | MK Ultra | High THC | MPLX65, MPLX70 | 19106 |
| 406 | Luminarium | High THC | MPLX65, MPLX70 | 19191 |
| 407 | Blue Dream/Lemon | High THC | MPLX65, MPLX70, MPLX91-91b | 19285 |
| | Haze-1 | | | |
| 408 | White Rhino | High THC | MPLX70 | 19264 |
| 409 | Blue Dream | High THC | MPLX70 | 19208 |
| 410 | LA Confidential | High THC | MPLX70 | 19102 |
| 411 | 91 Krypt OG-Melon | High THC | MPLX70 | 19168 |
| 412 | 91 Krypt OG-Melon | High THC | MPLX70 | 19168 |
| 413 | 91 Krypt OG-Melon | High THC | MPLX70 | 19168 |
| 414 | 91 Krypt OG-Melon | High THC | MPLX70 | 19168 |
| 415 | Nepal Jam | High THC | MPLX70 | 19238 |
| 416 | Kanteris | High THC | MPLX70 | 19286 |
| 417 | UnknownA | Unknown | MPLX70 | 19287 |
| 418 | LA Confidential | High THC | MPLX70 | 19102 |
| 419 | Cherry Punch | High THC | MPLX70 | 19303 |
| 420 | Wedding Crasher | High THC | MPLX70, MPLX91 | 19304 |
| 421 | Gabriola | High THC | MPLX70 | 19300 |
| 422 | Sativa-1 | High THC | MPLX70 | 19306 |
| 423 | Wappa | High THC | MPLX70 | 19307 |
| 424 | Lemon Riot | High THC | MPLX70 | 19308 |
| 425 | Sour Cookies | High THC | MPLX70 | 19166 |
| 426 | White Rhino | High THC | MPLX70 | 19264 |
| 427 | Pink Kush | High THC | MPLX70 | 19188 |
| 428 | Cherry Punch | High THC | MPLX70 | 19303 |
| 429 | Sunday Special | High THC | MPLX70 | 19296 |
| 430 | Sour Kush | High THC | MPLX70 | 19309 |
| 431 | Saturna | High THC | MPLX70 | 19292 |
| 432 | Hawaii Heartbreak | High THC | MPLX70 | 19300 |
| 433 | DT81 Super Lemon | High THC | MPLX70 | 19310 |
| | Haze | | | |
| 434 | Monkey Glue | High THC | MPLX70 | 19311 |
| 435 | Jean Guy | High THC | MPLX70 | 19312 |
| 436 | Balance | Balanced | MPLX70 | 19313 |
| 437 | Unplug | High THC | MPLX70 | 19314 |
| 438 | Free | High CBD | MPLX70 | 19315 |
| 439 | Renew | High THC | MPLX70 | 19243 |
| 440 | Renew | High THC | MPLX70 | 19243 |
| 441 | Sour Kush | High THC | MPLX70 | 19309 |
| 442 | Sedamen/Pink Kush | High THC | MPLX70 | 19159 |
| 443 | Pink Kush | High THC | MPLX70 | 19224 |
| 444 | Pink Kush | High THC | MPLX70 | 19224 |
| 445 | Pink Kush | High THC | MPLX70 | 19224 |
| 446 | Black Garlic | High THC | MPLX70 | 19316 |
| 447 | Black Garlic | High THC | MPLX70 | 19316 |
| 448 | Black Garlic | High THC | MPLX70 | 19316 |
| 449 | Jack Haze | High THC | MPLX70 | 19317 |
| 450 | Tangerine Dream | High THC | MPLX70 | 19196 |
| 451 | Rio Bravo | High THC | MPLX70 | 19318 |
| 452 | BC Diesel | High THC | MPLX70 | 19319 |
| 453 | SFV Kush | High THC | MPLX70 | 19320 |
| 454 | Chemdog | High THC | MPLX70 | 19294 |
| 455 | Pink Kush | High THC | MPLX70 | 19224 |
| 456 | Pink Kush | High THC | MPLX70 | 19224 |
| 457 | Purple Chitral | High THC | MPLX70 | 19322 |
| 458 | Gabriola | High THC | MPLX70 | 19300 |
| 459 | Chocolate Fondue | High THC | MPLX70 | 19323 |
| 460 | Blueberry Seagal | High THC | MPLX70 | 19324 |
| 461 | LA Confidential | High THC | MPLX70 | 19102 |
| 462 | BC Rockstar | High THC | MPLX70 | 19224 |
| 463 | SOT005 | High THC | MPLX70 | 19330 |
| 464 | SOT006 | High THC | MPLX70 | 19331 |
| 465 | SOT004 | High THC | MPLX70 | 19332 |
| 466 | Sativa-2 | High THC | MPLX70 | 19325 |
| 467 | Afghan Kush | High THC | MPLX70 | 19262 |
| 468 | Redees Cold Creek | High THC | MPLX70 | 19133 |
| | Kush | | | |
| 469 | Apple Toffee | High THC | MPLX70 | 19326 |
| 470 | Wedding Crasher | High THC | MPLX70, MPLX91-91b | 19304 |
| 471 | Pipe Dream | High THC | MPLX70, MPLX91 | 19327 |
| 472 | Craft Collective Pink | High THC | MPLX70 | 19224 |
| | Kush | | | |
| 473 | Death Bubba | High THC | MPLX70, MPLX91-91b | 19328 |
| 474 | Granddaddy Purple | High THC | MPLX70, MPLX91 | 19329 |
| 475 | GMO | High THC | MPLX70 | 19333 |
| 476 | Grandaddy Stash | High THC | MPLX70 | 19334 |
| 477 | Vespera | Balanced | MPLX91 | 19186 |
| 478 | CSCJ | High CBD | MPLX91 | 19134 |
| 479 | Crystal Kush | High THC | MPLX91 | 19335 |
| 480 | Crystal Kush | High THC | MPLX91 | 19335 |
| 481 | Crystal Kush | High THC | MPLX91 | 19335 |
| 482 | Crystal Kush | High THC | MPLX91 | 19335 |
| 483 | Wedding Crasher | High THC | MPLX91 | 19304 |
| 484 | BC Rockstar | High THC | MPLX91 | 19224 |
| 485 | Pink Kush | High THC | MPLX91 | 19224 |
| 486 | Chocolate Fondue | High THC | MPLX70, MPLX91 | 19323 |
| 487 | Pipe Dream | High THC | MPLX91 | 19327 |
| 488 | Ice Cream Cake | High THC | MPLX91 | 19336 |
| 489 | White Rhino | High THC | MPLX91 | 19264 |
| 490 | Subway Scientist | High THC | MPLX91 | 19297 |
| 491 | Garlic Jelly | High THC | MPLX91 | 19337 |
| 492 | Melon Gum | High THC | MPLX91 | 19301 |
| 493 | Zookies | High THC | MPLX91 | 19338 |
| 494 | Slurricane | High THC | MPLX91 | 19339 |
| 495 | Wedding | High THC | MPLX91 | 19336 |
| 496 | BC Rockstar | High THC | MPLX70 | 19224 |

TABLE 37
Parameters for PCR amplifications from Genomic DNA.

| | | | | Number | |
|---|---|---|---|---|---|
| Step | Temperature | Time | Go To | of Cycles | Notes |
| 1 | 94° C. | 1 min | | | |
| 2 | 95° C. | 15 sec | | | |
| 3 | 65° C. | 15 sec | | | Decrease 1° C. each |
| | | | | | cycle |
| 4 | 72° C. | 30 sec | 2 | 15 | |
| 5 | 95° C. | 15 sec | | | |
| 6 | 50° C. | 15 sec | | | |
| 7 | 72° C. | 30 sec | 2 | 20 | |
| 8 | 72° C. | 5 min | | | |
| 9 | 4° C. | hold | | | |

TABLE 38a
Confirmatory genotyping of three samples attributed to varieties
Blue Dream, Luminarium ™, and CSC using multiplex 36.

| | Samples | | | | | |
|---|---|---|---|---|---|---|
| | Blue | Sam- | | Sam- | | Sam- |
| SSR locus | Dream | ple 1 | Luminarium ™ | ple 2 | CSC | ple 3 |
| ANUCS501 | 93 | 93 | 93 | 93 | 98 | 98 |
| ANUCS501 | 103 | 103 | 98 | 98 | 98 | 98 |
| B01-CANN1 | 330 | 330 | 330 | 330 | 327 | 327 |
| B01-CANN1 | 330 | 330 | 370 | 370 | 330 | 330 |
| C11-CANN1 | 159 | 159 | 156 | 156 | 159 | 159 |
| C11-CANN1 | 159 | 159 | 156 | 156 | 159 | 159 |
| CAN0009 | 127 | 127 | 137 | 137 | 127 | 127 |
| CAN0009 | 137 | 137 | 137 | 137 | 127 | 127 |
| CAN0055 | 278 | 278 | 283 | 283 | 278 | 278 |
| CAN0055 | 283 | 283 | 283 | 283 | 283 | 283 |
| CAN0509 | 300 | 300 | 292 | 292 | 306 | 306 |
| CAN0509 | 300 | 300 | 300 | 300 | 306 | 306 |
| CAN0717 | 310 | 310 | 304 | 304 | 310 | 310 |
| CAN0717 | 310 | 310 | 310 | 310 | 310 | 310 |
| CAN2550 | 304 | 304 | 304 | 304 | 304 | 304 |
| CAN2550 | 304 | 304 | 307 | 307 | 304 | 304 |
| D02-CANN1 | 113 | 113 | 116 | 116 | 113 | 113 |
| D02-CANN1 | 116 | 116 | 116 | 116 | 113 | 113 |
| E07-CANN1 | 111 | 111 | 111 | 111 | 114 | 114 |
| E07-CANN1 | 114 | 114 | 114 | 114 | 114 | 114 |

TABLE 38b
Confirmatory genotyping of three samples attributed to varieties Blue
Dream, Luminarium ™, and CSC using multiplex 70.
Samples

| | Blue | Sam- | | Sam- | | Sam- |
|---|---|---|---|---|---|---|
| SSR locus | Dream | ple 1 | Luminarium ™ | ple 2 | CSC | ple 3 |
| C11- | 190 | 190 | 190 | 190 | 190 | 190 |
| CANN1ana2 | | | | | | |
| C11- | 193 | 193 | 190 | 190 | 193 | 193 |
| CANN1ana2 | | | | | | |
| CAN0055ana4 | 110 | 110 | 115 | 115 | 110 | 110 |
| CAN0055ana4 | 115 | 115 | 115 | 115 | 115 | 115 |
| CAN0089ana | 307 | 307 | 299 | 299 | 299 | 299 |
| CAN0089ana | 307 | 307 | 309 | 309 | 299 | 299 |
| CAN0103ana | 240 | 240 | 240 | 240 | 240 | 240 |
| CAN0103ana | 240 | 240 | 240 | 240 | 246 | 246 |
| CAN0156ana | 445 | 445 | 437 | 437 | 437 | 437 |
| CAN0156ana | 445 | 445 | 437 | 437 | 437 | 437 |
| CAN0509ana | 310 | 310 | 302 | 302 | 316 | 316 |
| CAN0509ana | 310 | 310 | 310 | 310 | 316 | 316 |
| CAN0577ana | 478 | 478 | 490 | 490 | 478 | 478 |
| CAN0577ana | 490 | 490 | 490 | 490 | 478 | 478 |
| CAN0802ana | 330 | 330 | 324 | 324 | 324 | 324 |
| CAN0802ana | 330 | 330 | 330 | 330 | 330 | 330 |
| CAN0907ana | 226 | 226 | 226 | 226 | 226 | 226 |
| CAN0907ana | 229 | 229 | 226 | 226 | 232 | 232 |
| CAN1539ana | 510 | 510 | 520 | 520 | 510 | 510 |
| CAN1539ana | 510 | 510 | 520 | 520 | 510 | 510 |
| CAN1832ana | 127 | 127 | 127 | 127 | 133 | 133 |
| CAN1832ana | 133 | 133 | 133 | 133 | 133 | 133 |
| CAN3008ana | 367 | 367 | 367 | 367 | 364 | 364 |
| CAN3008ana | 367 | 367 | 367 | 367 | 367 | 367 |
| E07- | 263 | 263 | 263 | 263 | 263 | 263 |
| CANN1ana2 | | | | | | |
| E07- | 284 | 284 | 281 | 281 | 263 | 263 |
| CANN1ana2 | | | | | | |
| H06- | 151 | 151 | 151 | 151 | 151 | 151 |
| CANN2ana | | | | | | |
| H06- | 151 | 151 | 157 | 157 | 157 | 157 |
| CANN2ana | | | | | | |

TABLE 39a
Cannabis SSR Markers, SSR-specific primers, and reverse complement
sequences for SSR-specific primers from multiplexes 20 and 21, 34 and 35 or 36.

| | | SEQ ID | | SEQ ID |
|---|---|---|---|---|
| | | NO of | | NO of |
| | Primer Name/ | Primer | Reverse Complement | Reverse |
| Marker | Primer Sequence | Sequence | of Primer Sequence | Complement |
| ANUCS501 | EG221r/ | 2 | GGAATCTCAATTTCTT | 101 |
| | AGAGATCAAGAAATTGAG | | GATCTCT | |
| | ATTCC | | | |
| C11-CANN1 | EG225r/ | 4 | CGCCATCGTAACCAA | 102 |
| | TGAATTGGTTACGATGGC | | TTCA | |
| | G | | | |
| E07-CANN1 | EG227r/ | 6 | CTACCTATACCTGGC | 103 |
| | GTAGGTAGCCAGGTATA | | TACCTAC | |
| | GGTAG | | | |
| H06-CANN2 | EG229r/ | 8 | CTCGTGTCATCACTC | 104 |
| | ACGTGAGTGATGACACG | | ACGT | |
| | AG | | | |
| B01-CANN1 | EG233r/ | 10 | CTTGAGTGGGATAAT | 105 |
| | CCATAGCATTATCCCACT | | GCTATGG | |
| | CAAG | | | |
| D02-CANN1 | EG239r/ | 12 | CCATCAGGACCTTGG | 106 |
| | AGAAATCCAAGGTCCTGA | | ATTTCT | |
| | TGG | | | |
| CAN0055 | EG267r/ | 14 | GGACCCAATTCGAGT | 107 |
| | CTGTTACTCGAATTGGGT | | AACAG | |
| | CC | | | |
| CAN0111 | EG301r/ | 16 | ACAAGTTCTGATCCC | 108 |
| | CTGGTGGGATCAGAACT | | ACCAG | |
| | TGT | | | |
| CAN0509 | EG307r/ | 18 | ACCTGCAACAGCAAC | 109 |
| | TAACTGTTGCTGTTGCAG | | AGTTA | |
| | GT | | | |
| CAN0717 | EG317r/ | 20 | AGTCCCTCAAAAACA | 110 |
| | GCTCATGTTTTTGAGGGA | | TGAGC | |
| | CT | | | |
| CAN2550 | EG341r/ | 22 | CACCACTTTCAGGCC | 111 |
| | GATTAGGCCTGAAAGTG | | TAATC | |
| | GTG | | | |
| CAN0009 | EG353r/ | 24 | TCTCAACTCTCATGG | 112 |
| | TCAAGCCATGAGAGTTGA | | CTTGA | |
| | GA | | | |
| ANUCS301 | EG211r/ | 26 | ACCCTCACGAAACTT | 113 |
| | TAACAAAGTTTCGTGAGG | | TGTTA | |
| | GT | | | |
| ANUCS303 | EG215r/ | 28 | TATCGTCGAGGACCT | 114 |
| | GATTAAGGTCCTCGACG | | TAATC | |
| | ATA | | | |
| ANUCS304 | EG217r/ | 30 | ACGAGTCCCGCTTAA | 115 |
| | TCTTTAAGCGGGACTCGT | | AGA | |
| B02-CANN2 | EG235r/ | 32 | GGGTGCAGTGAAGAA | 116 |
| | TGTTTTCTTCACTGCACC | | AACA | |
| | C | | | |
| B05-CANN1 | EG223r/ | 34 | GGGTTGAGATTGAGA | 117 |
| | CCCCAATCTCAATCTCAA | | TTGGGG | |
| | CCC | | | |
| CAN0126 | EG253r/ | 36 | GGGGTTTTCTGTTAC | 118 |
| | CCTGTGTAACAGAAAACC | | ACAGG | |
| | CC | | | |
| CAN1347 | EG257r/ | 38 | ACACTTGCTTTACCTT | 119 |
| | GGCAAAGGTAAAGCAAG | | TGCC | |
| | TGT | | | |
| CAN1482 | EG289r/ | 40 | AGCCTAACCCCTTCT | 120 |
| | ATTTGAGAAGGGGTTAG | | CAAAT | |
| | GCT | | | |
| CAN2913 | EG261r/ | 42 | AAGCTCAAGGTAGAT | 121 |
| | CGGTCATCTACCTTGAGC | | GACCG | |
| | TT | | | |
| NM101 | EG293r/ | 44 | CGTTCTCACTCGTCT | 122 |
| | TAATGATGAGACGAGTGA | | CATCATTA | |
| | GAACG | | | |
| ANUCS305 | EG219r/ | 46 | TGTTGTCGAAAGTTC | 123 |
| | CCTAGGAACTTTCGACAA | | CTAGG | |
| | CA | | | |
| CAN0006 | EG295r/ | 125 | GCTGATGAGGATGAT | 152 |
| | TCATCATCATCCTCATCA | | GATGA | |
| | GC | | | |
| CAN0051 | EG357r/ | 127 | CGTGTACTCACCTTG | 153 |
| | CTCAGCAAGGTGAGTAC | | CTGAG | |
| | ACG | | | |
| CAN0061 | EG299r/ | 129 | CAAATCAGGAGTGTG | 154 |
| | CTCTGCACACTCCTGATT | | CAGAG | |
| | TG | | | |
| CAN0118 | EG303r/ | 131 | AGAACGAACAATGCT | 155 |
| | GGAGAAGCATTGTTCGTT | | TCTCC | |
| | CT | | | |
| CAN0189 | EG305r/ | 133 | GACCTGATGAGCCAG | 156 |
| | AATAGCTGGCTCATCAG | | CTATT | |
| | GTC | | | |
| CAN0590 | EG309r/ | 135 | TCTACCTGTGGTGCA | 157 |
| | TCTTCTGCACCACAGGTA | | GAAGA | |
| | GA | | | |
| CAN0716 | EG315r/ | 137 | TCCACTTCTTCTTCGG | 158 |
| | GAAACCGAAGAAGAAGT | | TTTC | |
| | GGA | | | |
| CAN0795 | EG319r/ | 139 | TGTGAAATCCAGCTC | 159 |
| | CATCTGAGCTGGATTTCA | | AGATG | |
| | CA | | | |
| CAN1455 | EG327r/ | 141 | CATCTTGGGGATTGG | 160 |
| | CTTAACCAATCCCCAAGA | | TTAAG | |
| | TG | | | |
| CAN1481 | EG329r/ | 143 | AGCCTAACCCCTTCT | 161 |
| | ATTTGAGAAGGGGTTAG | | CAAAT | |
| | GCT | | | |
| CAN1832 | EG335r/ | 145 | GAGCCTTCAACTTTC | 162 |
| | GTTTCGAAAGTTGAAGGC | | GAAAC | |
| | TC | | | |
| CAN2166 | EG337r/ | 147 | CTTCATTAGGTACGC | 163 |
| | ACGAGGCGTACCTAATG | | CTCGT | |
| | AAG | | | |
| CAN3105 | EG349r/ | 149 | GCAGTGACCTCATAT | 164 |
| | TGTCCATATGAGGTCACT | | GGACA | |
| | GC | | | |
| CAN3321 | EG351r/ | 151 | CAGGCCAAGTTTTCA | 165 |
| | ATAGGTGAAAACTTGGCC | | CCTAT | |
| | TG | | | |

TABLE 39b
Cannabis SSR Markers, SSR-specific primers, and reverse complement
sequences for SSR-specific primers from multiplexes 62, 64, 65, 66, 70, 91 and 91b.

| | | | SEQ ID | | SEQ ID |
|---|---|---|---|---|---|
| | | | NO of | | NO of |
| | Primer | | Primer | Reverse Complement | Reverse |
| Marker | Name | Primer Sequence | Sequence | of Primer Sequence | Complement |
| E07- | EG227r | GTAGGTAGCCAGGTATA | 6 | CTACCTATACCTGGCTAC | 103 |
| CANN1ana4 | | GGTAG | | CTAC | |
| ANUCS304ana | EG497r | AAAGAAGGACCTCCCAT | 167 | GGATATGGGAGGTCCTT | 252 |
| | | ATCC | | CTTT | |
| ANUCS501ana | EG425r | CAAGAAATTGAGATTCCC | 169 | CCATTGGGAATCTCAATT | 253 |
| | | AATGG | | TCTTG | |
| C11- | EG537r | ATGGGCGTCCTTGAATT | 171 | CGTAACCAATTCAAGGAC | 254 |
| CANN1ana2 | | GGTTACG | | GCCCAT | |
| CAN0009ana2 | EG467r | TCCGAATAGAAAAAAAAA | 173 | CCCAATTTTTTTTTTCTAT | 255 |
| | | ATTGGG | | TCGGA | |
| CAN0055ana4 | EG538r | CTTAATCTAACCTCTAAC | 175 | GCGGTCGTTAGAGGTTA | 256 |
| | | GACCGC | | GATTAAG | |
| CAN0057ana | EG544r | GAATTTTTGTTGCATTTA | 177 | CTAAAGTGAGTTAAATGC | 257 |
| | | ACTCACTTTAG | | AACAAAAATTC | |
| CAN0075ana | EG491r | TTCTTTTCTGGGGCTCTC | 179 | GAGAGAGCCCCAGAAAA | 258 |
| | | TC | | GAA | |
| CAN0089ana | EG518r | CGCCAACATTTACTCACT | 181 | GAGTTTAGTGAGTAAATG | 259 |
| | | AAACTC | | TTGGCG | |
| CAN0103ana | EG524r | GGAGCTTCTTTTTGAGAG | 183 | CAGACTCTCAAAAAGAAG | 260 |
| | | TCTG | | CTCC | |
| CAN0124ana | EG510r | ATAAACACAAGGAAGTTG | 185 | CTTCATCATCAACTTCCT | 261 |
| | | ATGATGAAG | | TGTGTTTAT | |
| CAN0138ana2 | EG471r | ATAATAAGAAATTAATGG | 187 | CAACAACACCATTAATTT | 262 |
| | | TGTTGTTG | | CTTATTAT | |
| CAN0156ana | EG514r | TTGCTAAATAACTCAGTA | 189 | TCTGTTTTGTACTGAGTT | 263 |
| | | CAAAACAGA | | ATTTAGCAA | |
| CAN0180ana | EG487r | AGACGATAGGACTAAAAT | 191 | GATTAGATTTTAGTCCTA | 264 |
| | | CTAATC | | TCGTCT | |
| CAN0202ana | EG477r | ACTACCTACAACGGTTCC | 193 | CAGGAACCGTTGTAGGT | 265 |
| | | TG | | AGT | |
| CAN0244ana | EG481r | AACTCAGTTCAAGGAAAA | 195 | CCCTTTTTCCTTGAACTG | 266 |
| | | AGGG | | AGTT | |
| CAN0271ana | EG473r | ACGACGATGATGATGAT | 197 | GCCATCATCATCATCGTC | 267 |
| | | GGC | | GT | |
| CAN0278ana | EG493r | AAGTGGGCAACGACTTG | 199 | CCACAAGTCGTTGCCCA | 268 |
| | | TGG | | CTT | |
| CAN0291ana | EG483r | AGAAGACCGGTGGTTAC | 201 | CAGGTAACCACCGGTCT | 269 |
| | | CTG | | TCT | |
| CAN0298ana | EG485r | AAACCCTCATTAGATGTG | 203 | GTGAACACATCTAATGAG | 270 |
| | | TTCAC | | GGTTT | |
| CAN0308ana | EG489r | TTCTTGAACATGTCTTGG | 205 | GTGCCAAGACATGTTCAA | 271 |
| | | CAC | | GAA | |
| CAN0338- | EG504r | TCAATGGTGGCAAGTAG | 207 | GTTGCTACTTGCCACCAT | 272 |
| 339ana | | CAAC | | TGA | |
| CAN0442ana | EG479r | TTGTTCTTCTCTGAGCTG | 209 | CCCAGCTCAGAGAAGAA | 273 |
| | | GG | | CAA | |
| CAN0509ana | EG431r | TTGAAACGTTATTTTGCG | 211 | GCTCCGCAAAATAACGTT | 274 |
| | | GAGC | | TCAA | |
| CAN0577ana | EG551r | CTCTTTTGTTTGTTCTGT | 213 | CAAGTCCAACAGAACAAA | 275 |
| | | TGGACTTG | | CAAAAGAG | |
| CAN0716ana | EG500r | AAGTGATGAGCAGTTTG | 215 | GTATCTCAAACTGCTCAT | 276 |
| | | AGATAC | | CACTT | |
| CAN0717ana | EG424r | GTCCACTATACAAAAACT | 217 | GCTTCAGTTTTTGTATAG | 277 |
| | | GAAGC | | TGGAC | |
| CAN0802ana | EG522r | AATTAGAATGATGTCAGT | 219 | CCAGTGAAAACTGACATC | 278 |
| | | TTTCACTGG | | ATTCTAATT | |
| CAN0907ana | EG516r | CACTTTGACTAGTGATCC | 221 | CTGTTTGGATCACTAGTC | 279 |
| | | AAACAG | | AAAGTG | |
| CAN1409ana | EG549r | ATGGGTTTTATTCAACAA | 223 | GTAACCGGTTGTTGAATA | 280 |
| | | CCGGTTAC | | AAACCCAT | |
| CAN1539ana | EG508r | CTGTATGCGTCTTTGTAA | 225 | CAAATAGTTACAAAGACG | 281 |
| | | CTATTTG | | CATACAG | |
| CAN1542ana | EG475r | GAGAAGTACAGGAACCA | 227 | CTCTTGGTTCCTGTACTT | 282 |
| | | AGAG | | CTC | |
| CAN1832ana | EG502r | TCATTCCTGAGAATATAA | 229 | CAAGCTTTATATTCTCAG | 283 |
| | | AGCTTG | | GAATGA | |
| CAN2039ana | EG542r | CAGTAGTACAAGATCCAA | 231 | CACTTCCTTGGATCTTGT | 284 |
| | | GGAAGTG | | ACTACTG | |
| CAN2418ana | EG547r | AGCTTAACTCAACCAAGA | 233 | GCAAAAGTATCTTGGTTG | 285 |
| | | TACTTTTGC | | AGTTAAGCT | |
| CAN2550ana2 | EG415r | GGTGGTCGTATTATTCG | 235 | CTATGCGAATAATACGAC | 286 |
| | | CATAG | | CACC | |
| CAN2829ana | EG526r | TCCGGTTCCCATACAATT | 237 | CAAGAATTGTATGGGAAC | 287 |
| | | CTTG | | CGGA | |
| CAN2846ana | EG534r | GCAGGGATTGGACCCAT | 239 | CAAATGGGTCCAATCCCT | 288 |
| | | TTG | | GC | |
| CAN2902ana | EG530r | CCAACTGCACGTACAGA | 241 | TTTTGTTTCTGTACGTGC | 289 |
| | | AACAAAA | | AGTTGG | |
| CAN2913ana | EG528r | TGGTCTTGACGCCATTCT | 243 | CGAGAATGGCGTCAAGA | 290 |
| | | CG | | CCA | |
| CAN3008ana | EG532r | GCTCCTCCGGTGGTCTA | 245 | GTTTAGACCACCGGAGG | 291 |
| | | AAC | | AGC | |
| D02- | EG413r | TTTTAAATTTAGGTGTGT | 247 | CCAAAGTACACACCTAAA | 292 |
| CANN1ana | | ACTTTGG | | TTTAAAA | |
| E07- | EG420r | TATATGTGGGTGTGTTAG | 249 | GAAAATTCTAACACACCC | 293 |
| CANN1ana2 | | AATTTTC | | ACATATA | |
| H06- | EG495r | TTCCCTGTCGTGGTTGCT | 251 | CAAGCAACCACGACAGG | 294 |
| CANN2ana | | TG | | GAA | |
| ANUCS501ana2 | EG417r | TGTCACTCACTGCCATCT | 295 | GAGAGATGGCAGTGAGT | 314 |
| | | CTC | | GACA | |
| CAN0067ana | EG658r | TTTAGACAAACCACACAA | 297 | GTTTTCGGTTGTGTGGTT | 315 |
| | | CCGAAAAC | | TGTCTAAA | |
| CAN0097ana | EG686r | AGTAAAGCCTCTGCGAG | 299 | GAGGATACTCGCAGAGG | 316 |
| | | TATCCTC | | CTTTACT | |
| CAN0202ana2 | EG591r | AACCTTGTACGCTTTCCA | 300 | GCAAGTGGAAAGCGTAC | 317 |
| | | CTTGC | | AAGGTT | |
| CAN0250ana | EG582r | CAGGGTAACAATCACAT | 302 | GTAGTTTCATGTGATTGT | 318 |
| | | GAAACTAC | | TACCCTG | |
| CAN0504ana2 | EG683r | AAGGAAATCAACTTTGGA | 304 | CTGAGATGATCCAAAGTT | 319 |
| | | TCATCTCAG | | GATTTCCTT | |
| CAN1539ana2 | EG588r | GAGTCTGCTGTACATTTG | 305 | CGTCCAAATGTACAGCA | 320 |
| | | GACG | | GACTC | |
| CAN0067ana2 | EG694r | TTAGACAAACCACACAAC | 308 | CTGTTTTCGGTTGTGTGG | 321 |
| | | CGAAAACAG | | TTTGTCTAA | |
| CAN0802ana2 | EG691r | TTTTGGTGTGTGGAAGG | 311 | CGTCAAGTTCCCTTCCAC | 322 |
| | | GAACTTGACG | | ACACCAAAA | |
| CAN0055ana5 | EG689r | CCAAAACTTAATCTAACC | 313 | GGTCGTTAGAGGTTAGA | 323 |
| | | TCTAACGACC | | TTAAGTTTTGG | |

TABLE 40
Multiplex 39

| | | Min and | Forward | Reverse |
|---|---|---|---|---|
| Marker | Label | Max | Primer | Primer |
| ANUCS304ana | 6FAM | 345-423 | EG496f_6FAM | EG497r |
| ANUCS501ana | 6FAM | 83-103 | EG426f_6FAM | EG425r |
| CAN0138ana2 | VIC | 180-200 | EG498f_VIC | EG471r |
| CAN0180ana | PET | 290-306 | EG486f_PET | EG487r |
| CAN0291ana | VIC | 329-345 | EG482f_VIC | EG483r |
| CAN0338-339ana | NED | 451-478 | EG503f_NED | EG504r |
| CAN0716ana | 6FAM | 232-264 | EG499f_6FAM | EG500r |
| CAN1539ana | VIC | 491-520 | EG507f_VIC | EG508r |
| CAN1832ana | NED | 127-133 | EG501f_NED | EG502r |
| H06-CANN2ana | PET | 151-157 | EG494f_PET | EG495r |

TABLE 41
Multiplex 40

| | | Min and | Forward | Reverse |
|---|---|---|---|---|
| Marker | Label | Max | Primer | Primer |
| ANUCS304ana | 6FAM | 345-423 | EG496f_6FAM | EG497r |
| ANUCS501ana | 6FAM | 83-103 | EG426f_6FAM | EG425r |
| CAN0138ana2 | VIC | 180-200 | EG498f_VIC | EG471r |
| CAN0291ana | VIC | 329-345 | EG482f_VIC | EG483r |
| CAN0338-339ana | NED | 451-478 | EG503f_NED | EG504r |
| CAN0509ana | 6FAM | 302-322 | EG432f_6FAM | EG431r |
| CAN0716ana | 6FAM | 232-264 | EG499f_6FAM | EG500r |
| CAN1539ana | VIC | 491-520 | EG507f_VIC | EG508r |
| CAN2550ana2 | VIC | 417-441 | EG434f_VIC | EG415r |
| H06-CANN2ana | PET | 151-157 | EG494f_PET | EG495r |

TABLE 42
Multiplex 41

| | | Min and | Forward | Reverse |
|---|---|---|---|---|
| Marker | Label | Max | Primer | Primer |
| ANUCS304ana | 6FAM | 345-423 | EG496f_6FAM | EG497r |
| ANUCS501ana | 6FAM | 83-103 | EG426f_6FAM | EG425r |
| CAN0180ana | PET | 290-306 | EG486f_PET | EG487r |
| CAN0291ana | VIC | 329-345 | EG482f_VIC | EG483r |
| E07-CANN1ana2 | NED | 263-287 | EG469f_NED | EG420r |

TABLE 43
Multiplex 42

| | | Min and | Forward | Reverse |
|---|---|---|---|---|
| Marker | Label | Max | Primer | Primer |
| CAN0138ana2 | VIC | 180-200 | EG498f_VIC | EG471r |
| CAN0716ana | 6FAM | 232-264 | EG499f_6FAM | EG500r |
| CAN1539ana | VIC | 491-520 | EG507f_VIC | EG508r |
| CAN1832ana | NED | 127-133 | EG501f_NED | EG502r |
| H06-CANN2ana | PET | 151-157 | EG494f_PET | EG495r |

TABLE 44
Multiplex 43

| | | Min and | Forward | Reverse |
|---|---|---|---|---|
| Marker | Label | Max | Primer | Primer |
| ANUCS501ana | 6FAM | 83-103 | EG426f_6FAM | EG425r |
| CAN1832ana | NED | 127-133 | EG501f_NED | EG502r |
| CAN0138ana2 | VIC | 180-200 | EG498f_VIC | EG471r |
| CAN0180ana | PET | 290-306 | EG486f_PET | EG487r |
| CAN0291ana | VIC | 329-345 | EG482f_VIC | EG483r |

TABLE 45
Multiplex 44

| | | Min and | Forward | Reverse |
|---|---|---|---|---|
| Marker | Label | Max | Primer | Primer |
| H06-CANN2ana | PET | 151-157 | EG494f_PET | EG495r |
| CAN0716ana | 6FAM | 232-264 | EG499f_6FAM | EG500r |
| E07-CANN1ana2 | NED | 263-287 | EG469f_NED | EG420r |
| ANUCS304ana | 6FAM | 345-423 | EG496f_6FAM | EG497r |
| CAN1539ana | VIC | 491-520 | EG507f_VIC | EG508r |

TABLE 46
Multiplex 45

| | | Min and | Forward | Reverse |
|---|---|---|---|---|
| Marker | Label | Max | Primer | Primer |
| ANUCS304ana | 6FAM | 345-423 | EG496f_6FAM | EG497r |
| ANUCS501ana | 6FAM | 83-103 | EG426f_6FAM | EG425r |
| CAN0138ana2 | VIC | 180-200 | EG498f_VIC | EG471r |
| CAN0180ana | PET | 290-306 | EG486f_PET | EG487r |
| CAN0291ana | VIC | 329-345 | EG482f_VIC | EG483r |

TABLE 47
Multiplex 46

| | | Min and | Forward | Reverse |
|---|---|---|---|---|
| Marker | Label | Max | Primer | Primer |
| CAN0716ana | 6FAM | 232-264 | EG499f_6FAM | EG500r |
| CAN1539ana | VIC | 491-520 | EG507f_VIC | EG508r |
| CAN1832ana | NED | 127-133 | EG501f_NED | EG502r |
| E07-CANN1ana2 | NED | 263-287 | EG469f_NED | EG420r |
| H06-CANN2ana | PET | 151-157 | EG494f_PET | EG495r |

TABLE 48
Multiplex 48

| | | Min and | Forward | Reverse |
|---|---|---|---|---|
| Marker | Label | Max | Primer | Primer |
| H06-CANN2ana | PET | 151-157 | EG494f_PET | EG495r |
| CAN0124ana | VIC | 160-193 | EG509f_VIC | EG510r |
| CAN0907ana | NED | 223-235 | EG515f_NED | EG516r |
| CAN0716ana | 6FAM | 232-264 | EG499f_6FAM | EG500r |
| CAN2846ana | PET | 281-308 | EG533f_PET | EG534r |
| CAN0291ana | VIC | 329-345 | EG482f_VIC | EG483r |
| CAN3008ana | 6FAM | 353-373 | EG531f_6FAM | EG532r |
| CAN0156ana | NED | 437-445 | EG513f_NED | EG514r |
| CAN2902ana | PET | 486-507 | EG529f_PET | EG530r |
| CAN1539ana | VIC | 491-520 | EG507f_VIC | EG508r |

TABLE 49
Multiplex 59

| | | Min and | Forward | Reverse |
|---|---|---|---|---|
| Marker | Label | Max | Primer | Primer |
| CAN0089ana | PET | 299-313 | EG517f_PET | EG518r |
| CAN0103ana | 6FAM | 234-246 | EG523f_6FAM | EG524r |
| CAN0156ana | NED | 437-445 | EG513f_NED | EG514r |
| CAN0271ana | NED | 180-280 | EG472f_NED | EG473r |
| CAN0509ana | 6FAM | 302-322 | EG432f_6FAM | EG431r |
| CAN0716ana | 6FAM | 232-264 | EG499f_6FAM | EG500r |
| CAN0802ana | VIC | 324-353 | EG521f_VIC | EG522r |
| CAN1539ana | VIC | 491-520 | EG507f_VIC | EG508r |
| CAN1832ana | NED | 127-133 | EG501f_NED | EG502r |
| CAN2913ana | PET | 130-180 | EG527f_PET | EG528r |
| CAN3008ana | 6FAM | 353-373 | EG531f_6FAM | EG532r |
| E07-CANN1ana2 | NED | 263-287 | EG469f_NED | EG420r |

TABLE 50
Multiplex 60

| | | Min and | Forward | Reverse |
|---|---|---|---|---|
| Marker | Label | Max | Primer | Primer |
| CAN0089ana | PET | 299-313 | EG517f_PET | EG518r |
| CAN0103ana | 6FAM | 234-246 | EG523f_6FAM | EG524r |
| CAN0156ana | NED | 437-445 | EG513f_NED | EG514r |
| CAN0509ana | 6FAM | 302-322 | EG432f_6FAM | EG431r |
| CAN0802ana | VIC | 324-353 | EG521f_VIC | EG522r |
| CAN0907ana | NED | 223-235 | EG515f_NED | EG516r |
| CAN1539ana | VIC | 491-520 | EG507f_VIC | EG508r |
| CAN1832ana | NED | 127-133 | EG501f_NED | EG502r |
| CAN2913ana | PET | 130-180 | EG527f_PET | EG528r |
| CAN3008ana | 6FAM | 353-373 | EG531f_6FAM | EG532r |
| E07-CANN1ana2 | NED | 263-287 | EG469f_NED | EG420r |

TABLE 51
Multiplex 64

| | | Min and | Forward | Reverse |
|---|---|---|---|---|
| Marker | Label | Max | Primer | Primer |
| CAN0089ana | PET | 299-313 | EG517f_PET | EG518r |
| CAN0103ana | 6FAM | 234-246 | EG523f_6FAM | EG524r |
| CAN0156ana | NED | 437-445 | EG513f_NED | EG514r |
| CAN0509ana | 6FAM | 302-322 | EG432f_6FAM | EG431r |
| CAN0802ana | VIC | 324-353 | EG521f_VIC | EG522r |
| CAN0907ana | NED | 223-235 | EG515f_NED | EG516r |
| CAN1539ana | VIC | 491-520 | EG507f_VIC | EG508r |
| CAN1832ana | NED | 127-133 | EG501f_NED | EG502r |
| CAN3008ana | 6FAM | 353-373 | EG531f_6FAM | EG532r |
| E07-CANN1ana2 | NED | 263-287 | EG469f_NED | EG420r |
| H06-CANN2ana | PET | 151-157 | EG494f_PET | EG495r |

TABLE 52
Multiplex 65

| | | Min and | Forward | Reverse |
|---|---|---|---|---|
| Marker | Label | Max | Primer | Primer |
| C11-CANN1ana2 | VIC | 187-211 | EG429f_VIC | EG537r |
| CAN0089ana | PET | 289-319 | EG517f_PET | EG518r |
| CAN0103ana | 6FAM | 234-246 | EG523f_6FAM | EG524r |
| CAN0156ana | NED | 437-445 | EG513f_NED | EG514r |
| CAN0509ana | 6FAM | 302-322 | EG432f_6FAM | EG431r |
| CAN0802ana | VIC | 324-353 | EG521f_VIC | EG522r |
| CAN0907ana | NED | 223-235 | EG515f_NED | EG516r |
| CAN1539ana | VIC | 491-520 | EG507f_VIC | EG508r |
| CAN1832ana | NED | 127-133 | EG501f_NED | EG502r |
| CAN3008ana | 6FAM | 353-373 | EG531f_6FAM | EG532r |
| E07-CANN1ana2 | NED | 263-287 | EG469f_NED | EG420r |
| H06-CANN2ana | PET | 151-157 | EG494f_PET | EG495r |

TABLE 53
Multiplex 66

| | | Min and | Forward | Reverse |
|---|---|---|---|---|
| Marker | Label | Max | Primer | Primer |
| C11-CANN1ana2 | VIC | 187-211 | 429f_VIC | 537r |
| CAN0055ana4 | 6FAM | 110-115 | 430f_6FAM | 538r |
| CAN0089ana | PET | 299-313 | 517f_PET | 518r |
| CAN0103ana | 6FAM | 234-246 | 523f_6FAM | 524r |
| CAN0156ana | NED | 437-445 | 513f_NED | 514r |
| CAN0509ana | 6FAM | 302-322 | 432f_6FAM | 431r |
| CAN0802ana | VIC | 324-353 | 521f_VIC | 522r |
| CAN0907ana | NED | 223-235 | 515f_NED | 516r |
| CAN1539ana | VIC | 491-520 | 507f_VIC | 508r |
| CAN1832ana | NED | 127-133 | 501f_NED | 502r |
| CAN3008ana | 6FAM | 353-373 | 531f_6FAM | 532r |
| E07-CANN1ana2 | NED | 263-287 | 469f_NED | 420r |
| H06-CANN2ana | VIC | 151-157 | 494f_VIC | 495r |

TABLE 54
Multiplex 67

| | | Min and | Forward | Reverse |
|---|---|---|---|---|
| Marker | Label | Max | Primer | Primer |
| C11-CANN1ana2 | VIC | 187-211 | 429f_VIC | 537r |
| CAN0055ana4 | 6FAM | 110-115 | 430f_6FAM | 538r |
| CAN0089ana | PET | 299-313 | 517f_PET | 518r |
| CAN0103ana | 6FAM | 234-246 | 523f_6FAM | 524r |
| CAN0156ana | NED | 437-445 | 513f_NED | 514r |
| CAN0509ana | 6FAM | 302-322 | 432f_6FAM | 431r |
| CAN0802ana | VIC | 324-353 | 521f_VIC | 522r |
| CAN0907ana | NED | 223-235 | 515f_NED | 516r |
| CAN1539ana | VIC | 491-520 | 507f_VIC | 508r |
| CAN1832ana | NED | 127-133 | 501f_NED | 502r |
| CAN2039ana | VIC | 372-408 | EG541f_VIC | 542r |
| CAN3008ana | 6FAM | 353-373 | 531f_6FAM | 532r |
| E07-CANN1ana2 | NED | 263-287 | 469f_NED | 420r |
| H06-CANN2ana | VIC | 151-157 | 494f_VIC | 495r |

TABLE 55
Multiplex 68

| | | Min and | Forward | Reverse |
|---|---|---|---|---|
| Marker | Label | Max | Primer | Primer |
| C11-CANN1ana2 | VIC | 187-211 | 429f_VIC | 537r |
| CAN0055ana4 | 6FAM | 110-115 | 430f_6FAM | 538r |
| CAN0057ana | VIC | 391-415 | EG543f_VIC | 544r |
| CAN0089ana | PET | 289-319 | 517f_PET | 518r |
| CAN0103ana | 6FAM | 234-246 | 523f_6FAM | 524r |
| CAN0156ana | NED | 437-445 | 513f_NED | 514r |
| CAN0509ana | 6FAM | 302-322 | 432f_6FAM | 431r |
| CAN0802ana | VIC | 324-353 | 521f_VIC | 522r |
| CAN0907ana | NED | 223-235 | 515f_NED | 516r |
| CAN1539ana | VIC | 491-520 | 507f_VIC | 508r |
| CAN1832ana | NED | 127-133 | 501f_NED | 502r |
| CAN3008ana | 6FAM | 353-373 | 531f_6FAM | 532r |
| E07-CANN1ana2 | NED | 263-287 | 469f_NED | 420r |
| H06-CANN2ana | VIC | 151-157 | 494f_VIC | 495r |

TABLE 56
Multiplex 70

| | | Min and | Forward | Reverse |
|---|---|---|---|---|
| Marker | Label | Max | Primer | Primer |
| C11-CANN1ana2 | VIC | 187-211 | 429f_VIC | 537r |
| CAN0055ana4 | 6FAM | 110-115 | 430f_6FAM | 538r |
| CAN0089ana | PET | 299-313 | 517f_PET | 518r |
| CAN0103ana | 6FAM | 234-246 | 523f_6FAM | 524r |
| CAN0156ana | NED | 437-445 | 513f_NED | 514r |
| CAN0509ana | 6FAM | 302-322 | 432f_6FAM | 431r |
| CAN0577ana | 6FAM | 474-490 | 550f_6FAM | 551r |
| CAN0802ana | VIC | 324-353 | 521f_VIC | 522r |
| CAN0907ana | NED | 223-235 | 515f_NED | 516r |
| CAN1539ana | VIC | 491-520 | 507f_VIC | 508r |
| CAN1832ana | NED | 127-133 | 501f_NED | 502r |
| CAN3008ana | 6FAM | 353-373 | 531f_6FAM | 532r |
| E07-CANN1ana2 | NED | 263-287 | 469f_NED | 420r |
| H06-CANN2ana | VIC | 151-157 | 494f_VIC | 495r |

TABLE 57
Multiplex 71

| | | Min and | Forward | Reverse |
|---|---|---|---|---|
| Marker | Label | Max | Primer | Primer |
| C11-CANN1ana2 | VIC | 187-211 | 429f_VIC | 537r |
| CAN0055ana4 | 6FAM | 110-115 | 430f_6FAM | 538r |
| CAN0089ana | PET | 299-313 | 517f_PET | 518r |
| CAN0103ana | 6FAM | 234-246 | 523f_6FAM | 524r |
| CAN0156ana | NED | 437-445 | 513f_NED | 514r |
| CAN0509ana | 6FAM | 302-322 | 432f_6FAM | 431r |
| CAN0802ana | VIC | 324-353 | 521f_VIC | 522r |
| CAN0907ana | NED | 223-235 | 515f_NED | 516r |
| CAN1409ana | 6FAM | 155-188 | 550f_6FAM | 551r |
| CAN1539ana | VIC | 491-520 | 507f_VIC | 508r |
| CAN1832ana | NED | 127-133 | 501f_NED | 502r |
| CAN3008ana | 6FAM | 353-373 | 531f_6FAM | 532r |
| E07-CANN1ana2 | NED | 263-287 | 469f_NED | 420r |
| H06-CANN2ana | VIC | 151-157 | 494f_VIC | 495r |

TABLE 58
Multiplex 72

| | | Min and | Forward | Reverse |
|---|---|---|---|---|
| Marker | Label | Max | Primer | Primer |
| C11-CANN1ana2 | VIC | 187-211 | 429f_VIC | 537r |
| CAN0055ana4 | 6FAM | 110-115 | 430f_6FAM | 538r |
| CAN0089ana | PET | 299-313 | 517f_PET | 518r |
| CAN0103ana | 6FAM | 234-246 | 523f_6FAM | 524r |
| CAN0156ana | NED | 437-445 | 513f_NED | 514r |
| CAN0509ana | 6FAM | 302-322 | 432f_6FAM | 431r |
| CAN0802ana | VIC | 324-353 | 521f_VIC | 522r |
| CAN0907ana | NED | 223-235 | 515f_NED | 516r |
| CAN1539ana | VIC | 491-520 | 507f_VIC | 508r |
| CAN1832ana | NED | 127-133 | 501f_NED | 502r |
| CAN2418ana | 6FAM | 450-489 | 546f_6FAM | 547r |
| CAN3008ana | 6FAM | 353-373 | 531f_6FAM | 532r |
| E07-CANN1ana2 | NED | 263-287 | 469f_NED | 420r |
| H06-CANN2ana | VIC | 156-162 | 494f_VIC | 495r |

TABLE 59
Multiplex 91

| | | Min and | Forward | Reverse |
|---|---|---|---|---|
| MPLX91 | Label | Max | Primers | Primers |
| ANUCS501ana2 | 6FAM | 137-157 | 426f_6FAM | 417r |
| C11-CANN1ana2 | VIC | 187-211 | 429f_VIC | 537r |
| CAN0055ana4 | 6FAM | 110-115 | 430f_6FAM | 538r |
| CAN0067ana | 6FAM | 503-555 | 657f_6FAM | 658r |
| CAN0089ana | PET | 299-313 | 517f_PET | 518r |
| CAN0097ana | PET | 140-167 | 685f_PET | 686r |
| CAN0202ana2 | 6FAM | 426-444 | 476f_6FAM | 591r |
| CAN0250ana | 6FAM | 404-422 | 581f_6FAM | 582r |
| CAN0504ana2 | NED | 133-195 | 602f_NED | 683r |
| CAN0509ana | 6FAM | 302-322 | 432f_6FAM | 431r |
| CAN0577ana | 6FAM | 474-490 | 550f_6FAM | 551r |
| CAN0716ana | 6FAM | 240-264 | 499f_6FAM | 500r |
| CAN0717ana | NED | 243-252 | 433f_NED | 424r |
| CAN0802ana | VIC | 324-356 | 521f_VIC | 522r |
| CAN0907ana | NED | 223-235 | 515f_NED | 516r |
| CAN1539ana2 | VIC | 269-298 | 507f_VIC | 588r |
| CAN3008ana | 6FAM | 353-373 | 531f_6FAM | 532r |
| E07-CANN1ana4 | NED | 494-521 | 587f_NED | 227r |

TABLE 60
Multiplex 91b

| | | Min and | Forward | Reverse |
|---|---|---|---|---|
| MPLX91b | Label | Max | Primers | Primers |
| ANUCS501ana2 | 6FAM | 137-157 | 426f_6FAM | 417r |
| C11-CANN1ana2 | VIC | 184-214 | 429f_VIC | 537r |
| CAN0055ana5 | 6FAM | 107-118 | 688f_6FAM | 689r |
| CAN0067ana2 | 6FAM | 544-567 | 693f_6FAM | 694r |
| CAN0089ana | PET | 299-313 | 517f_PET | 518r |
| CAN0097ana | PET | 157-178 | 685f_PET | 686r |
| CAN0202ana3 | 6FAM | 434-457 | 692f_6FAM | 591r |
| CAN0250ana | 6FAM | 403-424 | 581f_6FAM | 582r |
| CAN0504ana2 | NED | 170-197 | 602f_NED | 683r |
| CAN0509ana | 6FAM | 302-325 | 432f_6FAM | 431r |
| CAN0577ana | 6FAM | 471-493 | 550f_6FAM | 551r |
| CAN0716ana | 6FAM | 240-264 | 499f_6FAM | 500r |
| CAN0717ana | NED | 243-252 | 433f_NED | 424r |
| CAN0802ana2 | VIC | 321-359 | 690f_VIC | 691r |
| CAN0907ana | NED | 223-235 | 515f_NED | 516r |
| CAN1539ana2 | VIC | 279-303 | 507f_VIC | 588r |
| CAN3008ana | 6FAM | 353-373 | 531f_6FAM | 532r |
| E07-CANN1ana4 | NED | 479-512 | 587f_NED | 227r |

### REFERENCES

Barcaccia, G., Palumbo, F., Scariolo, F., Vannozzi, A., Bohn, M., & Bona, S. (2020). Potentials and Challenges of Genomics for Breeding Cannabis Cultivars. In Frontiers in Plant Science (Vol. 11, p. 1472). Frontiers Media S. A.
Garcia, C., Guichoux, E., & Hampe, A. (2018). A comparative analysis between SNPs and SSRs to investigate genetic variation in a juniper species (Juniperus phoenicea ssp. turbinate). Tree Genetics & Genomes 2018 14:6, 14(6), 1-9.
Guichoux, E., Lagache, L., Wagner, S., Chaumeil, P., Léger, P., Lepais, O., Lepoittevin, C., Malausa, T., Revardel, E., Salin, F., & Petit, R. J. (2011). Current trends in microsatellite genotyping. Molecular Ecology Resources, 11(4), 591-611.

1-176. (canceled)

177. A method for constructing a genetic profile for a Cannabis plant, the method comprising testing a deoxyribonucleic acid (DNA) sample from the plant to determine the length of at least two polymorphic target sequences, thereby constructing the genetic profile of the plant, wherein each polymorphic target sequence comprises a simple sequence repeat (SSR) sequence, wherein the at least two polymorphic target sequences comprise two or more of:
a target sequence amplifiable by a primer pair having the sequences of SEQ ID NO: 1 and SEQ ID NO: 2;
a target sequence amplifiable by a primer pair having the sequences of SEQ ID NO: 7 and SEQ ID NO: 8;
a target sequence amplifiable by a primer pair having the sequences of SEQ ID NO: 17 and SEQ ID NO: 18;
a target sequence amplifiable by a primer pair having the sequences of SEQ ID NO: 19 and SEQ ID NO: 20; and
a target sequence amplifiable by a primer pair having the sequences of SEQ ID NO: 23 and SEQ ID NO: 24;
wherein testing the DNA sample from the plant to determine the length of at least two polymorphic target sequences comprises: amplifying the DNA sample in at least one multiplex reaction with a plurality of primers that specifically hybridize to the at least two polymorphic target sequences to generate amplification products corresponding to the target sequences; and determining the length of each amplification product of the at least two polymorphic target sequences,
wherein the at least one multiplex reaction comprises:
a primer pair having the sequences of SEQ ID NO: 1 and SEQ ID NO: 2;
a primer pair having the sequences of SEQ ID NO: 7 and SEQ ID NO: 8;
a primer pair having the sequences of SEQ ID NO: 17 and SEQ ID NO: 18;
a primer pair having the sequences of SEQ ID NO: 19 and SEQ ID NO: 20; and
a primer pair having the sequences of SEQ ID NO: 23 and SEQ ID NO: 24.

178. The method of claim 177, wherein the sizes of the amplification products indicate the length of the at least two polymorphic target sequences.

179. The method of claim 177 wherein at least one of each pair of primers comprises a tag sequence.

180. The method of claim 177, wherein the DNA sample is amplified by polymerase chain reaction (PCR).

181. The method of claim 177, wherein the DNA sample is extracted from seed tissue, leaf tissue, stem tissue, root tissue, flower tissue, or any cell in the plant.

182. The method of claim 177, wherein the Cannabis plant is a Cannabis sativa plant, a Cannabis indica plant, or a Cannabis ruderalis plant.

183. The method of claim 177, wherein the lengths of the at least two polymorphic target sequences indicate the provenance of the plant.

184. The method of claim 177, wherein the genetic profile indicates the variety of the plant.

185. The method of claim 177, wherein the genetic profile indicates the parentage of the plant.

186. A method of producing a Cannabis plant with a desired genetic profile, the method comprising:
selecting a first parent having a first parent genetic profile constructed according to a method as defined in claim 177;
selecting a second parent having a second parent genetic profile constructed according to a method as defined in claim 177;
performing a cross of the first parent with the second parent;
constructing genetic profiles of a plurality of progeny plants of the cross according to a method as defined in claim 177; and
identifying a progeny plant from the cross that has the desired genetic profile.

187. A Cannabis plant or plant cell generated according to the method defined in claim claim 186.

188. The plant or plant cell of claim 187, wherein the plant or plant cell is a seed or seed cell.

189. Seed produced by the Cannabis plant or plant cell as defined in claim 187.

190. Plant material of the Cannabis plant or plant cell as defined in claim 187.

191. A set of primers for constructing a genetic profile for a Cannabis plant, the set comprising a plurality of primer pairs for specifically amplifying at least five polymorphic target sequences, wherein the at least five polymorphic target sequences comprise:
a target sequence flanked by SEQ ID NO: 1 on the 5′ end and SEQ ID NO: 101 on the 3′ end;
a target sequence flanked by SEQ ID NO: 7 on the 5′ end and SEQ ID NO: 104 on the 3′ end;
a target sequence flanked by SEQ ID NO: 17 on the 5′ end and SEQ ID NO: 109 on the 3′ end;
a target sequence flanked by SEQ ID NO: 19 on the 5′ end and SEQ ID NO: 110 on the 3′ end; and
a target sequence flanked by SEQ ID NO: 23 on the 5′ end and SEQ ID NO: 112 on the 3′ end.

192. The set of primers of claim 191, wherein at least one primer of each primer pair comprises sequences comprising a primer tag, a capture tag, a sequencing tag, a unique molecular identifier tag, or a combination thereof.

193. A kit for constructing a genetic profile for a Cannabis plant, the kit comprising a set of primers as defined in claim 191.

194. The kit of claim 193, further comprising at least one reagent for an amplification reaction.

195. The kit of claim 194, wherein the reagent is an amplification buffer.

196. The kit of claim 194, wherein the amplification reaction is a polymerase chain reaction (PCR) amplification.
